SEQUENCE LISTING (92 sequences)

SEQ ID NO: 1 - DNA, 31 bp, Artificial Sequence
  Completely Synthetic DNA Sequence
ctgaggagtcagatatcagctcaatctccat

SEQ ID NO: 2 - DNA, 27 bp, Artificial Sequence
  Completely Synthetic DNA Sequence
tccggctcgtatgttgtgtggaattgt

SEQ ID NO: 3 - DNA, 32 bp, Artificial Sequence
  Completely Synthetic DNA Sequence
ctggatgtttgatgggttcagtttcagctgga

SEQ ID NO: 4 - DNA, 29 bp, Artificial Sequence
  Completely Synthetic DNA Sequence
ggcaatagtcgcgagaatccttaaaccat

SEQ ID NO: 5 - DNA, 28 bp, Artificial Sequence
  Completely Synthetic DNA Sequence
cctcgtaaagatctgcggtttgcaaagt

SEQ ID NO: 6 - DNA, 27 bp, Artificial Sequence
  Completely Synthetic DNA Sequence
cctcccactggaaccgatgatatggaa

SEQ ID NO: 7 - DNA, 33 bp, Artificial Sequence
  Completely Synthetic DNA Sequence
gatgcgaagttaagtgcgcagaaagtaatatca

SEQ ID NO: 8 - DNA, 33 bp, Artificial Sequence
  Completely Synthetic DNA Sequence
cgtgtgtaccttgaaacgtcaatgatactttga

SEQ ID NO: 9 - DNA, 30 bp, Artificial Sequence
  Completely Synthetic DNA Sequence
cagactaagactgcttctccacctgctaag

SEQ ID NO: 10 - DNA, 32 bp, Artificial Sequence
  Completely Synthetic DNA Sequence
caacagtagaaccagaagcctcgtaagtacag

SEQ ID NO: 11 - DNA, 2577 bp, Artificial Sequence
  Completely Synthetic DNA Sequence
atgggtaaaagaaagggaaactccttgggagattctggttctgctgctactgcttccagagaggcttctgctcaagctgaagatgctgcttcccagactaagactgcttctccacctgctaaggttatcttgttgccaaagactttgactgacgagaaggacttcatcggtatcttcccatttccattctggccagttcacttcgttttgactgttgttgctttgttcgttttggctgcttcctgtttccaggctttcactgttagaatgatctccgttcaaatctacggttacttgatccacgaatttgacccatggttcaactacagagctgctgagtacatgtctactcacggatggagtgcttttttctcctggttcgattacatgtcctggtatccattgggtagaccagttggttctactacttacccaggattgcagttgactgctgttgctatccatagagctttggctgctgctggaatgccaatgtccttgaacaatgtttgtgttttgatgccagcttggtttggtgctatcgctactgctactttggctttctgtacttacgaggcttctggttctactgttgctgctgctgcagctgctttgtccttctccattatccctgctcacttgatgagatccatggctggtgagttcgacaacgagtgtattgctgttgctgctatgttgttgactttctactgttgggttcgttccttgagaactagatcctcctggccaatcggtgttttgacaggtgttgcttacggttacatggctgctgcttggggaggttacatcttcgttttgaacatggttgctatgcacgctggtatctcttctatggttgactgggctagaaacacttacaacccatccttgttgagagcttacactttgttctacgttgttggtactgctatcgctgtttgtgttccaccagttggaatgtctccattcaagtccttggagcagttgggagctttgttggttttggttttcttgtgtggattgcaagtttgtgaggttttgagagctagagctggtgttgaagttagatccagagctaatttcaagatcagagttagagttttctccgttatggctggtgttgctgctttggctatctctgttttggctccaactggttactttggtccattgtctgttagagttagagctttgtttgttgagcacactagaactggtaacccattggttgactccgttgctgaacatcaaccagcttctccagaggctatgtgggctttcttgcatgtttgtggtgttacttggggattgggttccattgttttggctgtttccactttcgttcactactccccatctaaggttttctggttgttgaactccggtgctgtttactacttctccactagaatggctagattgttgttgttgtccggtccagctgcttgtttgtccactggtatcttcgttggtactatcttggaggctgctgttcaattgtctttctgggactccgatgctactaaggctaagaagcagcaaaagcaggctcaaagacaccaaagaggtgctggtaaaggttctggtagagatgacgctaagaacgctactactgctagagctttctgtgacgttttcgctggttcttctttggcttggggtcacagaatggttttgtccattgctatgtgggctttggttactactactgctgtttccttcttctcctccgaatttgcttctcactccactaagttcgctgaacaatcctccaacccaatgatcgttttcgctgctgttgttcagaacagagctactggaaagccaatgaacttgttggttgacgactacttgaaggcttacgagtggttgagagactctactccagaggacgctagagttttggcttggtgggactacggttaccaaatcactggtatcggtaacagaacttccttggctgatggtaacacttggaaccacgagcacattgctactatcggaaagatgttgacttccccagttgttgaagctcactcccttgttagacacatggctgactacgttttgatttgggctggtcaatctggtgacttgatgaagtctccacacatggctagaatcggtaactctgtttaccacgacatttgtccagatgacccattgtgtcagcaattcggtttccacagaaacgattactccagaccaactccaatgatgagagcttccttgttgtacaacttgcacgaggctggaaaaagaaagggtgttaaggttaacccatctttgttccaagaggtttactcctccaagtacggacttgttagaatcttcaaggttatgaacgtttccgctgagtctaagaagtgggttgcagacccagctaacagagtttgtcacccacctggttcttggatttgtcctggtcaatacccacctgctaaagaaatccaagagatgttggctcacagagttccattcgaccaggttacaaacgctgacagaaagaacaatgttggttcctaccaagaggaatacatgagaagaatgagagagtccgagaacagaagataatag

SEQ ID NO: 12 - protein, 857 aa, Artificial Sequence
  Completely Synthetic Amino Acid Sequence
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVVEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRR

SEQ ID NO: 13 - DNA, 57 bp, Artificial Sequence
  Completely Synthetic DNA Sequence
atgagattcccatccatcttcactgctgttttgttcgctgcttcttctgctttggct

SEQ ID NO: 14 - protein, 19 aa, Artificial Sequence
  Completely Synthetic Amino Acid Sequence
MRFPSIFTAVLFAASSALA

SEQ ID NO: 15 - DNA, 1350 bp, Artificial Sequence
  Completely Synthetic DNA Sequence
gaggttcagttggttgaatctggaggaggattggttcaacctggtggttctttgagattgtcctgtgctgcttccggtttcaacatcaaggacacttacatccactgggttagacaagctccaggaaagggattggagtgggttgctagaatctacccaactaacggttacacaagatacgctgactccgttaagggaagattcactatctctgctgacacttccaagaacactgcttacttgcagatgaactccttgagagctgaggatactgctgtttactactgttccagatggggtggtgatggtttctacgctatggactactggggtcaaggaactttggttactgtttcctccgcttctactaagggaccatctgttttcccattggctccatcttctaagtctacttccggtggtactgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctggttaa

SEQ ID NO: 16 - protein, 449 aa, Artificial Sequence
  Completely Synthetic Amino Acid Sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - DNA, 645 bp, Artificial Sequence
  Completely Synthetic DNA Sequence
gacatccaaatgactcaatccccatcttctttgtctgcttccgttggtgacagagttactatcacttgtagagcttcccaggacgttaatactgctgttgcttggtatcaacagaagccaggaaaggctccaaagttgttgatctactccgcttccttcttgtactctggtgttccatccagattctctggttccagatccggtactgacttcactttgactatctcctccttgcaaccagaagatttcgctacttactactgtcagcagcactacactactccaccaactttcggacagggtactaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 18 - protein, 214 aa, Artificial Sequence
  Completely Synthetic Amino Acid Sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 1353 bp, Artificial Sequence
  Completely Synthetic DNA Sequence
gaggtccaattggttgaatctggtggaggtttggtccaaccaggtggatctctgagactttcttgtgctgcctctggtttcaacattaaggatacttacatccactgggttagacaggctccaggtaagggtttggagtgggttgctagaatctacccaaccaacggttacaccagatacgctgattccgttaagggtagattcaccatttccgctgacacttccaagaacactgcttacttgcaaatgaactctttgagagctgaggacactgccgtctactactgttccagatggggtggtgacggtttctacgccatggactactggggtcaaggtaccttggttactgtctcttccgcttctactaagggaccatccgtttttccattggctccatcctctaagtctacttccggtggtactgctgctttgggatgtttggttaaggactacttcccagagcctgttactgtttcttggaactccggtgctttgacttctggtgttcacactttcccagctgttttgcaatcttccggtttgtactccttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaaaggattctacccatccgacattgctgttgagtgggaatctaacggtcaaccagagaacaactacaagactactccaccagttttggattctgacggttccttcttcttgtactccaagttgactgttgacaagtccagatggcaacagggtaacgttttctcctgttccgttatgcatgaggctttgcacaaccactacactcaaaagtccttgtctttgtccccaggtaagtaa

SEQ ID NO: 20 - protein, 450 aa, Artificial Sequence
  Completely Synthetic Amino Acid Sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 60 bp, Artificial Sequence
  Completely Synthetic DNA Sequence
atggttgcttggtggtccttgttcttgtacggattgcaagttgctgctccagctttggct

SEQ ID NO: 22 - protein, 497 aa, Artificial Sequence
  Completely Synthetic Amino Acid Sequence
RAGSPNPTRAAAVKAAFQTSWNAYHHFAFPHDDLHPVSNSFDDERNGWGSSAIDGLDTAILMGDADIVNTILQYVPQINFTTTAVANQGISVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLAADSTIAHLASHPSTRKDLTFLSSYNGQSTSPNSGHLASFAGGNFILGGILLNEQKYIDFGIKLASSYFATYNQTASGIGPEGFAWVDSVTGAGGSPPSSQSGFYSSAGFWVTAPYYILRPETLESLYYAYRVTGDSKWQDLAWEAFSAIEDACRAGSAYSSINDVTQANGGGASDDMESFWFAEALKYAYLIFAEESDVQVQANGGNKFVFNTEAHPFSIRSSSRRGGHLA

SEQ ID NO: 23 - DNA, 934 bp, Artificial Sequence
  Completely Synthetic DNA Sequence
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 24 - DNA, 293 bp, Artificial Sequence
  Completely Synthetic DNA Sequence
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 25 - DNA, 600 bp, Artificial Sequence
  Completely Synthetic DNA Sequence
gttcttcgcttggtcttgtatctccttacactgtatcttcccatttgcgtttaggtggttatcaaaaactaaaaggaaaaatttcagatgtttatctctaaggttttttctttttacagtataacacgtgatgcgtcacgtggtactagattacgtaagttattttggtccggtgggtaagtgggtaagaatagaaagcatgaaggtttacaaaaacgcagtcacgaattattgctacttcgagcttggaaccaccccaaagattatattgtactgatgcactaccttctcgattttgctcctccaagaacctacgaaaaacatttcttgagccttttcaacctagactacacatcaagttatttaaggtatgttccgttaacatgtaagaaaaggagaggatagatcgtttatggggtacgtcgcctgattcaagcgtgaccattcgaagaataggccttcgaaagctgaataaagcaaatgtcagttgcgattggtatgctgacaaattagcataaaaagcaatagactttctaaccacctgtttttttccttttactttatttatattttgccaccgtactaacaagttcagacaaa

SEQ ID NO: 26 - DNA, 486 bp, Artificial Sequence
  Completely Synthetic DNA Sequence
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 27 - DNA, 600 bp, Artificial Sequence
  Completely Synthetic DNA Sequence
ttaaggtttggaacaacactaaactaccttgcggtactaccattgacactacacatccttaattccaatcctgtctggcctccttcaccttttaaccatcttgcccattccaactcgtgtcagattgcgtatcaagtgaaaaaaaaaaaattttaaatctttaacccaatcaggtaataactgtcgcctcttttatctgccgcactgcatgaggtgtccccttagtgggaaagagtactgagccaaccctggaggacagcaagggaaaaatacctacaacttgcttcataatggtcgtaaaaacaatccttgtcggatataagtgttgtagactgtcccttatcctctgcgatgttcttcctctcaaagtttgcgatttctctctatcagaattgccatcaagagactcaggactaatttcgcagtcccacacgcactcgtacatgattggctgaaatttccctaaagaatttctttttcacgaaaattttttttttacacaagattttcagcagatataaaatggagagcaggacctccgctgtgactcttcttttttttcttttattctcactacatacattttagttattcgccaac

SEQ ID NO: 28 - DNA, 301 bp, Artificial Sequence
  Completely Synthetic DNA Sequence
attgcttgaagctttaatttattttattaacataataataatacaagcatgatatatttgtattttgttcgttaacattgatgttttcttcatttactgttattgtttgtaactttgatcgatttatcttttctactttactgtaatatggctggcgggtgagccttgaactccctgtattactttaccttgctattacttaatctattgactagcagcgacctcttcaaccgaagggcaagtacacagcaagttcatgtctccgtaagtgtcatcaaccctggaaacagtgggccatgtc

SEQ ID NO: 29 - DNA, 376 bp, Artificial Sequence
  Completely Synthetic DNA Sequence
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtag

SEQ ID NO: 30 - DNA, 672 bp, Artificial Sequence
  Completely Synthetic DNA Sequence
gcggaaacggcagtaaacaatggagcttcattagtgggtgttattatggtccctggccgggaacgaacggtgaaacaagaggttgcgagggaaatttcgcagatggtgcgggaaaagagaatttcaaagggctcaaaatacttggattccagacaactgaggaaagagtgggacgactgtcctctggaagactggtttgagtacaacgtgaaagaaataaacagcagtggtccatttttagttggagtttttcgtaatcaaagtatagatgaaatccagcaagctatccacactcatggtttggatttcgtccaactacatgggtctgaggattttgattcgtatatacgcaatatcccagttcctgtgattaccagatacacagataatgccgtcgatggtcttaccggagaagacctcgctataaatagggccctggtgctactggacagcgagcaaggaggtgaaggaaaaaccatcgattgggctcgtgcacaaaaatttggagaacgtagaggaaaatatttactagccggaggtttgacacctgataatgttgctcatgctcgatctcatactggctgtattggtgttgacgtctctggtggggtagaaacaaatgcctcaaaagatatggacaagatcacacaatttatcagaaacgctacataa

SEQ ID NO: 31 - DNA, 834 bp, Artificial Sequence
  Completely Synthetic DNA Sequence
aagtcaattaaatacacgcttgaaaggacattacatagctttcgatttaagcagaaccagaaatgtagaaccacttgtcaatagattggtcaatcttagcaggagcggctgggctagcagttggaacagcagaggttgctgaaggtgagaaggatggagtggattgcaaagtggtgttggttaagtcaatctcaccagggctggttttgccaaaaatcaacttctcccaggcttcacggcattcttgaatgacctcttctgcatacttcttgttcttgcattcaccagagaaagcaaactggttctcaggttttccatcagggatcttgtaaattctgaaccattcgttggtagctctcaacaagcccggcatgtgcttttcaacatcctcgatgtcattgagcttaggagccaatgggtcgttgatgtcgatgacgatgaccttccagtcagtctctccctcatccaacaaagccataacaccgaggaccttgacttgcttgacctgtccagtgtaacctacggcttcaccaatttcgcaaacgtccaatggatcattgtcacccttggccttggtctctggatgagtgacgttagggtcttcccatgtctgagggaaggcaccgtagttgtgaatgtatccgtggtgagggaaacagttacgaacgaaacgaagttttcccttctttgtgtcctgaagaattgggttcagtttctcctccttggaaatctccaacttggcgttggtccaacgggggacttcaacaaccatgttgagaaccttcttggattcgtcagcataaagtgggatgtcgtggaaaggagatacgactt

SEQ ID NO: 32 - DNA, 1215 bp, Artificial Sequence
  Completely Synthetic DNA Sequence
atgtcagaagatcaaaaaagtgaaaattccgtaccttctaaggttaatatggtgaatcgcaccgatatactgactacgatcaagtcattgtcatggcttgacttgatgttgccatttactataattctctccataatcattgcagtaataatttctgtctatgtgccttcttcccgtcacacttttgacgctgaaggtcatcccaatctaatgggagtgtccattcctttgactgttggtatgattgtaatgatgattcccccgatctgcaaagtttcctgggagtctattcacaagtacttctacaggagctatataaggaagcaactagccctctcgttatttttgaattgggtcatcggtcctttgttgatgacagcattggcgtggatggcgctattcgattataaggaataccgtcaaggcattattatgatcggagtagctagatgcattgccatggtgctaatttggaatcagattgctggaggagacaatgatctctgcgtcgtgcttgttattacaaactcgcttttacagatggtattatatgcaccattgcagatattttactgttatgttatttctcatgaccacctgaatacttcaaatagggtattattcgaagaggttgcaaagtctgtcggagtttttctcggcataccactgggaattggcattatcatacgtttgggaagtcttaccatagctggtaaaagtaattatgaaaaatacattttgagatttatttctccatgggcaatgatcggatttcattacactttatttgttatttttattagtagaggttatcaatttatccacgaaattggttctgcaatattgtgctttgtcccattggtgctttacttctttattgcatggtttttgaccttcgcattaatgaggtacttatcaatatctaggagtgatacacaaagagaatgtagctgtgaccaagaactacttttaaagagggtctggggaagaaagtcttgtgaagctagcttttctattacgatgacgcaatgtttcactatggcttcaaataattttgaactatccctggcaattgctatttccttatatggtaacaatagcaagcaagcaatagctgcaacatttgggccgttgctagaagttccaattttattgattttggcaatagtcgcgagaatccttaaaccatattatatatggaacaatagaaattaa

SEQ ID NO: 33 - DNA, 1144 bp, Artificial Sequence
  Completely Synthetic DNA Sequence
caaatgcaagaggacattagaaatgtgtttggtaagaacatgaagccggaggcatacaaacgattcacagatttgaaggaggaaaacaaactgcatccaccggaagtgccagcagccgtgtatgccaaccttgctctcaaaggcattcctacggatctgagtgggaaatatctgagattcacagacccactattggaacagtaccaaacctagtttggccgatccatgattatgtaatgcatatagtttttgtcgatgctcacccgtttcgagtctgtctcgtatcgtcttacgtataagttcaagcatgtttaccaggtctgttagaaactcctttgtgagggcaggacctattcgtctcggtcccgttgtttctaagagactgtacagccaagcgcagaatggtggcattaaccataagaggattctgatcggacttggtctattggctattggaaccaccctttacgggacaaccaaccctaccaagactcctattgcatttgtggaaccagccacggaaagagcgtttaaggacggagacgtctctgtgatttttgttctcggaggtccaggagctggaaaaggtacccaatgtgccaaactagtgagtaattacggatttgttcacctgtcagctggagacttgttacgtgcagaacagaagagggaggggtctaagtatggagagatgatttcccagtatatcagagatggactgatagtacctcaagaggtcaccattgcgctcttggagcaggccatgaaggaaaacttcgagaaagggaagacacggttcttgattgatggattccctcgtaagatggaccaggccaaaacttttgaggaaaaagtcgcaaagtccaaggtgacacttttctttgattgtcccgaatcagtgctccttgagagattacttaaaagaggacagacaagcggaagagaggatgataatgcggagagtatcaaaaaaagattcaaaacattcgtggaaacttcgatgcctgtggtggactatttcgggaagcaaggacgcgttttgaaggtatcttgtgaccaccctgtggatcaagtgtattcacaggttgtgtcggtgctaaaagagaaggggatctttgccgataacgagacggagaataaataa

SEQ ID NO: 34 - DNA, 582 bp, Artificial Sequence
  Completely Synthetic DNA Sequence
atgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactg

SEQ ID NO: 35 - DNA, 375 bp, Artificial Sequence
  Completely Synthetic DNA Sequence
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 36 - DNA, 260 bp, Artificial Sequence
  Completely Synthetic DNA Sequence
tcaagaggatgtcagaatgccatttgcctgagagatgcaggcttcattttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 37 - DNA, 427 bp, Artificial Sequence
  Completely Synthetic DNA Sequence
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 38 - DNA, 3029 bp, Artificial Sequence
  Completely Synthetic DNA Sequence
aggcctcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacggtcgac

SEQ ID NO: 39 - DNA, 898 bp, Artificial Sequence
  Completely Synthetic DNA Sequence
atcggcctttgttgatgcaagttttacgtggatcatggactaaggagttttatttggaccaagttcatcgtcctagacattacggaaagggttctgctcctctttttggaaactttttggaacctctgagtatgacagcttggtggattgtacccatggtatggcttcctgtgaatttctattttttctacattggattcaccaatcaaaacaaattagtcgccatggctttttggcttttgggtctatttgtttggaccttcttggaatatgctttgcatagatttttgttccacttggactactatcttccagagaatcaaattgcatttaccattcatttcttattgcatgggatacaccactatttaccaatggataaatacagattggtgatgccacctacacttttcattgtactttgctacccaatcaagacgctcgtcttttctgttctaccatattacatggcttgttctggatttgcaggtggattcctgggctatatcatgtatgatgtcactcattacgttctgcatcactccaagctgcctcgttatttccaagagttgaagaaatatcatttggaacatcactacaagaattacgagttaggctttggtgtcacttccaaattctgggacaaagtctttgggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactcgaggggtaccaaaaaaggtttttggatgctgcagtggcttcgc

SEQ ID NO: 40 - DNA, 1060 bp, Artificial Sequence
  Completely Synthetic DNA Sequence
ggtcttttcaacaaagctccattagtgagtcagctggctgaatcttatgcacaggccatcattaacagcaacctggagatagacgttgtatttggaccagcttataaaggtattcctttggctgctattaccgtgttgaagttgtacgagctcggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggtagtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcgggtggtgcgggctcaggttccctttcataaacagatttagtacttggatgcttaatagtgaatggcgaatgcaaaggaacaatttcgttcatctttaaccctttcactcggggtacacgttctggaatgtacccgccctgttgcaactcaggtggaccgggcaattcttgaactttctgtaacgttgttggatgttcaaccagaaattgtcctaccaactgtattagtttccttttggtcttatattgttcatcgagatacttcccactctccttgatagccactctcactcttcctggattaccaaaatcttgaggatgagtcttttcaggctccaggatgcaaggtatatccaagtacctgcaagcatctaatattgtctttgccagggggttctccacaccatactccttttggcgcatgc

SEQ ID NO: 41 - DNA, 957 bp, Artificial Sequence
  Completely Synthetic DNA Sequence
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 42 - DNA, 709 bp, Artificial Sequence
  Completely Synthetic DNA Sequence
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 43 - DNA, 2875 bp, Artificial Sequence
  Completely Synthetic DNA Sequence
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 44 - DNA, 997 bp, Artificial Sequence
  Completely Synthetic DNA Sequence
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 45 - DNA, 2159 bp, Artificial Sequence
  Completely Synthetic DNA Sequence
aaacgtaacgcctggcactctattttctcaaacttctgggacggaagagctaaatattgtgttgcttgaacaaacccaaaaaaacaaaaaaatgaacaaactaaaactacacctaaataaaccgtgtgtaaaacgtagtaccatattactagaaaagatcacaagtgtatcacacatgtgcatctcatattacatcttttatccaatccattctctctatcccgtctgttcctgtcagattctttttccataaaaagaagaagaccccgaatctcaccggtacaatgcaaaactgctgaaaaaaaaagaaagttcactggatacgggaacagtgccagtaggcttcaccacatggacaaaacaattgacgataaaataagcaggtgagcttctttttcaagtcacgatccctttatgtctcagaaacaatatatacaagctaaacccttttgaaccagttctctcttcatagttatgttcacataaattgcgggaacaagactccgctggctgtcaggtacacgttgtaacgttttcgtccgcccaattattagcacaacattggcaaaaagaaaaactgctcgttttctctacaggtaaattacaatttttttcagtaattttcgctgaaaaatttaaagggcaggaaaaaaagacgatctcgactttgcatagatgcaagaactgtggtcaaaacttgaaatagtaattttgctgtgcgtgaactaataaatatatatatatatatatatatatatttgtgtattttgtatatgtaattgtgcacgtcttggctattggatataagattttcgcgggttgatgacatagagcgtgtactactgtaatagttgtatattcaaaagctgctgcgtggagaaagactaaaatagataaaaagcacacattttgacttcggtaccgtcaacttagtgggacagtcttttatatttggtgtaagctcatttctggtactattcgaaacagaacagtgttttctgtattaccgtccaatcgtttgtcatgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagattttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtacgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagcaaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctgaaacaatccacgtctgtatgatactcgtttcagaatttttttgattttctgccggatatggtttctcatctttacaatcgcattcttaattataccagaacgtaattcaatgatcccagtgactcgtaactcttatatgtcaatttaagc

SEQ ID NO: 46 - DNA, 870 bp, Artificial Sequence
  Completely Synthetic DNA Sequence
ggccgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagcttcaacgacatggaggtcgactcctatgtcaccaacatctacgacaatgctccagtgctaggatgtacggatttgtcttatcatggattgttgaaagtcaccccaaagcatgacttagcttgcgatttggagttcataagagctcagattttggacattgacgtttactccgccataaaagacttagaagataaagccttgactgtaaaacaaaaggttgaaaaacactggtttacgttttatggtagttcagtctttctgcccgaacacgatgtgcattacctggttagacgagtcatcttttcggctgaaggaaaggcgaactctccagtaacatc

SEQ ID NO: 47 - DNA, 1733 bp, Artificial Sequence
  Completely Synthetic DNA Sequence
ccatatgatgggtgtttgctcactcgtatggatcaaaattccatggtttcttctgtacaacttgtacacttatttggacttttctaacggtttttctggtgatttgagaagtccttattttggtgttcgcagcttatccgtgattgaaccatcagaaatactgcagctcgttatctagtttcagaatgtgttgtagaatacaatcaattctgagtctagtttgggtgggtcttggcgacgggaccgttatatgcatctatgcagtgttaaggtacatagaatgaaaatgtaggggttaatcgaaagcatcgttaatttcagtagaacgtagttctattccctacccaaataatttgccaagaatgcttcgtatccacatacgcagtggacgtagcaaatttcactttggactgtgacctcaagtcgttatcttctacttggacattgatggtcattacgtaatccacaaagaattggatagcctctcgttttatctagtgcacagcctaatagcacttaagtaagagcaatggacaaatttgcatagacattgagctagatacgtaactcagatcttgttcactcatggtgtactcgaagtactgctggaaccgttacctcttatcatttcgctactggctcgtgaaactactggatgaaaaaaaaaaaagagctgaaagcgagatcatcccattttgtcatcatacaaattcacgcttgcagttttgcttcgttaacaagacaagatgtctttatcaaagacccgttttttcttcttgaagaatacttccctgttgagcacatgcaaaccatatttatctcagatttcactcaacttgggtgcttccaagagaagtaaaattcttcccactgcatcaacttccaagaaacccgtagaccagtttctcttcagccaaaagaagttgctcgccgatcaccgcggtaacagaggagtcagaaggtttcacacccttccatcccgatttcaaagtcaaagtgctgcgttgaaccaaggttttcaggttgccaaagcccagtctgcaaaaactagttccaaatggcctattaattcccataaaagtgttggctacgtatgtatcggtacctccattctggtatttgctattgttgtcgttggtgggttgactagactgaccgaatccggtctttccataacggagtggaaacctatcactggttcggttcccccactgactgaggaagactggaagttggaatttgaaaaatacaaacaaagccctgagtttcaggaactaaattctcacataacattggaagagttcaagtttatattttccatggaatggggacatagattgttgggaagggtcatcggcctgtcgtttgttcttcccacgttttacttcattgcccgtcgaaagtgttccaaagatgttgcattgaaactgcttgcaatatgctctatgataggattccaaggtttcatcggctggtggatggtgtattccggattggacaaacagcaattggctgaacgtaactccaaaccaactgtgtctccatatcgcttaactacccatcttggaactgcatttgttatttactgttacatgatttacacagggcttcaagttttgaagaactataagatcatgaaacagcctgaagcgtatgttcaaattttcaagcaaattgcgtctccaaaattgaaaactttcaagagactctcttcagttctattaggcctggtg

SEQ ID NO: 48 - DNA, 981 bp, Artificial Sequence
  Completely Synthetic DNA Sequence
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 49 - DNA, 1128 bp, Artificial Sequence
  Completely Synthetic DNA Sequence
gatctggccattgtgaaacttgacactaaagacaaaactcttagagtttccaatcacttaggagacgatgtttcctacaacgagtacgatccctcattgatcatgagcaatttgtatgtgaaaaaagtcatcgaccttgacaccttggataaaagggctggaggaggtggaaccacctgtgcaggcggtctgaaagtgttcaagtacggatctactaccaaatatacatctggtaacctgaacggcgtcaggttagtatactggaacgaaggaaagttgcaaagctccaaatttgtggttcgatcctctaattactctcaaaagcttggaggaaacagcaacgccgaatcaattgacaacaatggtgtgggttttgcctcagctggagactcaggcgcatggattctttccaagctacaagatgttagggagtaccagtcattcactgaaaagctaggtgaagctacgatgagcattttcgatttccacggtcttaaacaggagacttctactacagggcttggggtagttggtatgattcattcttacgacggtgagttcaaacagtttggtttgttcactccaatgacatctattctacaaagacttcaacgagtgaccaatgtagaatggtgtgtagcgggttgcgaagatggggatgtggacactgaaggagaacacgaattgagtgatttggaacaactgcatatgcatagtgattccgactagtcaggcaagagagagccctcaaatttacctctctgcccctcctcactccttttggtacgcataattgcagtataaagaacttgctgccagccagtaatcttatttcatacgcagttctatatagcacataatcttgcttgtatgtatgaaatttaccgcgttttagttgaaattgtttatgttgtgtgccttgcatgaaatctctcgttagccctatccttacatttaactggtctcaaaacctctaccaattccattgctgtacaacaatatgaggcggcattactgtagggttggaaaaaaattgtcattccagctagagatcacacgacttcatcacgcttattgctcctcattgctaaatcatttactcttgacttcgacccagaaaagttcgcc

SEQ ID NO: 50 - DNA, 1231 bp, Artificial Sequence
  Completely Synthetic DNA Sequence
gcatgtcaaacttgaacacaacgactagatagttgttttttctatataaaacgaaacgttatcatctttaataatcattgaggtttacccttatagttccgtattttcgtttccaaacttagtaatcttttggaaatatcatcaaagctggtgccaatcttcttgtttgaagtttcaaactgctccaccaagctacttagagactgttctaggtctgaagcaacttcgaacacagagacagctgccgccgattgttcttttttgtgtttttcttctggaagaggggcatcatcttgtatgtccaatgcccgtatcctttctgagttgtccgacacattgtccttcgaagagtttcctgacattgggcttcttctatccgtgtattaattttgggttaagttcctcgtttgcatagcagtggatacctcgatttttttggctcctatttacctgacataatattctactataatccaacttggacgcgtcatctatgataactaggctctcctttgttcaaaggggacgtcttcataatccactggcacgaagtaagtctgcaacgaggcggcttttgcaacagaacgatagtgtcgtttcgtacttggactatgctaaacaaaaggatctgtcaaacatttcaaccgtgtttcaaggcactctttacgaattatcgaccaagaccttcctagacgaacatttcaacatatccaggctactgcttcaaggtggtgcaaatgataaaggtatagatattagatgtgtttgggacctaaaacagttcttgcctgaagattcccttgagcaacaggcttcaatagccaagttagagaagcagtaccaaatcggtaacaaaagggggaagcatataaaacctttactattgcgacaaaatccatccttgaaagtaaagctgtttgttcaatgtaaagcatacgaaacgaaggaggtagatcctaagatggttagagaacttaacgggacatactccagctgcatcccatattacgatcgctggaagacttttttcatgtacgtatcgcccaccaacctttcaaagcaagctaggtatgattttgacagttctcacaatccattggttttcatgcaacttgaaaaaacccaactcaaacttcatggggatccatacaatgtaaatcattacgagagggcgaggttgaaaagtttccattgcaatcacgtcgcatcatggctactgaaaggccttaac

SEQ ID NO: 51 - DNA, 937 bp, Artificial Sequence
  Completely Synthetic DNA Sequence
tcattctatatgttcaagaaaagggtagtgaaaggaaagaaaaggcatataggcgagggagagttagctagcatacaagataatgaaggatcaatagcggtagttaaagtgcacaagaaaagagcacctgttgaggctgatgataaagctccaattacattgccacagagaaacacagtaacagaaataggaggggatgcaccacgagaagagcattcagtgaacaactttgccaaattcataaccccaagcgctaataagccaatgtcaaagtcggctactaacattaatagtacaacaactatcgattttcaaccagatgtttgcaaggactacaaacagacaggttactgcggatatggtgacacttgtaagtttttgcacctgagggatgatttcaaacagggatggaaattagatagggagtgggaaaatgtccaaaagaagaagcataatactctcaaaggggttaaggagatccaaatgtttaatgaagatgagctcaaagatatcccgtttaaatgcattatatgcaaaggagattacaaatcacccgtgaaaacttcttgcaatcattatttttgcgaacaatgtttcctgcaacggtcaagaagaaaaccaaattgtattatatgtggcagagacactttaggagttgctttaccagcaaagaagttgtcccaatttctggctaagatacataataatgaaagtaataaagtttagtaattgcattgcgttgactattgattgcattgatgtcgtgtgatactttcaccgaaaaaaaacacgaagcgcaataggagcggttgcatattagtccccaaagctatttaattgtgcctgaaactgttttttaagctcatcaagcataattgtatgcattgcgacgtaaccaacgtttaggcgcagtttaatcatagcccactgctaagcc

SEQ ID NO: 52 - DNA, 1906 bp, Artificial Sequence
  Completely Synthetic DNA Sequence
cggaggaatgcaaataataatctccttaattacccactgataagctcaagagacgcggtttgaaaacgatataatgaatcatttggattttataataaaccctgacagtttttccactgtattgttttaacactcattggaagctgtattgattctaagaagctagaaatcaatacggccatacaaaagatgacattgaataagcaccggcttttttgattagcatataccttaaagcatgcattcatggctacatagttgttaaagggcttcttccattatcagtataatgaattacataatcatgcacttatatttgcccatctctgttctctcactcttgcctgggtatattctatgaaattgcgtatagcgtgtctccagttgaaccccaagcttggcgagtttgaagagaatgctaaccttgcgtattccttgcttcaggaaacattcaaggagaaacaggtcaagaagccaaacattttgatccttcccgagttagcattgactggctacaattttcaaagccagcagcggatagagccttttttggaggaaacaaccaagggagctagtacccaatgggctcaaaaagtatccaagacgtgggattgctttactttaataggatacccagaaaaaagtttagagagccctccccgtatttacaacagtgcggtacttgtatcgcctcagggaaaagtaatgaacaactacagaaagtccttcttgtatgaagctgatgaacattggggatgttcggaatcttctgatgggtttcaaacagtagatttattaattgaaggaaagactgtaaagacatcatttggaatttgcatggatttgaatccttataaatttgaagctccattcacagacttcgagttcagtggccattgcttgaaaaccggtacaagactcattttgtgcccaatggcctggttgtcccctctatcgccttccattaaaaaggatcttagtgatatagagaaaagcagacttcaaaagttctaccttgaaaaaatagataccccggaatttgacgttaattacgaattgaaaaaagatgaagtattgcccacccgtatgaatgaaacgttggaaacaattgactttgagccttcaaaaccggactactctaatataaattattggatactaaggttttttccctttctgactcatgtctataaacgagatgtgctcaaagagaatgcagttgcagtcttatgcaaccgagttggcattgagagtgatgtcttgtacggaggatcaaccacgattctaaacttcaatggtaagttagcatcgacacaagaggagctggagttgtacgggcagactaatagtctcaaccccagtgtggaagtattgggggcccttggcatgggtcaacagggaattctagtacgagacattgaattaacataatatacaatatacaataaacacaaataaagaatacaagcctgacaaaaattcacaaattattgcctagacttgtcgttatcagcagcgacctttttccaatgctcaatttcacgatatgccttttctagctctgctttaagcttctcattggaattggctaactcgttgactgcttggtcagtgatgagtttctccaaggtccatttctcgatgttgttgttttcgttttcctttaatctcttgatataatcaacagccttctttaatatctgagccttgttcgagtcccctgttggcaacagagcggccagttcctttattccgtggtttatattttctcttctacgcctttctacttctttgtgattctctttacgcatcttatgccattcttcagaaccagtggctggcttaaccgaatagccagagcctgaagaagccgcactagaagaagcagtggcattgttgactatgg

SEQ ID NO: 53 - DNA, 1224 bp, Artificial Sequence
  Completely Synthetic DNA Sequence
tcagtcagtgctcttgatggtgacccagcaagtttgaccagagaagtgattagattggcccaagacgcagaggtggagttggagagacaacgtggactgctgcagcaaatcggagatgcattgtctagtcaaagaggtagggtgcctaccgcagctcctccagcacagcctagagtgcatgtgacccctgcaccagctgtgattcctatcttggtcatcgcctgtgacagatctactgttagaagatgtctggacaagctgttgcattacagaccatctgctgagttgttccctatcatcgttagtcaagactgtggtcacgaggagactgcccaagccatcgcctcctacggatctgctgtcactcacatcagacagcctgacctgtcatctattgctgtgccaccagaccacagaaagttccaaggttactacaagatcgctagacactacagatgggcattgggtcaagtcttcagacagtttagattccctgctgctgtggtggtggaggatgacttggaggtggctcctgacttctttgagtactttagagcaacctatccattgctgaaggcagacccatccctgtggtgtgtctctgcctggaatgacaacggtaaggagcaaatggtggacgcttctaggcctgagctgttgtacagaaccgacttctttcctggtctgggatggttgctgttggctgagttgtgggctgagttggagcctaagtggccaaaggcattctgggacgactggatgagaagacctgagcaaagacagggtagagcctgtatcagacctgagatctcaagaaccatgacctttggtagaaagggagtgtctcacggtcaattctttgaccaacacttgaagtttatcaagctgaaccagcaatttgtgcacttcacccaactggacctgtcttacttgcagagagaggcctatgacagagatttcctagctagagtctacggagctcctcaactgcaagtggagaaagtgaggaccaatgacagaaaggagttgggagaggtgagagtgcagtacactggtagggactcctttaaggctttcgctaaggctctgggtgtcatggatgaccttaagtctggagttcctagagctggttacagaggtattgtcacctttcaattcagaggtagaagagtccacttggctcctccacctacttgggagggttatgatccttcttggaattag

SEQ ID NO: 54 - DNA, 99 bp, Artificial Sequence
  Completely Synthetic DNA Sequence
atgcccagaaaaatatttaactacttcattttgactgtattcatggcaattcttgctattgttttacaatggtctatagagaatggacatgggcgcgcc

SEQ ID NO: 55 - DNA, 435 bp, Artificial Sequence
  Completely Synthetic DNA Sequence
gaagtaaagttggcgaaactttgggaacctttggttaaaactttgtaatttttgtcgctacccattaggcagaatctgcatcttgggagggggatgtggtggcgttctgagatgtacgcgaagaatgaagagccagtggtaacaacaggcctagagagatacgggcataatgggtataacctacaagttaagaatgtagcagccctggaaaccagattgaaacgaaaaacgaaatcatttaaactgtaggatgttttggctcattgtctggaaggctggctgtttattgccctgttctttgcatgggaataagctattatatccctcacataatcccagaaaatagattgaagcaacgcgaaatccttacgtatcgaagtagccttcttacacattcacgttgtacggataagaaaactactcaaacgaacaatc

SEQ ID NO: 56 - DNA, 404 bp, Artificial Sequence
  Completely Synthetic DNA Sequence
aatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaacacaaacaccaagaagcaaaaactaaccccatataaaaacatcctggtagataatgctggtaacccgctctccttccatattctgggctacttcacgaagtctgaccggtctcagttgatcaacatgatcctcgaaatgg

SEQ ID NO: 57 - DNA, 1407 bp, Artificial Sequence
  Completely Synthetic DNA Sequence
gagcccgctgacgccaccatccgtgagaagagggcaaagatcaaagagatgatgacccatgcttggaataattataaacgctatgcgtggggcttgaacgaactgaaacctatatcaaaagaaggccattcaagcagtttgtttggcaacatcaaaggagctacaatagtagatgccctggatacccttttcattatgggcatgaagactgaatttcaagaagctaaatcgtggattaaaaaatatttagattttaatgtgaatgctgaagtttctgtttttgaagtcaacatacgcttcgtcggtggactgctgtcagcctactatttgtccggagaggagatatttcgaaagaaagcagtggaacttggggtaaaattgctacctgcatttcatactccctctggaataccttgggcattgctgaatatgaaaagtgggatcgggcggaactggccctgggcctctggaggcagcagtatcctggccgaatttggaactctgcatttagagtttatgcacttgtcccacttatcaggagacccagtctttgccgaaaaggttatgaaaattcgaacagtgttgaacaaactggacaaaccagaaggcctttatcctaactatctgaaccccagtagtggacagtggggtcaacatcatgtgtcggttggaggacttggagacagcttttatgaatatttgcttaaggcgtggttaatgtctgacaagacagatctcgaagccaagaagatgtattttgatgctgttcaggccatcgagactcacttgatccgcaagtcaagtgggggactaacgtacatcgcagagtggaaggggggcctcctggaacacaagatgggccacctgacgtgctttgcaggaggcatgtttgcacttggggcagatggagctccggaagcccgggcccaacactaccttgaactcggagctgaaattgcccgcacttgtcatgaatcttataatcgtacatatgtgaagttgggaccggaagcgtttcgatttgatggcggtgtggaagctattgccacgaggcaaaatgaaaagtattacatcttacggcccgaggtcatcgagacatacatgtacatgtggcgactgactcacgaccccaagtacaggacctgggcctgggaagccgtggaggctctagaaagtcactgcagagtgaacggaggctactcaggcttacgggatgtttacattgcccgtgagagttatgacgatgtccagcaaagtttcttcctggcagagacactgaagtatttgtacttgatattttccgatgatgaccttcttccactagaacactggatcttcaacaccgaggctcatcctttccctatactccgtgaacagaagaaggaaattgatggcaaagagaaatga

SEQ ID NO: 58 - DNA, 318 bp, Artificial Sequence
  Completely Synthetic DNA Sequence
atgaacactatccacataataaaattaccgcttaactacgccaactacacctcaatgaaacaaaaaatctctaaatttttcaccaacttcatccttattgtgctgctttcttacattttacagttctcctataagcacaatttgcattccatgcttttcaattacgcgaaggacaattttctaacgaaaagagacaccatctcttcgccctacgtagttgatgaagacttacatcaaacaactttgtttggcaaccacggtacaaaaacatctgtacctagcgtagattccataaaagtgcatggcgtggggcgcgcc

SEQ ID NO: 59 - DNA, 1250 bp, Artificial Sequence
  Completely Synthetic DNA Sequence
gagtcggccaagagatgataactgttactaagcttctccgtaattagtggtattttgtaacttttaccaataatcgtttatgaatacggatatttttcgaccttatccagtgccaaatcacgtaacttaatcatggtttaaatactccacttgaacgattcattattcagaaaaaagtcaggttggcagaaacacttgggcgctttgaagagtataagagtattaagcattaaacatctgaactttcaccgccccaatatactactctaggaaactcgaaaaattcctttccatgtgtcatcgcttccaacacactttgctgtatccttccaagtatgtccattgtgaacactgatctggacggaatcctacctttaatcgccaaaggaaaggttagagacatttatgcagtcgatgagaacaacttgctgttcgtcgcaactgaccgtatctccgcttacgatgtgattatgacaaacggtattcctgataagggaaagattttgactcagctctcagttttctggtttgattttttggcaccctacataaagaatcatttggttgcttctaatgacaaggaagtctttgctttactaccatcaaaactgtctgaagaaaaatacaaatctcaattagagggacgatccttgatagtaaaaaagcacagactgatacctttggaagccattgtcagaggttacatcactggaagtgcatggaaagagtacaagaactcaaaaactgtccatggagtcaaggttgaaaacgagaaccttcaagagagcgacgcctttccaactccgattttcacaccttcaacgaaagctgaacagggtgaacacgatgaaaacatctctattgaacaagctgctgagattgtaggtaaagacatttgtgagaaggtcgctgtcaaggcggtcgagttgtattctgctgcaaaaaacctcgcccttttgaaggggatcattattgctgatacgaaattcgaatttggactggacgaaaacaatgaattggtactagtagatgaagttttaactccagattcttctagattttggaatcaaaagacttaccaagtgggtaaatcgcaagagagttacgataagcagtttctcagagattggttgacggccaacggattgaatggcaaagagggcgtagccatggatgcagaaattgctatcaagagtaaagaaaagtatattgaagcttatgaagcaattactggcaagaaatgggcttga

SEQ ID NO: 60 - DNA, 882 bp, Artificial Sequence
  Completely Synthetic DNA Sequence
atgattagtaccctcctcgcctttttcagacatctgaaatttcccttattcttccaattccatataaaatcctatttaggtaattagtaaacaatgatcataaagtgaaatcattcaagtaaccattccgtttatcgttgatttaaaatcaataacgaatgaatgtcggtctgagtagtcaatttgttgccttggagctcattggcagggggtcttttggctcagtatggaaggttgaaaggaaaacagatggaaagtggttcgtcagaaaagaggtatcctacatgaagatgaatgccaaagagatatctcaagtgatagctgagttcagaattcttagtgagttaagccatcccaacattgtgaagtaccttcatcacgaacatatttctgagaataaaactgtcaatttatacatggaatactgtgatggtggagatctctccaagctgattcgaacacatagaaggaacaaagagtacatttcagaagaaaaaatatggagtatttttacgcaggttttattagcattgtatcgttgtcattatggaactgatttcacggcttcaaaggagtttgaatcgctcaataaaggtaatagacgaacccagaatccttcgtgggtagactcgacaagagttattattcacagggatataaaacccgacaacatctttctgatgaacaattcaaaccttgtcaaactgggagattttggattagcaaaaattctggaccaagaaaacgattttgccaaaacatacgtcggtacgccgtattacatgtctcctgaagtgctgttggaccaaccctactcaccattatgtgatatatggtctcttgggtgcgtcatgtatgagctatgtgcattgaggcctcctt

SEQ ID NO: 61 - DNA, 2100 bp, Artificial Sequence
  Completely Synthetic DNA Sequence
atgacagctcagttacaaagtgaaagtacttctaaaattgttttggttacaggtggtgctggatacattggttcacacactgtggtagagctaattgagaatggatatgactgtgttgttgctgataacctgtcgaattcaacttatgattctgtagccaggttagaggtcttgaccaagcatcacattcccttctatgaggttgatttgtgtgaccgaaaaggtctggaaaaggttttcaaagaatataaaattgattcggtaattcactttgctggtttaaaggctgtaggtgaatctacacaaatcccgctgagatactatcacaataacattttgggaactgtcgttttattagagttaatgcaacaatacaacgtttccaaatttgttttttcatcttctgctactgtctatggtgatgctacgagattcccaaatatgattcctatcccagaagaatgtcccttagggcctactaatccgtatggtcatacgaaatacgccattgagaatatcttgaatgatctttacaatagcgacaaaaaaagttggaagtttgctatcttgcgttattttaacccaattggcgcacatccctctggattaatcggagaagatccgctaggtataccaaacaatttgttgccatatatggctcaagtagctgttggtaggcgcgagaagctttacatcttcggagacgattatgattccagagatggtaccccgatcagggattatatccacgtagttgatctagcaaaaggtcatattgcagccctgcaatacctagaggcctacaatgaaaatgaaggtttgtgtcgtgagtggaacttgggttccggtaaaggttctacagtttttgaagtttatcatgcattctgcaaagcttctggtattgatcttccatacaaagttacgggcagaagagcaggtgatgttttgaacttgacggctaaaccagatagggccaaacgcgaactgaaatggcagaccgagttgcaggttgaagactcctgcaaggatttatggaaatggactactgagaatccttttggttaccagttaaggggtgtcgaggccagattttccgctgaagatatgcgttatgacgcaagatttgtgactattggtgccggcaccagatttcaagccacgtttgccaatttgggcgccagcattgttgacctgaaagtgaacggacaatcagttgttcttggctatgaaaatgaggaagggtatttgaatcctgatagtgcttatataggcgccacgatcggcaggtatgctaatcgtatttcgaagggtaagtttagtttatgcaacaaagactatcagttaaccgttaataacggcgttaatgcgaatcatagtagtatcggttctttccacagaaaaagatttttgggacccatcattcaaaatccttcaaaggatgtttttaccgccgagtacatgctgatagataatgagaaggacaccgaatttccaggtgatctattggtaaccatacagtatactgtgaacgttgcccaaaaaagtttggaaatggtatataaaggtaaattgactgctggtgaagcgacgccaataaatttaacaaatcatagttatttcaatctgaacaagccatatggagacactattgagggtacggagattatggtgcgttcaaaaaaatctgttgatgtcgacaaaaacatgattcctacgggtaatatcgtcgatagagaaattgctacctttaactctacaaagccaacggtcttaggccccaaaaatccccagtttgattgttgttttgtggtggatgaaaatgctaagccaagtcaaatcaatactctaaacaatgaattgacgcttattgtcaaggcttttcatcccgattccaatattacattagaagttttaagtacagagccaacttatcaattttataccggtgatttcttgtctgctggttacgaagcaagacaaggttttgcaattgagcctggtagatacattgatgctatcaatcaagagaactggaaagattgtgtaaccttgaaaaacggtgaaacttacgggtccaagattgtctacagattttcctga

SEQ ID NO: 62 - DNA, 512 bp, Artificial Sequence
  Completely Synthetic DNA Sequence
taagcttcacgatttgtgttccagtttatcccccctttatataccgttaaccctttccctgttgagctgactgttgttgtattaccgcaatttttccaagtttgccatgcttttcgtgttatttgaccgatgtcttttttcccaaatcaaactatatttgttaccatttaaaccaagttatcttttgtattaagagtctaagtttgttcccaggcttcatgtgagagtgataaccatccagactatgattcttgttttttattgggtttgtttgtgtgatacatctgagttgtgattcgtaaagtatgtcagtctatctagatttttaatagttaattggtaatcaatgacttgtttgttttaacttttaaattgtgggtcgtatccacgcgtttagtatagctgttcatggctgttagaggagggcgatgtttatatacagaggacaagaatgaggaggcggcgtgtatttttaaaatggagacgcgactcctgtacaccttatcggttgg

SEQ ID NO: 63 - DNA, 1068 bp, Artificial Sequence
  Completely Synthetic DNA Sequence
ggtagagatttgtctagattgccacagttggttggtgtttccactccattgcaaggaggttctaactctgctgctgctattggtcaatcttccggtgagttgagaactggtggagctagaccacctccaccattgggagcttcctctcaaccaagaccaggtggtgattcttctccagttgttgactctggtccaggtccagcttctaacttgacttccgttccagttccacacactactgctttgtccttgccagcttgtccagaagaatccccattgttggttggtccaatgttgatcgagttcaacatgccagttgacttggagttggttgctaagcagaacccaaacgttaagatgggtggtagatacgctccaagagactgtgtttccccacacaaagttgctatcatcatcccattcagaaacagacaggagcacttgaagtactggttgtactacttgcacccagttttgcaaagacagcagttggactacggtatctacgttatcaaccaggctggtgacactattttcaacagagctaagttgttgaatgttggtttccaggaggctttgaaggattacgactacacttgtttcgttttctccgacgttgacttgattccaatgaacgaccacaacgcttacagatgtttctcccagccaagacacatttctgttgctatggacaagttcggtttctccttgccatacgttcaatacttcggtggtgtttccgctttgtccaagcagcagttcttgactatcaacggtttcccaaacaattactggggatggggtggtgaagatgacgacatctttaacagattggttttcagaggaatgtccatctctagaccaaacgctgttgttggtagatgtagaatgatcagacactccagagacaagaagaacgagccaaacccacaaagattcgacagaatcgctcacactaaggaaactatgttgtccgacggattgaactccttgacttaccaggttttggacgttcagagatacccattgtacactcagatcactgttgacatcggtactccatcctag

SEQ ID NO: 64 - DNA, 183 bp, Artificial Sequence
  Completely Synthetic DNA Sequence
atggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatcgggcgcgcc

SEQ ID NO: 65 - DNA, 1074 bp, Artificial Sequence
  Completely Synthetic DNA Sequence
atgaatagcatacacatgaacgccaatacgctgaagtacatcagcctgctgacgctgaccctgcagaatgccatcctgggcctcagcatgcgctacgcccgcacccggccaggcgacatcttcctcagctccacggccgtactcatggcagagttcgccaaactgatcacgtgcctgttcctggtcttcaacgaggagggcaaggatgcccagaagtttgtacgctcgctgcacaagaccatcattgcgaatcccatggacacgctgaaggtgtgcgtcccctcgctggtctatatcgttcaaaacaatctgctgtacgtctctgcctcccatttggatgcggccacctaccaggtgacgtaccagctgaagattctcaccacggccatgttcgcggttgtcattctgcgccgcaagctgctgaacacgcagtggggtgcgctgctgctcctggtgatgggcatcgtcctggtgcagttggcccaaacggagggtccgacgagtggctcagccggtggtgccgcagctgcagccacggccgcctcctctggcggtgctcccgagcagaacaggatgctcggactgtgggccgcactgggcgcctgcttcctctccggattcgcgggcatctactttgagaagatcctcaagggtgccgagatctccgtgtggatgcggaatgtgcagttgagtctgctcagcattcccttcggcctgctcacctgtttcgttaacgacggcagtaggatcttcgaccagggattcttcaagggctacgatctgtttgtctggtacctggtcctgctgcaggccggcggtggattgatcgttgccgtggtggtcaagtacgcggataacattctcaagggcttcgccacctcgctggccatcatcatctcgtgcgtggcctccatatacatcttcgacttcaatctcacgctgcagttcagcttcggagctggcctggtcatcgcctccatatttctctacggctacgatccggccaggtcggcgccgaagccaactatgcatggtcctggcggcgatgaggagaagctgctgccgcgcgtctag

SEQ ID NO: 66 - DNA, 798 bp, Artificial Sequence
  Completely Synthetic DNA Sequence
tggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagcc

SEQ ID NO: 67 - DNA, 302 bp, Artificial Sequence
  Completely Synthetic DNA Sequence
aatatatacctcatttgttcaatttggtgtaaagagtgtggcggatagacttcttgtaaatcaggaaagctacaattccaattgctgcaaaaaataccaatgcccataaaccagtatgagcggtgccttcgacggattgcttactttccgaccctttgtcgtttgattcttctgcctttggtgagtcagtttgtttcgactttatatctgactcatcaacttcctttacggttgcgtttttaatcataattttagccgttggcttattatcccttgagttggtaggagttttgatgatgctg

SEQ ID NO: 68 - DNA, 461 bp, Artificial Sequence
  Completely Synthetic DNA Sequence
taactggccctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgc

SEQ ID NO: 69 - DNA, 1841 bp, Artificial Sequence
  Completely Synthetic DNA Sequence
gtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgctgacaataatcagtggaattcaaggaaggacaatcccgacctcaatctgttcattaatgaagagttcgaatcgtccttaaatcaagcgctagactcaattgtcaatgagaaccctttctttgaccaagaaactataaatagatcgaatgacaaagttggaaatgagtccattagcttacatgatattgagcaggcagaccaaaataaaccgtcctttgagagcgatattgatggttcggcgccgttgataagagacgacaaattgccaaagaaacaaagctgggggctgagcaattttttttcaagaagaaatagcatatgtttaccactacatgaaaatgattcaagtgttgttaagaccgaaagatctattgcagtgggaacaccccatcttcaatactgcttcaatggaatctccaatgccaagtacaatgcatttacctttttcccagtcatcctatacgagcaattcaaattttttttcaatttatactttactttagtggctctctctcaagcgataccgcaacttcgcattggatatctttcttcgtatgtcgtcccacttttgtttgtactcatagtgaccatgtcaaaagaggcgatggatgatattcaacgccgaagaagggatagagaacagaacaatgaaccatatgaggttctgtccagcccatcaccagttttgtccaaaaacttaaaatgtggtcacttggttcgattgcataagggaatgagagtgcccgcagatatggttcttgtccagtcaagcgaatccaccggagagtcatttatcaagacagatcagctggatggtgagactgattggaagcttcggattgtttctccagttacacaatcgttaccaatgactgaacttcaaaatgtcgccatcactgcaagcgcaccctcaaaatcaattcactcctttcttggaagattgacctacaatgggcaatcatatggtcttacgatagacaacacaatgtggtgtaatactgtattagcttctggttcagcaattggttgtataatttacacaggtaaagatactcgacaatcgatgaacacaactcagcccaaactgaaaacgggcttgttagaactggaaatcaatagtttgtccaagatcttatgtgtttgtgtgtttgcattatctgtcatcttagtgctattccaaggaatagctgatgattggtacgtcgatatcatgcggtttctcattctattctccactattatcccagtgtctctgagagttaaccttgatcttggaaagtcagtccatgctcatcaaatagaaactgatagctcaatacctgaaaccgttgttagaactagtacaataccggaagacctgggaagaattgaatacctattaagtgacaaaactggaactcttactcaaaatgatatggaaatgaaaaaactacacctaggaacagtctcttatgctggtgataccatggatattatttctgatcatgttaaaggtcttaataacgctaaaacatcgaggaaagatcttggtatgagaataagagatttggttacaactctggccatctg

SEQ ID NO: 70 - DNA, 3105 bp, Artificial Sequence
  Completely Synthetic DNA Sequence
agagacgatccaattagacctccattgaaggttgctagatccccaagaccaggtcaatgtcaagatgttgttcaggacgtcccaaacgttgatgtccagatgttggagttgtacgatagaatgtccttcaaggacattgatggtggtgtttggaagcagggttggaacattaagtacgatccattgaagtacaacgctcatcacaagttgaaggtcttcgttgtcccacactcccacaacgatcctggttggattcagaccttcgaggaatactaccagcacgacaccaagcacatcttgtccaacgctttgagacatttgcacgacaacccagagatgaagttcatctgggctgaaatctcctacttcgctagattctaccacgatttgggtgagaacaagaagttgcagatgaagtccatcgtcaagaacggtcagttggaattcgtcactggtggatgggtcatgccagacgaggctaactcccactggagaaacgttttgttgcagttgaccgaaggtcaaacttggttgaagcaattcatgaacgtcactccaactgcttcctgggctatcgatccattcggacactctccaactatgccatacattttgcagaagtctggtttcaagaatatgttgatccagagaacccactactccgttaagaaggagttggctcaacagagacagttggagttcttgtggagacagatctgggacaacaaaggtgacactgctttgttcacccacatgatgccattctactcttacgacattcctcatacctgtggtccagatccaaaggtttgttgtcagttcgatttcaaaagaatgggttccttcggtttgtcttgtccatggaaggttccacctagaactatctctgatcaaaatgttgctgctagatccgatttgttggttgatcagtggaagaagaaggctgagttgtacagaaccaacgtcttgttgattccattgggtgacgacttcagattcaagcagaacaccgagtgggatgttcagagagtcaactacgaaagattgttcgaacacatcaactctcaggctcacttcaatgtccaggctcagttcggtactttgcaggaatacttcgatgctgttcaccaggctgaaagagctggacaagctgagttcccaaccttgtctggtgacttcttcacttacgctgatagatctgataactactggtctggttactacacttccagaccataccataagagaatggacagagtcttgatgcactacgttagagctgctgaaatgttgtccgcttggcactcctgggacggtatggctagaatcgaggaaagattggagcaggctagaagagagttgtccttgttccagcaccacgacggtattactggtactgctaaaactcacgttgtcgtcgactacgagcaaagaatgcaggaagctttgaaagcttgtcaaatggtcatgcaacagtctgtctacagattgttgactaagccatccatctactctccagacttctccttctcctacttcactttggacgactccagatggccaggttctggtgttgaggactctagaactaccatcatcttgggtgaggatatcttgccatccaagcatgttgtcatgcacaacaccttgccacactggagagagcagttggttgacttctacgtctcctctccattcgtttctgttaccgacttggctaacaatccagttgaggctcaggtttctccagtttggtcttggcaccacgacactttgactaagactatccacccacaaggttccaccaccaagtacagaatcatcttcaaggctagagttccaccaatgggtttggctacctacgttttgaccatctccgattccaagccagagcacacctcctacgcttccaatttgttgcttagaaagaacccaacttccttgccattgggtcaatacccagaggatgtcaagttcggtgatccaagagagatctccttgagagttggtaacggtccaaccttggctttctctgagcagggtttgttgaagtccattcagttgactcaggattctccacatgttccagttcacttcaagttcttgaagtacggtgttagatctcatggtgatagatctggtgcttacttgttcttgccaaatggtccagcttctccagtcgagttgggtcagccagttgtcttggtcactaagggtaaattggagtcttccgtttctgttggtttgccatctgtcgttcaccagaccatcatgagaggtggtgctccagagattagaaatttggtcgatattggttctttggacaacactgagatcgtcatgagattggagactcatatcgactctggtgatatcttctacactgatttgaatggattgcaattcatcaagaggagaagattggacaagttgccattgcaggctaactactacccaattccatctggtatgttcattgaggatgctaataccagattgactttgttgaccggtcaaccattgggtggatcttctttggcttctggtgagttggagattatgcaagatagaagattggcttctgatgatgaaagaggtttgggtcagggtgttttggacaacaagccagttttgcatatttacagattggtcttggagaaggttaacaactgtgtcagaccatctaagttgcatccagctggttacttgacttctgctgctcacaaagcttctcagtctttgttggatccattggacaagttcatcttcgctgaaaatgagtggatcggtgctcagggtcaattcggtggtgatcatccatctgctagagaggatttggatgtctctgtcatgagaagattgaccaagtcttctgctaaaacccagagagttggttacgttttgcacagaaccaatttgatgcaatgtggtactccagaggagcatactcagaagttggatgtctgtcacttgttgccaaatgttgctagatgtgagagaactaccttgactttcttgcagaatttggagcacttggatggtatggttgctccagaagtttgtccaatggaaaccgctgcttacgtctcttctcactcttcttga

SEQ ID NO: 71 - DNA, 108 bp, Artificial Sequence
  Completely Synthetic DNA Sequence
atgctgcttaccaaaaggttttcaaagctgttcaagctgacgttcatagttttgatattgtgcgggctgttcgtcattacaaacaaatacatggatgagaacacgtcg

SEQ ID NO: 72 - DNA, 1729 bp, Artificial Sequence
  Completely Synthetic DNA Sequence
caagttgcgtccggtatacgtaacgtctcacgatgatcaaagataatacttaatcttcatggtctactgaataactcatttaaacaattgactaattgtacattatattgaacttatgcatcctattaacgtaatcttctggcttctctctcagactccatcagacacagaatatcgttctctctaactggtcctttgacgtttctgacaatagttctagaggagtcgtccaaaaactcaactctgacttgggtgacaccaccacgggatccggttcttccgaggaccttgatgaccttggctaatgtaactggagttttagtatccattttaagatgtgtgtttctgtaggttctgggttggaaaaaaattttagacaccagaagagaggagtgaactggtttgcgtgggtttagactgtgtaaggcactactctgtcgaagttttagataggggttacccgctccgatgcatgggaagcgattagcccggctgttgcccgtttggtttttgaagggtaattttcaatatctctgtttgagtcatcaatttcatattcaaagattcaaaaacaaaatctggtccaaggagcgcatttaggattatggagttggcgaatcacttgaacgatagactattatttgctgttcctaaagagggcagattgtatgagaaatgcgttgaattacttaggggatcagatattcagtttcgaagatccagtagattggatatagctttgtgcactaacctgcccctggcattggttttccttccagctgctgacattcccacgtttgtaggagagggtaaatgtgatttgggtataactggtattgaccaggttcaggaaagtgacgtagatgtcatacctttattagacttgaatttcggtaagtgcaagttgcagattcaagttcccgagaatggtgacttgaaagaacctaaacagctaattggtaaagaaattgtttcctcctttactagcttaaccaccaggtactttgaacaactggaaggagttaagcctggtgagccactaaagacaaaaatcaaatatgttggagggtctgttgaggcctcttgtgccctaggagttgccgatgctattgtggatcttgttgagagtggagaaaccatgaaagcggcagggctgatcgatattgaaactgttctttctacttccgcttacctgatctcttcgaagcatcctcaacacccagaactgatggatactatcaaggagagaattgaaggtgtactgactgctcagaagtatgtcttgtgtaattacaacgcacctagaggtaaccttcctcagctgctaaaactgactccaggcaagagagctgctaccgtttctccattagatgaagaagattgggtgggagtgtcctcgatggtagagaagaaagatgttggaagaatcatggacgaattaaagaaacaaggtgccagtgacattcttgtctttgagatcagtaattgtagagcatagatagaataatattcaagaccaacggcttctcttcggaagctccaagtagcttatagtgatgagtaccggcatatatttataggcttaaaatttcgagggttcactatattcgtttagtgggaagagttcctttcactcttgttatctatattgtcagcgtggactgtttataactgtaccaacttagtttctttcaactccaggttaagagacataaatgtcctttgatgc

SEQ ID NO: 73 - DNA, 1068 bp, Artificial Sequence
  Completely Synthetic DNA Sequence
tccttggtttaccaattgaacttcgaccagatgttgagaaacgttgacaaggacggtacttggtctcctggtgagttggttttggttgttcaggttcacaacagaccagagtacttgagattgttgatcgactccttgagaaaggctcaaggtatcagagaggttttggttatcttctcccacgatttctggtctgctgagatcaactccttgatctcctccgttgacttctgtccagttttgcaggttttcttcccattctccatccaattgtacccatctgagttcccaggttctgatccaagagactgtccaagagacttgaagaagaacgctgctttgaagttgggttgtatcaacgctgaatacccagattctttcggtcactacagagaggctaagttctcccaaactaagcatcattggtggtggaagttgcactttgtttgggagagagttaaggttttgcaggactacactggattgatcttgttcttggaggaggatcattacttggctccagacttctaccacgttttcaagaagatgtggaagttgaagcaacaagagtgtccaggttgtgacgttttgtccttgggaacttacactactatcagatccttctacggtatcgctgacaaggttgacgttaagacttggaagtccactgaacacaacatgggattggctttgactagagatgcttaccagaagttgatcgagtgtactgacactttctgtacttacgacgactacaactgggactggactttgcagtacttgactttggcttgtttgccaaaagtttggaaggttttggttccacaggctccaagaattttccacgctggtgactgtggaatgcaccacaagaaaacttgtagaccatccactcagtccgctcaaattgagtccttgttgaacaacaacaagcagtacttgttcccagagactttggttatcggagagaagtttccaatggctgctatttccccaccaagaaagaatggtggatggggtgatattagagaccacgagttgtgtaaatcctacagaagattgcagtag

SEQ ID NO: 74 - DNA, 300 bp, Artificial Sequence
  Completely Synthetic DNA Sequence
atgctgcttaccaaaaggttttcaaagctgttcaagctgacgttcatagttttgatattgtgcgggctgttcgtcattacaaacaaatacatggatgagaacacgtcggtcaaggagtacaaggagtacttagacagatatgtccagagttactccaataagtattcatcttcctcagacgccgccagcgctgacgattcaaccccattgagggacaatgatgaggcaggcaatgaaaagttgaaaagcttctacaacaacgttttcaactttctaatggttgattcgcccgggcgcgcc

SEQ ID NO: 75 - DNA, 1373 bp, Artificial Sequence
  Completely Synthetic DNA Sequence
gatctggccttccctgaatttttacgtccagctatacgatccgttgtgactgtatttcctgaaatgaagtttcaacctaaagttttggttgtacttgctccacctaccacggaaactaatatcgaaaccaatgaaaaagtagaactggaatcgtcaatcgaaattcgcaaccaagtggaacccaaagacttgaatctttctaaagtctattctagtgacactaatggcaacagaagatttgagctgacttttcaaatgaatctcaataatgcaatatcaacatcagacaatcaatgggctttgtctagtgacacaggatcaattatagtagtgtcttctgcaggaagaataacttccccgatcctagaagtcggggcatccgtctgtgtcttaagatcgtacaacgaacaccttttggcaataacttgtgaaggaacatgcttttcatggaatttaaagaagcaagaatgtgttctaaacagcatttcattagcacctatagtcaattcacacatgctagttaagaaagttggagatgcaaggaactattctattgtatctgccgaaggagacaacaatccgttaccccagattctagactgcgaactttccaaaaatggcgctccaattgtggctcttagcacgaaagacatctactcttattcaaagaaaatgaaatgctggatccatttgattgattcgaaatactttgaattgttgggtgctgacaatgcactgtttgagtgtgtggaagcgctagaaggtccaattggaatgctaattcatagattggtagatgagttcttccatgaaaacactgccggtaaaaaactcaaactttacaacaagcgagtactggaggacctttcaaattcacttgaagaactaggtgaaaatgcgtctcaattaagagagaaacttgacaaactctatggtgatgaggttgaggcttcttgacctcttctctctatctgcgtttctttttttttttttttttttttttttttcagttgagccagaccgcgctaaacgcataccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagac

SEQ ID NO: 76 - DNA, 1470 bp, Artificial Sequence
  Completely Synthetic DNA Sequence
gggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtagcaataattaggagcggaccacacagtgacgacatctttctctttgaaatggtatctgaagccttccatgaccaattgatgggctctagcgatgagttgcaagttattaatgtggttgaactcacgtgctactcgagcaccgaataaccagccagctccacgaggagaaacagcccaactgtcgacttcatctgggtcagaccaaaccaagtcacaaaatcctccttcatgagggacctcttgcgctcggctgagaactctgatttgatctaacatgcgaatatcgggagagagaccaccatggatacataatattttaccatcaatgatggcactaagggttaaaaagtcgaacacctggcaacagtacttccagacagtggtggaaccatatttattgagacattcctcataaaatccataaacctgagtgatctgtctggattcatgatttccccttaccaatgtgatatgttgaggaaacttaatttttaaaatcatgagtaacgtgaacgtctccaacgagaaatagcctctatccacatagtctcctaggaagatatagttctgttttattccattagaggaggatccgggaaacccaccactaatcttgaaaagttccagtagatcgtgaaattggccgtgaatatctccgcatactgtcactggactctgcactggctgtatattggattcctccatcagcaaatccttcacccgttcgcaaagatgcttcatatcattttcacttaaagccttgcagcttttgacttcttcaaaccactgatctggtcctctttctggcatgattaaggtctataatatttctgagctgagatgtaaaaaaaaataataaaaatggggagtgaaaaagtgtgtagcttttaggagtttgggattgataccccaaaatgatctttatgagaattaaaaggtagatacgcttttaataagaacacctatctatagtactttgtggtcttgagtaattgagatgttcagcttctgaggtttgccgttattctgggatagtagtgcgcgaccaaacaacccgccaggcaaagtgtgttgtgctcgaagacgattgccagaagagtaagtccgtcctgcctcagatgttacacactttcttccctagacagtcgatgcatcatcggatttaaacctgaaactttgatgccatgatacgcctagtcacgtcgactgagattttagataagccccgatccctttagtacattcctgttatccatggatggaatggcctgata

SEQ ID NO: 77 - DNA, 1043 bp, Artificial Sequence
  Completely Synthetic DNA Sequence
aagcttgttcaccgttgggacttttccgtggacaatgttgactactccaggagggattccagctttctctactagctcagcaataatcaatgcagccccaggcgcccgttctgatggcttgatgaccgttgtattgcctgtcactatagccaggggtagggtccataaaggaatcatagcagggaaattaaaagggcatattgatgcaatcactcccaatggctctcttgccattgaagtctccatatcagcactaacttccaagaaggaccccttcaagtctgacgtgatagagcacgcttgctctgccacctgtagtcctctcaaaacgtcaccttgtgcatcagcaaagactttaccttgctccaatactatgacggaggcaattctgtcaaaattctctctcagcaattcaaccaacttgaaagcaaattgctgtctcttgatgatggagacttttttccaagattgaaatgcaatgtgggacgactcaattgcttcttccagctcctcttcggttgattgaggaacttttgaaaccacaaaattggtcgttgggtcatgtacatcaaaccattctgtagatttagattcgacgaaagcgttgttgatgaaggaaaaggttggatacggtttgtcggtctctttggtatggccggtggggtatgcaattgcagtagaagataattggacagccattgttgaaggtagagaaaaggtcagggaacttgggggttatttataccattttaccccacaaataacaactgaaaagtacccattccatagtgagaggtaaccgacggaaaaagacgggcccatgttctgggaccaatagaactgtgtaatccattgggactaatcaacagacgattggcaatataatgaaatagttcgttgaaaagccacgtcagctgtcttttcattaactttggtcggacacaacattttctactgttgtatctgtcctactttgcttatcatctgccacagggcaagtggatttccttctcgcgcggctgggtgaaaacggttaacgtgaa

SEQ ID NO: 78 - DNA, 695 bp, Artificial Sequence
  Completely Synthetic DNA Sequence
gccttgggggacttcaagtctttgctagaaactagatgaggtcaggccctcttatggttgtgtcccaattgggcaatttcactcacctaaaaagcatgacaattatttagcgaaataggtagtatattttccctcatctcccaagcagtttcgtttttgcatccatatctctcaaatgagcagctacgactcattagaaccagagtcaagtaggggtgagctcagtcatcagccttcgtttctaaaacgattgagttcttttgttgctacaggaagcgccctagggaactttcgcactttggaaatagattttgatgaccaagagcgggagttgatattagagaggctgtccaaagtacatgggatcaggccggccaaattgattggtgtgactaaaccattgtgtacttggacactctattacaaaagcgaagatgatttgaagtattacaagtcccgaagtgttagaggattctatcgagcccagaatgaaatcatcaaccgttatcagcagattgataaactcttggaaagcggtatcccattttcattattgaagaactacgataatgaagatgtgagagacggcgaccctctgaacgtagacgaagaaacaaatctacttttggggtacaatagagaaagtgaatcaagggaggtatttgtggccataatactcaactctatcattaatg

SEQ ID NO: 79 - DNA, 411 bp, Artificial Sequence
  Completely Synthetic DNA Sequence
catatggtgagagccgttctgcacaactagatgttttcgagcttcgcattgtttcctgcagctcgactattgaattaagatttccggatatctccaatctcacaaaaacttatgttgaccacgtgctttcctgaggcgaggtgttttatatgcaagctgccaaaaatggaaaacgaatggccatttttcgcccaggcaaattattcgattactgctgtcataaagacagtgttgcaaggctcacatttttttttaggatccgagataaagtgaatacaggacagcttatctctatatcttgtaccattcgtgaatcttaagagttcggttagggggactctagttgagggttggcactcacgtatggctgggcgcagaaataaaattcaggcgcagcagcacttatcgatg

SEQ ID NO: 80 - DNA, 692 bp, Artificial Sequence
  Completely Synthetic DNA Sequence
gaattcacagttataaataaaaacaaaaactcaaaaagtttgggctccacaaaataacttaatttaaatttttgtctaataaatgaatgtaattccaagattatgtgatgcaagcacagtatgcttcagccctatgcagctactaatgtcaatctcgcctgcgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagctt

SEQ ID NO: 81 - DNA, 546 bp, Artificial Sequence
  Completely Synthetic DNA Sequence
gatatctccctggggacaatatgtgttgcaactgttcgttgttggtgccccagtcccccaaccggtactaatcggtctatgttcccgtaactcatattcggttagaactagaacaataagtgcatcattgttcaacattgtggttcaattgtcgaacattgctggtgcttatatctacagggaagacgataagcctttgtacaagagaggtaacagacagttaattggtatttctttgggagtcgttgccctctacgttgtctccaagacatactacattctgagaaacagatggaagactcaaaaatgggagaagcttagtgaagaagagaaagttgcctacttggacagagctgagaaggagaacctgggttctaagaggctggactttttgttcgagagttaaactgcataattttttctaagtaaatttcatagttatgaaatttctgcagcttagtgtttactgcatcgtttactgcatcaccctgtaaataatgtgagcttttttccttccattgcttggtatcttccttgctgctgttt

SEQ ID NO: 82 - DNA, 378 bp, Artificial Sequence
  Completely Synthetic DNA Sequence
acaaaacagtcatgtacagaactaacgcctttaagatgcagaccactgaaaagaattgggtcccatttttcttgaaagacgaccaggaatctgtccattttgtttactcgttcaatcctctgagagtactcaactgcagtcttgataacggtgcatgtgatgttctatttgagttaccacatgattttggcatgtcttccgagctacgtggtgccactcctatgctcaatcttcctcaggcaatcccgatggcagacgacaaagaaatttgggtttcattcccaagaacgagaatatcagattgcgggtgttctgaaacaatgtacaggccaatgttaatgctttttgttagagaaggaacaaacttttttgctgagc

SEQ ID NO: 83 - DNA, 1494 bp, Artificial Sequence
  Completely Synthetic DNA Sequence
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 84 - DNA, 1792 bp, Artificial Sequence
  Completely Synthetic DNA Sequence
taccaattgccaaatcaggcaattgtgagacagtggtaaaaaagatgcctgcaaagttagattcacacagtaagagagatcctactcataaatgaggcgcttatttagtagctagtgatagccactgcggttctgctttatgctatttgttgtatgccttactatctttgtttggctcctttttcttgacgttttccgttggagggactccctattctgagtcatgagccgcacagattatcgcccaaaattgacaaaatcttctggcgaaaaaagtataaaaggagaaaaaagctcacccttttccagcgtagaaagtatatatcagtcattgaagactattatttaaataacacaatgtctaaaggaaaagtttgtttggcctactccggtggtttggatacctccatcatcctagcttggttgttggagcagggatacgaagtcgttgcctttttagccaacattggtcaagaggaagactttgaggctgctagagagaaagctctgaagatcggtgctaccaagtttatcgtcagtgacgttaggaaggaatttgttgaggaagttttgttcccagcagtccaagttaacgctatctacgagaacgtctacttactgggtacctctttggccagaccagtcattgccaaggcccaaatagaggttgctgaacaagaaggttgttttgctgttgcccacggttgtaccggaaagggtaacgatcaggttagatttgagctttccttttatgctctgaagcctgacgttgtctgtatcgccccatggagagacccagaattcttcgaaagattcgctggtagaaatgacttgctgaattacgctgctgagaaggatattccagttgctcagactaaagccaagccatggtctactgatgagaacatggctcacatctccttcgaggctggtattctagaagatccaaacactactcctccaaaggacatgtggaagctcactgttgacccagaagatgcaccagacaagccagagttctttgacgtccactttgagaagggtaagccagttaaattagttctcgagaacaaaactgaggtcaccgatccggttgagatctttttgactgctaacgccattgctagaagaaacggtgttggtagaattgacattgtcgagaacagattcatcggaatcaagtccagaggttgttatgaaactccaggtttgactctactgagaaccactcacatcgacttggaaggtcttaccgttgaccgtgaagttagatcgatcagagacacttttgttaccccaacctactctaagttgttatacaacgggttgtactttaccccagaaggtgagtacgtcagaactatgattcagccttctcaaaacaccgtcaacggtgttgttagagccaaggcctacaaaggtaatgtgtataacctaggaagatactctgaaaccgagaaattgtacgatgctaccgaatcttccatggatgagttgaccggattccaccctcaagaagctggaggatttatcacaacacaagccatcagaatcaagaagtacggagaaagtgtcagagagaagggaaagtttttgggactttaactcaagtaaaaggatagttgtacaattatatatacgaagaataaatcattacaaaaagtattcgtttctttgattcttaacaggattcattttctgggtgtcatcaggtacagcgctgaatatcttgaagttaacatcgagctcatcatcgacgttcatcacactagccacgtttccgcaacggtag

SEQ ID NO: 85 - DNA, 414 bp, Artificial Sequence
  Completely Synthetic DNA Sequence
ccggccatttaaatatgtgacgactgggtgatccgggttagtgagttgttctcccatctgtatatttttcatttacgatgaatacgaaatgagtattaagaaatcaggcgtagcaatatgggcagtgttcagtcctgtcatagatggcaagcactggcacatccttaataggttagagaaaatcattgaatcatttgggtggtgaaaaaaaattgatgtaaacaagccacccacgctgggagtcgaacccagaatcttttgattagaagtcaaacgcgttaaccattacgctacgcaggcatgtttcacgtccatttttgattgctttctatcataatctaaagatgtgaactcaattagttgcaatttgaccaattcttccattacaagtcgtgcttcctccgttgatgcaac

SEQ ID NO: 86 - DNA, 388 bp, Artificial Sequence
  Completely Synthetic DNA Sequence
gatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacc

SEQ ID NO: 87 - DNA, 247 bp, Artificial Sequence
  Completely Synthetic DNA Sequence
taatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaac

SEQ ID NO: 88 - protein, 20 aa, Artificial Sequence
  Completely Synthetic Amino Acid Sequence
MVAWWSLFLYGLQVAAPALA

SEQ ID NO: 89 - DNA, 1037 bp, Artificial Sequence
  Completely Synthetic DNA Sequence
aaatgcgtacctcttctacgagattcaagcgaatgagaataatgtaatatgcaagatcagaaagaatgaaaggagttgaaaaaaaaaaccgttgcgttttgaccttgaatggggtggaggtttccattcaaagtaaagcctgtgtcttggtattttcggcggcacaagaaatcgtaattttcatcttctaaacgatgaagatcgcagcccaacctgtatgtagttaaccggtcggaattataagaaagattttcgatcaacaaaccctagcaaatagaaagcagggttacaactttaaaccgaagtcacaaacgataaaccactcagctcccacccaaattcattcccactagcagaaaggaattatttaatccctcaggaaacctcgatgattctcccgttcttccatgggcgggtatcgcaaaatgaggaatttttcaaatttctctattgtcaagactgtttattatctaagaaatagcccaatccgaagctcagttttgaaaaaatcacttccgcgtttcttttttacagcccgatgaatatccaaatttggaatatggattactctatcgggactgcagataatatgacaacaacgcagattacattttaggtaaggcataaacaccagccagaaatgaaacgcccactagccatggtcgaatagtccaatgaattcagatagctatggtctaaaagctgatgttttttattgggtaatggcgaagagtccagtacgacttccagcagagctgagatggccatttttgggggtattagtaactttttgagctcttttcacttcgatgaagtgtcccattcgggatataatcggatcgcgtcgttttctcgaaaatacagcttagcgtcgtccgcttgttgtaaaagcagcaccacattcctaatctcttatataaacaaaacaacccaaattatcagtgctgttttcccaccagatataagtttcttttctcttccgctttttgattttttatctctttcctttaaaaacttctttaccttaaagggcggcc

SEQ ID NO: 90 - DNA, 1231 bp, Artificial Sequence
  Completely Synthetic DNA Sequence
gaagggccatcgaattgtcatcgtctcctcaggtgccatcgctgtgggcatgaagagagtcaacatgaagcggaaaccaaaaaagttacagcaagtgcaggcattggctgctataggacaaggccgtttgataggactttgggacgaccttttccgtcagttgaatcagcctattgcgcagattttactgactagaacggatttggtcgattacacccagtttaagaacgctgaaaatacattggaacagcttattaaaatgggtattattcctattgtcaatgagaatgacaccctatccattcaagaaatcaaatttggtgacaatgacaccttatccgccataacagctggtatgtgtcatgcagactacctgtttttggtgactgatgtggactgtctttacacggataaccctcgtacgaatccggacgctgagccaatcgtgttagttagaaatatgaggaatctaaacgtcaataccgaaagtggaggttccgccgtaggaacaggaggaatgacaactaaattgatcgcagctgatttgggtgtatctgcaggtgttacaacgattatttgcaaaagtgaacatcccgagcagattttggacattgtagagtacagtatccgtgctgatagagtcgaaaatgaggctaaatatctggtcatcaacgaagaggaaactgtggaacaatttcaagagatcaatcggtcagaactgagggagttgaacaagctggacattcctttgcatacacgtttcgttggccacagttttaatgctgttaataacaaagagttttggttactccatggactaaaggccaacggagccattatcattgatccaggttgttataaggctatcactagaaaaaacaaagctggtattcttccagctggaattatttccgtagagggtaatttccatgaatacgagtgtgttgatgttaaggtaggactaagagatccagatgacccacattcactagaccccaatgaagaactttacgtcgttggccgtgcccgttgtaattaccccagcaatcaaatcaacaaaattaagggtctacaaagctcgcagatcgagcaggttctaggttacgctgacggtgagtatgttgttcacagggacaacttggctttcccagtatttgccgatccagaactgttggatgttgttgagagtaccctgtctgaacaggagagagaatccaaaccaaataaatag

SEQ ID NO: 91 - DNA, 1425 bp, Artificial Sequence
  Completely Synthetic DNA Sequence
aatttcacatatgctgcttgattatgtaattataccttgcgttcgatggcatcgatttcctcttctgtcaatcgcgcatcgcattaaaagtatacttttttttttttcctatagtactattcgccttattataaactttgctagtatgagttctacccccaagaaagagcctgatttgactcctaagaagagtcagcctccaaagaatagtctcggtgggggtaaaggctttagtgaggagggtttctcccaaggggacttcagcgctaagcatatactaaatcgtcgccctaacaccgaaggctcttctgtggcttcgaacgtcatcagttcgtcatcattgcaaaggttaccatcctctggatctggaagcgttgctgtgggaagtgtgttgggatcttcgccattaactctttctggagggttccacgggcttgatccaaccaagaataaaatagacgttccaaagtcgaaacagtcaaggagacaaagtgttctttctgacatgatttccacttctcatgcagctagaaatgatcactcagagcagcagttacaaactggacaacaatcagaacaaaaagaagaagatggtagtcgatcttctttttctgtttcttcccccgcaagagatatccggcacccagatgtactgaaaactgtcgagaaacatcttgccaatgacagcgagatcgactcatctttacaacttcaaggtggagatgtcactagaggcatttatcaatgggtaactggagaaagtagtcaaaaagataacccgcctttgaaacgagcaaatagttttaatgatttttcttctgtgcatggtgacgaggtaggcaaggcagatgctgaccacgatcgtgaaagcgtattcgacgaggatgatatctccattgatgatatcaaagttccgggagggatgcgtcgaagttttttattacaaaagcatagagaccaacaactttctggactgaataaaacggctcaccaaccaaaacaacttactaaacctaatttcttcacgaacaactttatagagtttttggcattgtatgggcattttgcaggtgaagatttggaggaagacgaagatgaagatttagacagtggttccgaatcagtcgcagtcagtgatagtgagggagaattcagtgaggctgacaacaatttgttgtatgatgaagagtctctcctattagcacctagtacctccaactatgcgagatcaagaataggaagtattcgtactcctacttatggatctttcagttcaaatgttggttcttcgtctattcatcagcagttaatgaaaagtcaaatcccgaagctgaagaaacgtggacagcacaagcataaaacacaatcaaaaatacgctcgaagaagcaaactaccaccgtaaaagcagtgttgctgctattaaa

SEQ ID NO: 92 - DNA, 1793 bp, Artificial Sequence
  Completely Synthetic DNA Sequence
ggtttctcaattactatatactactaaccatttacctgtagcgtatttcttttccctcttcgcgaaagctcaagggcatcttcttgactcatgaaaaatatctggatttcttctgacagatcatcacccttgagcccaactctctagcctatgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatg

(19) United States
(12) Patent Application Publication
(10) Pub. No.: US 2011/0313137 A1
(43) Pub. Date: Dec. 22, 2011

(54) HER2 ANTIBODY COMPOSITIONS

(76) Inventor: Dongxing Zha, Etna, NH (US)

(21) Appl. No.: 13/203,090

(22) PCT Filed: Feb. 24, 2010
(86) PCT No.: PCT/US10/25211

§ 371 Date: Aug. 24, 2011

Related U.S. Application Data
(60) Provisional application No. 61/208,582, filed on Feb. 25, 2009.
(60) Provisional application No. 61/256,396, filed on Oct. 30, 2009.

(51) Int. Cl. C07K 16/40

(52) U.S. Cl. 530/387.3

(57) ABSTRACT

The invention relates to compositions of Her2 antibody molecules with pre-selected N-linked glycosylation forms.

### FIELD OF THE INVENTION

The present invention relates to the field of molecular biology, in particular the invention provides compositions of Her2 antibody molecules with desired N-glycoforms.

### BACKGROUND OF THE INVENTION

Currently, monoclonal immunoglobulins are almost entirely produced using mammalian expression systems such as Chinese hamster ovary cells (CHO). While CHO cells produce immunoglobulins with mammalian glycosylation patterns, the glycosylation pattern is still a mixed spectrum of glycoforms (Sethuraman & Stadheim, Curr. Opin. Biotechnol. 17: 341-346 (2006); Wildt & Gerngross, Nat. Rev. Microbiol. 3: 119-128 (2005)). Maintaining a constant glycosylation pattern ensures lot-to-lot stability and functionality of the immunoglobulins. Industry has responded to this challenge by developing engineered CHO cells designed to produce more stable glycosylation patterns (Imai-Nishiya et al., BMC Biotechnol. 7: 84 (2007); Rademacher, Biologicals 21: 103-104 (1993)).

Another biologics production vehicle is yeast, e.g., Pichia pastoris. While it has been shown that this yeast is able to produce biologics at marketable levels, the glycosylation pattern of proteins produced in wild type P. pastoris is distinctly non-mammalian (Sethuraman & Stadheim, Curr. Opin. Biotechnol. 17: 341-346 (2006); Wildt & Gerngross, Nat. Rev. Microbiol. 3: 119-128 (2005)). However, several different strains of P. pastoris have been genetically engineered to produce different human glycoforms of an immunoglobulin (Li et al., Nat. Biotechnol. 24 (2):210-215, 2006). The genetically engineered P. pastoris yeasts can produce very stable and discreet glycosylation patterns relative to their CHO produced counterparts (Wildt & Gerngross, Nat. Rev. Microbiol. 3: 119-128 (2005)).

It is understood that different glycoforms can profoundly affect the properties of a therapeutic glycoprotein, including pharmacokinetics, pharmacodynamics, receptor-interaction and tissue-specific targeting (See, Graddis et al., Curr Pharm Biotechnol. 3: 285-297 (2002)). In particular, for immunoglobulins, the oligosaccharide structure can affect properties relevant to protease resistance, the serum half-life of the immunoglobulin mediated by the FcRn receptor, binding to the complement complex C1, which induces complement-dependent cytoxicity (CDC), and binding to FcγR receptors, which are responsible for modulating the antibody-dependent cell mediated cytoxicity (ADCC) pathway, phagocytosis and immunoglobulin feedback (Carter et al., Proc. Natl. Acad. Sci. USA, 89: 4285-4289 (1992); Leatherbarrow & Dwek, FEBS Lett. 164: 227-230 (1983); Leatherbarrow et al., Molec. Immunol. 22: 407-41 (1985); Nose & Wigzell, Proc. Natl. Acad. Sci. USA 80: 6632-6636 (1983): Walker et al., Biochem. J. 259: 347-353 (1989); Walker et al., Molec. Immunol. 26: 403-411 (1989)). In addition, glycosylation differences in antibodies are generally confined to the constant domain and may influence the antibodies structure (Weitzhandler et al., (1994) T. Pharm. Sci. 83:1760).

Herceptin®, an anti-Her2 IgG antibody, is produced in Chinese hamster ovary (CHO) cells and is N-glycosylated on asparagine 297 in the Fc domain. The proto-oncogene HER2 (human epidermal growth factor receptor 2) encodes a protein tyrosine kinase (p185^{HER2}). Amplification and/or overexpression of HER2 is associated with multiple human malignancies and appears to be integrally involved in the progression of 25-30% of human breast and ovarian cancers (Simon, D. J., et al., Science 235:177-182 (1987)). It is desirable to produce Her2 antibodies that retain favorable in-vivo properties from the genetically engineered P. pastoris yeasts, which provides a very stable and discreet glycosylation pattern.

### SUMMARY OF THE INVENTION

The present invention provides lower eukaryotic host cells that have been engineered to produce Her2 antibodies comprising pre-selected desired N-glycan structures.

The present invention provides a composition comprising Her2 antibody molecules with N-glycans, wherein less than 20 mole % of the N-glycans comprise a Man5 core structure, and the N-glycan G0+G1+G2 content of the Her2 antibody molecules is more than 75 mole %.

### BRIEF DESCRIPTION OF THE DRAWINGS

FIG. 1 illustrates the N-glycosylation pathways in humans and P. pastoris. Early events in the ER are highly conserved, including removal of three glucose residues by glucosidases I and II and trimming of a single specific α-1,2-linked mannose residue by the ER mannosidase leading to the same core structure, Man₈GlcNAc₂ (Man8B). However, processing events diverge in the Golgi. Mns, α-1,2-mannosidase; MnsII, mannosidase II; GnT I, α-1,2-N-acetylglucosaminyltransferase I; GnT II, α-1,2-N-acetylglucosaminyltransferase II; MnT, mannosyltransferase. The two core GlcNAc residues, though present in all cases, were omitted in the nomenclature.

FIG. 2 illustrates the key intermediate steps in N-glycosylation as well as a shorthand nomenclature referring to the genetically engineered Pichia pastoris strains producing the respective glycan structures (GS).

FIG. 3 shows the construction of P. pastoris glycoengineered strain YDX477. P. pastoris strain YGLY16-3 (Δoch1, Δpno1, Δbmt2, Δmnn4a, Δmnn4b) was generated by knock-out of five yeast glycosyltransferases. Subsequent knock-in of eight heterologous genes, yielded RDP697-1, a strain capable of transferring the human N-glycan Gal₂GlcNAc₂Man₃GlcNAc₂ to secreted proteins. Introduction of a plasmid expressing a secreted antibody and a plasmid expressing a secreted form of Trichoderma reesei MNS1 yielded strain YDX477. CS, counterselect.

FIG. 4A-C shows a MALDI-TOF MS analysis of N-glycans on an anti-Her2 antibody produced in strain YDX477 either induced in BMMY medium alone or in medium containing galactose. Strains were cultivated in 150 mL of BMGY for 72 hours, then split and 50 mL aliquots of culture broths were centrifuged and induced for 24 hours in 25 mL of BMMY, 25 mL of BMMY+0.1% galactose, or 25 mL of BMMY+0.5% galactose. Protein A purified protein was subjected to Protein N-glycosidase F digestion and the released N-glycans analyzed by MALDI-TOF MS.

FIG. 5 shows a feature diagram of plasmid pRCD742a. This plasmid is a KINKO plasmid that integrates into the P. pastoris ADE1 locus without deleting the gene, and contains the PpURA5 selectable marker. The plasmid contains an expression cassette encoding a secretory pathway targeted fusion protein (FB8 MannI) comprising the ScSEC12 leader peptide fused to the N-terminus of the mouse Mannosidase I catalytic domain under the control of the PpGAPDH promoter, an expression cassette encoding a secretory pathway targeted fusion protein (CONA10) comprising the PpSEC12 leader peptide fused to the N-terminus of the human GlcNAc Transferase I (GnT I) catalytic domain under the control of the PpPMA1 promoter, and an expression cassette encoding the full length mouse Golgi UDP-GlcNAc transporter (MmSLC35A3) under the control of the PpSEC4 promoter. TT refers to transcription termination sequence.

FIG. 6 shows a feature diagram of plasmid pRCD1006. This plasmid is a P. pastoris his1 knock-out plasmid that contains the PpURA5 gene as a selectable marker. The plasmid contains an expression cassette encoding a secretory pathway targeted fusion protein (XB33) comprising the ScMnt1 (ScKre2) leader peptide fused to the N-terminus of the human Galactosyl Transferase I catalytic domain under the control of the PpGAPDH promoter and expression cassettes encoding the full-length D. melanogaster Golgi UDP-galactose transporter (DmUGT) and the S. pombe UDP-galactose C4-epimerase (SpGALE) under the control of the PpOCH1 and PpPMA1 promoters, respectively. TT refers to transcription termination sequence.

FIG. 7 shows a feature diagram of plasmid pGLY167b. The plasmid is a P. pastoris arg1 knock-out plasmid that contains the PpURA3 selectable marker and contains an expression cassette encoding a secretory pathway targeted fusion protein (C0-KD53) comprising the ScMNN2 leader peptide fused to the N-terminus of the Drosophila melanogaster Mannosidase II catalytic domain under the control of the PpGAPDH promoter and an expression cassette encoding a secretory pathway targeted fusion protein (C0-TC54) comprising the ScMnn2 leader peptide fused to the N-terminus of the rat GlcNAc Transferase II (GnT II) catalytic domain under the control of the PpPMA1 promoter. TT refers to transcription termination sequence.

FIG. 8 shows a feature diagram of plasmid pGLY510. The plasmid is a roll-in plasmid that integrates into the P. pastoris TRP2 gene while duplicating the gene and contains an AOX1 promoter-SeCYC1 terminator expression cassette as well as the PpARG1 selectable marker. TT refers to transcription termination sequence.

FIG. 9 shows a feature diagram of plasmid pDX459-1. The plasmid is a roll-in plasmid that targets and integrates into the P. pastoris AOX2 promoter and contains the Zeo^{R} while duplicating the promoter. The plasmid contains separate expression cassettes encoding an anti-HER2 antibody Heavy chain and an anti-HER2 antibody Light chain, each fused at the N-terminus to the Aspergillus niger alpha-amylase signal sequence and under the control of the P. pastoris AOX1 promoter. TT refers to transcription termination sequence.

FIG. 10 shows a feature diagram of plasmid pGLY1138. This plasmid is a roll-in plasmid that integrates into the P. pastoris ADE1 locus while duplicating the gene and contains a ScARR3 selectable marker gene cassette that confers arsenite resistance as well as an expression cassette encoding a secreted Trichoderma reesei MNS1 comprising the MNS1 catalytic domain fused at its N-terminus to the S. cerevisiae alpha factor pre signal sequence under the control of the PpAOX1 promoter. TT refers to transcription termination sequence.

FIG. 11A-I shows the genealogy of P. pastoris strains YGLY13992 (FIG. 11F), YGLY12501 (FIG. 11G) and YGLY13979 (FIG. 11H) beginning from wild-type strain NRRL-Y11430 (FIG. 11A).

FIG. 12 shows a map of plasmid pGLY6301 encoding the LmSTT3D ORF under the control of the Pichia pastoris alcohol oxidase I (AOX1) promoter and S. cereviseae CYC transcription termination sequence. The plasmid is a roll-in vector that targets the URA6 locus. The selection of transformants uses arsenic resistance encoded by the S. cerevisiae ARR3 ORF under the control of the P. pastoris RPL10 promoter and S. cereviseae CYC transcription termination sequence.

FIG. 13 shows a map of plasmid pGLY6294 encoding the LmSTT3D ORF under the control of the P. pastoris GAPDH promoter and S. cereviseae CYC transcription termination sequence. The plasmid is a KINKO vector that targets the TRP1 locus: the 3′ end of the TRP10RF is adjacent to the P. pastoris ALG3 transcription termination sequence. The selection of transformants uses nourseothricin resistance encoded by the Streptomyces noursei nourseothricin acetyltransferase (NAT) ORF under the control of the Ashbya gossypii TEF1 promoter (PTEF) and Ashbya gossypii TEF1 termination sequence (TTEF).

FIG. 14 shows a map of plasmid pGLY6. Plasmid pGLY6 is an integration vector that targets the URA5 locus and contains a nucleic acid molecule comprising the S. cerevisiae invertase gene or transcription unit (ScSUC2) flanked on one side by a nucleic acid molecule comprising a nucleotide sequence from the 5′ region of the P. pastoris URA5 gene (PpURA5-5′) and on the other side by a nucleic acid molecule comprising the nucleotide sequence from the 3′ region of the P. pastoris URA5 gene (PpURA5-3′).

FIG. 15 shows a map of plasmid pGLY40. Plasmid pGLY40 is an integration vector that targets the OCH1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the OCH1 gene (PpOCH1-5′) and on the other side by a nucleic acid molecule comprising a nucleotide sequence from the 3′ region of the OCH1 gene (PpOCH1-3′).

FIG. 16 shows a map of plasmid pGLY43a. Plasmid pGLY43a is an integration vector that targets the BMT2 locus and contains a nucleic acid molecule comprising the K. lactis UDP-N-acetylglucosamine (UDP-GlcNAc) transporter gene or transcription unit (KlGlcNAc Transp.) adjacent to a nucleic acid molecule comprising the P. pastoris URA5 gene or transcription unit (PpURA5) flanked by nucleic acid molecules comprising lacZ repeats (lacZ repeat). The adjacent genes are flanked on one side by a nucleic acid molecule comprising a nucleotide sequence from the 5′ region of the BMT2 gene (PpPBS2-5′) and on the other side by a nucleic acid molecule comprising a nucleotide sequence from the 3′ region of the BMT2 gene (PpPBS2-3′).

FIG. 17 shows a map of plasmid pGLY48. Plasmid pGLY48 is an integration vector that targets the MNN4L1 locus and contains an expression cassette comprising a nucleic acid molecule encoding the mouse homologue of the UDP-GlcNAc transporter (MmGlcNAc Transp.) open reading frame (ORF) operably linked at the 5′ end to a nucleic acid molecule comprising the P. pastoris GAPDH promoter (PpGAPDH Prom) and at the 3′ end to a nucleic acid molecule comprising the S. cerevisiae CYC termination sequence (ScCYC TT) adjacent to a nucleic acid molecule comprising the P. pastoris URA5 gene or transcription unit (PpURA5) flanked by lacZ repeats (lacZ repeat) and in which the expression cassettes together are flanked on one side by a nucleic acid molecule comprising a nucleotide sequence from the 5′ region of the P. Pastoris MNN4L1 gene (PpMNN4L1-5′) and on the other side by a nucleic acid molecule comprising a nucleotide sequence from the 3′ region of the MNN4L1 gene (PpMNN4L1-3′).

FIG. 18 shows as map of plasmid pGLY45. Plasmid pGLY45 is an integration vector that targets the PNO1/MNN4 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the PNO1 gene (PpPN0′-5′) and on the other side by a nucleic acid molecule comprising a nucleotide sequence from the 3′ region of the MNN4 gene (PpMNN4-3′).

FIG. 19 shows a map of plasmid pGLY1430. Plasmid pGLY1430 is a KINKO integration vector that targets the ADE1 locus without disrupting expression of the locus and contains in tandem four expression cassettes encoding (1) the human GlcNAc transferase I catalytic domain (codon optimized) fused at the N-terminus to P. pastoris SEC12 leader peptide (CO-NA10), (2) mouse homologue of the UDP-GlcNAc transporter (MmTr), (3) the mouse mannosidase IA catalytic domain (FB) fused at the N-terminus to S. cerevisiae SEC12 leader peptide (FB8), and (4) the P. pastoris URA5 gene or transcription unit (PpURA5) flanked by lacZ repeats (lacZ). All flanked by the 5′ region of the ADE1 gene and ORF (ADE1 5′ and ORF) and the 3′ region of the ADE1 gene (PpADE1-3′). PpPMA1 prom is the P. pastoris PMA1 promoter; PpPMA1 TT is the P. pastoris PMA1 termination sequence; SEC4 is the P. pastoris SEC4 promoter; OCH1 TT is the P. pastoris OCH1 termination sequence; ScCYC TT is the S. cerevisiae CYC termination sequence; PpOCH1 Prom is the P. pastoris OCH1 promoter; PpALG3 TT is the P. pastoris ALG3 termination sequence; and PpGAPDH is the P. pastoris GADPH promoter.

FIG. 20 shows a map of plasmid pGLY582. Plasmid pGLY582 is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33), (3) the P. pastoris URA5 gene or transcription unit (PpURA5) flanked by lacZ repeats (lacZ repeat), and (4) the D. melanogaster UDP-galactose transporter (DmUGT). All flanked by the 5′ region of the HIS1 gene (PpHIS1-5′) and the 3′ region of the HIS1 gene (PpHIS1-3′). PMA1 is the P. pastoris PMA1 promoter; PpPMA1 TT is the P. pastoris PMA1 termination sequence; GAPDH is the P. pastoris GADPH promoter and ScCYC TT is the S. cerevisiae CYC termination sequence; PpOCH1 Prom is the P. pastoris OCH1 promoter and PpALG12 TT is the P. pastoris ALG12 termination sequence.

FIG. 21 shows a map of plasmid pGLY167b. Plasmid pGLY167b is an integration vector that targets the ARG1 locus and contains in tandem three expression cassettes encoding (1) the D. melanogaster mannosidase II catalytic domain (codon optimized) fused at the N-terminus to S. cerevisiae MNN2 leader peptide (C0-KD53), (2) the P. pastoris HIS1 gene or transcription unit, and (3) the rat N-acetylglucosamine (GlcNAc) transferase II catalytic domain (codon optimized) fused at the N-terminus to S. cerevisiae MNN2 leader peptide (CO-TC54). All flanked by the 5′ region of the ARG1 gene (PpARG1-5′) and the 3′ region of the ARG1 gene (PpARG1-3′). PpPMA1 prom is the P. pastoris PMA1 promoter; PpPMA1 TT is the P. pastoris PMA1 termination sequence; PpGAPDH is the P. pastoris GADPH promoter; ScCYC TT is the S. cerevisiae CYC termination sequence; PpOCH1 Prom is the P. pastoris OCH1 promoter; and PpALG12 TT is the P. pastoris ALG12 termination sequence.

FIG. 22 shows a map of plasmid pGLY3411 (pSH1092). Plasmid pGLY3411 (pSH1092) is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris BMT4 gene (PpPBS4 5′) and on the other side with the 3′ nucleotide sequence of the P. pastoris BMT4 gene (PpPBS4 3′).

FIG. 23 shows a map of plasmid pGLY3419 (pSH1110). Plasmid pGLY3430 (pSH1115) is an integration vector that contains an expression cassette comprising the P. pastoris URA5 gene or transcription unit (PpURA5) flanked by lacZ repeats (lacZ repeat) flanked on one side with the 5′ nucleotide sequence of the P. pastoris BMT1 gene (PBS1 5′) and on the other side with the 3′ nucleotide sequence of the P. pastoris BMT1 gene (PBS1 3′)

FIG. 24 shows a map of plasmid pGLY3421 (pSH1106). Plasmid pGLY4472 (pSH1186) contains an expression cassette comprising the P. pastoris URA5 gene or transcription unit (PpURA5) flanked by lacZ repeats (lacZ repeat) flanked on one side with the 5′ nucleotide sequence of the P. pastoris BMT3 gene (PpPBS3 5′) and on the other side with the 3′ nucleotide sequence of the P. pastoris BMT3 gene (PpPBS3 3′).

FIG. 25 shows a map of plasmid pGLY3673. Plasmid pGLY3673 is a KINKO integration vector that targets the PRO1 locus without disrupting expression of the locus and contains expression cassettes encoding the T. reesei α-1,2-mannosidase catalytic domain fused at the N-terminus to S. cerevisiae aMATpre signal peptide (aMATTrMan) to target the chimeric protein to the secretory pathway and secretion from the cell.

FIG. 26 shows a map of pGLY6833 encoding the light and heavy chains of an anti-Her2 antibody. The plasmid is a roll-in vector that targets the TRP2 locus. The ORFs encoding the light and heavy chains are under the control of a P. pastoris AOX1 promoter and the P. pastoris CIT1 3UTR transcription termination sequence. Selection of transformants uses zeocin resistance encoded by the zeocin resistance protein (Zeocin^{R}) ORF under the control of the S. cereviseae TEF promoter and S. cereviseae CYC termination sequence.

FIG. 27 shows a map of pGLY5883 encoding the light and heavy chains of an anti-Her2 antibody. The plasmid is a roll-in vector that targets the TRP2 locus. The ORFs encoding the light and heavy chains are under the control of a P. pastoris AOX1 promoter and the S. cereviseae CYC transcription termination sequence. Selection of transformants uses zeocin resistance encoded by the zeocin resistance protein (Zeocin^{R}) ORF under the control of the S. cereviseae TEF promoter and S. cereviseae CYC termination sequence.

FIG. 28 shows a map of pGLY6830 encoding the light and heavy chains of an anti-Her2 antibody. The plasmid is a roll-in vector that targets the TRP2 locus. The ORFs encoding the light and heavy chains are under the control of a P. pastoris AOX1 promoter and the P. pastoris AOX1 transcription termination sequence. Selection of transformants uses zeocin resistance encoded by the zeocin resistance protein (Zeocin^{R}) ORF under the control of the S. cereviseae TEF promoter and S. cereviseae CYC termination sequence

FIG. 29 ADCC activities of trastuzumab, Her2 antibodies from strains YGLY12501, YGL13992 and YGLY13979 using human NK cells as effector cells.

FIG. 30 Serum concentration vs time curve after single IV administration (5 mg/kg) of Her2 antibody from strain YGLY12501 and Herceptin® in Cynomolgus monkeys (Data expressed as mean±SD, N=3).

FIG. 31 Plasma concentration vs time curve of Anti-Her2 expressed in GFI5.0 Pichia, GFI2.0 Pichia and wild-type pichia and commercial Herceptin produced in CHO cells.

FIG. 32 Plasma time vs-concentration curve after single IV administration of Anti-Her2 from strains YGLY13992(2), YGLY13979(2), YGLY13979 or Herceptin® in C57B6 mice (N=5).

FIG. 33 Her2 antibodies from strains YGLY13979, YGLY12501 and YGLY13992 binding to C1q in comparison with Herceptin®.

FIG. 34 Her2 antibodies from strains YGLY13979, YGLY12501 and YGLY13992 mediated C3b deposition in comparison with Herceptin®.

### DETAILED DESCRIPTION OF THE INVENTION

Unless otherwise defined herein, scientific and technical terms and phrases used in connection with the present invention shall have the meanings that are commonly understood by those of ordinary skill in the art. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

The following terms, unless otherwise indicated, shall be understood to have the following meanings:

The term “G0” when used herein refers to a complex bi-antennary oligosaccharide without galactose and fucose, GlcNAc₂Man₃GlcNAc₂.

The term “G1” when used herein refers to a complex bi-antennary oligosaccharide without fucose and containing one galactosyl residue, GalGlcNAc₂Man₃GlcNAc₂.

The term “G2” when used herein refers to a complex bi-antennary oligosaccharide without fucose and containing two galactosyl residues, Gal₂GlcNAc₂Man₃GlcNAc₂.

The term “G0F” when used herein refers to a complex bi-antennary oligosaccharide containing a core fucose and without galactose, GlcNAc₂Man₃GlcNAc₂F.

The term “G1F” when used herein refers to a complex bi-antennary oligosaccharide containing a core fucose and one galactosyl residue, GalGlcNAc₂Man₃GlcNAc₂F.

The term “G2F” when used herein refers to a complex bi-antennary oligosaccharide containing a core fucose and two galactosyl residues, Gal₂GlcNAc₂Man₃GlcNAc₂F.

The term “Man5” when used herein refers to the oligosaccharide structure shown as

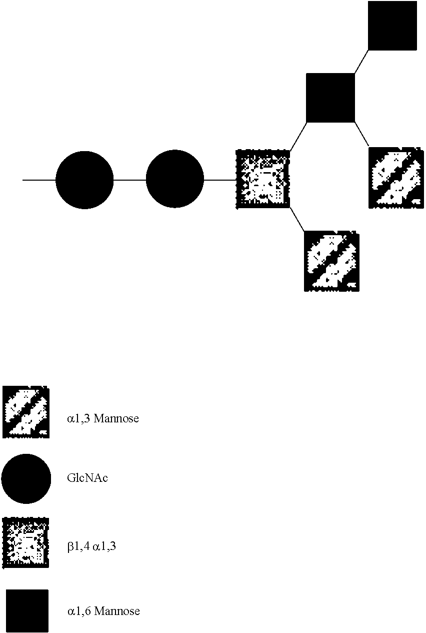

The term “GFI 5.0” when used herein refers to glycoengineered Pichia pastoris strains that produce glycoproteins having predominantly Gal₂GlcNAc₂Man₃GlcNAc₂ N-glycans.

The term “wild type” or “wt” when used herein refers to a native Pichia pastoris strain that has not been subjected to genetic modification to control glycosylation.

As used herein, the term “predominantly” or variations such as “the predominant” or “which is predominant” will be understood to mean the glycan species that has the highest mole percent (%) of total neutral N-glycans after the glycoprotein has been treated with PNGase and released glycans analyzed by mass spectroscopy, for example, MALDI-TOF MS or HPLC. In other words, the phrase “predominantly” is defined as an individual entity, such as a specific glycoform, is present in greater mole percent than any other individual entity. For example, if a composition consists of species A in 40 mole percent, species 13 in 35 mole percent and species C in 25 mole percent, the composition comprises predominantly species A, and species B would be the next most predominant species. Some host cells may produce compositions comprising neutral N-glycans and charged N-glycans such as mannosylphosphate. Therefore, a composition of glycoproteins can include a plurality of charged and uncharged or neutral N-glycans. In the present invention, it is within the context of the total plurality of neutral N-glycans in the composition in which the predominant N-glycan determined. Thus, as used herein, “predominant N-glycan” means that of the total plurality of neutral N-glycans in the composition, the predominant N-glycan is of a particular structure.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent.

As used herein, a glycoprotein composition “lacks” or “is lacking” a particular sugar residue, such as fucose or galactose, when no detectable amount of such sugar residue is present on the N-glycan structures at any time. For example, in embodiments of the present invention, the glycoprotein compositions are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues.

The term “high mannose” type N-glycan when used herein refers to an N-glyan having five or more mannose residues.

“O-mannose” refers to O-linked mannose at a Serine or Theoronine residue on the antibody. At a single O-glycosylation site, there can be multiple or single mannose linked.

The term “complex” type N-glycan when used herein refers to an N-glycan having at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). As an example, when a N-glycan comprises a bisecting GlcNAc on the trimannose core, the structure can be represented as Man₃GlcNAc₂(GlcNAc) or Man₃GlcNAc₃. When an N-glycan comprises a core fucose attached to the trimannose core, the structure may be represented as Man₃GlcNAc₂(Fuc). Complex N-glycans may also have multiple antennae on the “trimannose core,” often referred to as “multiple antennary glycans.”

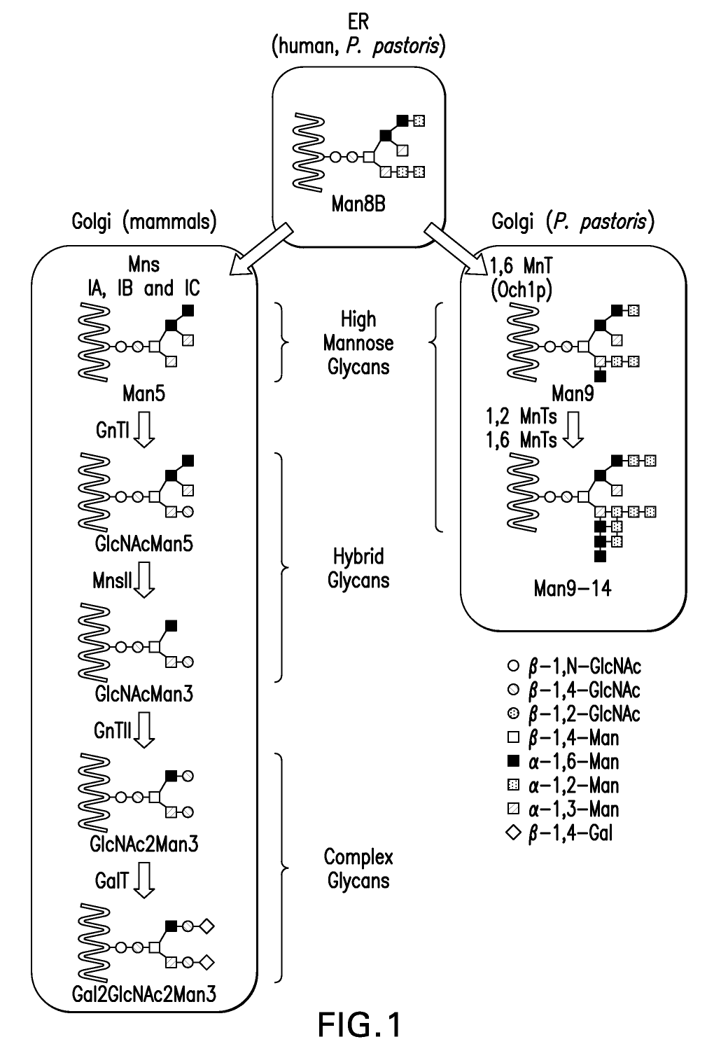

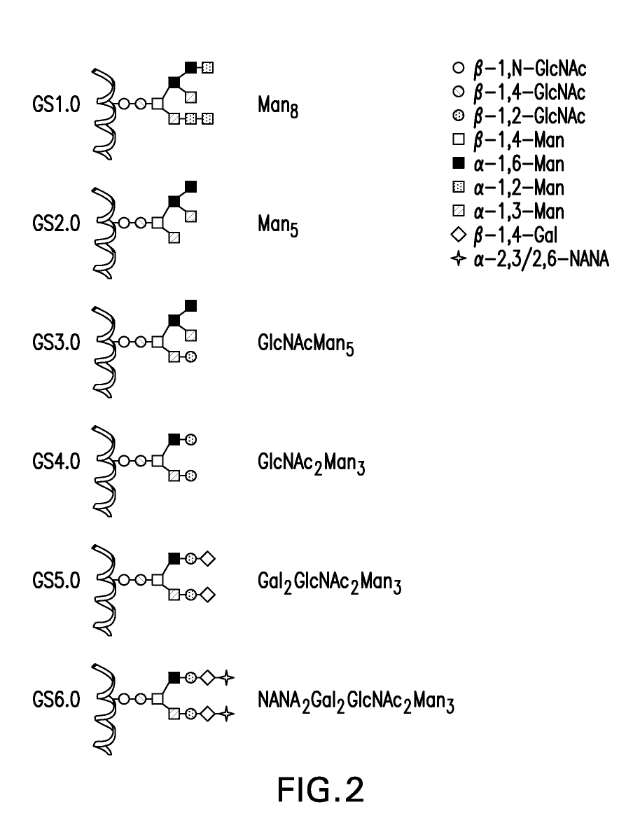

The term “hybrid” N-glycan when used herein refers to an N-glycan having at least one GlcNAc on the terminal of the 1,3 mannose arm of the trimannose core and zero or more than one mannose on the 1,6 mannose arm of the trimannose core. In one embodiment, the hybrid form is GlcNAcMan₅GlcNAc₂ with the structure (see FIG. 1 for annotations):

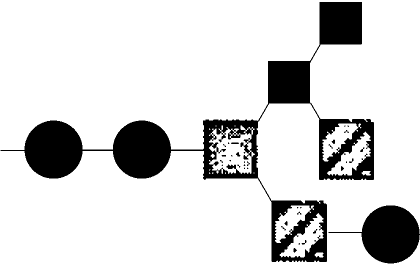

In another embodiment, the hybrid form is GalGlcNAcMan₅GlcNAc₂ with the structure

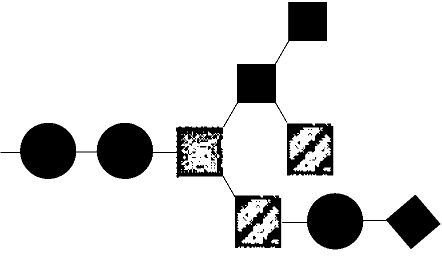

When referring to “mole percent” of a glycan present in a preparation of a glycoprotein, the term means the molar percent of a particular glycan present in the pool of N linked oligosaccharides released when the protein preparation is treated with PNG′ase and then quantified by a method that is not affected by glycoform composition, (for instance, labeling a PNG'ase released glycan pool with a fluorescent tag such as 2-aminobenzamide and then separating by high performance liquid chromatography or capillary electrophoresis and then quantifying glycans by fluorescence intensity). For example, 50 mole percent GlcNAc₂Man₃GlcNAc₂Ga12NANA2 means that 50 percent of the released glycans are GlcNAc₂Man₃GleNAc₂Ga12NANA2 and the remaining 50 percent are comprised of other N-linked oligosaccharides.

The term “Her2 antibody” or“Anti-Her2” when used herein refers to a humanized anti-Her2 antibody comprising the light chain amino acid sequence of SEQ ID NO:18 and the heavy chain amino acid sequence of SEQ ID NO: 16 or 20 or amino acid sequence variants thereof which retain the ability to bind the Her2 epitope that trastuzumab binds and inhibits growth of tumor cells that overexpress HER2. In one embodiment, the Fc region is substituted with another native Fc region of different allotype. In another embodiment, the amino acid sequence variants are conservative mutations.

As used herein, the terms “antibody,” “immunoglobulin,” “immunoglobulins”, “IgG1”, “antibodies”, and “immunoglobulin molecule” are used interchangeably. Each immunoglobulin molecule has a unique structure that allows it to bind its specific antigen, but all immunoglobulins have the same overall structure as described herein. The basic immunoglobulin structural unit is known to comprise a tetramer of subunits. Each tetramer has two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Light chains are classified as either kappa or lambda. Heavy chains are classified as gamma, mu, alpha, delta, or epsilon, and define the antibody's isotype as IgG, IgM, IgA, IgD, and IgE, respectively.

The light and heavy chains are subdivided into variable regions and constant regions (See generally, Fundamental Immunology (Paul, W., ed., 2nd ed. Raven Press, N.Y., 1989), Ch. 7). The variable regions of each light/heavy chain pair form the antibody binding site. Thus, an intact antibody has two binding sites. Except in bifunctional or bispecific immunoglobulins, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. The terms include naturally occurring forms, as well as fragments and derivatives. Included within the scope of the term are classes of immunoglobulins (Igs), namely, IgG, IgA, IgE, IgM, and IgD. Also included within the scope of the terms are the subtypes of IgGs, namely, IgG1, IgG2, IgG3, and IgG4. The term is used in the broadest sense and includes single monoclonal immunoglobulins (including agonist and antagonist immunoglobulins) as well as antibody compositions which will bind to multiple epitopes or antigens. The terms specifically cover monoclonal immunoglobulins (including full length monoclonal immunoglobulins), polyclonal immunoglobulins, multispecific immunoglobulins (for example, bispecific immunoglobulins), and antibody fragments so long as they contain or are modified to contain at least the portion of the CH₂ domain of the heavy chain immunoglobulin constant region which comprises an N-linked glycosylation site of the CH₂ domain, or a variant thereof.

The term “monoclonal antibody” (mAb) as used herein refers to an antibody obtained from a population of substantially homogeneous immunoglobulins, i.e., the individual immunoglobulins comprising the population are identical except for possible naturally occurring mutations that may be present in minor amounts. Monoclonal immunoglobulins are highly specific, being directed against a single antigenic site. Furthermore, in contrast to conventional (polyclonal) antibody preparations which typically include different immunoglobulins directed against different determinants (epitopes), each mAb is directed against a single determinant on the antigen. In addition to their specificity, monoclonal immunoglobulins are advantageous in that they can be synthesized by hybridoma culture, uncontaminated by other immunoglobulins. The term “monoclonal” indicates the character of the antibody as being obtained from a substantially homogeneous population of immunoglobulins, and is not to be construed as requiring production of the antibody by any particular method. For example, the monoclonal immunoglobulins to be used in accordance with the present invention may be made by the hybridoma method first described by Kohler et al., Nature, 256:495 (1975), or may be made by recombinant DNA methods (See, for example, U.S. Pat. No. 4,816,567 to Cabilly et al.).

“Humanized antibodies” are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

The term “fragments” within the scope of the terms “antibody” or “immunoglobulin” include those produced by digestion with various proteases, those produced by chemical cleavage and/or chemical dissociation and those produced recombinantly, so long as the fragment remains capable of specific binding to a target molecule. Among such fragments are Fc, Fab, Fab′, Fv, F(ab′)₂, and single chain Fv (scFv) fragments. Hereinafter, the term “immunoglobulin” also includes the term “fragments” as well.

Immunoglobulins further include immunoglobulins or fragments that have been modified in sequence but remain capable of specific binding to a target molecule, including: interspecies chimeric and humanized immunoglobulins; antibody fusions; heteromeric antibody complexes and antibody fusions, such as diabodies (bispecific immunoglobulins), single-chain diabodies, and intrabodies (See, for example, Intracellular Immunoglobulins: Research and Disease Applications, (Marasco, ed., Springer-Verlag New York, Inc., 1998).

The term “Fc” fragment refers to the ‘fragment crystallized’ C-terminal region of the antibody containing the CH₂ and CH₃ domains. The term “Fab” fragment refers to the ‘fragment antigen binding’ region of the antibody containing the V_{H}, C_{H}1, V_{L} and C_{L} domains.

A “native Fc region” comprises an amino acid sequence identical to the amino acid sequence of a Fc region found in nature, which includes allotypes of the human Fc regions.

“Antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII.

The terms “purified” or “isolated” protein or polypeptide refers to a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free or purified of naturally associated components by isolation, using protein purification techniques well known in the art. As thus defined, “isolated” does not necessarily require that the protein, polypeptide, peptide or oligopeptide so described has been physically removed from its native environment.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) In a preferred embodiment, a homologous protein is one that exhibits at least 65% sequence homology to the wild type protein, more preferred is at least 70% sequence homology. Even more preferred are homologous proteins that exhibit at least 75%, 80%, 85% or 90% sequence homology to the wild type protein. In the most preferred embodiment, a homologous protein exhibits at least 95%, 98%, 99% or 99.9% sequence identity. As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule.

As used herein, the term “comprise” or variations such as “comprises” or “comprising”, will be understood to imply the inclusion of a stated integer or group of integers but not the exclusion of any other integer or group of integers.

The term “eukaryotic” refers to a nucleated cell or organism, and includes insect cells, plant cells, mammalian cells, animal cells and lower eukaryotic cells.

The term “lower eukaryotic cells” includes yeast, fungi, collar-flagellates, microsporidia, alveolates (e.g., dinoflagellates), stramenopiles (e.g, brown algae, protozoa), rhodophyta (e.g., red algae), plants (e.g., green algae, plant cells, moss) and other protists.

The terms “yeast” and “fungi” include, but are not limited to: Pichia sp., Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Saccharomyces sp., Saccharomyces cerevisiae, Hansenula polymorpha, Kluyveromyces sp., Kluyveromyces lactis, Candida albicans, Aspergillus sp., Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa.

### I. Glycosylation

N-glycosylation in most eukaryotes begins in the endoplasmic reticulum (ER) with the transfer of a lipid-linked Glc₃Man₉GlcNAc₂ oligosaccharide structure onto specific Asn residues of a nascent polypeptide (Lehle and Tanner, Biochim. Biophys. Acta 399: 364-74 (1975); Kornfeld and Kornfeld, Annu. Rev. Biochem 54: 631-64 (1985); Burda and Aebi, Biochim. Biophys. Acta-General Subjects 1426: 239-257 (1999)). Trimming of all three glucose moieties and a single specific mannose sugar from the N-linked oligosaccharide results in Man₈GlcNAc₂ (See FIG. 1), which allows translocation of the glycoprotein to the Golgi apparatus where further oligosaccharide processing occurs (Herscovics, Biochim. Biophys. Acta 1426: 275-285 (1999); Moremen et al., Glycobiology 4: 113-125 (1994)). It is in the Golgi apparatus that mammalian N-glycan processing diverges from yeast and many other eukaryotes, including plants and insects. Mammals process N-glycans in a specific sequence of reactions involving the removal of three terminal α-1,2-mannose sugars from the oligosaccharide before adding GlcNAc to form the hybrid intermediate N-glycan GlcNAcMan₅GlcNAc₂ (Schachter, Glycoconj. J. 17: 465-483 (2000)) (See FIG. 1). This hybrid structure is the substrate for mannosidase II, which removes the terminal α-1,3- and α-1,6-mannose sugars on the oligosaccharide to yield the N-glycan GlcNAcMan₃GlcNAc₂ (Moremen, Biochim. Biophys. Acta 1573(3): 225-235 (1994)). Finally, as shown in FIG. 1, complex N-glycans are generated through the addition of at least one more GlcNAc residue followed by addition of galactose and sialic acid residues (Schachter, (2000), above), although sialic acid is often absent on certain human proteins, including IgGs (Keusch et al., Clin. Chim. Acta 252: 147-158 (1996); Creus et al., Clin. Endocrinol. (Oxf) 44: 181-189 (1996)).

In Saccharomyces cerevisiae, N-glycan processing involves the addition of mannose sugars to the oligosaccharide as it passes throughout the entire Golgi apparatus, sometimes leading to hypermannosylated glycans with over 100 mannose residues (Trimble and Verostek, Trends Glycosci. Glycotechnol. 7: 1-30 (1995); Dean, Biochim. Biophys. Acta-General Subjects 1426: 309-322 (1999)) (See FIG. 1). Following the addition of the first α-1,6-mannose to Man₈GlcNAc₂ by α-1,6-mannosyltransferase (Och1p), additional mannosyltransferases extend the Man₉GlcNAc₂ glycan with α-1,2-, α-1,6-, and terminal α-1,3-linked mannose as well as mannosyiphosphate. Pichia pastoris is a methylotrophic yeast frequently used for the expression of heterologous proteins, which has glycosylation machinery similar to that in S. cerevisiae, (Bretthauer and Castellino, Biotechnol. Appl. Biochem. 30: 193-200 (1999); Cereghino and Cregg, Ferns Microbiol. Rev. 24: 45-66 (2000); Verostek and Trimble, Glycobiol. 5: 671-681 (1995)). However, consistent with the complexity of N-glycosylation, glycosylation in P. pastoris differs from that in S. cerevisiae in that it lacks the ability to add terminal α-1,3-linked mannose, but instead adds other mannose residues including phosphornannose and β-linked mannose (Miura et al., Gene 324: 129-137 (2004); Blanchard et al., Glycoconj. J. 24: 33-47 (2007); Mille et al., J. Biol. Chem. 283: 9724-9736 (2008)).

The maturation of complex N-glycans involves the addition of galactose to terminal GlcNAc moieties, a reaction that can be catalyzed by several galactosyltransferases (Galls). In humans, there are seven isoforms of GalTs (I-VII), at least four of which have been shown to transfer galactose to terminal GlcNAc in the presence of UDP-galactose in vitro (Guo, et al., Glycobiol. 11: 813-820 (2001)). The first enzyme identified, known as GalTI, is generally regarded as the primary enzyme acting on N-glycans, which is supported by in vitro experiments, mouse knock-out studies, and tissue distribution analysis (Berger and Rohrer, Biochimie 85: 261-74 (2003); Furukawa and Sato, Biochim. Biophys. Acta 1473: 54-66 (1999)).

IgG antibodies have a single N-linked biantennary carbohydrate at Asn297 of the CH₂ domain. For human IgG, the core oligosaccharide normally consists of GlcNAc₂Man₃GlcNAc, with differing numbers of outer residues, such as attachment of galactose and/or galactose-sialic acid at the two terminal GlcNac or via attachment of a third GlcNAc arm (bisecting GlcNAc). The presence of absence of terminal galactose residues has been reported to affect function (Wright et al., J. Immunol. 160:3393-3402 (1998)).

The invention provides methods and materials for the transformation, expression and selection of recombinant proteins, particularly Her2 antibody, in lower eukaryotic host cells, which have been genetically engineered to produce glycoproteins with desired N-glycans. In certain embodiments, the eukaryotic host cells have been genetically engineered to produce Her2 antibody, or a variant of Her2 antibody, with desired N-glycans.

The present invention provides a composition comprising Her2 antibody molecules with N-glycans, wherein less than 20 mole % of the N-glycans comprise a Man5 core structure, and the N-glycan G0+G1+G2 content of the Her2 antibody molecules is more than 75 mole %. In one embodiment, the N-glycan is attached to Asn297 of the CH₂ domain of a Her2 antibody molecule.

In one embodiment, 17 mole % or less of the N-glycans comprise a Man5 core structure. In another embodiment, 15 mole % or less of the N-glycans comprise a Man5 core structure. In another embodiment, 12 mole % or less of the N-glycans comprise a Man5 core structure.

In another embodiment, 10 mole % or less of the N-glycans comprise a Man5 core structure. In yet another embodiment, 9 mole % or less of the N-glycans comprise a Man5 core structure. In another embodiment, 8 mole % or less of the N-glycans comprise a Man5 core structure. In a further embodiment, 6-9 mole % or less of the N-glycans comprise a Man5 core structure. In a further embodiment, 7-8 mole % or less of the N-glycans comprise a Man5 core structure. In a further embodiment, 5-12 mole % or less of the N-glycans comprise a Man5 core structure.

With respect to complex N-glycan content, in one embodiment, the N-glycan G0+G1+G2 content of the Her2 antibody molecules is 80 mole % or more. In another embodiment, 50-65 mole % of the N-glycan is G0, 5-25 mole % of the N-glycan is G1 and 1-10 mole % of the N-glycan is G2. In another embodiment, 50-61 mole % of the N-glycan is G0, 15-25 mole % of the N-glycan is G1 and 2-5 mole % of the N-glycan is G2. In a further embodiment, 59-60 mole % of the N-glycan is G0, 21-23 mole % of the N-glycan is G1 and 2-3 mole % of the N-glycan is G2.

Many wild-type lower eukaryotic cells, including yeasts and fungi, such as Pichia pastoris, produce glycoproteins without any core fucose. Thus, in the above embodiments, the antibodies produced in accordance with the present invention may lack fucose, or be essentially free of fucose. In a particular embodiment, the Her2 antibody molecules lack fucose. Alternatively, in certain embodiments, the recombinant lower eukaryotic host cells may be genetically modified to include a fucosylation pathway, thus resulting in the production of antibody compositions in which the predominant N-glycan species is fucosylated. Unless specifically noted, the antibody compositions of the present invention may be produced either in afucosylated form, or with core fucosylation present.

The Her2 antibody molecules of the invention may also comprise hybrid N-glycans of 12 mole % or less. The Her2 antibody molecules of the invention may also comprise hybrid N-glycans of 10 mole % or less. In one embodiment, the Her2 antibody molecules comprise hybrid N-glycans of 6-10 mole %. In another embodiment, the hybrid N-glycan is GlcNAcMan₅GlcNAc₂ or GalGlcNAcMan₅GlcNAc₂.

The Her2 antibody molecules of the invention can also have an N-glycosylation site occupancy of 75% or more. In another embodiment, the N-glycosylation site occupancy is 75-89 mole %. In another embodiment, the N-glycosylation site occupancy is 80-85 mole %.

In another embodiment, the Her2 antibody molecules in the composition comprise O-mannose, wherein the occupancy of the O-mannose is 1-3 mol/antibody mol. In another embodiment, more than 99% of the O-mannose contains a single mannose at the O-glycosylation site. In a further embodiment, the occupancy of the O-mannose is 1-2 mol/antibody mol. In a further embodiment, the occupancy of the O-mannose is 1 mol/antibody mol.

The Her2 antibody molecules of the above invention can also be characterized by functional properties. In one embodiment, the K_{D} for Her2 binding of the Her2 antibody molecules is 0.5-0.8 nM. In another embodiment, the relative potency of Her2 binding for the Her2 antibody molecules of the present invention as compared to Herceptin® is 1.5-2.0 fold higher. In a further embodiment, the relative potency of Her2 binding as compared to Herceptin® is 1.2-2.0 fold higher. In another embodiment, the ADCC activity is 4-6 fold higher than that of Herceptin®.

In a particular embodiment, the Her2 antibody has a light chain amino acid sequence according to SEQ ID NO: 18 and a heavy chain amino acid sequence according to SEQ ID NO: 16 or SEQ ID NO: 20. In a further embodiment, the heavy chain amino acid sequence is SEQ ID NO: 16 with a C-terminal lysine added. In another embodiment, the heavy chain amino acid sequence is SEQ ID NO: 20 with the C-terminal lysine deleted.

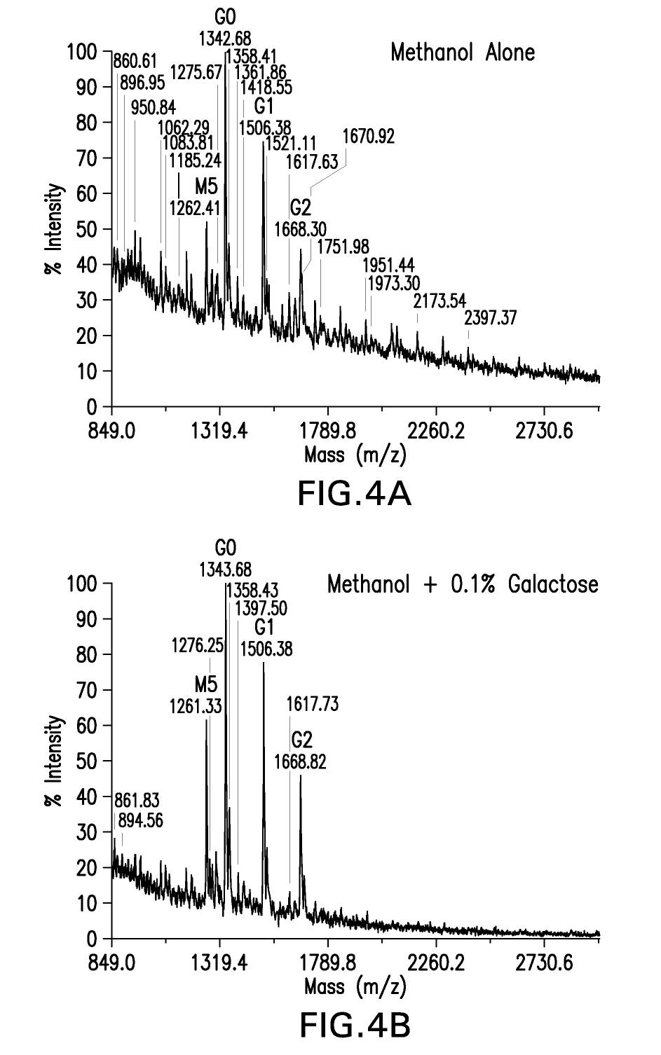

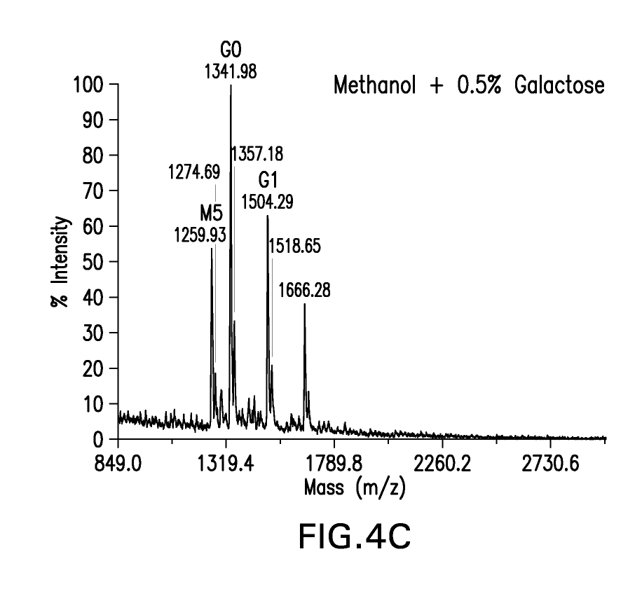

In a particular embodiment, the Her2 antibody molecules have an N-glycan profile substantially similar to FIG. 4A, 4B or 4C. In another particular embodiment, the Her2 antibody molecules have an N-glycan profile of 60% G0, 17% G1, 5% G2, 12% higher mannose, 7% hybrid N-glycans, and lack fucose. In another particular embodiment, the Her2 antibody molecules have an N-glycan profile of 80% G0+G1+ G2, 12% higher mannose, 7% hybrid N-glycans, and lack fucose. In another particular embodiment, the Her2 antibody molecules have an N-glycan profile of 60% G0, 21% G1, 3% G2, 8% Man5 and 8% Hybrid. In another particular embodiment, the Her2 antibody molecules have an N-glycan profile of 59% G0, 23% G1, 2% G2, 8% Man5 and 8% Hybrid. In another particular embodiment, the Her2 antibody molecules have an N-glycan profile of 59% G0, 23% G1, 3% G2, 7% Man5 and 8% Hybrid.

In a further embodiment, the present invention provides a composition comprising Her2 antibody molecules with N-glycans, wherein 5-12 mole % of the N-glycans comprise a Man5 core structure, the N-glycan G0+G1+G2 content of the Her2 antibody molecules is more than 75 mole %, the hybrid N-glycans is 11 mole % or less, the N-glycosylation site occupancy is 80-88 mole %, the N-glycans lack fucose, and the Her2 antibody has a light chain amino acid sequence according to SEQ ID NO: 18 and a heavy chain amino acid sequence according to SEQ ID NO: 16 or 20. In a further embodiment, the Her2 antibody molecules in the composition comprise O-mannose, wherein the occupancy of the O-mannose is 1 mol/antibody mol.

In another embodiment, the present invention provides a composition comprising Her2 antibody molecules with N-glycans, wherein 5-12 mole % of the N-glycans comprise a Man5 core structure, the N-glycan G0+G1+G2 content of the Her2 antibody molecules is 77-86 mole %, the hybrid N-glycans is 9-11 mole %, the N-glycosylation site occupancy is 82-88 mole %, the N-glycans lack fucose and the Her2 antibody has a light chain amino acid sequence according to SEQ ID NO: 18 and a heavy chain amino acid sequence according to SEQ ID NO: 16 or 20. In a further embodiment, the Her2 antibody molecules in the composition comprise O-mannose, wherein the occupancy of the O-mannose is 1 mol/antibody mol.

In another embodiment, the present invention provides a composition comprising Her2 antibody molecules with N-glycans, wherein 1-15 mole % of the N-glycans comprise a Man5 core structure, the N-glycan G0+G1+G2 content of the Her2 antibody molecules is 75-90 mole %, the hybrid N-glycans is 1-12 mole %, the N-glycosylation site occupancy is 80-90 mole %, the N-glycans lack fucose and the Her2 antibody has a light chain amino acid sequence according to SEQ ID NO: 18 and a heavy chain amino acid sequence according to SEQ ID NO: 16 or 20. In a further embodiment, the Her2 antibody molecules in the composition comprise O-mannose, wherein the occupancy of the O-mannose is 1 mol/antibody mol.

In a further embodiment, the present invention provides a composition comprising Her2 antibody molecules with N-glycans, wherein 8 mole % or less of the N-glycans comprise a Man5 core structure, the N-glycan G0+G1+G2 content of the Her2 antibody molecules is 77-84 mole %, the hybrid N-glycans is 9-11 mole %, the N-glycosylation site occupancy is 84-88 mole %, and the Her2 antibody has a light chain amino acid sequence according to SEQ ID NO: 18 and a heavy chain amino acid sequence according to SEQ ID NO: 16. In a further embodiment, the Her2 antibody molecules in the composition comprise O-mannose, wherein the occupancy of the O-mannose is 1 mol/antibody mol. In one embodiment, the N-glycan lacks fucose.

### II. Formulations

The compositions of the present invention can be formulated in a pharmaceutical composition in lyophilized or liquid form. Protein stabilizers, buffers, surfactants may be included in the pre-lyophilized formulations to enhance stability during the freeze drying process and/or improve stability of the lyophilized product upon storage.

Depending on the desired dose volumes, one can determine the amount of antibody present in the pre-lyophilized formulation. In one embodiment, the starting concentration of the antibody is about 10 mg/ml to about 50 mg/ml. In another embodiment, the starting concentration of the antibody is about 20 mg/ml to about 30 mg/ml. In a further embodiment, the starting concentration of the antibody is about 21 mg/ml.

The antibody may be present in a pH buffered solution pre-lyophilized formulation at pH from about 4-8 or 5-7. In one embodiment, the pH is 6. Exemplary buffers include histidine, phosphate, Tris, citrate, succinate and other organic acids. The buffer concentration can be from about 1 mM to about 100 mM, or from about 5 mM to about 50 mM. In one embodiment, the buffer is histidine.

Stablizers such as non-reducing sugars can be added to the pre-lyophilized formulation. In one embodiment, the non-reducing sugar is sucrose or trehalose. Other stabilizers include but are not limited to amino acids such as arginine, histidine, lysine and proline, polymers such as PEG, dextran and cyclodextrin, and polyols such as glycerol, mannitol and sorbitol. Exemplary concentrations of stablizers range from about 10 mM to about 400 mM, from about 30 mM to about 300 mM, or from about 50 mM to about 150 mM.

A surfactant can be added to the pre-lyophilized formulation, lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; polyethyl glycol, polypropyl glycol, and copolymers of ethylene and propylene glycol (e.g. Pluronics, PF68 etc). The amount of surfactant added is such that it reduces aggregation of the reconstituted protein and minimizes the formation of particulates after reconstitution. For example, the surfactant may be present in the pre-lyophilized formulation in an amount from about 0.001-0.5%, and preferably from about 0.005-0.05%.

In one embodiment, the lyophilized formulation comprises 21 mg/ml of Her2 antibody, 60 mM trehalose, 5 mM Histidine, pH 6 and 0.009% polysorbate-20. In one embodiment, the lyophilized formulation comprises 21 mg/ml of Her2 antibody, 50 mM sucrose, 5 mM Histidine, pH 6, 20 mM Arginine and 0.005% polysorbate-20. In another embodiment, the lyophilized formulation comprises 21 mg/ml of Her2 antibody, 30 mM trehalose, 20 mM Histidine, pH 6, 50 mM Arginine and 0.005% polysorbate-20. In another embodiment, the lyophilized formulation comprises 21 mg/ml of Her2 antibody, 1% sucrose, 50 mM Histidine, pH 6, 20 mM Arginine and 0.005% polysorbate-20. In a further embodiment, the lyophilized formulation comprises 21 mg/ml of Her2 antibody, 2% sucrose, 50 mM Histidine, pH 6, 30 mM Arginine and 0.005% polysorbate-20. In a further embodiment, the lyophilized formulation comprises 21 mg/ml of Her2 antibody, 3% sucrose, 50 mM Histidine, pH 6, 50 mM Arginine and 0.005% polysorbate-20. In a further embodiment, the lyophilized formulation comprises 21 mg/ml of Her2 antibody, 4% sucrose, 50 mM Histidine, pH 6, 50 mM Arginine and 0.005% polysorbate-20. In yet a further embodiment, the lyophilized formulation comprises 21 mg/ml of Her2 antibody, 5% sucrose, 5 mM Phosphate, pH 6, 50 mM Arginine and 0.005% polysorbate-20.

### III. Administration

Prior to administration to a patient, the lyophilized formulation can be reconstituted to generate a stable reconsistuted formulation for administration, for example, intravenous or subcutaneous delivery.

The therapeutically effective amount of antibody needed to elicit the therapeutic response can be determined based on the age, health, size and sex of the subject. Optimal amounts can also be determined based on monitoring of the subject's response to treatment.

As used herein, the term “therapeutically effective amount” means that amount of active antibody that elicits the biological or medicinal response in a tissue, system, animal or human that is being sought by a researcher, veterinarian, medical doctor or other clinician. The therapeutic effect is dependent upon the disease or disorder being treated or the biological effect desired. As such, the therapeutic effect can be a decrease in the severity of symptoms associated with the disease or disorder and/or inhibition (partial or complete) of progression of the disease.

In the present invention, when the antibody is used to treat or prevent cancer, the desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

The Her2 antibody of the invention can be administered at 0.1-20 mg/kg in one or more separate administrations. In one embodiment, the dosage is 1-10 mg/kg. In an embodiment of the invention, the initial dose of anti-Her2 is 6 mg/kg, 8 mg/kg, or 12 mg/kg. The subsequent maintenance doses are 2 mg/kg delivered once per week by intravenous infusion, intravenous bolus injection, subcutaneous infusion, or subcutaneous bolus injection. In another embodiment, the invention includes an initial dose of 12 mg/kg anti-Her2 antibody, followed by subsequent maintenance doses of 6 mg/kg once per 3 weeks. In still another embodiment, the invention includes an initial dose of 8 mg/kg anti-Her2 antibody, followed by 6 mg/kg once per 3 weeks. In yet another embodiment, the invention includes an initial dose of 8 mg/kg anti-Her2 antibody, followed by subsequent maintenance doses of 8 mg/kg once per week or 8 mg/kg once every 2 to 3 weeks. In another embodiment, the invention includes an initial dose of 4 mg/kg anti-Her2 antibody, followed by subsequent maintenance doses of 2 mg/kg once per week.

The anti-Her2 antibody may be used for the treatment of metastatic breast cancer as single agent or in combination with paclitaxel, docetaxel or an aromatase inhibitor. The anti-Her2 antibody may also be used for the treatment of early breast cancer as single agent; as part of treatment regimen consisting of doxorubicin, cyclophosphamide, and either paclitaxel or docetaxel; or in combination with docetaxel and carboplatin, in a neoadjuvant or adjuvant setting. The anti-Her2 antibody may also be used to treat ovarian, stomach, endometrial, salivary gland, lung, kidney, colon and/or bladder cancer.

### IV. Nucleic Acid Encoding the Glycoprotein

The Her2 antibodies of the present invention are encoded by nucleic acids. The nucleic acids can be DNA or RNA, typically DNA. The nucleic acid encoding the glycoprotein is operably linked to regulatory sequences that allow expression of the glycoprotein. Such regulatory sequences include a promoter and optionally an enhancer upstream, or 5′, to the nucleic acid encoding the fusion protein and a transcription termination site 3′ or down stream from the nucleic acid encoding the glycoprotein. The nucleic acid also typically encodes a 5′ UTR region having a ribosome binding site and a 3′ untranslated region. The nucleic acid is often a component of a vector which transfers to nucleic acid into host cells in which the glycoprotein is expressed. The vector can also contain a marker to allow recognition of transformed cells. However, some host cell types, particularly yeast, can be successfully transformed with a nucleic acid lacking extraneous vector sequences.

Nucleic acids encoding desired Her2 antibody of the present invention can be obtained from several sources. cDNA sequences can be amplified from cell lines known to express the glycoprotein using primers to conserved regions (see, e.g., Marks et al., J. Mol. Biol. 581-596 (1991)). Nucleic acids can also be synthesized de novo based on sequences in the scientific literature. Nucleic acids can also be synthesized by extension of overlapping oligonucleotides spanning a desired sequence of a larger nucleic acid, e.g., genomic DNA (see, e.g., Caldas et al., Protein Engineering, 13, 353-360 (2000)).

### V. Host Cells

In one embodiment, expression of the Her2 antibody of the present invention is in Lower eukaryotic cells, such as yeast and fungi, because they can be economically cultured, provide high yields, and when appropriately modified are capable of suitable glycosylation. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired.

In one embodiment, various yeasts, such as K. lactis, Pichia pastoris, Pichia methanolica, and Hansenula polymorpha are used for cell culture because they are able to grow to high cell densities and secrete large quantities of recombinant protein. Likewise, filamentous fungi, such as Trichoderma reesei, Aspergillus niger, Fusarium sp, Neurospora crassa and others can be used to produce glycoproteins of the invention.

Lower eukaryotes, particularly yeast and fungi, can be genetically modified so that they express glycoproteins in which the glycosylation pattern is human-like or humanized. This can be achieved by eliminating selected endogenous glycosylation enzymes and/or supplying exogenous enzymes as described by Gemgross et al., US 20040018590 and U.S. Pat. No. 7,029,872, the disclosures of which are hereby incorporated herein by reference. For example, a host cell can be selected or engineered to be depleted in 1,6-mannosyl transferase activities, which would otherwise add mannose residues onto the N-glycan on a glycoprotein.

In certain embodiments, a vector can be constructed with one or more selectable marker gene(s), and one or more desired genes encoding the Her2 antibody which is to be transformed into an appropriate host cell. For example, one or more genes selectable marker gene(s) can be physically linked with one or more gene(s), expressing a desired Her2 antibody for isolation or a fragment of said Her2 antibody having the desired activity can be associated with the selectable gene(s) within the vector. The selectable marker gene(s) and Her2 antibody gene(s) can be arranged on one or more transformation vectors so that presence of the Her2 antibody gene(s) in a transformed host cell is correlated with expression of the selectable marker gene(s) in the transformed cells. For example, the two genes can be inserted into the same physical plasmid, under control of a single promoter, or under the control of two separate promoters. It may also be desired to insert the genes into distinct plasmids and co-transformed into the cells.

Other cells useful as host cells in the present invention include prokaryotic cells, such as E. coli, and eukaryotic host cells in cell culture, including mammalian cells, such as Chinese Hamster Ovary (CHO).

The invention is illustrated in the examples in the Experimental Details Section that follows. This section is set forth to aid in an understanding of the invention but is not intended to, and should not be construed to limit in any way the invention as set forth in the claims which follow thereafter.

### EXAMPLES

### Example 1

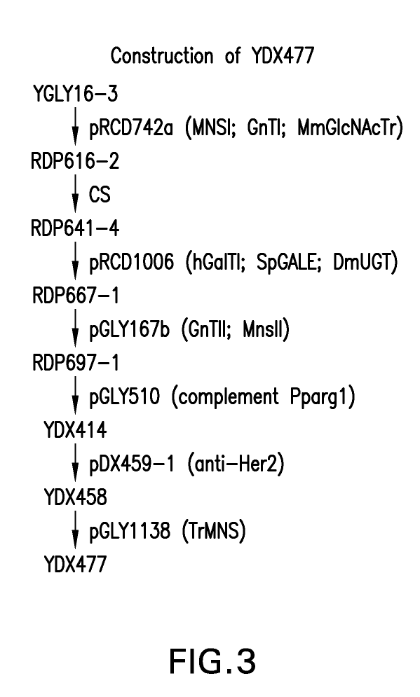

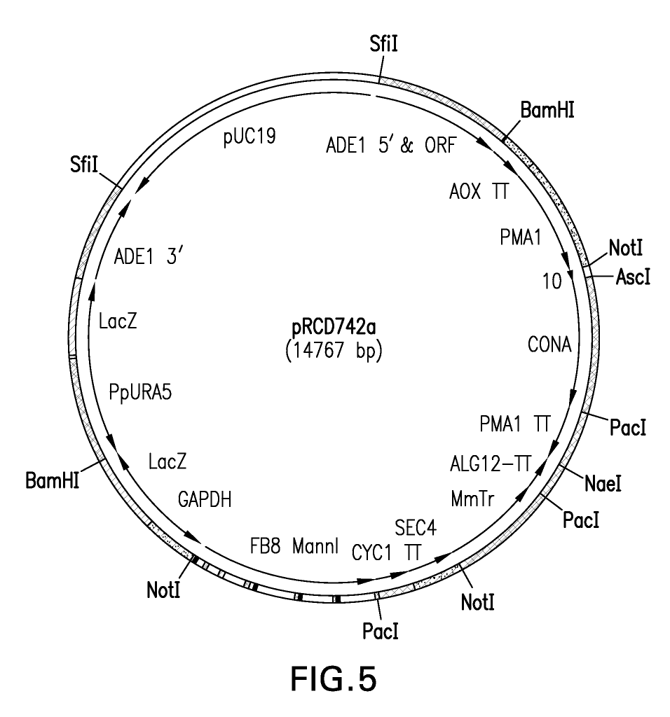

Construction of strain GFI5.0 YDX477 is shown in FIG. 3. The starting strain was YGLY16-3. Strain YGLY16-3 was transformed with plasmid pRCD742a (See FIG. 5) to make strain RDP616-2. Plasmid pRCD742a (See FIG. 5) is a KINKO plasmid that integrates into the P. pastoris ADE1 gene without deleting the open reading frame encoding the ade1p. The plasmid also contains the PpURA5 selectable marker and includes expression cassettes encoding the chimeric mouse alpha-1,2-mannosyltransferase (FB8 MannI), the chimeric human GlcNAc Transferase I (CONA10), and the full length mouse Golgi UDP-GlcNAc transporter (MmSLC35A3). The plasmid is the same as plasmid pRCD742b except that the orientation of the expression cassette encoding the chimeric human GlcNAc Transferase I is in the opposite orientation. Transfection of plasmid pRCD742a into strain YGLY16-3 resulted in strain RDP616-2. This strain is capable of making glycoproteins that have GlcNAcMan₅GlcNAc₂ N-glycans.

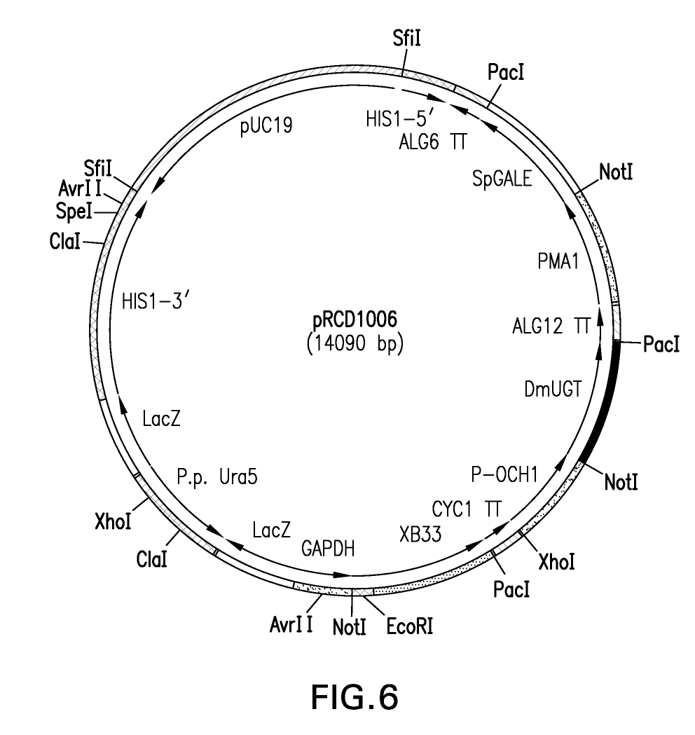

After counterselecting strain RDP616-2 to produce ura-strain RDP641-4, plasmid pRCD1006 was then transformed into the strain to make strain RDP667-1. Plasmid pRCD1006 (See FIG. 6) is a P. pastoris his1 knock-out plasmid that contains the PpURA5 gene as a selectable marker. The plasmid contains an expression cassette encoding a secretory pathway targeted fusion protein (XB33) comprising the first 58 amino acids of ScMnt1p (ScKre2p) (33) fused to the N-terminus of the human Galactosyl Transferase I catalytic domain (hGalTIβ43) under control of the PpGAPDH promoter; an expression cassette encoding the full-length D. melanogaster Golgi UDP-galactose transporter (DmUGT) under control of the PpOCH1 promoter; and an expression cassette encoding the full-length S. pombe UDP-galactose 4-epimerase (SpGALE) under control of the PpPMA1 promoter.

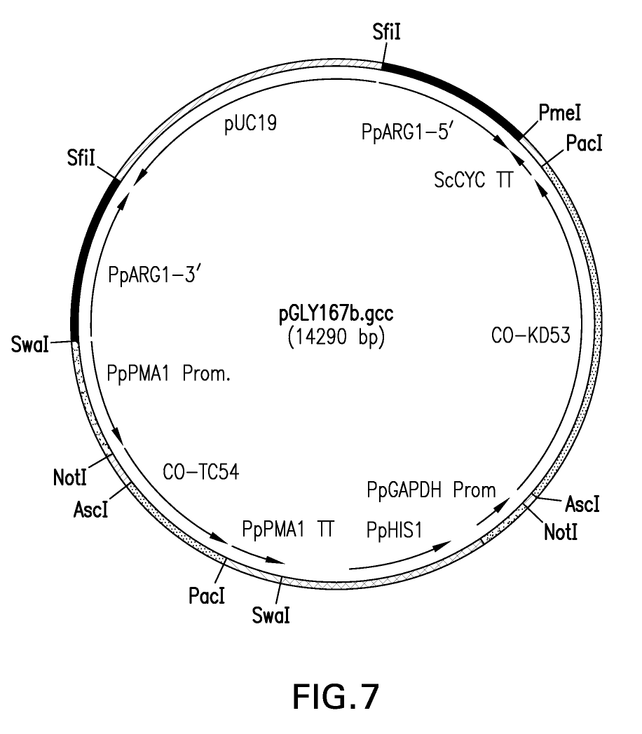

Strain RDP667-1 was transformed with plasmid pGLY167b to make strain RDP697-1. Plasmid pGLY167b (See FIG. 7) is a P. pastoris arg1 knock-out plasmid that contains the PpURA3 selectable marker. The plasmid contains an expression cassette encoding a secretory pathway targeted fusion protein (C0-KD53) comprising the first 36 amino acids of ScMnn2p (53) fused to N-terminus of the Drosophila melanogaster Mannosidase II catalytic domain (KD) under the control of PpGAPDH promoter and an expression cassette expressing a secretory pathway targeted fusion protein (C0-TC54) comprising the first 97 amino acids of ScMnn2p (54) fused to the N-terminus of the rat GlcNAc Transferase II catalytic domain under the control of the PpPMA1 promoter. The nucleic acid molecules encoding the mannosidase II and GnT II catalytic domains were codon-optimized for expression in Pichia pastoris (SEQ ID NO:70 and 73, respectively). This strain can make glycoproteins that have N-glycans that have terminal galactose residues.

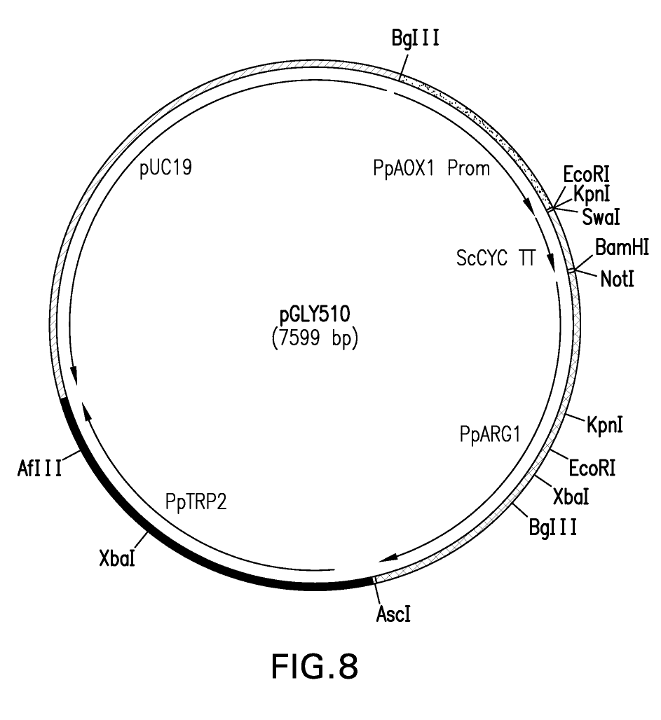

Strain RDP697-1 was transformed with plasmid pGLY510 to make strain YDX414. Plasmid pGLY510 (See FIG. 8) is a roll-in plasmid that integrates into the P. pastoris TRP2 locus while duplicating the gene and contains an AOX1 promoter-ScCYC1 terminator expression cassette as well as the PpARG1 selectable marker.

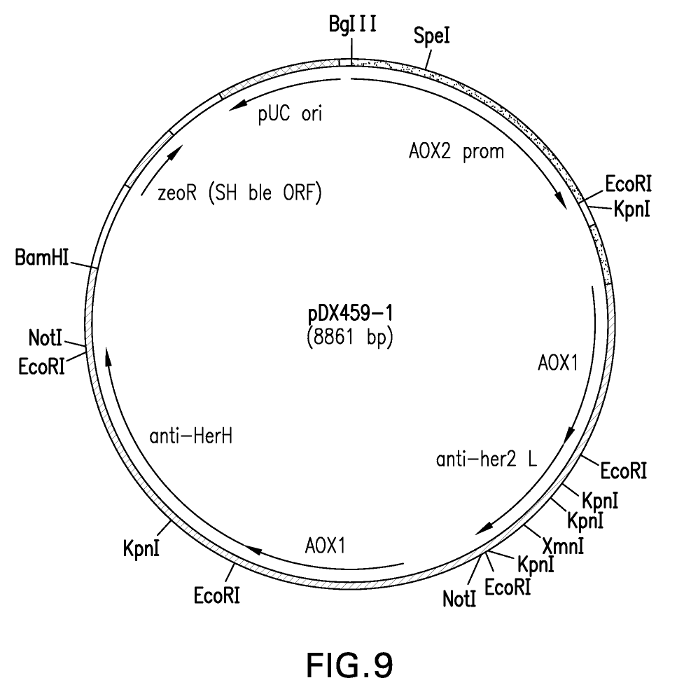

Strain YDX414 was transformed with plasmid pDX459-1 (anti-Her2) to make strain YDX458. Plasmid pDX459-1 (See FIG. 9) is a roll-in plasmid that targets and integrates into the P. pastoris AOX2 promoter and contains the ZeoR while duplicating the promoter. The plasmid contains separate expression cassettes encoding an anti-HER2 antibody heavy chain and an anti-HER2 antibody light chain (SEQ ID NOs:20 and 18, respectively), each fused at the N-terminus to the Aspergillus niger alpha-amylase signal sequence (SEQ ID NO:88) and controlled by the P. pastoris AOX1 promoter. The nucleic acid sequences encoding the heavy and light chains are shown in SEQ ID NOs:19 and 17, respectively, and the nucleic acid sequence encoding the Aspergillus niger alpha-amylase signal sequence is shown in SEQ ID NO:21.

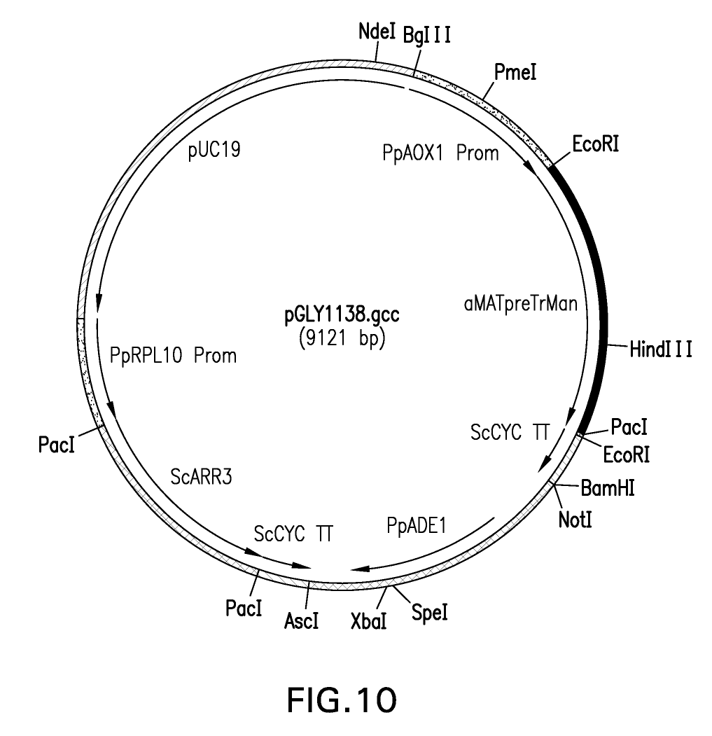

Strain YDX458 was transformed with plasmid pGLY1138 to make strain YDX477. Plasmid pGLY1138 (See FIG. 10) is a roll-in plasmid that integrates into the P. pastoris ADE1 locus while duplicating the gene. The plasmid contains a ScARR3 selectable marker gene cassette. The ARR3 gene from S. cerevisiae confers arsenite resistance to cells that are grown in the presence of arsenite (Bobrowicz et al., Yeast, 13:819-828 (1997); Wysocki et al., J. Biol. Chem. 272:30061-30066 (1997)). The plasmid contains an expression cassette encoding a secreted fusion protein comprising the S. cerevisiae alpha factor pre signal sequence (SEQ ID NO:14) fused to the N-terminus of the Trichoderma reesei (MNS1) catalytic domain (SEQ ID NO:22 encoded by the nucleotide sequence in SEQ ID NO:83) under the control of the PpAOX1 promoter. The fusion protein is secreted into the culture medium.

### Example 2

### Bioreactor Cultivations of YDX477 Strain

A 500 mL baffled volumetric flask with 150 mL of BMGY media was inoculated with 1 mL of seed culture (see flask cultivations). The inoculum was grown to an OD₆₀₀ of 4-6 at 24° C. (approx 18 hours). The cells from the inoculum culture were then centrifuged and resuspended into 50 mL of fermentation media (per liter of media: CaSO₄.2H₂O 0.30 g, K₂SO₄ 6.00 g, MgSO₄.7H₂O 5.00 g, Glycerol 40.0 g, PTM₁ salts 2.0 mL, Biotin 4×10⁻³ g, H₃PO₄ (85%) 30 mL, PTM1 salts per liter: CuSO₄.H₂O 6.00 g, NaI 0.08 g, MnSO₄.7H₂O 3.00 g, NaMoO₄.2H₂O 0.20 g, H₃BO₃ 0.02 g, CoCl₂.6H₂O 0.50 g, ZnCl₂ 20.0 g, FeSO₄.7H₂O 65.0 g, Biotin 0.20 g, H₂SO₄ (98%) 5.00 mL).

Fermentations were conducted in three-liter dished bottom (1.5 liter initial charge volume) Applikon bioreactors. The fermenters were run in a fed-batch mode at a temperature of 24° C., and the pH was controlled at 4.5±0.1 using 30% ammonium hydroxide. The dissolved oxygen was maintained above 40% relative to saturation with air at 1 atm by adjusting agitation rate (450-900 rpm) and pure oxygen supply. The air flow rate was maintained at 1 vvm. When the initial glycerol (40 g/L) in the batch phase is depleted, which is indicated by an increase of DO, a 50% glycerol solution containing 12 ml/L of PTM₁ salts was fed at a feed rate of 12 mL/L/h until the desired biomass concentration was reached. After a half an hour starvation phase, the methanol feed (100% methanol with 12 mL/L PTM₁) is initiated. The methanol feed rate is used to control the methanol concentration in the fermenter between 0.2 and 0.5%. The methanol concentration is measured online using a TGS gas sensor (TGS822 from Figaro Engineering Inc.) located in the offgas from the fermenter. The fermenters were sampled every eight hours and analyzed for biomass (OD₆₀₀, wet cell weight and cell counts), residual carbon source level (glycerol and methanol by HPLC using Aminex 87H) and extracellular protein content (by SDS page, and Bic-Rad protein assay).

Alternatively, fermentations in 15 L and 40 L bioreactors can be conducted according to methods described previously (Li et al, Nat Biotechnol, 24, 210, 2006).

### Example 3

### MALDI-TOF Analysis of Glycans of Anti-Her2 from GFI2.0 and GFI5.0 YDX477

N-glycans were analyzed as described in Choi et al., Proc. Natl. Acad. Sci. USA 100: 5022-5027 (2003) and Hamilton et al., Science 301: 1244-1246 (2003). After the glycoproteins were reduced and carboxymethylated, N-glycans were released by treatment with peptide-N-glycosidase F. The released oligosaccharides were recovered after precipitation of the protein with ethanol. Molecular weights were determined by using a Voyager PRO linear MALDI-TOF (Applied Biosystems) mass spectrometer with delayed extraction according to the manufacturer's instructions. The N-glycan analysis of Anti-Her2 is illustrated in FIG. 4, and Table 1 below.

TABLE 1

| Sample | G0% | G1% | G2% | Man5% | Man6, 7, 8% | Mang8 plus % | % Hybrid |
|---|---|---|---|---|---|---|---|
| GFI2.0 | ND | ND | ND | 95.61% | 4.39% | ND | ND |
| GFI5.0 YDX477 | 60.14% | 16.81% | 4.45% | 8.51% | 1.09% | 2.24% | 6.76% |

### Example 4

### Construction of Strains YGLY13992, YGLY13979 and YGLY12501

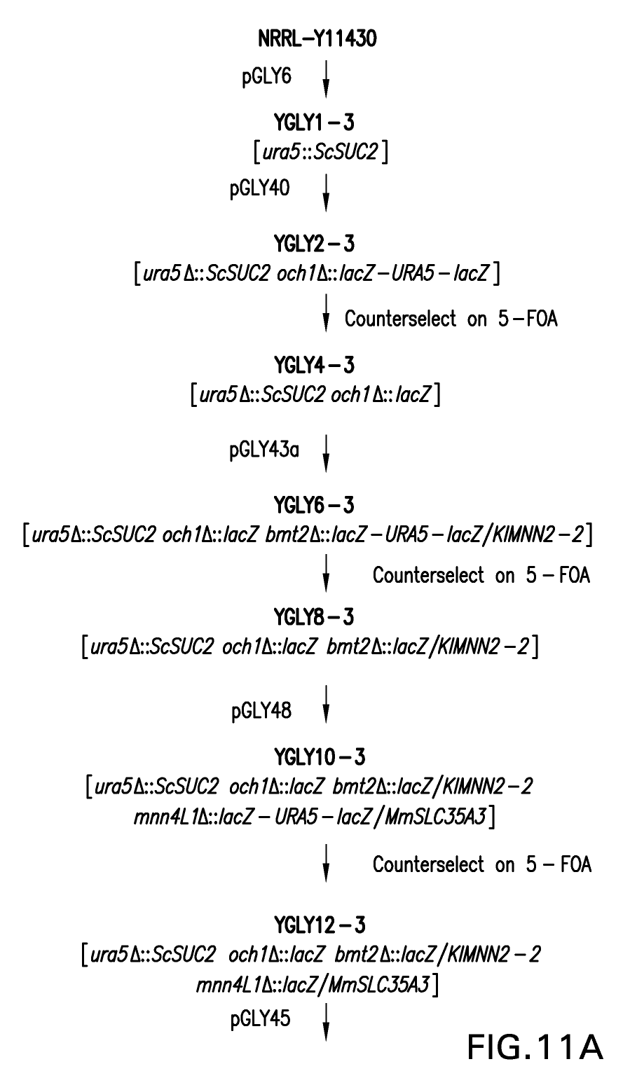

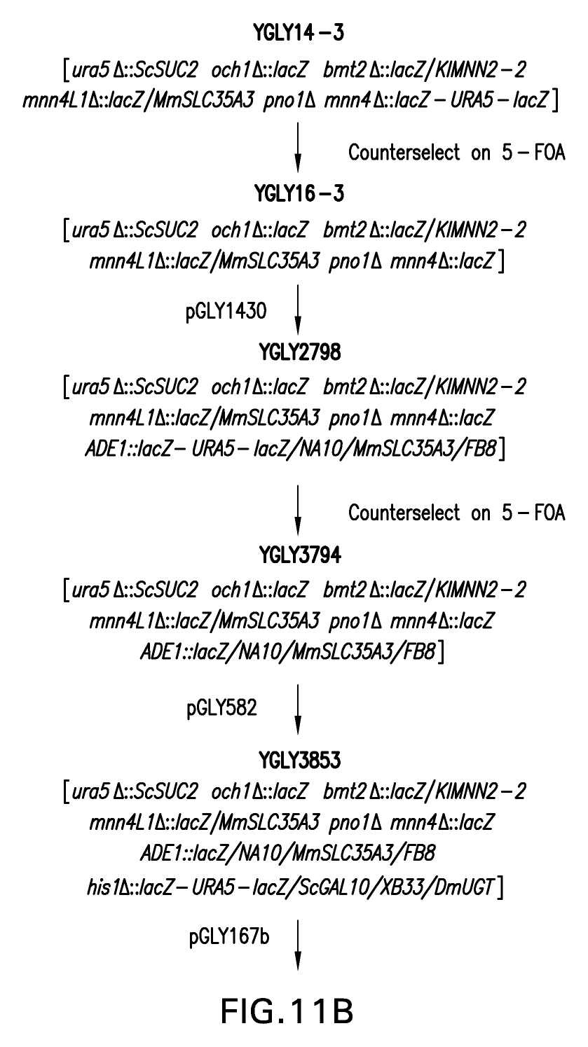

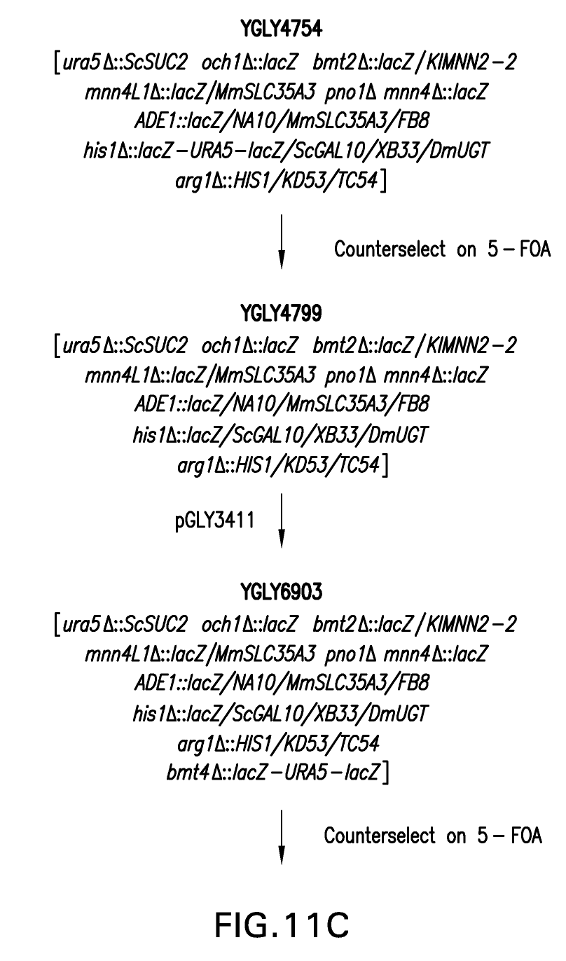

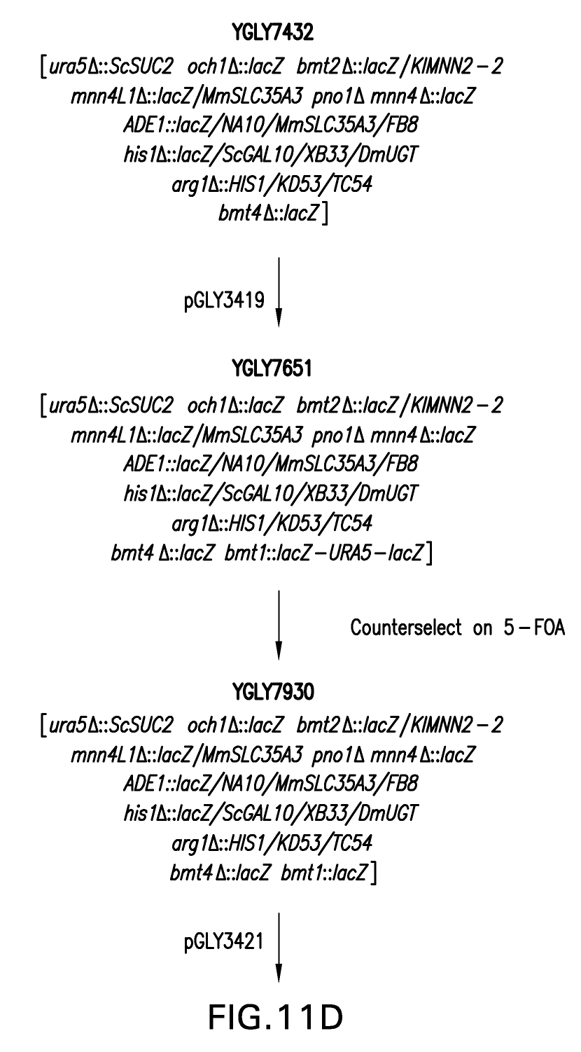

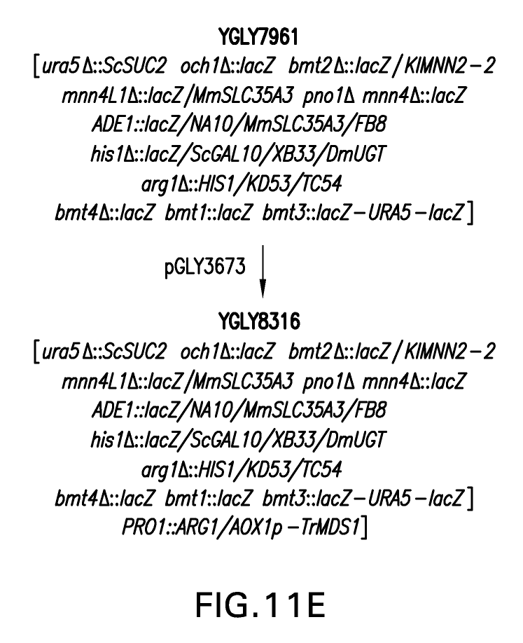

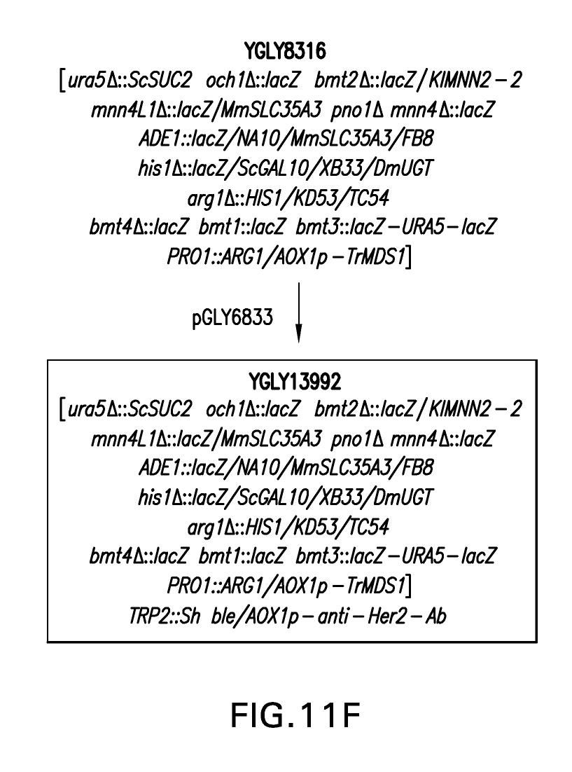

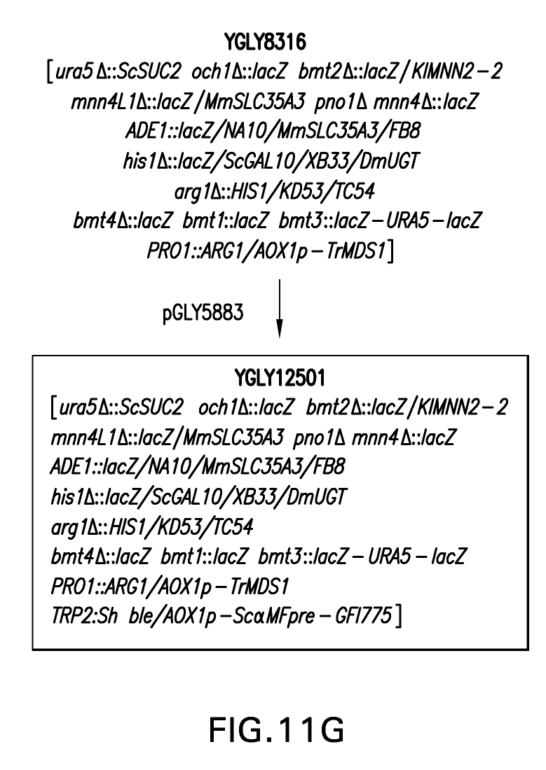

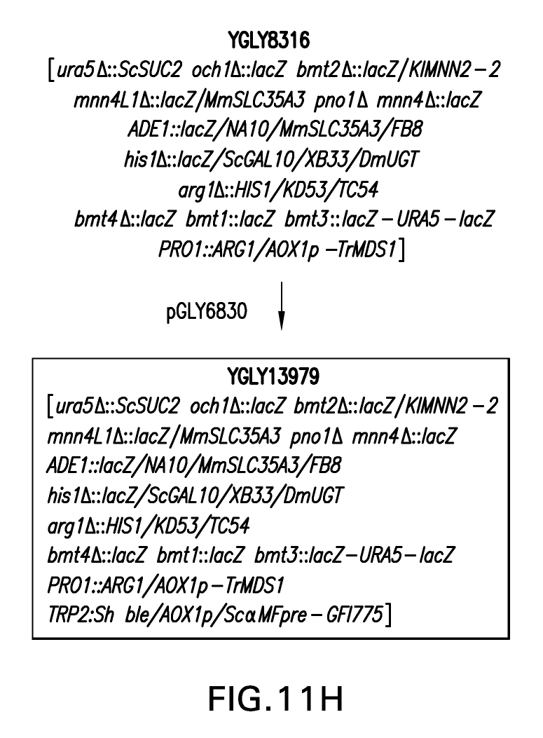

Genetically engineered Pichia pastoris strains YGLY13992, YGLY12501, YGLY13979 produce recombinant human anti-Her2 antibodies. Construction of the strains is illustrated schematically in FIGS. 11A-1111. Briefly, the strains were constructed as follows.

The strain YGLY8316 was constructed from wild-type Pichia pastoris strain NRRL-Y 11430 using methods described earlier (See for example, U.S. Pat. No. 7,449,308; U.S. Pat. No. 7,479,389; U.S. Published Application No. 20090124000; Published PCT Application No. WO2009085135; Nett and Gemgross, Yeast 20:1279 (2003); Choi et al., Proc. Natl. Acad. Sci. USA 100:5022 (2003); Hamilton et al., Science 301:1244 (2003)). All plasmids were made in a pUC19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression. Yeast strains were transformed by electroporation (using standard techniques as recommended by the manufacturer of the electroporator BioRad).

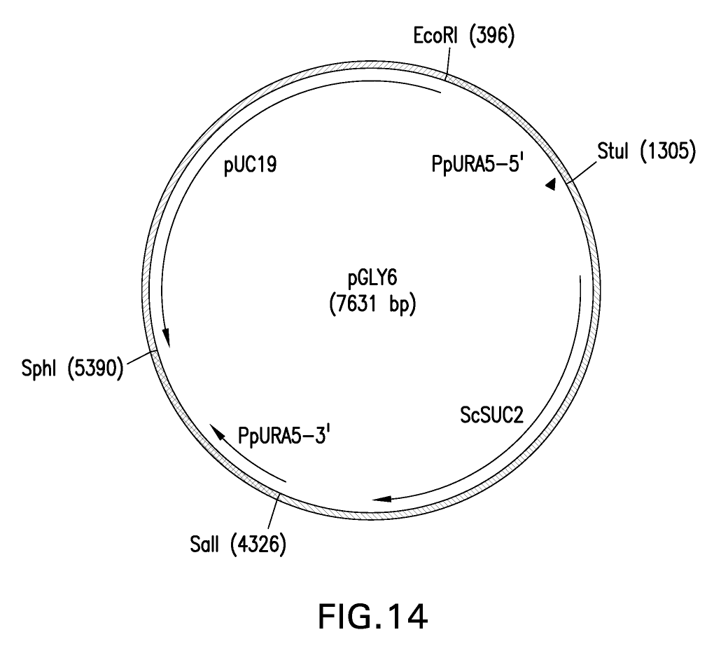

Plasmid pGLY6 (FIG. 14) is an integration vector that targets the URA5 locus containing a nucleic acid molecule comprising the S. cerevisiae invertase gene or transcription unit (ScSUC2; SEQ ID NO:38) flanked on one side by a nucleic acid molecule comprising a nucleotide sequence from the 5′ region of the P. pastoris URA5 gene (SEQ ID NO:39) and on the other side by a nucleic acid molecule comprising the nucleotide sequence from the 3′ region of the P. pastoris URA5 gene (SEQ ID NO:40). Plasmid pGLY6 was linearized and the linearized plasmid transformed into wild-type strain NRRL-Y11430 to produce a number of strains in which the ScSUC2 gene was inserted into the URA5 locus by double-crossover homologous recombination. Strain YGLY1-3 was selected from the strains produced and is auxotrophic for uracil.

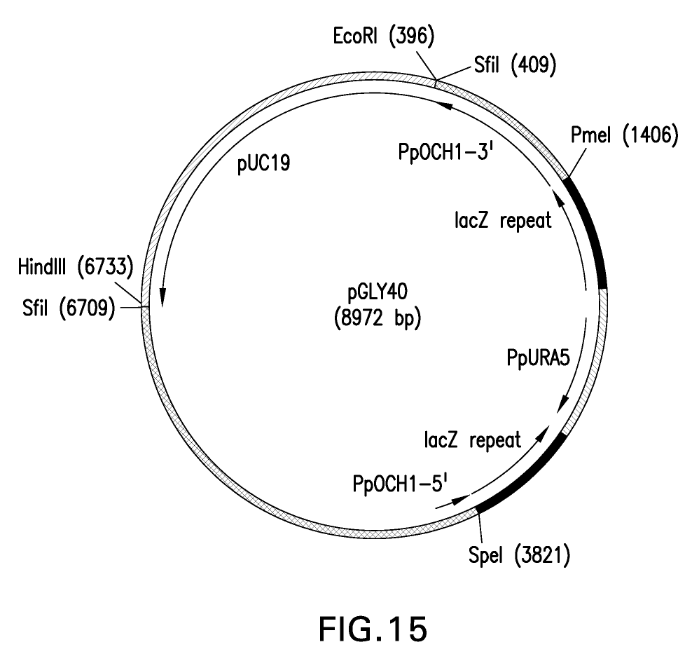

Plasmid pGLY40 (FIG. 15) is an integration vector that targets the OCH1 locus and contains a nucleic acid molecule comprising the P. pastoris URA5 gene or transcription unit (SEQ ID NO:41) flanked by nucleic acid molecules comprising lacZ repeats (SEQ ID NO:42) which in turn is flanked on one side by a nucleic acid molecule comprising a nucleotide sequence from the 5′ region of the OCH1 gene (SEQ ID NO:43) and on the other side by a nucleic acid molecule comprising a nucleotide sequence from the 3′ region of the OCH1 gene (SEQ ID NO:44). Plasmid pGLY40 was linearized with SfiI and the linearized plasmid transformed into strain YGLY1-3 to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus. This renders the strain auxotrophic for uracil. Strain YGLY4-3 was selected.

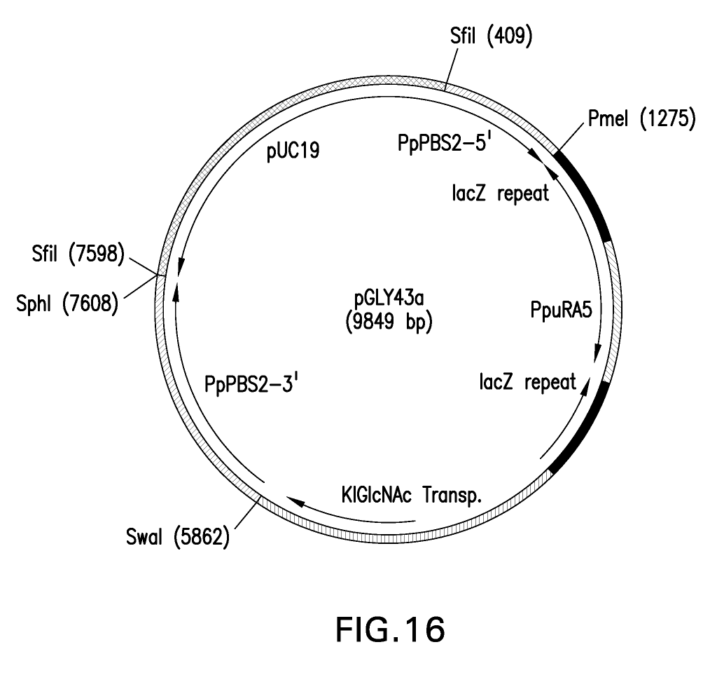

Plasmid pGLY43a (FIG. 16) is an integration vector that targets the BMT2 locus and contains a nucleic acid molecule comprising the K. lactis UDP-N-acetylglucosamine (UDP-GlcNAc) transporter gene or transcription unit (KlMNN2-2, SEQ ID NO:45) adjacent to a nucleic acid molecule comprising the P. pastoris URA5 gene or transcription unit flanked by nucleic acid molecules comprising lacZ repeats. The adjacent genes are flanked on one side by a nucleic acid molecule comprising a nucleotide sequence from the 5′ region of the BMT2 gene (SEQ ID NO: 46) and on the other side by a nucleic acid molecule comprising a nucleotide sequence from the 3′ region of the BMT2 gene (SEQ ID NO:47). Plasmid pGLY43a was linearized with SfiI and the linearized plasmid transformed into strain YGLY4-3 to produce a number of strains in which the KlMNN2-2 gene and URA5 gene flanked by the lacZ repeats has been inserted into the BMT2 locus by double-crossover homologous recombination. The BMT2 gene has been disclosed in Mille et al., J. Biol. Chem. 283: 9724-9736 (2008) and U.S. Pat. No. 7,465,557. Strain YGLY6-3 was selected from the strains produced and is prototrophic for uracil. Strain YGLY6-3 was counterselected in the presence of 5-FOA to produce strains in which the URA5 gene has been lost and only the lacZ repeats remain. This renders the strain auxotrophic for uracil. Strain YGLY8-3 was selected.

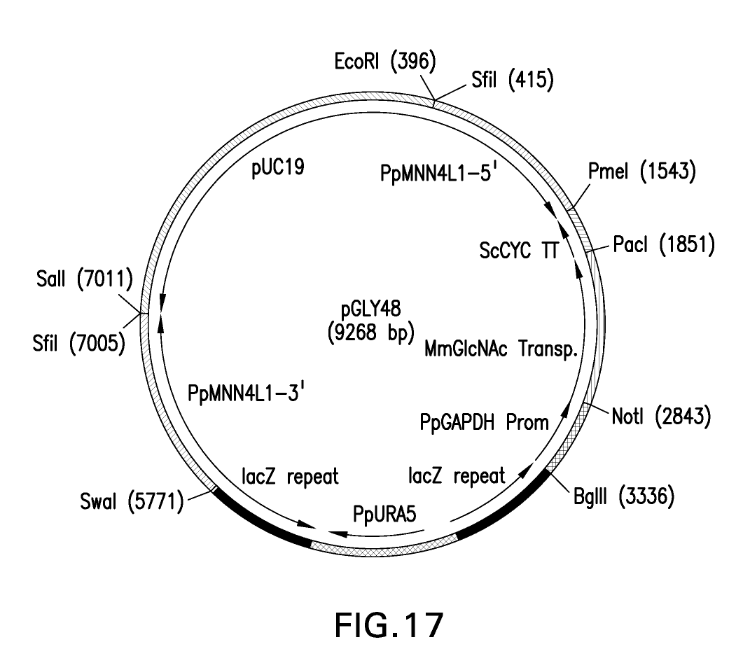

Plasmid pGLY48 (FIG. 17) is an integration vector that targets the MNN4L1 locus and contains an expression cassette comprising a nucleic acid molecule encoding the mouse homologue of the UDP-GlcNAc transporter (SEQ ID NO:48) open reading frame (ORF) operably linked at the 5′ end to a nucleic acid molecule comprising the P. pastoris GAPDH promoter (SEQ ID NO:26) and at the 3′ end to a nucleic acid molecule comprising the S. cerevisiae CYC termination sequences (SEQ ID NO:24) adjacent to a nucleic acid molecule comprising the P. pastoris URA5 gene flanked by lacZ repeats and in which the expression cassettes together are flanked on one side by a nucleic acid molecule comprising a nucleotide sequence from the 5′ region of the P. Pastoris MNN4L1 gene (SEQ ID NO:49) and on the other side by a nucleic acid molecule comprising a nucleotide sequence from the 3′ region of the MNN4L1 gene (SEQ ID NO:50). Plasmid pGLY48 was linearized with SfiI and the linearized plasmid transformed into strain YGLY8-3 to produce a number of strains in which the expression cassette encoding the mouse UDP-GlcNAc transporter and the URA5 gene have been inserted into the MNN4L1 locus by double-crossover homologous recombination. The MNN4L1 gene (also referred to as MNN4B) has been disclosed in U.S. Pat. No. 7,259,007. Strain YGLY10-3 was selected from the strains produced and then counterselected in the presence of 5-FOA to produce a number of strains in which the URA5 gene has been lost and only the lacZ repeats remain. Strain YGLY12-3 was selected.

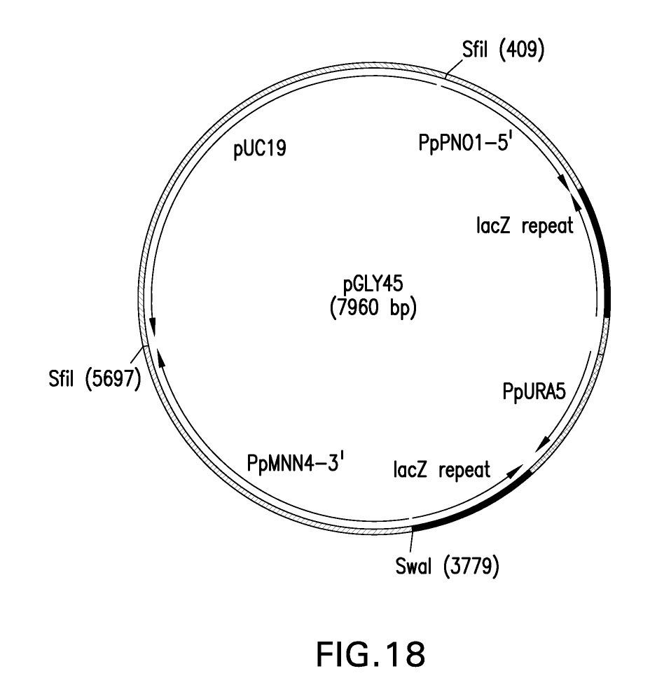

Plasmid pGLY45 (FIG. 18) is an integration vector that targets the PNO1/MNN4 loci contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the PNO1 gene (SEQ ID NO:51) and on the other side by a nucleic acid molecule comprising a nucleotide sequence from the 3′ region of the MNN4 gene (SEQ ID NO:52). Plasmid pGLY45 was linearized with SfiI and the linearized plasmid transformed into strain YGLY12-3 to produce to produce a number of strains in which the URA5 gene flanked by the lacZ repeats has been inserted into the PNO1/MNN4 loci by double-crossover homologous recombination. The PNO1 gene has been disclosed in U.S. Pat. No. 7,198,921 and the MNN4 gene (also referred to as MNN4B) has been disclosed in U.S. Pat. No. 7,259,007. Strain YGLY14-3 was selected from the strains produced and then counterselected in the presence of 5-FOA to produce a number of strains in which the URA5 gene has been lost and only the lacZ repeats remain. Strain YGLY16-3 was selected.

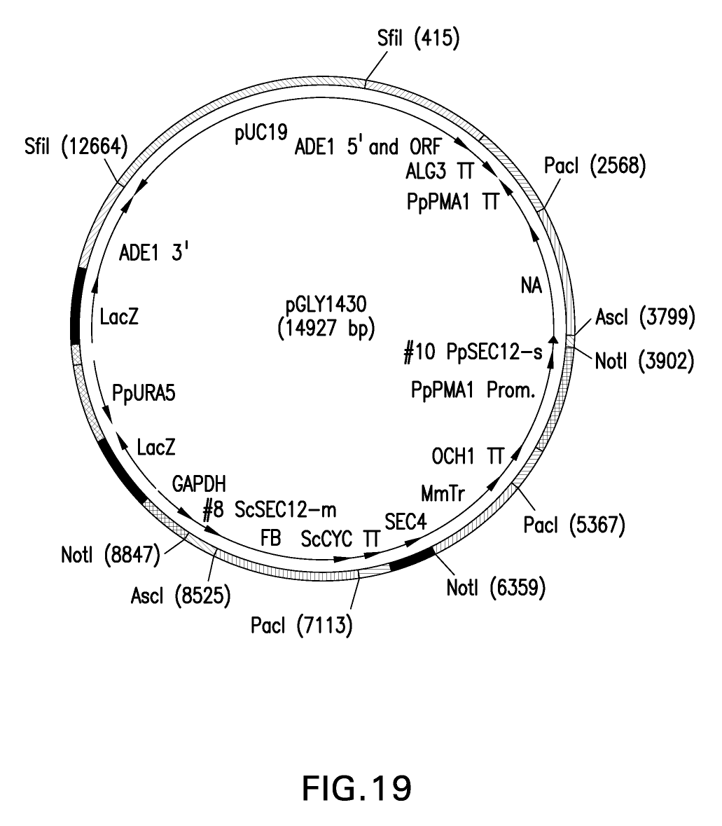

Plasmid pGLY1430 (FIG. 19) is a KINKO integration vector that targets the ADE1 locus without disrupting expression of the locus and contains in tandem four expression cassettes encoding (1) the human GlcNAc transferase I catalytic domain (NA) fused at the N-terminus to P. pastoris SEC12 leader peptide (10) to target the chimeric enzyme to the ER or Golgi, (2) mouse homologue of the UDP-GlcNAc transporter (MmTr), (3) the mouse mannosidase IA catalytic domain (FB) fused at the N-terminus to S. cerevisiae SEC12 leader peptide (8) to target the chimeric enzyme to the ER or Golgi, and (4) the P. pastoris URA5 gene or transcription unit. KINKO (Knock-In with little or No Knock-Out) integration vectors enable insertion of heterologous DNA into a targeted locus without disrupting expression of the gene at the targeted locus and have been described in U.S. Published Application No. 20090124000. The expression cassette encoding the NA10 comprises a nucleic acid molecule encoding the human GlcNAc transferase I catalytic domain codon-optimized for expression in P. pastoris (SEQ ID NO:53) fused at the 5′ end to a nucleic acid molecule encoding the SEC12 leader 10 (SEQ ID NO:54), which is operably linked at the 5′ end to a nucleic acid molecule comprising the P. pastoris PMA1 promoter and at the 3′ end to a nucleic acid molecule comprising the P. pastoris PMA1 transcription termination sequence. The expression cassette encoding MmTr comprises a nucleic acid molecule encoding the mouse homologue of the UDP-GlcNAc transporter ORF operably linked at the 5′ end to a nucleic acid molecule comprising the P. P. pastoris SEC4 promoter (SEQ ID NO:55) and at the 3′ end to a nucleic acid molecule comprising the P. pastoris OCH1 termination sequences (SEQ ID NO:56). The expression cassette encoding the FBS comprises a nucleic acid molecule encoding the mouse mannosidase IA catalytic domain (SEQ ID NO:57) fused at the 5′ end to a nucleic acid molecule encoding the SEC12-m leader 8 (SEQ ID NO:58), which is operably linked at the 5′ end to a nucleic acid molecule comprising the P. pastoris GADPH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The four tandem cassettes are flanked on one side by a nucleic acid molecule comprising a nucleotide sequence from the 5′ region and complete ORF of the ADE1 gene (SEQ ID NO:59) followed by a P. pastoris ALG3 termination sequence (SEQ ID NO:29) and on the other side by a nucleic acid molecule comprising a nucleotide sequence from the 3′ region of the ADE1 gene (SEQ ID NO:60). Plasmid pGLY 1430 was linearized with SfiI and the linearized plasmid transformed into strain YGLY16-3 to produce a number of strains in which the four tandem expression cassette have been inserted into the ADE1 locus immediately following the ADE1 ORF by double-crossover homologous recombination. The strain YGLY2798 was selected from the strains produced and is auxotrophic for arginine and now prototrophic for uridine, histidine, and adenine. The strain was then counterselected in the presence of 5-FOA to produce a number of strains now auxotrophic for uridine. Strain YGLY3794 was selected and is capable of making glycoproteins that have predominantly galactose terminated N-glcyans.

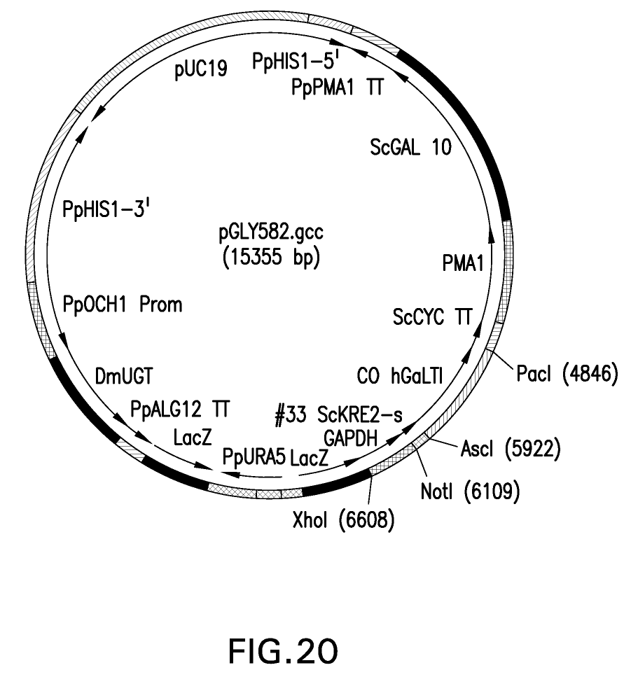

Plasmid pGLY582 (FIG. 20) is an integration vector that targets the HIS1 locus and contains in tandem four expression cassettes encoding (1) the S. cerevisiae UDP-glucose epimerase (ScGAL10), (2) the human galactosyltransferase I (hGalT) catalytic domain fused at the N-terminus to the S. cerevisiae KRE2-s leader peptide (33) to target the chimeric enzyme to the ER or Golgi, (3) the P. pastoris URA5 gene or transcription unit flanked by lacZ repeats, and (4) the D. melanogaster UDP-galactose transporter (DmUGT). The expression cassette encoding the ScGAL10 comprises a nucleic acid molecule encoding the ScGAL10 ORF (SEQ ID NO:61) operably linked at the 5′ end to a nucleic acid molecule comprising the P. pastoris PMA1 promoter (SEQ ID NO:45) and operably linked at the 3′ end to a nucleic acid molecule comprising the P. pastoris PMA1 transcription termination sequence (SEQ ID NO:62). The expression cassette encoding the chimeric galactosyltransferase I comprises a nucleic acid molecule encoding the hGalT catalytic domain codon optimized for expression in P. pastoris (SEQ ID NO:63) fused at the 5′ end to a nucleic acid molecule encoding the KRE2-s leader 33 (SEQ ID NO:64), which is operably linked at the 5′ end to a nucleic acid molecule comprising the P. pastoris GAPDH promoter and at the 3′ end to a nucleic acid molecule comprising the S. cerevisiae CYC transcription termination sequence. The URA5 expression cassette comprises a nucleic acid molecule comprising the P. pastoris URA5 gene or transcription unit flanked by nucleic acid molecules comprising lacZ repeats. The expression cassette encoding the DmUGT comprises a nucleic acid molecule encoding the DmUGT ORF (SEQ ID NO:65) operably linked at the 5′ end to a nucleic acid molecule comprising the P. pastoris OCH1 promoter (SEQ ID NO:66) and operably linked at the 3′ end to a nucleic acid molecule comprising the P. pastoris ALG12 transcription termination sequence (SEQ ID NO:67). The four tandem cassettes are flanked on one side by a nucleic acid molecule comprising a nucleotide sequence from the 5′ region of the HIS1 gene (SEQ ID NO:68) and on the other side by a nucleic acid molecule comprising a nucleotide sequence from the 3′ region of the HIS1 gene (SEQ ID NO:69). Plasmid pGLY582 was linearized and the linearized plasmid transformed into strain YGLY3794 to produce a number of strains in which the four tandem expression cassette have been inserted into the HIS1 locus by homologous recombination. Strain YGLY3853 was selected and is auxotrophic for histidine and prototrophic for uridine.

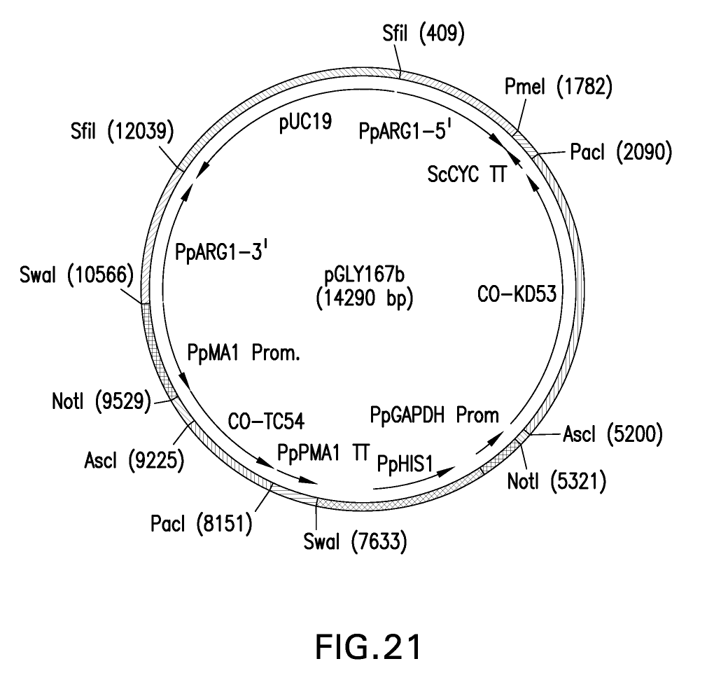

Plasmid pGLY167b (FIG. 21) is an integration vector that targets the ARG1 locus and contains in tandem three expression cassettes encoding (1) the D. melanogaster mannosidase II catalytic domain (KD) fused at the N-terminus to S. cerevisiae MNN2 leader peptide (53) to target the chimeric enzyme to the ER or Golgi, (2) the P. pastoris HIS1 gene or transcription unit, and (3) the rat N-acetylglucosamine (GlcNAc) transferase II catalytic domain (TC) fused at the N-terminus to S. cerevisiae MNN2 leader peptide (54) to target the chimeric enzyme to the ER or Golgi. The expression cassette encoding the KD53 comprises a nucleic acid molecule encoding the D. melanogaster mannosidase II catalytic domain codon-optimized for expression in P. pastoris (SEQ ID NO:70) fused at the 5′ end to a nucleic acid molecule encoding the MNN2 leader 53 (SEQ ID NO:71), which is operably linked at the 5′ end to a nucleic acid molecule comprising the P. pastoris GAPDH promoter and at the 3′ end to a nucleic acid molecule comprising the S. cerevisiae CYC transcription termination sequence. The HIS1 expression cassette comprises a nucleic acid molecule comprising the P. pastoris HIS1 gene or transcription unit (SEQ ID NO:72). The expression cassette encoding the TC54 comprises a nucleic acid molecule encoding the rat GlcNAc transferase II catalytic domain codon-optimized for expression in P. pastoris (SEQ ID NO:73) fused at the 5′ end to a nucleic acid molecule encoding the MNN2 leader 54 (SEQ ID NO:74), which is operably linked at the 5′ end to a nucleic acid molecule comprising the P. pastoris PMA1 promoter and at the 3′ end to a nucleic acid molecule comprising the P. pastoris PMA1 transcription termination sequence. The three tandem cassettes are flanked on one side by a nucleic acid molecule comprising a nucleotide sequence from the 5′ region of the ARG1 gene (SEQ ID NO:75) and on the other side by a nucleic acid molecule comprising a nucleotide sequence from the 3′ region of the ARG1 gene (SEQ ID NO:76). Plasmid pGLY167b was linearized with SfiI and the linearized plasmid transformed into strain YGLY3853 to produce a number of strains (in which the three tandem expression cassette have been inserted into the ARG1 locus by double-crossover homologous recombination. The strain YGLY4754 was selected from the strains produced and is auxotrophic for arginine and prototrophic for uridine and histidine. The strain was then counterselected in the presence of 5-FOA to produce a number of strains now auxotrophic for uridine. Strain YGLY4799 was selected.

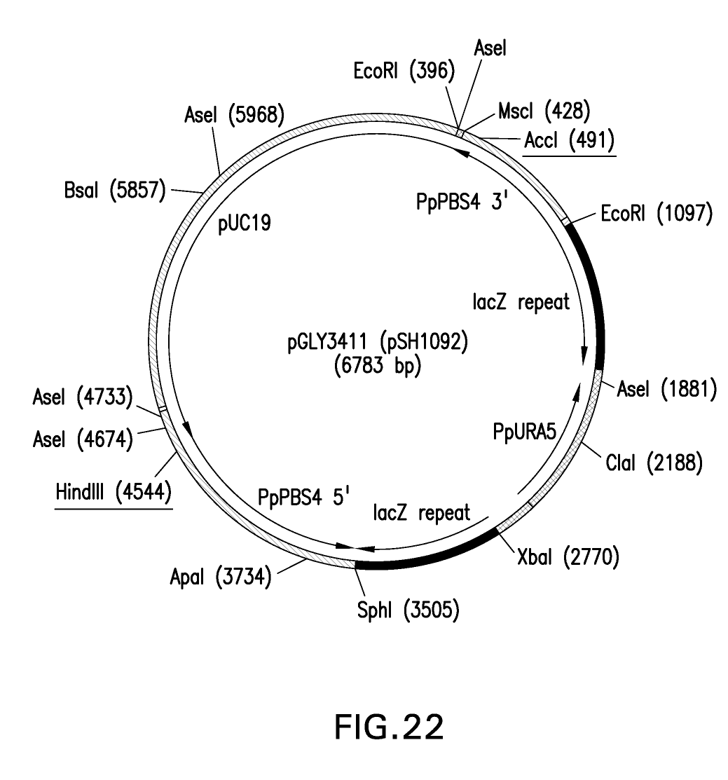

Plasmid pGLY3411 (FIG. 22) is an integration vector that contains the expression cassette comprising the P. pastoris URA5 gene flanked by lacZ repeats flanked on one side with the 5′ nucleotide sequence of the P. pastoris BMT4 gene (SEQ ID NO:77) and on the other side with the 3′ nucleotide sequence of the P. pastoris BMT4 gene (SEQ ID NO:78). Plasmid pGLY3411 was linearized and the linearized plasmid transformed into YGLY4799 to produce a number of strains in which the URA5 expression cassette has been inserted into the BMT4 locus by double-crossover homologous recombination. Strain YGLY6903 was selected from the strains produced and is prototrophic for uracil, adenine, histidine, proline, arginine, and tryptophan. The strain was then counterselected in the presence of 5-FOA to produce a number of strains now auxotrophic for uridine. Strain YGLY7432 was selected.

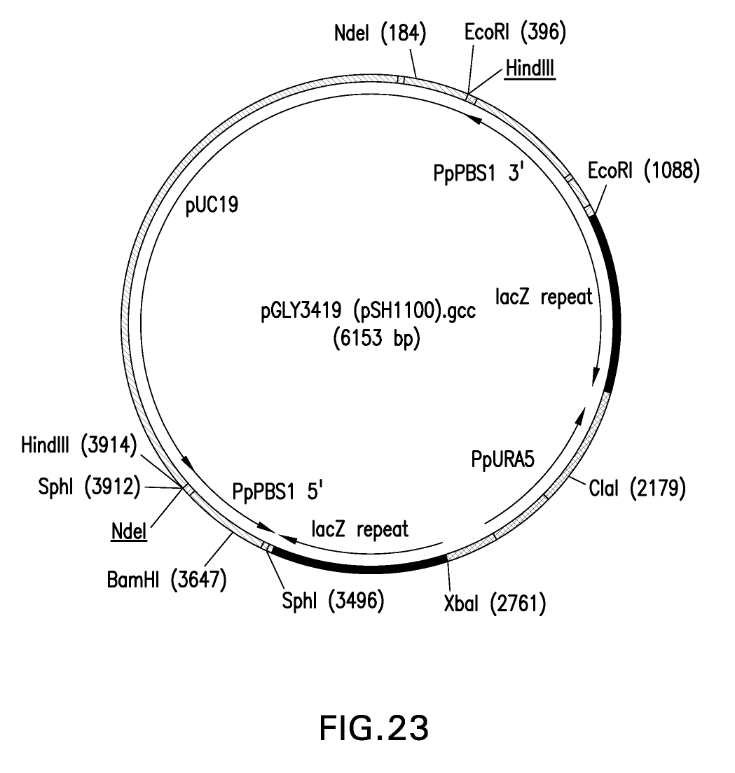

Plasmid pGLY3419 (FIG. 23) is an integration vector that contains an expression cassette comprising the P. pastoris URA5 gene flanked by lacZ repeats flanked on one side with the 5′ nucleotide sequence of the P. pastoris BMT1 gene (SEQ ID NO:79) and on the other side with the 3′ nucleotide sequence of the P. pastoris BMT1 gene (SEQ ID NO:80). Plasmid pGLY3419 was linearized and the linearized plasmid transformed into strain YGLY7432 to produce a number of strains in which the URA5 expression cassette has been inserted into the BMT1 locus by double-crossover homologous recombination. The strain YGLY7651 was selected from the strains produced and is prototrophic for uracil, adenine, histidine, proline, arginine, and tryptophan. The strains were then counterselected in the presence of 5-FOA to produce a number of strains now auxotrophic for uridine. Strain YGLY7930 was selected.

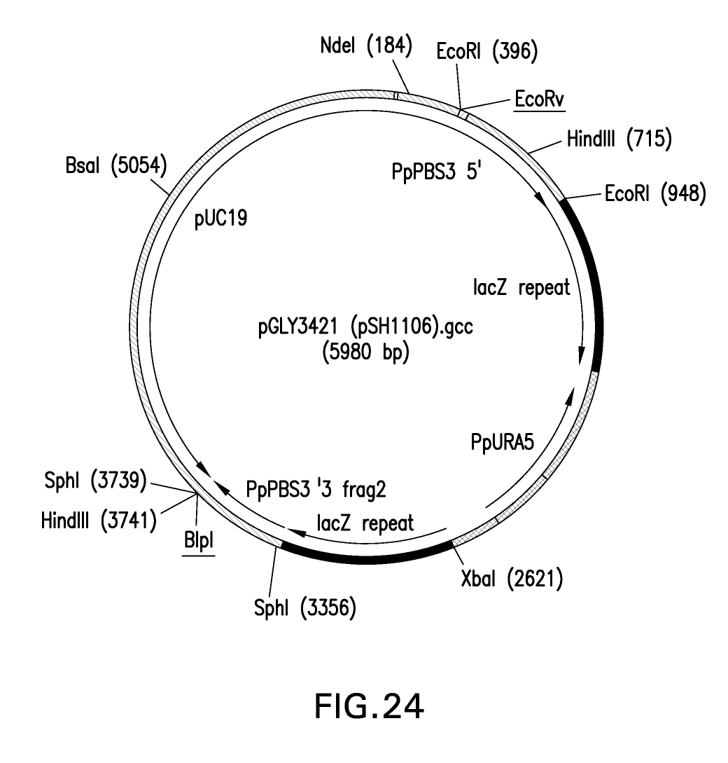

Plasmid pGLY3421 (FIG. 24) is an integration vector that contains an expression cassette comprising the P. pastoris URA5 gene flanked by lacZ repeats flanked on one side with the 5′ nucleotide sequence of the P. pastoris BMT3 gene (SEQ ID NO:81) and on the other side with the 3′ nucleotide sequence of the P. pastoris BMT3 gene (SEQ ID NO:82). Plasmid pGLY3419 was linearized and the linearized plasmid transformed into strain YGLY7930 to produce a number of strains in which the URA5 expression cassette has been inserted into the BMT1 locus by double-crossover homologous recombination. The strain YGLY7961 was selected from the strains produced and is prototrophic for uracil, adenine, histidine, praline, arginine, and tryptophan.

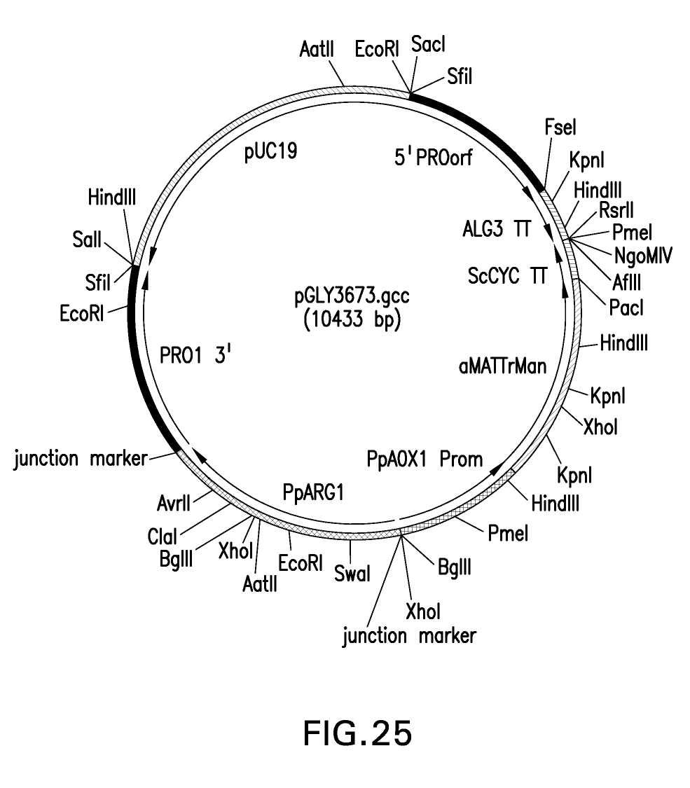

Plasmid pGLY3673 (FIG. 25) is a KINKO integration vector that targets the PRO1 locus without disrupting expression of the locus and contains expression cassettes encoding the T. reesei α-1,2-mannosidase catalytic domain fused at the N-terminus to S. cerevisiae aMATpre signal peptide (aMATTrMan) to target the chimeric protein to the secretory pathway and secretion from the cell. The expression cassette encoding the aMATTrMan comprises a nucleic acid molecule encoding the T. reesei catalytic domain (SEQ ID NO:83) fused at the 5′ end to a nucleic acid molecule encoding the S. cerevisiae αMATpre signal peptide (SEQ ID NO:13), which is operably linked at the 5′ end to a nucleic acid molecule comprising the P. pastoris AOX1 promoter (SEQ ID NO:23) and at the 3′ end to a nucleic acid molecule comprising the S. cerevisiae CYC transcription termination sequence (SEQ ID NO:24). The cassette is flanked on one side by a nucleic acid molecule comprising a nucleotide sequence from the 5′ region and complete ORF of the PRO1 gene (SEQ ID NO:90) followed by a P. pastoris ALG3 termination sequence and on the other side by a nucleic acid molecule comprising a nucleotide sequence from the 3′ region of the PRO1 gene (SEQ ID NO:91). Plasmid pGLY3673 was linearized and the linearized plasmid transformed into strain YGLY7961 to produce a number of strains in which the URA5 expression cassette has been inserted into the BMT1 locus by double-crossover homologous recombination. The strain YGLY8316 was selected from the strains produced and is prototrophic for uracil, adenine, histidine, proline, arginine, and tryptophan.

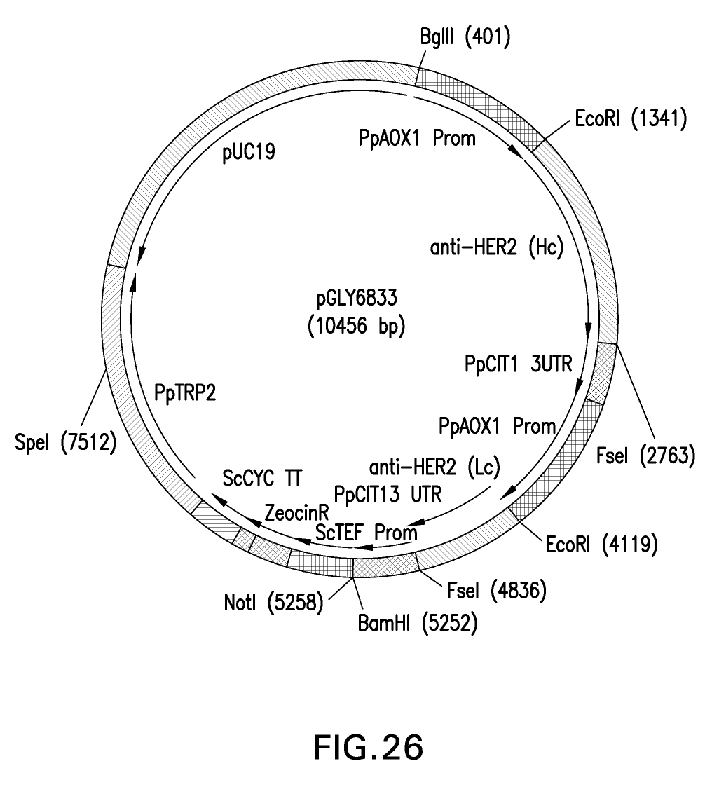

Plasmid pGLY6833 (FIG. 26) is a roll-in integration plasmid encoding the light and heavy chains of an anti-Her2 antibody that targets the TRP2 locus in P. pastoris. The expression cassette encoding the anti-Her2 heavy chain comprises a nucleic acid molecule encoding the heavy chain ORF codon-optimized for effective expression in P. pastoris (SEQ ID NO:15) operably linked at the 5′ end to a nucleic acid molecule encoding the Saccharomyces cerevisiae mating factor pre-signal sequence (SEQ ID NO:14) which in turn is fused at its N-terminus to a nucleic acid molecule that has the inducible P. pastoris AOX1 promoter sequence (SEQ ID NO:23) and at the 3′ end to a nucleic acid molecule that has the P. pastoris CIT1 transcription termination sequence (SEQ ID NO:85). The expression cassette encoding the anti-Her2 light chain comprises a nucleic acid molecule encoding the light chain ORF codon-optimized for effective expression in P. pastoris (SEQ ID NO:17) operably linked at the 5′ end to a nucleic acid molecule encoding the Saccharomyces cerevisiae mating factor pre-signal sequence (SEQ ID NO:14) which in turn is fused at its N-terminus to a nucleic acid molecule that has the inducible P. pastoris AOX1 promoter sequence (SEQ ID NO:23) and at the 3′ end to a nucleic acid molecule that has the P. pastoris CIT1 transcription termination sequence (SEQ ID NO:85). For selecting transformants, the plasmid comprises an expression cassette encoding the Zeocin ORF in which the nucleic acid molecule encoding the ORF (SEQ ID NO:35) is operably linked at the 5′ end to a nucleic acid molecule having the S. cerviseae TEF promoter sequence (SEQ ID NO:37) and at the 3′ end to a nucleic acid molecule having the S. cereviseae CYC transcription termination sequence (SEQ ID NO:24). The plasmid further includes a nucleic acid molecule for targeting the TRP2 locus (SEQ ID NO:92).

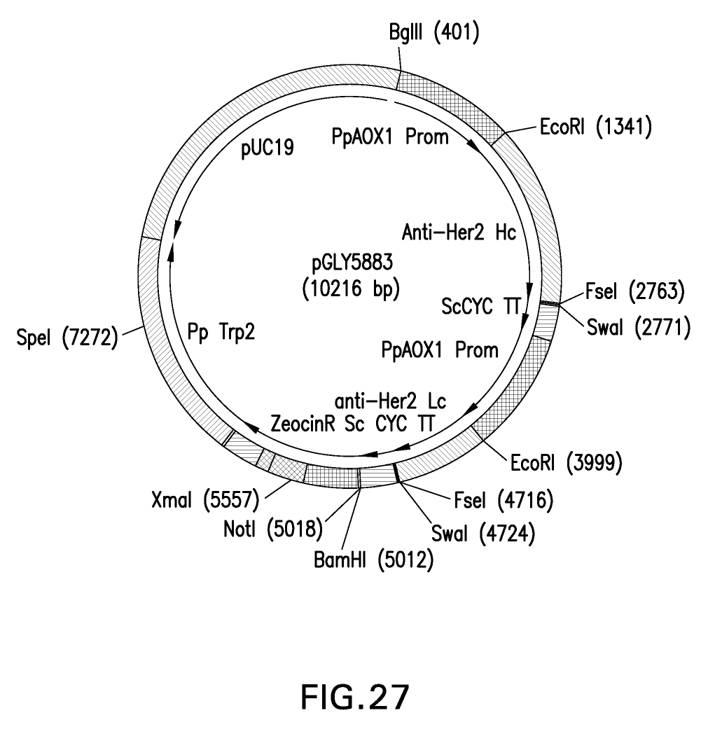

Plasmid pGLY5883 (FIG. 27) is a roll-in integration plasmid encoding the light and heavy chains of an anti-Her2 antibody that targets the TRP2 locus in P. pastoris. The expression cassette encoding the anti-Her2 heavy chain comprises a nucleic acid molecule encoding the heavy chain ORF codon-optimized for effective expression in P. pastoris (SEQ ID NO:15) operably linked at the 5′ end to a nucleic acid molecule encoding the Saccharomyces cerevisiae alpha-mating factor preregion signal sequence (SEQ ID NO:14) which in turn is fused at its N-terminus to a nucleic acid molecule that has the inducible P. pastoris AOX1 promoter sequence (SEQ ID NO:23) and at the 3′ end to a nucleic acid molecule that has the Saccharomyces cerevisiae CYC transcription termination sequence (SEQ ID NO:24). The expression cassette encoding the anti-Her2 light chain comprises a nucleic acid molecule encoding the light chain ORE codon-optimized for effective expression in P. pastoris (SEQ ID NO:17) operably linked at the 5′ end to a nucleic acid molecule encoding the Saccharomyces cerevisiae alpha-mating factor preregion signal sequence (SEQ ID NO:14) which in turn is fused at its N-terminus to a nucleic acid molecule that has the inducible P. pastoris AOX1 promoter sequence (SEQ ID NO:23) and at the 3′ end to a nucleic acid molecule that has the Saccharomyces cerevisiae CYC transcription termination sequence (SEQ ID NO:24). For selecting transformants, the plasmid comprises an expression cassette encoding the Zeocin ORF in which the nucleic acid molecule encoding the ORF (SEQ ID NO:35) is operably linked at the 5′ end to a nucleic acid molecule having the S. cerviseae TEF promoter sequence (SEQ ID NO:37) and at the 3′ end to a nucleic acid molecule having the S. cereviseae CYC transcription termination sequence (SEQ ID NO:24). The plasmid further includes a nucleic acid molecule for targeting the TRP2 locus (SEQ ID NO:92).

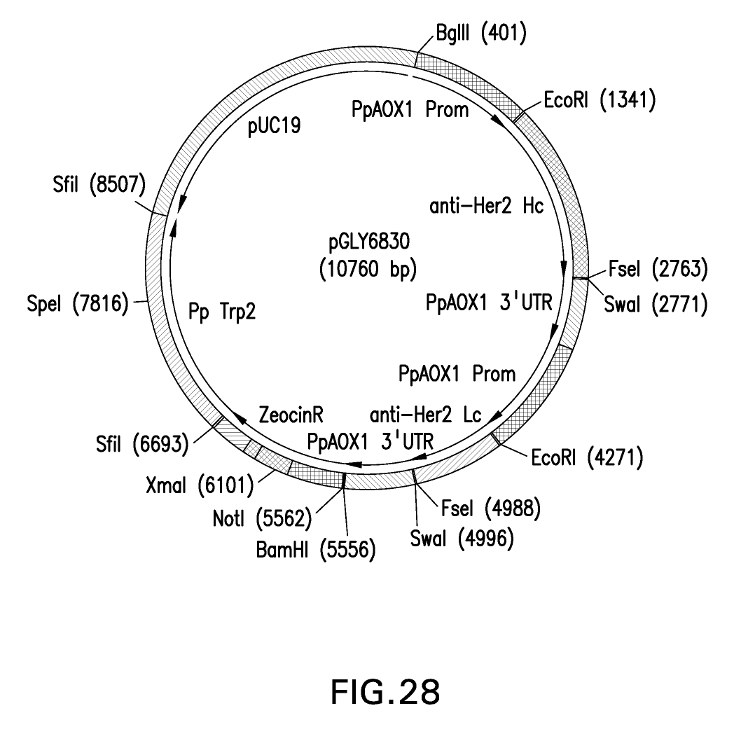

Plasmid pGLY6830 (FIG. 28) is a roll-in integration plasmid encoding the light and heavy chains of an anti-Her2 antibody that targets the TRP2 locus in P. pastoris. The expression cassette encoding the anti-Her2 heavy chain comprises a nucleic acid molecule encoding the heavy chain ORF codon-optimized for effective expression in P. pastoris (SEQ ID NO:15) operably linked at the 5′ end to a nucleic acid molecule encoding the Saccharomyces cerevisiae alpha-mating factor preregion signal sequence (SEQ ID NO:14) which in turn is fused at its N-terminus to a nucleic acid molecule that has the inducible P. pastoris AOX1 promoter sequence (SEQ ID NO:23) and at the 3′ end to a nucleic acid molecule that has the Pichia pastoris AOX1 transcription termination sequence (SEQ ID NO:36). The expression cassette encoding the anti-Her2 light chain comprises a nucleic acid molecule encoding the light chain ORE codon-optimized for effective expression in P. pastoris (SEQ ID NO:17) operably linked at the 5′ end to a nucleic acid molecule encoding the Saccharomyces cerevisiae alpha-mating factor preregion signal sequence (SEQ ID NO:14) which in turn is fused at its N-terminus to a nucleic acid molecule that has the inducible P. pastoris AOX1 promoter sequence (SEQ ID NO:23) and at the 3′ end to a nucleic acid molecule that has the Pichia pastoris AOX1 transcription termination sequence (SEQ ID NO:36). For selecting transformants, the plasmid comprises an expression cassette encoding the Zeocin ORF in which the nucleic acid molecule encoding the ORF (SEQ ID NO:35) is operably linked at the 5′ end to a nucleic acid molecule having the S. cerviseae TEE promoter sequence (SEQ ID NO:37) and at the 3′ end to a nucleic acid molecule having the S. cereviseae CYC transcription termination sequence (SEQ ID NO:24). The plasmid further includes a nucleic acid molecule for targeting the TRP2 locus (SEQ ID NO:92).

Strain YGLY13992 was generated by transforming pGLY6833, which encodes the anti-Her2 antibody, into YGLY8316. The strain YGLY13992 was selected from the strains produced. In this strain, the expression cassettes encoding the anti-Her2 heavy and light chains are targeted to the Pichia pastoris TRP2 locus (PpTRP2).

Strain YGLY13979 was generated by transforming pGLY6830, which encodes the anti-Her2 antibody, into YGLY8316. The strain YGLY13979 was selected from the strains produced. In this strain, the expression cassettes encoding the anti-Her2 heavy and light chains are targeted to the Pichia pastoris TRP2 locus (PpTRP2).

Strain YGLY12501 was generated by transforming pGLY5883, which encodes the anti-Her2 antibody, into YGLY8316. The strain YGLY12501 was selected from the strains produced. In this strain, the expression cassettes encoding the anti-Her2 heavy and light chains are targeted to the Pichia pastoris TRP2 locus (PpTRP2).

### Example 5

### Yeast Transformation and Screening

The glycoengineered Pichia pastoris strains were grown in YPD rich media (yeast extract 1%, peptone 2% and 2% dextrose), harvested in the logarithmic phase by centrifugation, and washed three times with ice-cold 1 M sorbitol. One to five μg of a Spe1 digested plasmid was mixed with competent yeast cells and electroporated using a Bio-Rad Gene Pulser Xcell™ (Bio-Rad, 2000 Alfred Nobel Drive, Hercules, Calif. 94547) preset Pichia pastoris electroporation program. After one hour in recovery rich media at 24° C., the cells were plated on a minimal dextrose media (1.34% YNB, 0.0004% biotin, 2% dextrose, 1.5% agar) plate containing 300 μg/ml Zeocin and incubated at 24° C. until the transformants appeared.

To screen for high titer strains, 96 transformants were inoculated in buffered glycerol-complex medium (BMGY) and grown for 72 hours followed by a 24 hour induction in buffered methanol-complex medium (BMMY). Secretion of antibody was assessed by a Protein A beads assay as follows. Fifty micro liter supernatant from 96 well plate cultures was diluted 1:1 with 50 mM Tris pH 8.5 in a non-binding 96 well assay plate. For each 96 well plate, 2 ml of magnetic BioMag Protein A suspension beads (Qiagen, Valencia, Calif.) were placed in a tube held in a magnetic rack. After 2-3 minutes when the beads collected to the side of the tube, the buffer was decanted off. The beads were washed three times with a volume of wash buffer equal to the original volume (100 mM Tris, 150 mM NaCl, pH 7.0) and resuspended in the same wash buffer. Twenty pi of beads were added to each well of the assay plate containing diluted samples. The plate was covered, vortexed gently and then incubated at room temperature for 1 hour, while vortexing every 15 minutes. Following incubation, the sample plate was placed on a magnetic plate inducing the beads to collect to one side of each well. On the Biomek NX Liquid Handler (Beckman Coulter, Fullerton, Calif.), the supernatant from the plate was removed to a waste container. The sample plate was then removed from the magnet and the beads were washed with 100 μl wash buffer. The plate was again placed on the magnet before the wash buffer was removed by aspiration. Twenty μl loading buffer (Invitrogen E-PAGE gel loading buffer containing 25 mM NEM (Pierce, Rockford, Ill.)) was added to each well and the plate was vortexed briefly. Following centrifugation at 500 rpm on the Beckman Allegra 6 centrifuge, the samples were incubated at 99° C. for five minutes and then run on an E-PAGE high-throughput pre-east gel (Invitrogen, Carlsbad, Calif.). Gels were covered with gel staining solution (0.5 g Coomassie G250 Brilliant Blue, 40% MeOH, 7.5% Acetic Acid), heated in a microwave for 35 seconds, and then incubated at room temperature for 30 minutes. The gels were de-stained in distilled water overnight. High titer colonies were selected for further Sixfors fermentation screening described in detail in Example 6.

### Example 6

### Bioreactor (Sixfors) Screening

Bioreactor fermentation screening was conducted as described as follows: Fed-batch fermentations of glycoengineered Pichia pastoris were executed in 0.5 liter bioreactors (Sixfors multi-fermentation system, ATR Biotech, Laurel, Md.) under the following conditions: pH 6.5, 24° C., 300 ml airflow/min, and an initial stirrer speed of 550 rpm with an initial working volume of 350 ml (330 ml BMGY medium [100 mM potassium phosphate, 10 g/l yeast extract, 20 g/l peptone (BD, Franklin Lakes, N.J.), 40 g/l glycerol, 18.2 g/l sorbitol, 13.4 g/l YNB (BD, Franklin Lakes, N.J.), 4 mg/l biotin] and 20 ml inoculum). IRIS multi-fermentor software (ATR Biotech, Laurel, Md.) was used to increase the stirrer speed from 550 rpm to 1200 rpm linearly between hours 1 and 10 of the fermentation. Consequently, the dissolved oxygen concentration was allowed to fluctuate during the fermentation. The fermentation was executed in batch mode until the initial glycerol charge (40 g/l) was consumed (typically 18-24 hours). A second batch phase was initiated by the addition of 17 ml of a glycerol feed solution to the bioreactor (50% [w/w] glycerol, 5 mg/l biotin and 12.5 ml/l PTM1 salts (65 g/l FeSO₄.7H₂O, 20 g/l ZnCl₂, 9 g/l H₂SO₄, 6 g/l CuSO₄.5H₂O, 5 g/l H₂SO₄, 3 g/l MnSO₄.7H₂O, 500 mg/l CoCl₂.6H₂O, 200 mg/l NaMo04.2H₂O, 200 mg/l biotin, 80 mg/l NaI, 20 mg/l H₃B04). The fermentation was again operated in batch mode until the added glycerol was consumed (typically 6-8 hours). The induction phase was initiated by feeding a methanol solution (100% [w/w] methanol, 5 mg/l biotin and 12.5 ml/l PTM1 salts) at 0.6 g/hr, typically for 36 hours prior to harvest. The entire volume was removed from the reactor and centrifuged in a Sorvall Evolution RC centrifuge equipped with a SLC-6000 rotor (Thermo Scientific, Milford, Mass.) for 30 minutes at 8,500 rpm. The cell mass was discarded and the supernatant retained for purification and analysis. Glycan quality is assessed by MALDI-Time-of-flight (TOF) spectrometry and 2-aminobenzidine (2-AB) labeling according to Li et al. Nat. Biotech. 24(2): 210-215 (2006), Epub 2006 Jan. 22. Glycans were released from the antibody by treatment with PNGase-F and analyzed by MALDI-TOF to confirm glycan structures. To quantitated the relative amounts of neutral and charged glycans present, the N-glycosidase F released glycans were labeled with 2-AB and analyzed by HPLC.

### Example 7

### Bioreactor Cultivations

Fermentations were carried out in 3 L (Applikon, Foster City, Calif.) and 15 L (Applikon, Foster City, Calif.) glass bioreactors and a 40 L (Applikon, Foster City, Calif.) stainless steel, steam in place bioreactor. Seed cultures were prepared by inoculating BMGY media directly with frozen stock vials at a 1% volumetric ratio. Seed flasks were incubated at 24° C. for 48 hours to obtain an optical density (OD₆₀₀) of 20±5 to ensure that cells are growing exponentially upon transfer. The cultivation medium contained 40 g glycerol, 18.2 g sorbitol, 2.3 g K₂HPO₄, 11.9 g KH₂PO₄, 10 g yeast extract (BD, Franklin Lakes, N.J.), 20 g peptone (BD, Franklin Lakes, N.J.), 4×10⁻³ g biotin and 13.4 g Yeast Nitrogen Base (BD, Franklin Lakes, N.J.) per liter. The bioreactor was inoculated with a 10% volumetric ratio of seed to initial media. Cultivations were done in fed-batch mode under the following conditions: temperature set at 24±0.5° C., pH controlled at 6.5±0.1 with NH₄OH, dissolved oxygen was maintained at 1.7±0.1 mg/L by cascading agitation rate on the addition of O₂. The airflow rate was maintained at 0.7 vvm. After depletion of the initial charge glycerol (40 g/L), a shot of 1.3 ml/L of a solution of 0.65 mg/mL PMTi-4 in methanol is added, and a 50% glycerol solution containing 12.5 mL/L of PTM2 salts was fed at a rate ranging from 5 g/L-h to 12 g/L-h for an interval of 8-20 hours until a wet cell weight of between 200-250 g/L was reached. Induction was initiated after a thirty minute starvation phase when a second shot of 1.3 ml/L of a solution of 0.65 mg/mL PMTi-4 in methanol is added, and a solution of methanol containing 12.5 mL/L of PTM2 salts was fed to the reactor at a rate ranging from 1 g/L-h to a maximum of 4 g/L-h, at either a fixed rate or an exponentially increasing rate with an exponent term ranging from 0.003 to 0.015 l/h. The methanol feed rate was capped if the oxygen uptake rate exceeded 150 mM/L/h. Additional shots of 1.3 ml/L of a solution of 0.65 mg/mL PMTi-4 in methanol are added every 24 hours into induction until harvest. Induction continues for 72 h to 200 h, when the methanol feed is stopped and harvest is initiated. Cell removal is done by centrifugation. The whole cell broth is transferred into 1000 mL centrifuge bottles and centrifuged at 4° C. for 30 minutes at 13,000 G. The supernatant is decanted for purification of antibody.

### Example 8

### Large Scale Fermentation of Strain YGLY13979

The seed train consisted of one flask and one seed fermenter stage. During the flask stage, two 3-L shake flasks containing 416±16 g (400 mL) of BYSS media with UCON were each inoculated with 0.4±0.02 mL of thawed working seed. These flasks were incubated until a broth pH between 5.5 to 5.0 was achieved at 48±2 h, then 156±16 g of culture was transferred to a seed fermenter containing 15±0.3 L of BYSS media.

Cell growth in the seed fermenter was maintained at a temperature of 24±1° C. and a pH of 6.5±0.2 for 35 A: 2 h until an oxygen uptake rate (OUR) of 50-60 mmol/L/h was achieved. Dissolved oxygen was maintained at 20±10% of saturation at 5 psig (24° C.). The production fermenter containing 15±1 L of BYSS media was inoculated with 1.56±0.2 kg of broth from the seed fermenter.

In the production fermenter, the pH was controlled at 6.5±0.2 with 14% (w/w) NH₄OH and 15% (w/w) H₃PO₄. Temperature was controlled at 24±1° C. while the level of dissolved oxygen was maintained at 20±10% of saturation at 5 psig (24° C.) by agitation rate cascaded on the addition of pure oxygen (0-20 SLPM) to the fixed airflow rate of 0.7 vvm (10.5 SLPM).

The production fermentation consisted of a batch phase, glycerol fed batch phase, transition phase and methanol induction phase. The batch phase ends when the initial supply of glycerol was depleted as signaled by a rapid decline in OUR. The biomass concentration was further increased during the glycerol fed batch phase where 50% (w/w) glycerol supplemented with PTM2 salts and biotin was exponentially fed for 8 hours. This was followed by the transition phase (a 30 minute starvation period). Protein production was initiated during the induction phase when methanol was fed exponentially. At the start of induction a 19±1 mL dose of PMTi-4 inhibitor solution was added to the fermenter. Production fermentation induction was continued for 80±5 hours of induction.

### A. Shake Flask Stage

BYSS shake flask media was formulated according to Table 2, pH adjusted to 6.3±0.2 and filter sterilized through a 0.2 μm EKV membrane or equivalent filter (PALL Cat No KA02EVKP2S).

The shake flasks were prepared by adding 416±16 g of BYSS flask media (400 mL assuming 1.04 g/mL density) into each of two 3-L baffled shake flasks (Corning Cat No 431253) (1 for seed inoculum generation and 1 for sampling). 10 mL of a 1:10 dilution of UCON in BYSS media was then formulated, and vigorously mixed by shaking prior to transfer of 1.0±0.1 mL into each shake flask. Two vials of Pichia pastoris YGLY13979 working seed were then thawed at room temperature, and each flask is inoculated with 0.4±0.02 mL of vial seed. These flasks were then incubated at 24±1° C. and 180 RPM (2 inch throw) until the pH is between 5.5 and 5.0. This typically takes 48±2 hrs with the Wet Cell Weight (WCW) at 100±25. 156±16 g (150 mL) of this broth was transferred to a seed fermenter containing 15.6±0.3 kg (15 L assuming density of 1.04 g/mL) of BYSS medium (Table 3).

TABLE 2
BYSS Shake Flask Medium pH 6.3 (density = 1.04 g/mL)

| Component | Supplier | Grade | Catalog # | Conc. | Units |
|---|---|---|---|---|---|
| Yeast Extract | Sensient | n/a | TT900 | 10 | g/L |
| | Flavors | | | | |
| Soy Peptone | Kerry Bio- | n/a | 5X59067 | 20 | g/L |
| | Science | | | | |
| Glycerol | DOW | USP/EP | OPTIM | 40 | g/L |
| | | | Glycerine | | |
| | | | 99.7% | | |
| D-Sorbitol | EMD | BP/JP/NF/EP | 1.11597 | 18.2 | g/L |
| | Chemicals | | | | |
| YNB w/o | Becton | n/a | 292739 | 3.4 | g/L |
| AA w/o | Dickinson | | | | |
| Ammonium | | | | | |
| Sulfate | | | | | |
| Ammonium | JT Baker | NF | 0792 | 10 | g/L |
| Sulfate | | | | | |
| Potassium | JT Baker | USP/EP | 3250 | 2.3 | g/L |
| Phosphate | | | | | |
| dibasic | | | | | |
| Potassium | Fisher | NF/FCC/EP/BP | P386 | 11.9 | g/L |
| Phosphate | | | | | |
| monobasic | | | | | |
| Biotin | DSM | USP/FCC/EP | 04 1745 9 | 8 | mg/L |
| UCON* | ChemPoint | n/a | 17015481 | 0.25 | mL/L |
| | | | or | | |
| | | | 17003079 | | |
| Potassium | Fisher | Multi | P258 | | |
| Hydroxide | | | | | |

*Sterile UCON is added during shake flask prep, before inoculation.

### B. Stirred Tank Seed Stage

To prepare the seed fermenter, 15.6±1 kg (15 L) of non-sterile BYSS Medium (Table 3) was transferred to the vessel followed by 0.7 mL/L of UCON antifoam. The vessel was then heat sterilized for 60 minutes above 125° C. followed by cooling to 24° C. The holding time for non-sterile media should not exceed 8 hours.

The flask inoculum was transferred to an inoculation bottle and 156±16 g (150 mL assuming density of 1.04 g/mL) of inoculum was delivered to the seed fermenter to achieve a 1% inoculation. This seed tank transfer should occur within 45 min of transfer to inoculation bottle. The seed fermenter cultivation continued until the OUR transfer criteria of 50-60 mmol/L/h was attained, which typically occurred within 35±2 h. The pH was controlled at 6.5±0.2 by the addition of 14% (w/w) NH₄OH. Temperature was controlled at 24±1° C., pressure at 19.7 psia (5 psig), aeration at 0.7 vvm (10.5 SLPM, based on 15 L pre-inoculation volume) and dissolved oxygen (DO) at 20±10% of saturation at 19.7 psia and 24° C. by agitation rate.

At transfer, a wet cell weight of 100±25 g/L was achieved. The residual glycerol remaining was 5-15 g/L. At this stage, 1.56±0.2 kg (1.5 L) of culture was transferred to the production fermenter through an inoculation bottle.

TABLE 3
BYSS Medium

| Component | Supplier | Grade | Catalog # | Conc. | Units |
|---|---|---|---|---|---|
| Yeast Extract | Sensient | n/a | TT900 | 10 | g/L |
| | Flavors | | | | |
| Soy Peptone | Kerry Bio- | n/a | 5X59067 | 20 | g/L |
| | Science | | | | |
| Glycerol | DOW | USP/EP | OPTIM | 40 | g/L |
| | | | Glycerine | | |
| | | | 99.7% | | |
| D-Sorbitol | EMD | BP/JP/NF/EP | 1.11597 | 18.2 | g/L |
| | Chemicals | | | | |
| YNB w/o | Becton | n/a | 292739 | 3.4 | g/L |
| AA w/o | Dickinson | | | | |
| Ammonium | | | | | |
| Sulfate | | | | | |
| Ammonium | JT Baker | NF | 0792 | 10 | g/L |
| Sulfate | | | | | |
| Potassium | JT Baker | USP/EP | 3250 | 2.3 | g/L |
| Phosphate | | | | | |
| dibasic | | | | | |
| Potassium | Fisher | NF/FCC/EP/BP | P386 | 11.9 | g/L |
| Phosphate | | | | | |
| monobasic | | | | | |
| Biotin | DSM | USP/FCC/EP | 04 1745 9 | 8 | mg/L |
| UCON* | ChemPoint | n/a | 17015481 | 0.7 | mL/L |
| | | | or | | |
| | | | 17003079 | | |
| Ammonium | JT Baker | NF/Multi | 9736 | | |
| Hydroxide | | | | | |
| (50% of 28% | | | | | |
| stock solution) | | | | | |

*UCON is added just prior to tank sterilization of the media

### C. Production Stage

To prepare the production bioreactor, 15.6±1 kg (15 L) of non-sterile BYSS Medium (Table 3) was transferred to the vessel followed by 0.7 mL/L of UCON antifoam. The vessel was then heat sterilized for 60 minutes above 125° C. followed by cooling to 24° C. The holding time for non-sterile media should not exceed 8 hours.

The cultivation was controlled at: a temperature of 24±1° C., a pH of 6.5±0.2 with the addition of 14% (w/w) NH₄OH and 15% (w/w) H₃PO₄, a pressure of 19.7 psia (5 psig), an airflow rate of 10.5 SLPM (0.7 vvm) and a dissolved oxygen concentration of 20±10% relative to saturation at 19.7 psia, 24° C. with agitation cascaded onto the addition of pure oxygen (0-20 SLPM) to the fixed airflow rate.

The cultivation progressed through four stages:

### Batch Phase

The batch phase began with the transfer of 1.56±0.2 kg (1.5 L assuming density of 1.04 g/mL) of seed tank inoculum to the production fermenter for a 10% inoculation. The OUR during this phase increased exponentially to 80±10 mmol/L/h in 20±2 h before the initial charge glycerol was consumed resulting in a decline in OUR below 55±10 mmol/L/h, signaling the end of batch phase. The biomass concentration at the end of the batch phase was 135±15 g/L of wet cell weight.

### Glycerol Fed Batch Phase

The end of batch phase was followed by the start of glycerol fed batch phase, with initiation of the exponential feed of 50% (w/w) glycerol feed solution (containing PTM2 salts and 25× Biotin) (Table 4) based on the following feed rate formula:

F_{Gly}=Fᵢe^{0.08t}

Where F_{Gly} is the glycerol solution feed rate in g/L*/h, Fᵢ the initial feed rate (5.33 g/L*/h), 0.08 the specific exponential feed rate (h⁻¹), and t the fed batch time in hours. Linearly interpolated feed rates divided into 1 h intervals were used to best fit the exponential feed curve. The glycerol feed is continued for 8 hours. Four hours into the glycerol fed batch phase, 10 mL of UCON was added to the fermenter as a prophylactic shot. During this phase the OUR peaked at 110±20 mmol/L/h. The biomass concentration at the end of the glycerol fed batch phase was 225±25 g/L of wet cell weight.

TABLE 4
50% (w/w) Glycerol Feed Solution*

| Component | Supplier | Grade | Catalog # | Conc. | Units |
|---|---|---|---|---|---|
| Glycerol | DOW | USP/EP | OPTIM | 550 | g/L |
| | | | Glycerine 99.7% | | |

| | | | |
|---|---|---|---|
| PTM2 Salts | See Table 5a | 58.3 | ml/L |
| Solution | | | |
| 25X Biotin Solution | See Table 5b | 58.3 | ml/L |

Dissolved in dH₂0
*Filter sterilize and store at 2-8° C. protected from light

TABLE 5a
PTM2 Salts Solution*

| Component | Supplier | Grade | Catalog # | Conc. | Units |
|---|---|---|---|---|---|
| CuSO₄•5H₂O | JT Baker | USP | 1846 | 0.6 | g/L |
| NaI | Sigma | USP | 383112 | 80 | mg/L |
| MnSO₄•H₂0 | EMD Chemicals | FCC/EP/USP | 1.05999 | 1.81 | g/L |
| H₃BO₃ | JT Baker | NF | 92 | 20 | mg/L |
| FeSO₄•7H₂O | JT Baker | USP | 2074 | 6.5 | g/L |
| ZnCl₂ | JT Baker | USP | 4326 | 2.0 | g/L |
| CoCl₂•6H₂O | Mallinckrodt | ACS | 4532 | 0.5 | g/L |
| Na₂MoO₄•2H₂O | EMD | USP/EP | 1.06524.1000 | 0.2 | g/L |
| Biotin | DSM | USP/FCC/EP | 04 1745 9 | 200 | mg/L |
| Sulfuric Acid | JT Baker | Multi | 9671 | 5 | mL/L |

Dissolved in dH₂0
*Filter sterilize and store at 2-8° C. protected from light

TABLE 5b
25X Biotin Solution*

| Component | Supplier | Grade | Catalog # | Conc. | Units |
|---|---|---|---|---|---|
| Biotin | DSM | USP/FCC/EP | 04 1745 9 | 400 | mg/L |

Dissolved in dH₂0
*Filter sterilize and store at 2-8° C. protected from light

### Transition Phase

After the 8 h glycerol fed batch phase, the glycerol feed was terminated and a 30 minute starvation period was initiated to ensure complete depletion of glycerol and metabolites fowled during the growth phase. This decrease in metabolic activity resulted in an OUR decrease to 30±10 mmol/h/L.

### Methanol Induction Phase

At the end of the 30 minute transition phase, a 18.75±1 mL dose (1.25 mL/L*; L* refers to pre-inoculation volume) of PMTi-4 inhibitor solution (Table 6) was added to the fermenter. At the same time, an exponential feed of 100% methanol was initiated based on the following feed rate formula:

F_{MeOH}=Fᵢe^{0.01t}

Where F_{MeOH} is the methanol feed rate in g/L*/h, Fᵢ the initial feed rate (1.33 g/L*/hr), 0.01 the specific exponential feed rate (h⁻¹), and t the induction time in hours. L* refers to pre-inoculation volume. Linear interpolated feed rates divided into 10 h intervals were used to best fit the exponential feed curve. Methanol induction continued for a total of 80±5 hours from start of the methanol feed. The biomass concentration at the end of methanol induction phase was 380±30 g/L of wet cell weight.

TABLE 6
PMTi-4 Inhibitor Solution

| Component | Supplier | Grade | Catalog # | Conc. | Units |
|---|---|---|---|---|---|
| PMTi-4 | WuXi | n/a | C08010802 | 1.66 | mg/mL |

Dissolved in 100% Methanol

### D. Harvest

Upon completion of the 80±5 hour methanol induction phase, the temperature was lowered to 4-6° C. within 2 hours.

### Example 9

### Purification of Anti-Her2

### Centrifugation

Continuous centrifugation (Westfalia) was performed with Anti-Her2. The broth was initially diluted 1:1 with 6 mM sodium phosphate, 100 mM NaCl, pH 7.2 buffer. CSA-6 was run at 0.75-0.8 L/min (700 mL bowl volume) for removal of solids. The operation was performed at 2-8° C. in order to avoid proteolysis. Turbidity was targeted to be <200 NTU in the centrate.

TABLE 7
Key Parameters for Continuous Centrifugation
Processing Parameters

| | | |
|---|---|---|
| Feed rate | 0.75-0.80 | L/min |
| Temp | 4° | C. |

### Depth Filtration

Depth filtration was performed after centrate is warmed up to >15° C. to further clarify the centrifugation product. Depth filtration should provide <10 NTU product turbidity. The temperature of the centrate was increased to remove additional antifoam prior to chromatography steps.

Depth filtration was performed using Cuno Zeta Plus EXT 60ZA05A in series with 90ZA08A filters. Prior to filtration of centrate, the depth filters were flushed with water (100 L/m2) at a rate of 250 L/m2/hr. The loading for the depth filtration step was kept at a maximum of 350 L/m2. The flow rate across depth filters was kept at 180 L/m2/hr during product filtration and post-use flush. Post-use flush was performed with 6 mM sodium phosphate, 100 mM NaCl, pH 7.2 (25 L/m2) at 180 L/m2/hr and combined with the product.

TABLE 8
Key Parameters for Microfiltration
Processing Parameters

| DF membrane | Cuno Zeta PLUS EXT 60ZA05A in | |
|---|---|---|
| | series with 90 ZA08A | |
| Target Loading | <=350 | L/m2 |
| Water flush | 100 | L/m2 |
| Water flush filtration rate | 250 | L/m2/hr |
| Product and post use filtration | 180 | L/m2/hr |
| rate | | |
| Post-use buffer flush | 25 | L/m2 |
| Starting Feed P | ~10 | psig |
| Ending Feed P | ~15 | psig |

TABLE 9
Processing Buffers used for DF

| Buffer | Use |
|---|---|
| 6 mM sodium phosphate, | Post use flush |
| 100 mM NaCl, pH 7.2 | |

0.22 μm Filtration

For removal of additional antifoam from depth filtered product and to protect the chromatography columns, a 0.22 um filtration was performed. 0.22 μm filtration was performed using a Sartopore 2 0.45/0.2 μm sterile filter from Sartorius at >15° C. in order to force antifoam out of solution. These filters were connected downstream of the depth filters. Filtration operation was then carried out in series with depth filtration. Target filter loading was <=500 L/m2. Collection vessel for filtrate was sterile and connected to filter in sterile environment. Key processing parameters for 0.22 μm filtration are shown in Table 10.

TABLE 10
Key Parameters for Sterile Filtration
Processing Parameters

| 0.22 μm membrane | Sartopore 2 sterile filter with 0.45/0.2 μm pore size | |
|---|---|---|
| Target Loading | <=500 | L/m2 |
| Target Flux | 180 | L/m2/hr |

### Protein A Chromatography

Protein A affinity chromatography was performed as a primary capture step. Bind-elute capture was performed using MabSelect resin from GE Healthcare. Operation was performed at room temperature and eluted product was quenched to pH 6.5 using 1 M Trizmabase. Product collection was based on the UV 280 nm signal and starts when the signal reaches OD 50 and ends when the signal returns to OD 50. Product volume collected from the column was ˜1.7 CV. Process parameters and buffers for this step are shown in Table 11.

The MabSelect column was flow-packed using 6 mM sodium phosphate, 100 mM NaCl, pH 7.2 buffer at 600 cm/hr and pulse tested at 6 min residence time with a volume of 5 M NaCl equivalent to ˜0.5% of the column volume. A well-packed column should have an asymmetry of 1.0-1.5 with >1500 plates/meter. The column was stored in 6 mM sodium phosphate, 100 mM NaCl, pH 7.2 buffer containing 20% ethanol between packing and use.

If proceeding immediately to Capto adhere step with no hold time, product could be quenched all the way to pH 7.8. Process flowrates could be reduced if pressure limitations were encountered.

TABLE 11
Processing parameters and step sequence for Protein A Chromatography
Processing Parameters

| | |
|---|---|
| Resin | GE Healthcare MabSelect |
| Column Loading | <=15 g mAb/L column |
| Column Bed Height | ~20 cm |
| Flowrate for | 6 min residence time |
| Loading/Wash1/Regen/Storage | |
| Flowrate for Equil/Wash2/Wash3/Elute | 4 min residence time |

Sequence of Operations

| Step | Buffer | Length (CV) |
|---|---|---|
| Equilibration | 6 mM sodium phosphate, 100 mM NaCl, pH | 5 CV |
| | 7.2 | |
| Load | 0.22 μm filtered material | |
| Wash 1 | 6 mM sodium phosphate, 100 mM NaCl, pH | 5 CV |
| | 7.2 | |
| Wash 2 | 25 mM sodium phosphate, 1M NaCl, pH 6.0 | 4 CV |
| Wash 3 | 6 mM sodium phosphate, pH 7.2 | 5 CV |
| Elution | 100 mM sodium citrate, pH 3.2 | 5 CV |
| | Collect product peak from OD50 to OD50 | |
| | Quench product to pH 6.5 with 1M | |
| | Trizmabase | |
| Regeneration | 50 mM NaOH, 1M NaCl | 5 CV |
| Storage | 6 mM sodium phosphate, 100 mM NaCl, pH | 3 CV |
| | 7.2 containing 20% Ethanol | |

### Captoadhere Chromatography

Flowthrough chromatography step using Capto adhere resin from GE Healthcare was performed as a polishing chromatography step to remove trace impurities. Operation was performed at room temperature and collected product was titrated to pH 6.5 using 100 mM sodium citrate, pH 3.0. Product collection start was based on the UV 280 nm signal and begins when the signal reaches OD200 and ends when the signal is <=OD200. Process parameters and buffers for this step are shown in Table 12.

The Captoadhere column was flow-packed using 6 mM sodium phosphate, 100 mM NaCl, pH 7.2 buffer at 600 cm/hr and pulse tested at 6 min residence time with a volume of 5 M NaCl equivalent to ˜0.5% of the column volume. A well-packed column should have an asymmetry of 1.0-1.5 with >1500 plates/meter. The column was stored in 0.1 N NaOH between packing and use.

If proceeding immediately to CEX step with no hold time, product can be titrated all the way to pH 5.0. Process flowrates can be reduced if pressure limitations are encountered.

TABLE 12
Processing parameters and step sequence for Capto adhere
Chromatography
Processing Parameters

| | |
|---|---|
| Resin | GE Healthcare Capto adhere |
| Column Loading | 100 g mAb/L column |
| Column Bed Height | ~20 cm |
| Flowrate for | 6 min residence time |
| Loading/Wash/Cleaning/Storage | |
| Flowrate for Equil/Regen | 3 min residence time |

Sequence of Operations

| | | Length |
|---|---|---|
| Step | Buffer | (CV or min) |
| Equilibration | 50 mM sodium phosphate, pH 7.8 | 5 CV |
| Load | 0.22 μm filtered Protein A Product | |
| | quenched to pH 7.8 with 1M Trizmabase | |
| | Product collection starts at OD200, and | |
| | ends at <=OD200 | |
| Wash | 50 mM sodium phosphate, pH 7.8 | 5 CV |
| Regeneration | 50 mM sodium acetate, pH 4.0 | 5 CV |
| Cleaning | 1N NaOH, 2M NaCl | Target 30 min |
| | | contact time |
| Storage | 50 mM sodium phosphate, pH 7.8 with | 4 CV |
| | 20% Ethanol | |

### Cation Exchange Chromatography

Bind-elute capture step using POROS 50HS resin from Applied Biosystems was utilized as the second polishing chromatography step to remove trace impurities. Operation was performed at room temperature. The product pool from Captoadhere chromatography (pH 6.5) step was brought to pH 5.0 using 0.1 M citrate, pH 3.0 (˜50% v/v ratio) prior to start of cation exchange step. Product collection was based on the UV 280 nm signal and starts after the pre-wash and when the signal reaches OD100 and ends when the signal returns to OD100. Product volume collected from the column is ˜5.0 CV. Process parameters and buffers for this step are shown in Table 13. Upon elution, the product pH was adjusted to 6.5 using 1M Trizmabase.

The POROS 50HS column was flow-packed using 50 mM sodium acetate, 1 M NaCl, pH 5.0 buffer at 600 cm/hr and pulse tested at 6 min residence time with a volume of 5 M NaCl equivalent to ˜0.5% of the column volume. A well-packed column should have an asymmetry of 1.0-1.5 with >1500 plates/meter. The column was stored in 0.1 N NaOH between packing and use.

TABLE 13
Processing parameters and step sequence for CEX Chromatography
Processing Parameters

| | |
|---|---|
| Resin | Applied Biosystems POROS 50HS |
| Column Loading | <=20 g mAb/L column |
| Column Bed Height | ~20 cm |
| Flowrate for all steps | 6 min residence time |

Sequence of Operations

| Step | Buffer | Length (CV) |
|---|---|---|
| Equilibration | 50 mM sodium acetate, pH 5.0 | 5 CV |
| Load | 0.22 μm filtered Capto Product titrated to pH | |
| | 5.0 with 100 mM sodium citrate, pH 3.0 | |
| Wash 1 | 50 mM sodium acetate, pH 5.0 | 5 CV |
| Wash 2 | 50 mM sodium acetate, 130 mM NaCl, | 5 CV |
| pH 5.0 | | |
| Elution | 50 mM sodium acetate, 160 mM NaCl, | 10 CV |
| pH 5.0 | | |
| | Collect product peak from OD100 to OD100 | |
| Regeneration | 50 mM sodium acetate, 1M NaCl, pH 5.0 | 5 CV |
| Cleaning | 1N NaOH, 1M NaCl | 5 CV |
| Storage | 0.1N NaOH | 5 CV |

### Ultrafiltration

Ultrafiltration was performed using Millipore Pellicon 2 C-screen regenerated cellulosed membranes with a pore size of 30 kDa to concentrate CEX product to desired concentration for filling and buffer exchange product into formulation buffer. Retentate was concentrated to the target value and then buffer exchanged with 4 diavolumes of formulation buffer. Crossflow rate was kept constant during UF and TMP at startup is ˜10 prig. TMP was controlled with retentate backpressure valve and permeate flow rate. Permeate pressure and flowrate were controlled with a permeate pump. Key processing parameters for ultrafiltration are shown in Table 14.

Prior to use, UF membranes were flushed with water, integrity tested, sanitized with NaOH, and pre-conditioned with diafiltration buffer. If membranes were to be reused, they were flushed with WFI and stored in NaOH following processing.

TABLE 14
Key Parameters for Ultrafiltration
Processing Parameters

| UF membrane | Millipore Pellicon 2 C-screen regenerated cellulose | |
|---|---|---|
| | membrane with 30 kDa pore size | |
| Target Loading | 150-300 | L/m² |
| Crossflow rate | ~6 | LPM/m² |
| Permeate flow rate | ~0.7 | LPM/m² |
| Target Retentate | 25 | mg/mL |
| Concentration | | |
| Diavolumes | 4 | DV |
| Starting Feed P | ~20 | psig |
| Starting Retentate P | ~10 | psig |
| Starting Permeate P | ~5 | psig |

### Bioburden Reduction Filtration

Bioburden reduction filtration is performed using a Sartopore 2 0.45/0.2 μm sterile filter from Sartorius to ensure minimal bioburden is present in final product. Target filter loading was >200 L/m2 at a flux of 200 LMH. Collection vessel for filtrate was sterile and connected to filter in sterile environment. Key processing parameters for the bioburden reduction filtration are shown in Table 15.

TABLE 15
Key Parameters for Bioburden Reduction Filtration
Processing Parameters

| 0.22 μm membrane | Sartopore 2 sterile filter with 0.45/0.2 μm pore size | |
|---|---|---|
| Target Loading | >200 | L/m² |
| Target Flux | 200 | LMH |

### Example 10

### N-Linked Glycan Analysis by HPLC of Anti-her2 from Strains YGLY13979, YGLY13992 and YGLY12501

To quantify the relative amount of each glycoform, the N-glycosidase F released glycans were labeled with 2-aminobenzidine (2-AB) and analyzed by HPLC as described in Choi et al., Proc. Natl. Acad. Sci. USA 100: 5022-5027 (2003) and Hamilton et al., Science 313: 1441-1443 (2006). The O-glycan was detected according to Stadheim et al., Nature Protocols, Vol 3. No. 6, (2008).

The glycan profiles from Her2 antibodies generated at 40 liter fermentation scale of strains YGLY13979, YGLY12501 and YGLY13992 are described below.

TABLE 16

| | O-Linked glycan | | N-Linked glycan | | | | | |
|---|---|---|---|---|---|---|---|---|
| | Occupancy | Single | | | | | | Complex |
| | (mol/mol) | mannose | G0 | G1 | G2 | Man5 | Hybrid** | (G0 + G1 + G2) |
| YGLY13979 | 1.2 | >99% | 60 | 21 | 3 | 8 | 8 | 84 |
| YGLY13992 | 2.0 | >99% | 59 | 23 | 2 | 8 | 8 | 85 |
| YGLY12501 | 1.6 | >99% | 59 | 23 | 3 | 7 | 8 | 85 |

**Hybrid form is GlcNAcMan₅GlcNAc₂ and/or GalGlcNAcMan₅GlcNAc₂

The glycan profiles from Her2 antibodies generated at large fermentation scale of strain YGLY13979 are described below.

TABLE 17

| Analysis | | 13979(2) |
|---|---|---|
| N-glycan | Occupancy | 84.7% |
| | G0/G1/G2 | 77.3% |
| | Man5 | 12.0% |
| | Hybrid | 10.8% |
| O-glycan | O-mannose occupancy | 1 mol/mol |

### Example 11

### Her2 Target Binding Affinity

Surface plasmon resonance measurements of binding affinity using BIAcore T100 instrument were performed at 25° C. at a flow rate of 40 μl/min. An anti-human IgG-Fc antibody (50 μg/ml each in acetate buffer, pH 5.0) was immobilized onto a carboxymethyl dextran sensorchip (CM5) using amine coupling procedures as described by the manufacturer (Biosystem). Close to 10000 resonance units (RU) of anti-IgG Fc antibodies were immobilized chemically respectively onto Flow cells (FC) 1 and 2. Purified anti-HER2 antibodies to be tested were diluted at a concentration of 5 μg/ml in 0.5% P20, HBS-EP buffer and injected on FC2 to reach 500 to 1000 RU. FC1 was used as the reference cell. Specific signals were measured as the differences of signals obtained on FC2 versus FC1. The recombinant human Her2 ECD as analyte was injected during 90 sec at series of concentrations 0-100 nM in 0.5% P20, HBS-EP buffer. The dissociation phase of the analyte was monitored over a 10 minutes period. Running buffer was also injected under the same conditions as a double reference. After each running cycle of capturing antibody and binding of HER2 ECD, both Flowcells were regenerated by injecting 45 μl of Glycine-HCl buffer pH 1.5. This regeneration is sufficient to eliminate all Mabs and Mabs/Her2 complexes captured on the sensorchip.

Anti-HER2 antibodies produced from YGLY12501, YGLY13992, and YGLY13979 were analyzed using Herceptin® as a comparator. The binding kinetics of anti-HER2 antibody to HER2ECD was characterized by both association and dissociation rate constants kₐ and k_{d}. The equilibrium dissociation constant (K_{D}) was calculated by the ratio between dissociation and association rate constants. Lower K_{E}, values were established for anti-HER2 from strains YGLY13979, YGLY12501 and YGLY13992 in comparison with Herceptin®. Table 18. Kinetic constants for HER2 ECD antigen binding of Her2 antibodies from strains YGLY13979, YGLY12501 and YGLY13992 in comparison with Herceptin® (n=6)

| | K_{D}, nM | |
|---|---|---|
| Antibody name | (mean ± stdev) | ¹RP |
| ²Herceptin ® | 1.15 ± 0.18 | 1.0 |
| YGLY13979 | 0.62 ± 0.10 | 1.9 |
| YGLY13979 (2) | 0.77 ± 0.05 | 1.5 |
| YGLY12501 | 0.77 ± 0.10 | 1.5 |
| YGLY13992 | 0.74 ± 0.04 | 1.6 |

¹RP: relative potency = K_{D} value of Herceptin ®/value of anti-HER2
²the value for Herceptin ® is generated with n = 45

### Example 12

### Inhibition of Cancer Cell Proliferation

Exponentially growing BT474.m1 cells were harvested and plated onto 96-well plates (Costar 3603, Corning Inc.) at 5,000 cells/well with 100 μl of cell culture medium (RPMI media with 10% FBS). After 24 h culturing, cells were treated with anti-HER2 antibodies in a series of 1:2 diluted antibody concentrations ranging from 33.3 to 0 nM (control). After 96 h incubation, 10 μl of AlamarBlue (Invitrogen, DAL1100) were added to each well and cultured for additional 4 h before reading the plates. Fluorescence emission intensity was then measured at Ex/Em of 535/590 nm. Inhibitions of proliferation of breast cancer cells (BT474M1) were determined using the output fluorescence signals and human irrelevant IgG as no treatment control. The IC50s were calculated using 4 parameter curve fitting with Graphpad program.

TABLE 19
Relative potency of anti-HER2 antibodies vs Herceptin ® for
inhibition of cell proliferation (n = 8)

| Name | RP |
|---|---|
| Herceptin ® | 1.0 |
| YGLY13979 | 1.5 ± 0.4 |
| YGLY13979 (2) | 1.3 ± 0.4 |
| YGLY12501 | 1.3 ± 0.2 |
| YGLY13992 | 1.2 ± 0.3 |

### Example 13

### Fc Gamma Receptor Binding Affinities

The binding of anti-HER2 to FcγRI, FcγRIIA (R, H), FcγRTIIIA(F, V), FcγRIIB/C, and FcγRIIIB was measured using BIAcore T100 with CM5 biosensor chips (GE Healthcare, USA). Running buffer contained 10 mM Hepes, 150 mM NaCl, 3 mM EDTA, 0.005% surfactant P20, pH 7.4. To immobilize the Goat F(ab′)2 anti-human Kappa on the chip, the chip surface was activated by the injection of EDC-NHS for 7 min at 10 μL/min, followed by the injection of Fab2 fragment antibody (5 μg/mL) in an acetate buffer (10 mM, pH 5). The immobilization reaction was then quenched by the addition of ethanolamine HCl (1M, pH 8.5) for 7 min at 10 μL/min. For affinity studies, anti-HER2 antibodies were captured on chip and individual Fey receptors at various concentrations (1600, 800, 400, 200, 100, 50, 25 and 0 nM) were injected into the cells at 60 μL/min for 2 min. To ensure a steady state of binding was reached, followed by 5 min dissociation. The sensor surface was regenerated through Glycine-HCl buffer pH 1.5. The data was then fitted into a 1:1 steady state binding model in the BIAcore T100 evaluation software and the equilibrium constant (K_{D}) was calculated.

Anti-HER2 antibodies showed superior FcγRIIII A & B binding affinities to trastuzumab and slight lower binding affinities to FcgRIIA (H) in comparison with trastuzumab. This improved FcγRIII binding affinities contributed to better ADCC activities discussed in the next example.

TABLE 20
Comparison of anti-HER2 and Herceptin ® binding affinities on different FcγRs,
expressed as relative potency (n = 6)

| | | | YGLY13979 | | |
|---|---|---|---|---|---|
| ¹RP | Herceptin ® | YGLY13979 | (2) | YGLY12501 | YGLY13992 |
| FcγRIIIA (F) | 1.0 | 5.2 | 4.3 | 5.7 | 5.7 |
| FcγRIIIA (V) | 1.0 | 4.1 | 3.7 | 7.7 | 5.2 |
| FcγRIIIB | 1.0 | 3.1 | 2.9 | 3.7 | 3.5 |
| FcγRIIA (H) | 1.0 | 0.7 | 0.6 | 0.6 | 0.7 |
| FcγRIIA (R) | 1.0 | 1.0 | 0.9 | 1.1 | 1.1 |
| FcγRIIB/C | 1.0 | 1.1 | 1.1 | 1.2 | 1.3 |
| FcγRI | 1.0 | 0.7 | 0.7 | 0.7 | 0.9 |

¹RP = K_{D} of Herceptin ®/K_{D} of anti-HER2

### Example 14

### ADCC Activities

ADCC activities were assayed with human ovarian adenocarcinoma cell line SKOV3 as target cells and human NK cells as effector cells. Target cells were grown as adherent in culture medium RPMI (Mediatech Catalog #10-040-CM) supplemented with 10% FBS. Effector NK cells were ordered from Biological Specialty (catalog #215-11-10) and used on the day delivered.

15,000 target cells (SKOV3)/well were seeded into 96 wells E-plate with 100 ul of media per well. Cell growth was monitored with the impedance based RT-CES system until they reached log growth stage and formed a monolayer (about 24 hours). Effector cells (NK cells) were added at 150,000/well (Effector:Target=10:1). Antibodies were added at a series of 4 fold titrations across the plate. Controls with target cell only, target plus NK cells and 100% lysis with detergent were run in each assay. The system took measurements every thirty minutes for the first 8 hours and then every hour for the next 16 hours. Cell lysis was quantified by exporting the data into Microsoft excel and percentage of lysis was determined according to the formula (CI target plus NK only−CI sample well)/(CI target plus NK only)*100 (CI stands for Cell Index, which is the arbitrary unit the assay system uses to express impedance). EC50 was determined from the dose response curve using Graft pad 4 parameter fitting model.

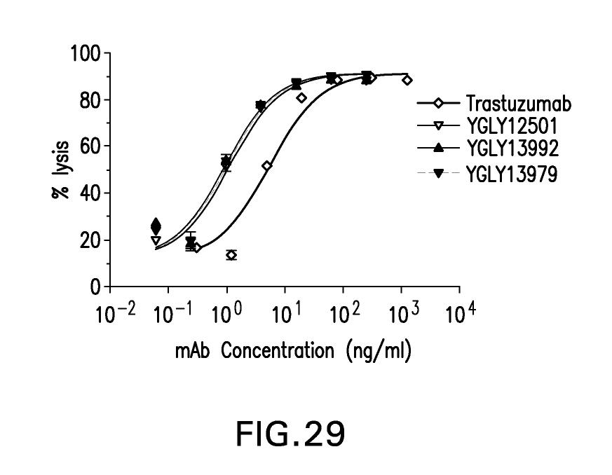

Her2 antibody from strain YGLY13979 showed an average of 4-fold increase of ADCC activity vs Herceptin®. Comparable ADCC was shown for Her2 antibodies from strains YGLY13979 and YGLY12501. (FIG. 29).

TABLE 21
Relative potency (RP) of ADCC activities of anti-HER2 antibodies
in comparison with Herceptin ® (n = 10)

| Name | ¹RP |
|---|---|
| Herceptin ® | 1.0 |
| YGLY13979 | 4.5 ± 0.8 |
| YGLY13979 (2) | 4.3 ± 1.0 |
| YGLY12501 | 5.3 ± 1.2 |
| YGLY13992 | 5.1 ± 0.5 |

¹RP = EC50 of Herceptin ®/EC50 of anti-HER2

### Example 15

### Pharmacokinetics

PK of Her2 Antibody from GFI5.0 in Cynomolgus Monkeys

Male rhesus nonhuman primates (Macaca mulatta) were dosed intravenously with 10 mg/kg (N=3) of anti-Her2 mAb produced from either CHO cells (commercial Herceptin), GFI2.0 Pichia, GFI5.0 Pichia or wild type Pichia. The light chain chain and heavy chain amino acid sequences of the Pichia produced Her2 antibodies are SEQ ID NOs:18 and 20, respectively. Serum samples were collected at the following intervals post dose 1 (0, 15 min, 2, 4, 8, 24, 48, 96, 168, 216, 264, 360, 432, 504 hours).

Human IgG levels were determined using a sandwich ELISA. Briefly, biotinylated mouse anti-human kappa chain (BD Pharmingen) (2.5 μg/ml) was applied to streptavidin-coated plates (Pierce) and incubated 2 hr at room temperature. Plates were washed and samples containing human IgG were applied and incubated for 2 hr at room temperature. Plates were washed and incubated with an HRP-conjugated mouse monoclonal antibody specific for human IgG Fc (Southern Biotech) (1:10,000 dilutions). After a final plate wash, TMB substrate (R&D Systems) was applied to the plate, incubated for 15 min and quenched with 1N sulfuric acid prior to reading on a Molecular Devices plate reader at OD450 nm. The standard curve was fit using a 4^{th} parameter equation in Softmax Pro and concentrations determined for QC and study samples. PK analysis was performed in WinNolin Enterprise Version 5.01 (Pharsight Corp, Mountian View, Calif.).

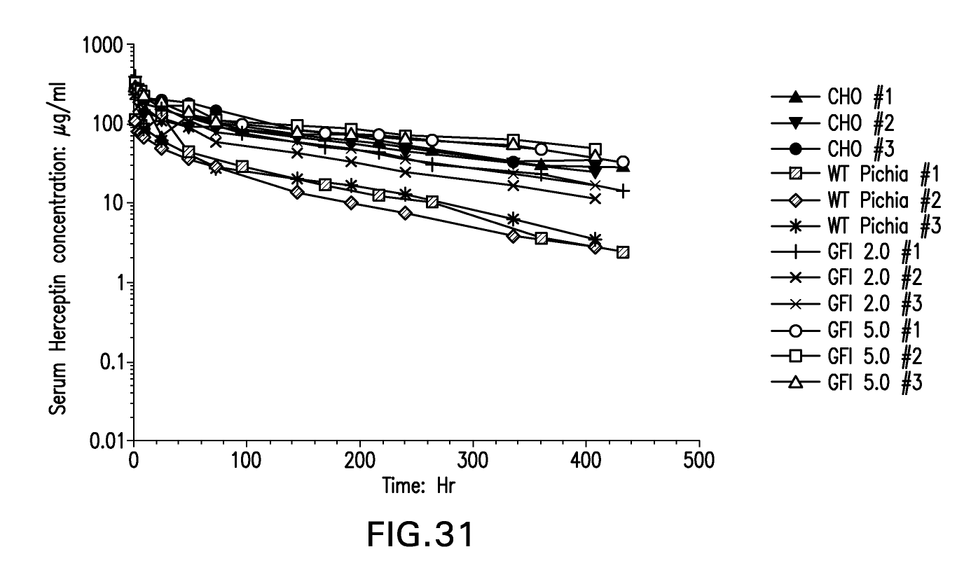

As shown in FIG. 31, Her2 antibody expressed in GFI5.0 Pichia exhibited similar PK profile to that of commercial Herceptin produced in CHO cells. Specifically, the systemic exposure, clearance, t½, MRT and Vss of Her2 antibody from GFI 5.0 were similar to those of commercial Herceptin. Her2 antibody expressed in wild type Pichia had dramatically lower systemic exposure clearance, t½, MRT and Vss than those of either Her2 antibody from GFI 5.0 or commercial Herceptin. Although OFT 2.0 Pichia produced Her2 antibody showed much better PK profile than that of Her2 antibody made in wild type Pichia, the systemic exposure and t½ were still significantly lower than those of Herceptin expressed in CHO or Her2 antibody from GFI-5.0. The extent of the exposure for Herceptin glycovariants appear to correlate with the content of terminal mannose. Her2 antibody expressed in wild type Pichia has the highest contents of terminal mannose followed by material produced in GFI 2.0.

TABLE 22
Key PK parameters of Herceptin Glycovariants in NHP

| | CHO- | WT-Her2 | GFI2.0-Her2 | GFI5.0-Her2 |
|---|---|---|---|---|
| | Herceptin | Antibody | Antibody | Antibody |
| AUC_{0-INF} (hr * ug/ml) | 39655 ± 8266 | 9028 ± 2442 | 25421 ± 4718 | 51091 ± 5883 |
| Cl (ml/hr/kg) | 0.26 ± 0.05 | 1.15 ± 0.3 | 0.4 ± 0.08 | 0.2 ± 0.02 |
| MRT_{0-INF} (hr) | 299 ± 11 | 117 ± 11 | 192 ± 9.2 | 347 ± 33 |
| t_{1/2} (hr) | 214 ± 20 | 98 ± 3 | 153 ± 6.7 | 263 ± 23 |
| Vₛₛ (ml/kg) | 77 ± 14 | 136 ± 49 | 77 ± 12 | 68 ± 2.6 |

PK of Her2 Antibody from YGLY12501 in Cynomolgus Monkeys

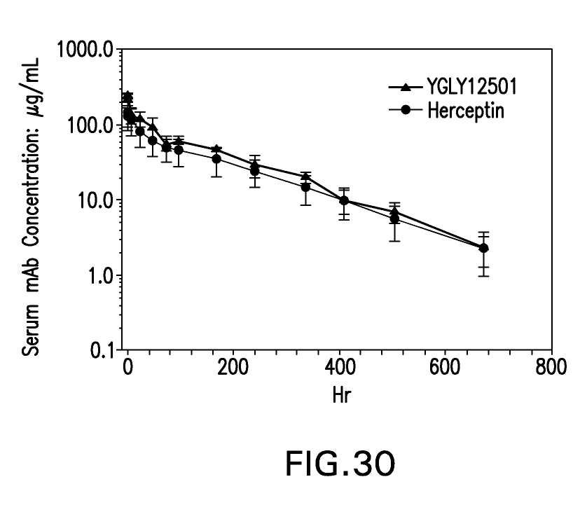

Cynomolgus monkeys were dosed with Her2 antibody from strain YGLY12501 or Herceptin® via intravenous administration at 5 mg/kg. The results showed that the serum time-concentration profile of Her2 antibody from YGLY12501 was comparable to that of Herceptin®(FIG. 30). The key PK parameters of Her2 antibody from YGLY12501 were largely comparable to those of Herceptin® although the exposure appeared to be slightly higher for Her2 antibody from YGLY12501. The t½ of Herceptin® is within the range of that reported for Herceptin®.

TABLE 23
Key PK parameters of Her2 antibody from YGLY12501 and
Herceptin ® after IV administration at 5 mg/kg in Cynomolgus
monkeys (Data expressed as mean ± SD, N = 3)

| | YGLY12501 | Herceptin ® |
|---|---|---|
| t_{1/2} (hr) | 124 ± 22 | 124 ± 11* |
| AUC_{Last} (hr * ug/mL) | 20420 ± 2780 | 15792 ± 6064 |
| AUC_{0-INF} (hr * ug/mL) | 20868 ± 2935 | 16197 ± 6186 |
| CL (mL/hr/kg) | 0.24 ± 0.04 | 0.34 ± 0.13 |
| Vₛₛ (mL/kg) | 41 ± 6.3 | 59 ± 19 |

*FOI data: t_{1/2} ranged from 6-10 days following IV administration at 1.5 mg/kg in NHP

PK of Her2 Antibodies from YGLY13979 and YGLY13992 in Wild-Type Mice

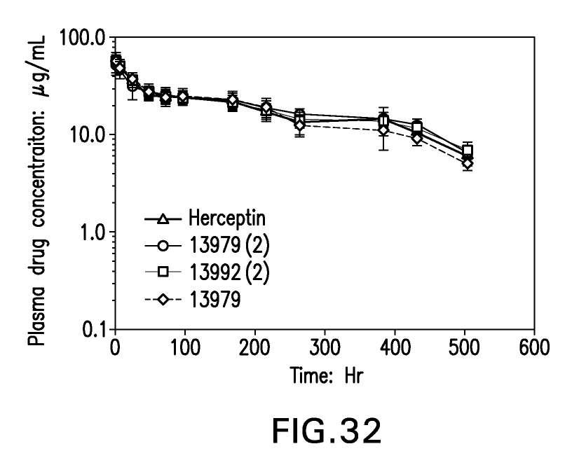

Her2 antibodies from YGLY13979 (2), YGLY13992 (2) and YGLY13979 were compared to Herceptin® in a pharmacokinetic study in C57B6 mice following intravenous administration at 4 mg/kg (n=5). The results showed that the plasma time-concentration profile of Her2 antibodies from YGLY13979 (2), YGLY13992 (2) and YGLY13979 were similar to that of Herceptin® and the key PK parameters such as AUC, CL and t_{1/2} were comparable to those of Herceptin® (FIG. 32).

TABLE 24
Key PK parameters of Her2 antibodies from YGLY13992 (2),
YGLY13979 (2), YGLY13979 and Herceptin ® after IV
administration in C57B6 mice (Data expressed as mean ± SD, N = 5).

| | Herceptin ® | 13979 (2) | 13992 (2) | 13979 |
|---|---|---|---|---|
| C₀ | 60 ± 8 | 55 ± 14 | 59 ± 5 | 59 ± 14 |
| (ug/mL) | | | | |
| t_{1/2} (hr) | 223 ± 26* | 241 ± 18 | 256 ± 47 | 201 ± 12 |
| AUCₗₐₛₜ | 7796 ± 1463 | 8247 ± 1255 | 7970 ± 919 | 7420 ± 1108 |
| (hr * | | | | |
| ug/mL) | | | | |
| AUC_{0-INF} | 9761 ± 2033 | 10491 ± 1282 | 10602 ± 576 | 8892 ± 1201 |
| (hr * | | | | |
| ug/mL) | | | | |
| CL | 0.43 ± 0.09 | 0.39 ± 0.05 | 0.38 ± 0.02 | 0.46 ± 0.07 |
| (ml/hr/kg) | | | | |
| Vₛₛ | 130 ± 19 | 130 ± 23 | 137 ± 30 | 127 ± 28 |
| (ml/kg) | | | | |

*FOI data: t_{1/2} ranged from 11-39 days following IV administration in mice

### Example 16

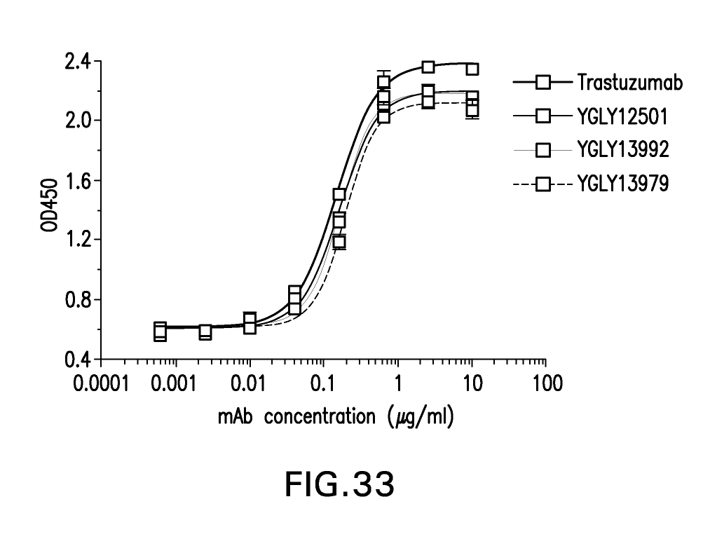

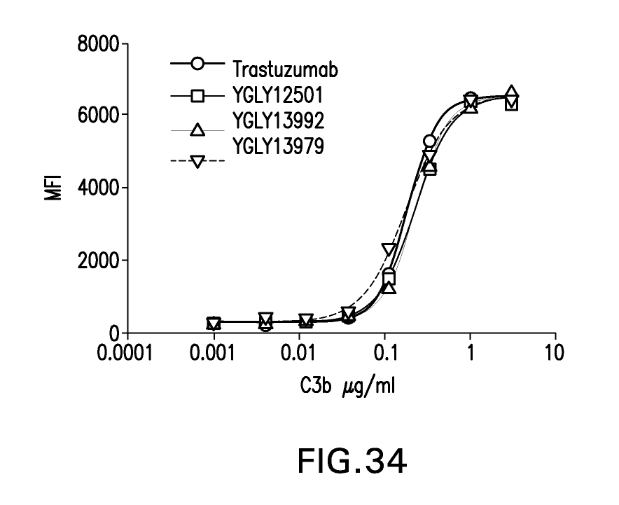

The binding of anti-HER2 from strains YGLY12501, YGLY13992 and YGLY13979 to human C1q (Quidel, San Diego, Calif.) and C3b was assessed in an ELISA format. MaxSorp 96-well plates were coated overnight at 4° C. with 2 ug/ml of HER2 ECD in PBS. Anti-HER2 and Herceptin® were captured on plates by HER2ECD. Human C1q or C1q titrated in human complement system (C1q depleted system) were incubated for 2 hrs. Binding of C1q or C3b deposition on the anti-HER2 plates was detected. Both C1q binding (FIG. 33) and C3b deposition (FIG. 34) to anti-HER2 were comparable to Herceptin®. There was no detectable CDC activity for both anti-Her2 and Herceptin® when using MCF7/her2-18 and BT474.M1 as target cells. This lack of detectable CDC activity is consistent with reported data for Herceptin® when assayed under similar conditions in vitro.

### Example 17

The below plasmids can be used to introduce the LmSTT3D expression cassettes into P. pastoris to increase the level of N-glycan occupancy on glycoproteins produced in example 4.

Plasmids comprising expression cassettes encoding the Leishmania major STT3D (LmSTT3D) open reading frame (ORF) operably linked to an inducible or constitutive promoter were constructed as follows.

The open reading frame encoding the LmSTT3D (SEQ ID NO:12) was codon-optimized for optimal expression in P. pastoris and synthesized by GeneArt AG, Brandenburg, Germany. The codon-optimized nucleic acid molecule encoding the LmSTT3D was designated pGLY6287 and has the nucleotide sequence shown in SEQ ID NO:11.

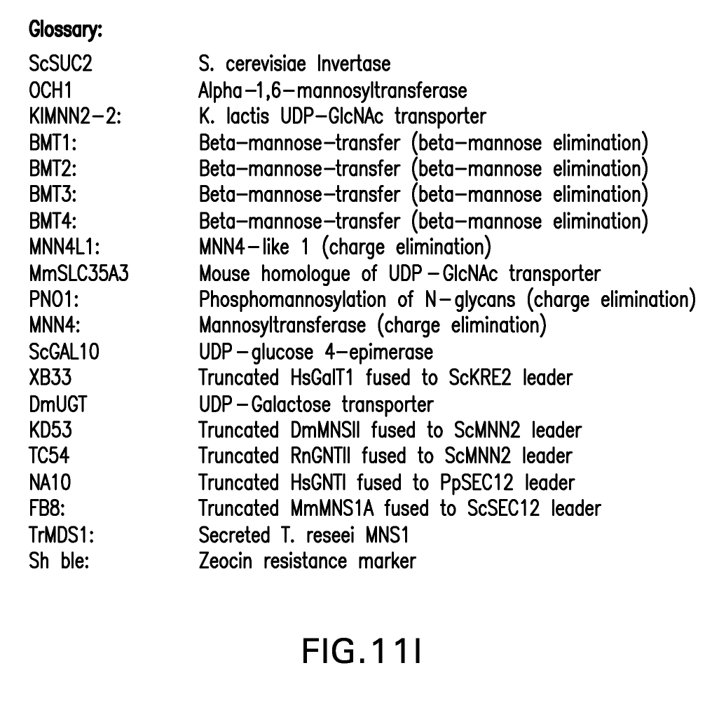

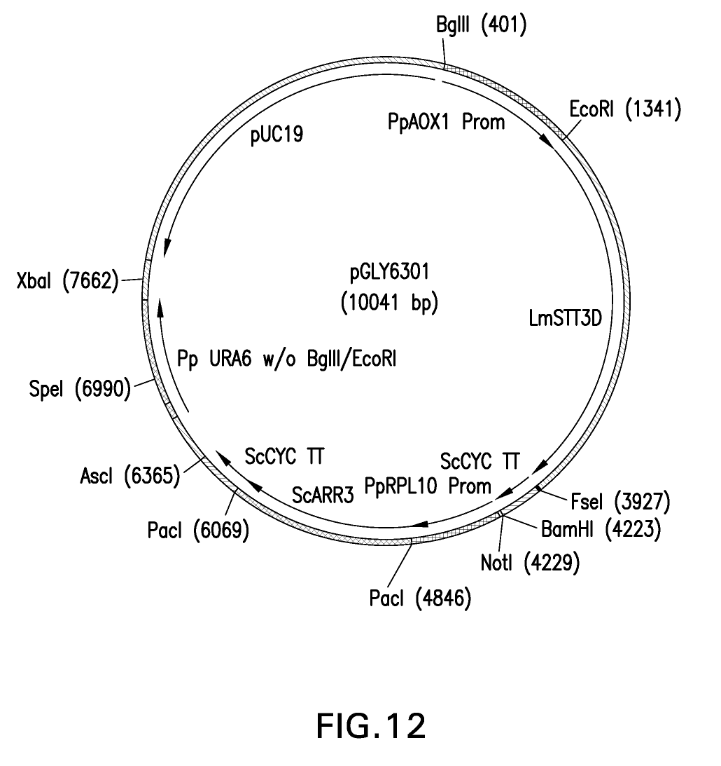

Plasmid pGLY6301 (FIG. 12) is a roll-in integration plasmid that targets the URA6 locus in P. pastoris. The expression cassette encoding the LmStt3D comprises a nucleic acid molecule encoding the LmSTT3D ORF codon-optimized for effective expression in P. P. pastoris operably linked at the 5′ end to a nucleic acid molecule that has the inducible P. pastoris AOX1 promoter sequence (SEQ ID NO:23) and at the 3′ end to a nucleic acid molecule that has the S. cereviseae CYC transcription termination sequence (SEQ ID NO:24). For selecting transformants, the plasmid comprises an expression cassette encoding the S. cerevisiae ARR3 ORF in which the nucleic acid molecule encoding the ORF (SEQ ID NO:32) is operably linked at the 5′ end to a nucleic acid molecule having the P. pastoris RPL10 promoter sequence (SEQ ID NO:25) and at the 3′ end to a nucleic acid molecule having the S. cereviseae CYC transcription termination sequence (SEQ ID NO:24). The plasmid further includes nucleic acid molecule for targeting the URA6 locus (SEQ ID NO:33). Plasmid pGLY6301 was constructed by cloning the DNA fragment encoding the codon-optimized LmSTT3D ORF (pGLY6287) flanked by an EcoRI site at the 5′ end and an FseI site at the 3′ end into plasmid pGFI30t, which had been digested with EcoRI and FseI.

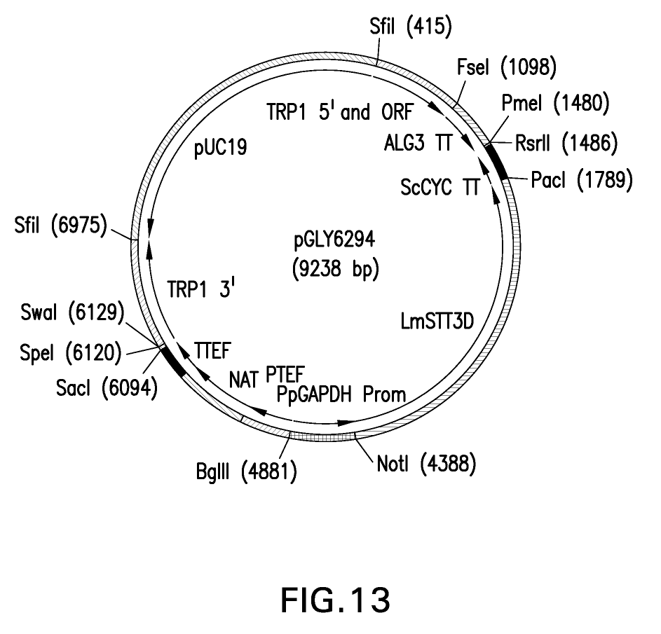

Plasmid pGLY6294 (FIG. 13) is a KINKO integration vector that targets the TRP1 locus in P. pastoris without disrupting expression of the locus. KINKO (Knock-In with little or No Knock-Out) integration vectors enable insertion of heterologous DNA into a targeted locus without disrupting expression of the gene at the targeted locus and have been described in U.S. Published Application No. 20090124000. The expression cassette encoding the LmStt3D comprises a nucleic acid molecule encoding the LmSTT3D ORE operably linked at the 5′ end to a nucleic acid molecule that has the constitutive P. pastoris GAPDH promoter sequence (SEQ ID NO:26) and at the 3′ end to a nucleic acid molecule having the S. cereviseae CYC transcription termination sequence (SEQ ID NO:24). For selecting transformants, the plasmid comprises an expression cassette encoding the Nourseothricin resistance (NATR) ORF (originally from pAG25 from EROSCARF, Scientific Research and Development GmbH, Daimlerstrasse 13a, D-61352 Bad Homburg, Germany, See Goldstein et al., Yeast 15: 1541 (1999)); wherein the nucleic acid molecule encoding the ORF (SEQ ID NO:34) is operably linked to at the 5′ end to a nucleic acid molecule having the Ashbya gossypii TEF1 promoter sequence (SEQ ID NO:86) and at the 3′ end to a nucleic acid molecule that has the Ashbya gossypii TEF1 termination sequence (SEQ ID NO:87). The two expression cassettes are flanked on one side by a nucleic acid molecule comprising a nucleotide sequence from the 5′ region of the ORF encoding Trp1p ending at the stop codon (SEQ ID NO:30) linked to a nucleic acid molecule having the P. pastoris ALG3 termination sequence (SEQ ID NO:29) and on the other side by a nucleic acid molecule comprising a nucleotide sequence from the 3′ region of the TRP1 gene (SEQ ID NO:31). Plasmid pGLY6294 was constructed by cloning the DNA fragment encoding the codon-optimized LmSTT3D ORF (pGLY6287) flanked by a Nod site at the 5′ end and a Pad site at the 3′ end into plasmid pGLY597, which had been digested with Nod and FseI. an expression cassette comprising a nucleic acid molecule encoding the Nourseothricin resistance ORF (NAT) operably linked to the Ashbya gossypii TEF1 promoter (PTEF) and Ashbya gossypii TEF1 termination sequence (TTEF).

Transformation of strain YGLY13992 with the above LmSTT3D expression/integration plasmid vectors was performed essentially as follows. Appropriate Pichia pastoris strains were grown in 50 mL YPD media (yeast extract (1%), peptone (2%), dextrose (2%)) overnight to an OD of between about 0.2 to 6. After incubation on ice for 30 minutes, cells were pelleted by centrifugation at 2500-3000 rpm for five minutes. Media was removed and the cells washed three times with ice cold sterile 1 M sorbitol before resuspension in 0.5 mL ice cold sterile 1 M sorbitol. Ten μL linearized DNA (5-20 μg) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (24° C.) before plating the cells on selective media.

Strain YGLY13992 was transformed with pGLY6301, which encodes the LmSTT3D under the control of the inducible AOX1 promoter, or pGLY6294, which encodes the LmSTT3D under the control of the constitutive GAPDH promoter, as described above to produce the strains described in the following example.

### Example 18

Integration/expression plasmid pGLY6301, which comprises the expression cassette in which the ORF encoding the LmSTT3D is operably-linked to the inducible PpAOX1 promoter, or pGLY6294, which comprises the expression cassette in which the ORF encoding the LmSTT3D is operably-linked to the constitutive PpGAPDH promoter, was linearized with SpeI or SfiI, respectively, and the linearized plasmids transformed into Pichia pastoris strain YGLY13992 to produce strains YGLY17351, YGLY17368 shown in Table 25. Transformations were performed essentially as described above.

TABLE 25

| Strain | Antibody | LmSTT3D expression |
|---|---|---|
| YGLY13992 | Anti-Her2 | None |
| YGLY17351 | Anti-Her2 | +−inducible |
| YGLY17368 | Anti-Her2 | +constitutive |

The genomic integration of pGLY6301 at the URA6 locus was confirmed by colony PCR (cPCR) using the primers, PpURA6out/UP (5′-CTGAGGAGTCAGATATCAGCTCAATCTCCAT-3′; SEQ ID NO: 1) and Puc19/LP (5′-TCCGGCTCGTATGTTGTGTGGAATTGT-3; SEQ ID NO: 2) or ScARR3/UP (5′-GGCAATAGTCGCGAGAATCCTTAAACCAT-3; SEQ ID NO: 3) and PpURA6out/LP (5-CTGGATGTTTGATGGGTTCAGTTTCAGCTGGA-3′; SEQ ID NO: 4).

The genomic integration of pGLY6294 at the TRP1 locus was confirmed by cPCR using the primers, PpTRP-5′ out/UP (5′-CCTCGTAAAGATCTGCGGTTTGCAAAGT-3′; SEQ ID NO: 5) and PpALG3TT/LP (5′-CCTCCCACTGGAACCGATGATATGGAA-3′; SEQ ID NO: 6) or PpTEFTT/UP (5′-GATGCGAAGTTAAGTGCGCAGAAAGTAATATCA-3′; SEQ ID NO: 7) and PpTRP1-3′ out/LP (5′-CGTGTGTACCTTGAAACGTCAATGATACTTTGA-3′; SEQ ID NO: 8). Integration of the expression cassette encoding the LmSTT3D into the genome was confirmed using cPCR primers, LmSTT3D/iUP (5′-GCGACTGGTTCCAATTGACAAGCTT-3′ (SEQ ID NO: 9) and LmSTT3D/iLP CAACAGTAGAACCAGAAGCCTCGTAAGTACAG-3′ (SEQ ID NO: 10). The PCR conditions were one cycle of 95° C. for two minutes, 35 cycles of 95° C. for 20 seconds, 55° C. for 20 seconds, and 72° C. for one minute; followed by one cycle of 72° C. for 10 minutes.

The strains were cultivated in a Sixfor fermentor to produce the antibodies for N-glycan occupancy analysis. Cell Growth conditions of the transformed strains for antibody production was generally as follows.

Protein expression for the transformed yeast strains was carried out at in shake flasks at 24° C. with buffered glycerol-complex medium (BMGY) consisting of 1% yeast extract, 2% peptone, 100 mM potassium phosphate buffer pH 6.0, 1.34% yeast nitrogen base, 4×10⁻⁵% biotin, and 1% glycerol. The induction medium for protein expression was buffered methanol-complex medium (BMMY) consisting of 1% methanol instead of glycerol in BMGY. Pmt inhibitor Pmti-3 in methanol was added to the growth medium to a final concentration of 18.3 μM at the time the induction medium was added. Cells were harvested and centrifuged at 2,000 rpm for five minutes.

SixFors Fermentor Screening Protocol followed the parameters shown in Table 26.

TABLE 26
SixFors Fermentor Parameters

| Parameter | Set-point | Actuated Element |
|---|---|---|
| pH | 6.5 ± 0.1 | 30% NH₄OH |
| Temperature | 24 ± 0.1 | Cooling Water & Heating Blanket |
| Dissolved O2 | n/a | Initial impeller speed of 550 rpm is |
| | | ramped to 1200 rpm over first 10 hr, then |
| | | fixed at 1200 rpm for remainder of run |

At time of about 18 hours post-inoculation, SixFors vessels containing 350 mL media A plus 4% glycerol were inoculated with strain of interest. A small dose (0.3 mL of 0.2 mg/mL in 100% methanol) of Pmti-3 (5-[[3-(1-Phenyl-2-hydroxy)ethoxy)-4-(2-phenylethoxy)]phenyl]methylene]-4-oxo-2-thioxo-3-thiazolidineacetic Acid) (See Published International Application No. WO 2007061631) was added with inoculum. At time about 20 hour, a bolus of 17 mL 50% glycerol solution (Glycerol Fed-Batch Feed) plus a larger dose (0.3 mL of 4 mg/mL) of Pmti-3 was added per vessel. At about 26 hours, when the glycerol was consumed, as indicated by a positive spike in the dissolved oxygen (DO) concentration, a methanol feed was initiated at 0.7 mL/hr continuously. At the same time, another dose of Pmti-3 (0.3 mL of 4 mg/mL stock) was added per vessel. At time about 48 hours, another dose (0.3 mL of 4 mg/mL) of Pmti-3 was added per vessel. Cultures were harvested and processed at time about 60 hours post-inoculation.

TABLE 27
Composition of Media A

| | | |
|---|---|---|
| Soytone L-1 | 20 | g/L |
| Yeast Extract | 10 | g/L |
| KH₂PO4 | 11.9 | g/L |
| K₂HPO₄ | 2.3 | g/L |
| Sorbitol | 18.2 | g/L |
| Glycerol | 40 | g/L |
| Antifoam Sigma 204 | 8 | drops/L |
| 10X YNB w/Ammonium Sulfate w/o | 100 | mL/L |
| Amino Acids (134 g/L) | | |
| 250X Biotin (0.4 g/L) | 10 | mL/L |
| 500X Chloramphenicol (50 g/L) | 2 | mL/L |
| 500X Kanamycin (50 g/L) | 2 | mL/L |

TABLE 28
Glycerol Fed-Batch Feed

| | | |
|---|---|---|
| Glycerol | 50% | m/m |
| PTM1 Salts | 12.5 | mL/L |
| 250X Biotin (0.4 g/L) | 12.5 | mL/L |

TABLE 29
Methanol Feed

| | | |
|---|---|---|
| Methanol | 100% | m/m |
| PTM1 Salts | 12.5 | mL/L |
| 250X Biotin (0.4 g/L) | 12.5 | mL/L |

TABLE 30
PTM1 Salts

| | | |
|---|---|---|
| CuSO₄—5H₂O | 6 | g/L |
| NaI | 80 | mg/L |
| MnSO₄—7H₂O | 3 | g/L |
| NaMoO₄—2H₂O | 200 | mg/L |
| H₃BO₃ | 20 | mg/L |
| CoCl₂—6H₂O | 500 | mg/L |
| ZnCl₂ | 20 | g/L |
| FeSO₄—7H₂O | 65 | g/L |
| Biotin | 200 | mg/L |
| H₂SO₄ (98%) | 5 | mL/L |

The occupancy of N-glycan on anti-Her2 antibodies was determined using capillary electrophoresis (CE) as follows. The antibodies were recovered from the cell culture medium and purified by protein A column chromatography. The protein A purified sample (100-200 μg) was concentrated to about 100 μL and then its buffer was exchanged with 100 mM Tris-HCl pH 9.0 with 1% SDS. Then, the sample along with 2 μL of 10 kDa internal standard provided by Beckman was reduced by addition of 5 μl β-mercaptoethanol and boiled for five minutes. About 20 μl of reduced sample was then resolved over a bare-fused silica capillary (about 70 mm, 50 um I.D.) according to the method recommended by Beckman Coulter.

Table 31 shows N-glycan occupancy of anti-HER2 antibodies was increased when LmSTT3D was overexpressed in the presence of intact Pichia pastoris oligosaccharyl transferase (OST) complex. To determine N-glycosylation site occupancy, antibodies were reduced and the N-glycan occupancy of the heavy chains determined. The table shows that in general, overexpression of the LmSTT3D under the control of an inducible promoter effected an increase of N-glycan occupancy from about 82-83% to about 99% for antibodies tested (about a 19% increase over the N-glycan occupancy in the absence of LmSTT3D overexpression). The expression of the LmSTT3D and the antibodies were under the control of the same inducible promoter. When overexpression of the LmSTT3D was under the control of a constitutive promoter the increase in N-glycan occupancy was increased to about 94% for antibodies tested (about a 13% increase over the N-glycan occupancy in the absence of LmSTT3D overexpression).

TABLE 31

| | | | | Heavy Chain |
|---|---|---|---|---|
| | | | | N- |
| | | | | glycosylation |
| | LmSTT3D | | | site |
| | AOX1 Prom. | GAPDH Prom. | | occupancy# |
| Strain | (pGLY6301) | (pGLY6294) | Antibody | (%) |
| YGLY13992 | None | None | Anti-HER2 | 83 |
| YGLY17368 | None | overexpressed | Anti-HER2 | 94 |
| YGLY17351 | over- | None | Anti-HER2 | 99 |
| | expressed | | | |

#N-glycosylation site occupancy based upon percent glycosylation site occupancy of total heavy chains from reduced antibodies

Table 32 shows the N-glycan composition of the anti-Her2 antibodies produced in strains that overexpress LmSTT3D compared to strains that do not overexpress LmSTT3D. Antibodies were produced from SixFors (0.5 L bioreactor) and N-glycans from protein A-purified antibodies were analyzed with 2AB labeling. Overall, overexpression of LmSTT3D did not appear to significantly affect the N-glycan composition of the antibodies.

TABLE 32

| | | N-glycans (%) | | | | |
|---|---|---|---|---|---|---|
| | LmSTT3D | G0 | G1 | G2 | Man5 | Hybrids |
| Anti- | None | 58.1 ± 1.8 | 20.50.6 | 3.0 ± 0.9 | 14.0 ± 2.1 | 4.3 ± 1.2 |
| Her2 | | | | | | |
| Anti- | over- | 53.9 ± 2.0 | 22.4 ± 3.0 | 4.5 ± 1.7 | 14.7 ± 1.5 | 4.2 ± 1.5 |
| body | expressed | | | | | |

G0—GlcNAc₂Man3GlcNAc₂
G1—GalGlcNAc₂Man₃GlcNAc₂
G2—Gal₂GlcNAc₂Man₃GlcNAc₂
Man5—Man₅GlcNAc₂
Hybrid—GlcNAcMan₅GlcNAc₂ and/or GalGlcNAcMan₅GlcNAc₂

The high performance liquid chromatography (HPLC) system used consisted of an Agilent 1200 equipped with autoinjector, a column-heating compartment and a UV detector detecting at 210 and 280 nm. All LC-MS experiments performed with this system were running at 1 mL/min. The flow rate was not split for MS detection. Mass spectrometric analysis was carried out in positive ion mode on Accurate-Mass Q-TOF LC/MS 6520 (Agilent technology). The temperature of dual ESI source was set at 350° C. The nitrogen gas flow rates were set at 13 L/h for the cone and 350 l/h and nebulizer was set at 45 psig with 4500 volt applied to the capillary. Reference mass of 922.009 was prepared from HP-0921 according to API-TOF reference mass solution kit for mass calibration and the protein mass measurements. The data for ion spectrum range from 300-3000 m/z were acquired and processed using Agilent Masshunter.

Sample preparation was as follows. An intact antibody sample (50 μg) was prepared 50 μL 25 mM NH₄HCO₃, pH 7.8. For deglycosylated antibody, a 50 μL aliquot of intact antibody sample was treated with PNGase F (10 units) for 18 hours at 37° C. Reduced antibody was prepared by adding 1 M DTT to a final concentration of 10 mM to an aliquot of either intact antibody or deglycosylated antibody and incubated for 30 min at 37° C.

Three microgram of intact or deglycosylated antibody sample was loaded onto a Poroshell 300SB-C3 column (2.1 mm×75 mm, 5 um) (Agilent Technologies) maintained at 70° C. The protein was first rinsed on the cartridge for 1 minutes with 90% solvent A (0.1% HCOOH), 5% solvent B (90% Acetonitrile in 0.1% HCOOH). E lution was then performed using a gradient of 5-100% of B over 26 minutes followed by a 3 minute regeneration at 100% B and by a final equilibration period of 10 minute at 5% B.

For reduced antibody, three microgram sample was loaded a Poroshell 300SB-C3 column (2.1 mm×75 mm, 5 μm) (Agilent Technologies) maintained at 40° C. The protein was first rinsed on the cartridge for 3 minutes with 90% solvent A, 5% solvent B. Elution was then performed using an gradient of 5-80% of B over 20 minutes followed by a 7 minute regeneration at 80% B and by a final equilibration period of 10 minutes at 5% B.

TABLE 33
BRIEF DESCRIPTION OF THE SEQUENCES

| | | |
|---|---|---|
| SEQ | | |
| ID | | |
| NO: | Description | Sequence |

| | | |
|---|---|---|
| 1 | PCR primer | CTGAGGAGTCAGATATCAGCTCAATCTCCAT |
| | PpURA6out/UP | |
| 2 | PCR primer | TCCGGCTCGTATGTTGTGTGGAATTGT |
| | Puc19/LP | |
| 3 | PCR primer | CTGGATGTTTGATGGGTTCAGTTTCAGCTGGA |
| | PpURA6out/LP | |
| 4 | PCR primer | GGCAATAGTCGCGAGAATCCTTAAACCAT |
| | ScARR3/UP | |
| 5 | PCR primer | CCTCGTAAAGATCTGCGGTTTGCAAAGT |
| | PpTRP1- | |
| | 5'out/UP | |
| 6 | PCR primer | CCTCCCACTGGAACCGATGATATGGAA |
| | PpALG3TT/LP | |
| 7 | PCR primer | GATGCGAAGTTAAGTGCGCAGAAAGTAATATCA |
| | PpTEFTT/UP | |
| 8 | PCR primer | CGTGTGTACCTTGAAACGTCAATGATACTTTGA |
| | PpTRP- | |
| | 3'1out/LP | |
| 9 | PCR primer | CAGACTAAGACTGCTTCTCCACCTGCTAAG |
| | LmSTT3D/iUP | |
| 10 | PCR primer | CAACAGTAGAACCAGAAGCCTCGTAAGTACAG |
| | LmSTT3D/iLP | |
| 11 | Leishmania | ATGGGTAAAAGAAAGGGAAACTCCTTGGGAGATTCTG |
| | major STT3D | GTTCTGCTGCTACTGCTTCCAGAGAGGCTTCTGCTCAA |
| | (DNA) | GCTGAAGATGCTGCTTCCCAGACTAAGACTGCTTCTCC |
| | | ACCTGCTAAGGTTATCTTGTTGCCAAAGACTTTGACTG |
| | | ACGAGAAGGACTTCATCGGTATCTTCCCATTTCCATTC |
| | | TGGCCAGTTCACTTCGTTTTGACTGTTGTTGCTTTGTTC |
| | | GTTTTGGCTGCTTCCTGTTTCCAGGCTTTCACTGTTAG |
| | | AATGATCTCCGTTCAAATCTACGGTTACTTGATCCACG |
| | | AATTTGACCCATGGTTCAACTACAGAGCTGCTGAGTA |
| | | CATGTCTACTCACGGATGGAGTGCTTTTTTCTCCTGGT |
| | | TCGATTACATGTCCTGGTATCCATTGGGTAGACCAGTT |
| | | GGTTCTACTACTTACCCAGGATTGCAGTTGACTGCTGT |
| | | TGCTATCCATAGAGCTTTGGCTGCTGCTGGAATGCCAA |
| | | TGTCCTTGAACAATGTTTGTGTTTTGATGCCAGCTTGG |
| | | TTTGGTGCTATCGCTACTGCTACTTTGGCTTTCTGTACT |
| | | TACGAGGCTTCTGGTTCTACTGTTGCTGCTGCTGCAGC |
| | | TGCTTTGTCCTTCTCCATTATCCCTGCTCACTTGATGAG |
| | | ATCCATGGCTGGTGAGTTCGACAACGAGTGTATTGCT |
| | | GTTGCTGCTATGTTGTTGACTTTCTACTGTTGGGTTCGT |
| | | TCCTTGAGAACTAGATCCTCCTGGCCAATCGGTGTTTT |
| | | GACAGGTGTTGCTTACGGTTACATGGCTGCTGCTTGGG |
| | | GAGGTTACATCTTCGTTTTGAACATGGTTGCTATGCAC |
| | | GCTGGTATCTCTTCTATGGTTGACTGGGCTAGAAACAC |
| | | TTACAACCCATCCTTGTTGAGAGCTTACACTTTGTTCT |
| | | ACGTTGTTGGTACTGCTATCGCTGTTTGTGTTCCACCA |
| | | GTTGGAATGTCTCCATTCAAGTCCTTGGAGCAGTTGGG |
| | | AGCTTTGTTGGTTTTGGTTTTCTTGTGTGGATTGCAAGT |
| | | TTGTGAGGTTTTGAGAGCTAGAGCTGGTGTTGAAGTTA |
| | | GATCCAGAGCTAATTTCAAGATCAGAGTTAGAGTTTT |
| | | CTCCGTTATGGCTGGTGTTGCTGCTTTGGCTATCTCTG |
| | | TTTTGGCTCCAACTGGTTACTTTGGTCCATTGTCTGTTA |
| | | GAGTTAGAGCTTTGTTTGTTGAGCACACTAGAACTGGT |
| | | AACCCATTGGTTGACTCCGTTGCTGAACATCAACCAG |
| | | CTTCTCCAGAGGCTATGTGGGCTTTCTTGCATGTTTGT |
| | | GGTGTTACTTGGGGATTGGGTTCCATTGTTTTGGCTGT |
| | | TTCCACTTTCGTTCACTACTCCCCATCTAAGGTTTTCTG |
| | | GTTGTTGAACTCCGGTGCTGTTTACTACTTCTCCACTA |
| | | GAATGGCTAGATTGTTGTTGTTGTCCGGTCCAGCTGCT |
| | | TGTTTGTCCACTGGTATCTTCGTTGGTACTATCTTGGA |
| | | GGCTGCTGTTCAATTGTCTTTCTGGGACTCCGATGCTA |
| | | CTAAGGCTAAGAAGCAGCAAAAGCAGGCTCAAAGAC |
| | | ACCAAAGAGGTGCTGGTAAAGGTTCTGGTAGAGATGA |
| | | CGCTAAGAACGCTACTACTGCTAGAGCTTTCTGTGAC |
| | | GTTTTCGCTGGTTCTTCTTTGGCTTGGGGTCACAGAAT |
| | | GGTTTTGTCCATTGCTATGTGGGCTTTGGTTACTACTA |
| | | CTGCTGTTTCCTTCTTCTCCTCCGAATTTGCTTCTCACT |
| | | CCACTAAGTTCGCTGAACAATCCTCCAACCCAATGAT |
| | | CGTTTTCGCTGCTGTTGTTCAGAACAGAGCTACTGGAA |
| | | AGCCAATGAACTTGTTGGTTGACGACTACTTGAAGGC |
| | | TTACGAGTGGTTGAGAGACTCTACTCCAGAGGACGCT |
| | | AGAGTTTTGGCTTGGTGGGACTACGGTTACCAAATCA |
| | | CTGGTATCGGTAACAGAACTTCCTTGGCTGATGGTAA |
| | | CACTTGGAACCACGAGCACATTGCTACTATCGGAAAG |
| | | ATGTTGACTTCCCCAGTTGTTGAAGCTCACTCCCTTGT |
| | | TAGACACATGGCTGACTACGTTTTGATTTGGGCTGGTC |
| | | AATCTGGTGACTTGATGAAGTCTCCACACATGGCTAG |
| | | AATCGGTAACTCTGTTTACCACGACATTTGTCCAGATG |
| | | ACCCATTGTGTCAGCAATTCGGTTTCCACAGAAACGA |
| | | TTACTCCAGACCAACTCCAATGATGAGAGCTTCCTTGT |
| | | TGTACAACTTGCACGAGGCTGGAAAAAGAAAGGGTGT |
| | | TAAGGTTAACCCATCTTTGTTCCAAGAGGTTTACTCCT |
| | | CCAAGTACGGACTTGTTAGAATCTTCAAGGTTATGAA |
| | | CGTTTCCGCTGAGTCTAAGAAGTGGGTTGCAGACCCA |
| | | GCTAACAGAGTTTGTCACCCACCTGGTTCTTGGATTTG |
| | | TCCTGGTCAATACCCACCTGCTAAAGAAATCCAAGAG |
| | | ATGTTGGCTCACAGAGTTCCATTCGACCAGGTTACAA |
| | | ACGCTGACAGAAAGAACAATGTTGGTTCCTACCAAGA |
| | | GGAATACATGAGAAGAATGAGAGAGTCCGAGAACAG |
| | | AAGATAATAG |
| 12 | Leishmania | MGKRKGNSLGDSGSAATASREASAQAEDAASQTKTASP |
| | major STT3D | PAKVILLPKTLTDEKDFIGIFPFPFWPVHFVLTVVALFVLA |
| | (protein) | ASCFQAFTVRMISVQIYGYLIHEFDPWFNYRAAEYMSTH |
| | | GWSAFFSWFDYMSWYPLGRPVGSTTYPGLQLTAVAIHR |
| | | ALAAAGMPMSLNNVCVLMPAWFGAIATATLAFCTYEAS |
| | | GSTVAAAAAALSFSIIPAHLMRSMAGEFDNECIAVAAML |
| | | LTFYCWVRSLRTRSSWPIGVLTGVAYGYMAAAWGGYIF |
| | | VLNMVAMHAGISSMVDWARNTYNPSLLRAYTLFYVVG |
| | | TAIAVCVPPVGMSPFKSLEQLGALLVLVFLCGLQVCEVL |
| | | RARAGVEVRSRANFKIRVRVFSVMAGVAALAISVLAPTG |
| | | YFGPLSVRVRALFVEHTRTGNPLVDSVAEHQPASPEAM |
| | | WAFLHVCGVTWGLGSIVLAVSTFVHYSPSKVFWLLNSG |
| | | AVYYFSTRMARLLLLSGPAACLSTGIFVGTILEAAVQLSF |
| | | WDSDATKAKKQQKQAQRHQRGAGKGSGRDDAKNATT |
| | | ARAFCDVFAGSSLAWGHRMVLSIAMWALVTTTAVSFFS |
| | | SEFASHSTKFAEQSSNPMIVFAAVVQNRATGKPMNLLVD |
| | | DYLKAYEWLRDSTPEDARVLAWWDYGYQITGIGNRTSL |
| | | ADGNTWNHEHIATIGKMLTSPVVEAHSLVRHMADYVLI |
| | | WAGQSGDLMKSPHMARIGNSVYHDICPDDPLCQQFGFH |
| | | RNDYSRPTPMMRASLLYNLHEAGKRKGVKVNPSLFQEV |
| | | YSSKYGLVRIFKVMNVSAESKKWVADPANRVCHPPGS |
| | | WICPGQYPPAKEIQEMLAHRVPFDQVTNADRKNNVGSY |
| | | QEEYMRRMRESENRR |
| 13 | Saccharomyces | ATGAGATTCCCATCCATCTTCACTGCTGTTTTGTTCGC |
| | cerevisiae | TGCTTCTTCTGCTTTGGCT |
| | mating factor | |
| | pre-signal | |
| | peptide (DNA) | |
| 14 | Saccharomyces | MRFPSIFTAVLFAASSALA |
| | cerevisiae | |
| | mating factor | |
| | pre-signal | |
| | peptide (protein) | |
| 15 | Anti-Her2 | GAGGTTCAGTTGGTTGAATCTGGAGGAGGATTGGTTC |
| | Heavy chain | AACCTGGTGGTTCTTTGAGATTGTCCTGTGCTGCTTCC |
| | (VH + IgG1 | GGTTTCAACATCAAGGACACTTACATCCACTGGGTTA |
| | constant region) | GACAAGCTCCAGGAAAGGGATTGGAGTGGGTTGCTAG |
| | (DNA), Lack C- | AATCTACCCAACTAACGGTTACACAAGATACGCTGAC |
| | terminal Lysine | TCCGTTAAGGGAAGATTCACTATCTCTGCTGACACTTC |
| | | CAAGAACACTGCTTACTTGCAGATGAACTCCTTGAGA |
| | | GCTGAGGATACTGCTGTTTACTACTGTTCCAGATGGGG |
| | | TGGTGATGGTTTCTACGCTATGGACTACTGGGGTCAA |
| | | GGAACTTTGGTTACTGTTTCCTCCGCTTCTACTAAGGG |
| | | ACCATCTGTTTTCCCATTGGCTCCATCTTCTAAGTCTA |
| | | CTTCCGGTGGTACTGCTGCTTTGGGATGTTTGGTTAAA |
| | | GACTACTTCCCAGAGCCAGTTACTGTTTCTTGGAACTC |
| | | CGGTGCTTTGACTTCTGGTGTTCACACTTTCCCAGCTG |
| | | TTTTGCAATCTTCCGGTTTGTACTCTTTGTCCTCCGTTG |
| | | TTACTGTTCCATCCTCTTCCTTGGGTACTCAGACTTAC |
| | | ATCTGTAACGTTAACCACAAGCCATCCAACACTAAGG |
| | | TTGACAAGAAGGTTGAGCCAAAGTCCTGTGACAAGAC |
| | | ACATACTTGTCCACCATGTCCAGCTCCAGAATTGTTGG |
| | | GTGGTCCATCCGTTTTCTTGTTCCCACCAAAGCCAAAG |
| | | GACACTTTGATGATCTCCAGAACTCCAGAGGTTACAT |
| | | GTGTTGTTGTTGACGTTTCTCACGAGGACCCAGAGGTT |
| | | AAGTTCAACTGGTACGTTGACGGTGTTGAAGTTCACA |
| | | ACGCTAAGACTAAGCCAAGAGAAGAGCAGTACAACT |
| | | CCACTTACAGAGTTGTTTCCGTTTTGACTGTTTTGCAC |
| | | CAGGACTGGTTGAACGGTAAAGAATACAAGTGTAAGG |
| | | TTTCCAACAAGGCTTTGCCAGCTCCAATCGAAAAGAC |
| | | TATCTCCAAGGCTAAGGGTCAACCAAGAGAGCCACAG |
| | | GTTTACACTTTGCCACCATCCAGAGAAGAGATGACTA |
| | | AGAACCAGGTTTCCTTGACTTGTTTGGTTAAAGGATTC |
| | | TACCCATCCGACATTGCTGTTGAGTGGGAATCTAACG |
| | | GTCAACCAGAGAACAACTACAAGACTACTCCACCAGT |
| | | TTTGGATTCTGATGGTTCCTTCTTCTTGTACTCCAAGTT |
| | | GACTGTTGACAAGTCCAGATGGCAACAGGGTAACGTT |
| | | TTCTCCTGTTCCGTTATGCATGAGGCTTTGCACAACCA |
| | | CTACACTCAAAAGTCCTTGTCTTTGTCCCCTGGTTAA |
| 16 | Anti-Her2 | EVQLVESGGGLVQPGGSLRLSCAASGFNIKDTYIHWVRQ |
| | Heavy chain | APGKGLEWVARIYPTNGYTRYADSVKGRFTISADTSKNT |
| | (VH + IgG1 | AYLQMNSLRAEDTAVYYCSRWGGDGFYAMDYWGQGT |
| | constant region) | LVTVSSASTKGPSVFPLAPSSKSTSGGTAALGCLVKDYFP |
| | (protein), Lack | EPVTVSWNSGALTSGVHTFPAVLQSSGLYSLSSVVTVPSS |
| | C-terminal | SLGTQTYICNVNHKPSNTKVDKKVEPKSCDKTHTCPPCP |
| | Lysine | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPREP |
| | | QVYTLPPSREEMTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPG |
| 17 | Anti-Her2 light | GACATCCAAATGACTCAATCCCCATCTTCTTTGTCTGC |
| | chain (VL + | TTCCGTTGGTGACAGAGTTACTATCACTTGTAGAGCTT |
| | Kappa constant | CCCAGGACGTTAATACTGCTGTTGCTTGGTATCAACAG |
| | region) (DNA) | AAGCCAGGAAAGGCTCCAAAGTTGTTGATCTACTCCG |
| | | CTTCCTTCTTGTACTCTGGTGTTCCATCCAGATTCTCTG |
| | | GTTCCAGATCCGGTACTGACTTCACTTTGACTATCTCC |
| | | TCCTTGCAACCAGAAGATTTCGCTACTTACTACTGTCA |
| | | GCAGCACTACACTACTCCACCAACTTTCGGACAGGGT |
| | | ACTAAGGTTGAGATCAAGAGAACTGTTGCTGCTCCAT |
| | | CCGTTTTCATTTTCCCACCATCCGACGAACAGTTGAAG |
| | | TCTGGTACAGCTTCCGTTGTTTGTTTGTTGAACAACTT |
| | | CTACCCAAGAGAGGCTAAGGTTCAGTGGAAGGTTGAC |
| | | AACGCTTTGCAATCCGGTAACTCCCAAGAATCCGTTA |
| | | CTGAGCAAGACTCTAAGGACTCCACTTACTCCTTGTCC |
| | | TCCACTTTGACTTTGTCCAAGGCTGATTACGAGAAGCA |
| | | CAAGGTTTACGCTTGTGAGGTTACACATCAGGGTTTGT |
| | | CCTCCCCAGTTACTAAGTCCTTCAACAGAGGAGAGTG |
| | | TTAA |
| 18 | Anti-Her2 light | DIQMTQSPSSLSASVGDRVTITCRASQDVNTAVAWYQQ |
| | chain (VL + | KPGKAPKLLIYSASFLYSGVPSRFSGSRSGTDFTLTISSLQ |
| | Kappa constant | PEDFATYYCQQHYTTPPTFGQGTKVEIKRTVAAPSVFIFP |
| | region) | PSDEQLKSGTASVVCLLNNFYPREAKVQWKVDNALQSG |
| | | NSQESVTEQDSKDSTYSLSSTLTLSKADYEKHKVYACEV |
| | | THQGLSSPVTKSFNRGEC |
| 19 | Anti-Her2 | GAGGTCCAATTGGTTGAATCTGGTGGAGGTTTGGTCC |
| | Heavy chain | AACCAGGTGGATCTCTGAGACTTTCTTGTGCTGCCTCT |
| | (VH + IgG1 | GGTTTCAACATTAAGGATACTTACATCCACTGGGTTAG |
| | constant region) | ACAGGCTCCAGGTAAGGGTTTGGAGTGGGTTGCTAGA |
| | (DNA), C- | ATCTACCCAACCAACGGTTACACCAGATACGCTGAtTC |
| | terminal Lysine, | CGTTAAGGGTAGATTCACCATTTCCGCTGACACTTCCA |
| | allotype | AGAACACTGCTTACTTGCAAATGAACTCTTTGAGAGC |
| | | TGAGGACACTGCCGTCTACTACTGTTCCAGATGGGGT |
| | | GGTGACGGTTTCTACGCCATGGACTACTGGGGTCAAG |
| | | GTACCTTGGTTACTGTCTCTTCCGCTTCTACTAAGGGA |
| | | CCATCCGTTTTTCCATTGGCTCCATCCTCTAAGTCTACT |
| | | TCCGGTGGTACTGCTGCTTTGGGATGTTTGGTTAAGGA |
| | | CTACTTCCCAGAGCCTGTTACTGTTTCTTGGAACTCCG |
| | | GTGCTTTGACTTCTGGTGTTCACACTTTCCCAGCTGTTT |
| | | TGCAATCTTCCGGTTTGTACTCCTTGTCCTCCGTTGTTA |
| | | CTGTTCCATCCTCTTCCTTGGGTACTCAGACTTACATC |
| | | TGTAACGTTAACCACAAGCCATCCAACACTAAGGTTG |
| | | ACAAGAAGGTTGAGCCAAAGTCCTGTGACAAGACACA |
| | | TACTTGTCCACCATGTCCAGCTCCAGAATTGTTGGGTG |
| | | GTCCATCCGTTTTCTTGTTCCCACCAAAGCCAAAGGAC |
| | | ACTTTGATGATCTCCAGAACTCCAGAGGTTACATGTGT |
| | | TGTTGTTGACGTTTCTCACGAGGACCCAGAGGTTAAGT |
| | | TCAACTGGTACGTTGACGGTGTTGAAGTTCACAACGC |
| | | TAAGACTAAGCCAAGAGAGGAGCAGTACAACTCCACT |
| | | TACAGAGTTGTTTCCGTTTTGACTGTTTTGCACCAGGA |
| | | TTGGTTGAACGGAAAGGAGTACAAGTGTAAGGTTTCC |
| | | AACAAGGCTTTGCCAGCTCCAATCGAAAAGACTATCT |
| | | CCAAGGCTAAGGGTCAACCAAGAGAGCCACAGGTTTA |
| | | CACTTTGCCACCATCCAGAGATGAGTTGACTAAGAAC |
| | | CAGGTTTCCTTGACTTGTTTGGTTAAAGGATTCTACCC |
| | | ATCCGACATTGCTGTTGAGTGGGAATCTAACGGTCAA |
| | | CCAGAGAACAACTACAAGACTACTCCACCAGTTTTGG |
| | | ATTCTGACGGTTCCTTCTTCTTGTACTCCAAGTTGACT |
| | | GTTGACAAGTCCAGATGGCAACAGGGTAACGTTTTCT |
| | | CCTGTTCCGTTATGCATGAGGCTTTGCACAACCACTAC |
| | | ACTCAAAAGTCCTTGTCTTTGTCCCCAGGTAAGtaa |
| 20 | Anti-Her2 | EVQLVESGGGLVQPGGSLRLSCAASGFNIKDTYIHWVRQ |
| | Heavy chain | APGKGLEWVARIYPTNGYTRYADSVKGRFTISADTSKNT |
| | (VH + IgG1 | AYLQMNSLRAEDTAVYYCSRWGGDGFYAMDYWGQGT |
| | constant region) | LVTVSSASTKGPSVFPLAPSSKSTSGGTAALGCLVKDYFP |
| | (protein), C- | EPVTVSWNSGALTSGVHTFPAVLQSSGLYSLSSVVTVPSS |
| | terminal Lysine, | SLGTQTYICNVNHKPSNTKVDKKVEPKSCDKTHTCPPCP |
| | allotype | APELLGGPSVFLFPPKPKDTLMISRTPEVTCVVVDVSHED |
| | | PEVKFNWYVDGVEVHNAKTKPREEQYNSTYRVVSVLT |
| | | VLHQDWLNGKEYKCKVSNKALPAPIEKTISKAKGQPREP |
| | | QVYTLPPSRDELTKNQVSLTCLVKGFYPSDIAVEWESNG |
| | | QPENNYKTTPPVLDSDGSFFLYSKLTVDKSRWQQGNVFS |
| | | CSVMHEALHNHYTQKSLSLSPGK |
| 21 | DNA encodes | ATGGTTGCTT GGTGGTCCTT GTTCTTGTAC |
| | alpha amylase | GGATTGCAAG TTGCTGCTCC AGCTTTGGCT |
| | signal sequence | |
| | (from | |
| | Aspergillus | |
| | niger α-amylase) | |
| | (DNA) | |
| 22 | Tr Man I | RAGSPNPTRAAAVKAAFQTSWNAYHHFAFPHDDLHPVS |
| | catalytic doman | NSFDDERNGWGSSAIDGLDTAILMGDADIVNTILQYVPQI |
| | | NFTTTAVANQGISVFETNIRYLGGLLSAYDLLRGPFSSLA |
| | | TNQTLVNSLLRQAQTLANGLKVAFTTPSGVPDPTVFFNP |
| | | TVRRSGASSNNVAEIGSLVLEWTRLSDLTGNPQYAQLAQ |
| | | KGESYLLNPKGSPEAWPGLIGTFVSTSNGTFQDSSGSWS |
| | | GLMDSFYEYLIKMYLYDPVAFAHYKDRWVLAADSTIAH |
| | | LASHPSTRKDLTFLSSYNGQSTSPNSGHLASFAGGNFILG |
| | | GILLNEQKYIDFGIKLASSYFATYNQTASGIGPEGFAWVD |
| | | SVTGAGGSPPSSQSGFYSSAGFWVTAPYYILRPETLESLY |
| | | YAYRVTGDSKWQDLAWEAFSAIEDACRAGSAYSSINDV |
| | | TQANGGGASDDMESFWFAEALKYAYLIFAEESDVQVQA |
| | | NGGNKFVFNTEAHPFSIRSSSRRGGHLA |
| 23 | Pp AOX1 | AACATCCAAAGACGAAAGGTTGAATGAAACCTTTTTG |
| | promoter | CCATCCGACATCCACAGGTCCATTCTCACACATAAGT |
| | | GCCAAACGCAACAGGAGGGGATACACTAGCAGCAGA |
| | | CCGTTGCAAACGCAGGACCTCCACTCCTCTTCTCCTCA |
| | | ACACCCACTTTTGCCATCGAAAAACCAGCCCAGTTATT |
| | | GGGCTTGATTGGAGCTCGCTCATTCCAATTCCTTCTAT |
| | | TAGGCTACTAACACCATGACTTTATTAGCCTGTCTATC |
| | | CTGGCCCCCCTGGCGAGGTTCATGTTTGTTTATTTCCG |
| | | AATGCAACAAGCTCCGCATTACACCCGAACATCACTC |
| | | CAGATGAGGGCTTTCTGAGTGTGGGGTCAAATAGTTT |
| | | CATGTTCCCCAAATGGCCCAAAACTGACAGTTTAAAC |
| | | GCTGTCTTGGAACCTAATATGACAAAAGCGTGATCTC |
| | | ATCCAAGATGAACTAAGTTTGGTTCGTTGAAATGCTA |
| | | ACGGCCAGTTGGTCAAAAAGAAACTTCCAAAAGTCGG |
| | | CATACCGTTTGTCTTGTTTGGTATTGATTGACGAATGC |
| | | TCAAAAATAATCTCATTAATGCTTAGCGCAGTCTCTCT |
| | | ATCGCTTCTGAACCCCGGTGCACCTGTGCCGAAACGC |
| | | AAATGGGGAAACACCCGCTTTTTGGATGATTATGCAT |
| | | TGTCTCCACATTGTATGCTTCCAAGATTCTGGTGGGAA |
| | | TACTGCTGATAGCCTAACGTTCATGATCAAAATTTAAC |
| | | TGTTCTAACCCCTACTTGACAGCAATATATAAACAGA |
| | | AGGAAGCTGCCCTGTCTTAAACCTTTTTTTTTATCATC |
| | | ATTATTAGCTTACTTTCATAATTGCGACTGGTTCCAAT |
| | | TGACAAGCTTTTGATTTTAACGACTTTTAACGACAACT |
| | | TGAGAAGATCAAAAAACAACTAATTATTCGAAACG |
| 24 | ScCYC TT | ACAGGCCCCTTTTCCTTTGTCGATATCATGTAATTAGT |
| | | TATGTCACGCTTACATTCACGCCCTCCTCCCACATCCG |
| | | CTCTAACCGAAAAGGAAGGAGTTAGACAACCTGAAGT |
| | | CTAGGTCCCTATTTATTTTTTTTAATAGTTATGTTAGTA |
| | | TTAAGAACGTTATTTATATTTCAAATTTTTCTTTTTTTT |
| | | CTGTACAAACGCGTGTACGCATGTAACATTATACTGA |
| | | AAACCTTGCTTGAGAAGGTTTTGGGACGCTCGAAGGC |
| | | TTTAATTTGCAAGCTGCCGGCTCTTAAG |
| 25 | PpRPL10 | GTTCTTCGCTTGGTCTTGTATCTCCTTACACTGTATCTT |
| | promoter | CCCATTTGCGTTTAGGTGGTTATCAAAAACTAAAAGG |
| | | AAAAATTTCAGATGTTTATCTCTAAGGTTTTTTCTTTTT |
| | | ACAGTATAACACGTGATGCGTCACGTGGTACTAGATT |
| | | ACGTAAGTTATTTTGGTCCGGTGGGTAAGTGGGTAAG |
| | | AATAGAAAGCATGAAGGTTTACAAAAACGCAGTCACG |
| | | AATTATTGCTACTTCGAGCTTGGAACCACCCCAAAGA |
| | | TTATATTGTACTGATGCACTACCTTCTCGATTTTGCTCC |
| | | TCCAAGAACCTACGAAAAACATTTCTTGAGCCTTTTCA |
| | | ACCTAGACTACACATCAAGTTATTTAAGGTATGTTCCG |
| | | TTAACATGTAAGAAAAGGAGAGGATAGATCGTTTATG |
| | | GGGTACGTCGCCTGATTCAAGCGTGACCATTCGAAGA |
| | | ATAGGCCTTCGAAAGCTGAATAAAGCAAATGTCAGTT |
| | | GCGATTGGTATGCTGACAAATTAGCATAAAAAGCAAT |
| | | AGACTTTCTAACCACCTGTTTTTTTCCTTTTACTTTATT |
| | | TATATTTTGCCACCGTACTAACAAGTTCAGACAAA |
| 26 | PpGAPDH | TTTTTGTAGAAATGTCTTGGTGTCCTCGTCCAATCAGG |
| | promoter | TAGCCATCTCTGAAATATCTGGCTCCGTTGCAACTCCG |
| | | AACGACCTGCTGGCAACGTAAAATTCTCCGGGGTAAA |
| | | ACTTAAATGTGGAGTAATGGAACCAGAAACGTCTCTT |
| | | CCCTTCTCTCTCCTTCCACCGCCCGTTACCGTCCCTAG |
| | | GAAATTTTACTCTGCTGGAGAGCTTCTTCTACGGCCCC |
| | | CTTGCAGCAATGCTCTTCCCAGCATTACGTTGCGGGTA |
| | | AAACGGAGGTCGTGTACCCGACCTAGCAGCCCAGGGA |
| | | TGGAAAAGTCCCGGCCGTCGCTGGCAATAATAGCGGG |
| | | CGGACGCATGTCATGAGATTATTGGAAACCACCAGAA |
| | | TCGAATATAAAAGGCGAACACCTTTCCCAATTTTGGTT |
| | | TCTCCTGACCCAAAGACTTTAAATTTAATTTATTTGTC |
| | | CCTATTTCAATCAATTGAACAACTATCAAAACACA |
| 27 | PpTEF1 | TTAAGGTTTGGAACAACACTAAACTACCTTGCGGTAC |
| | promoter | TACCATTGACACTACACATCCTTAATTCCAATCCTGTC |
| | | TGGCCTCCTTCACCTTTTAACCATCTTGCCCATTCCAA |
| | | CTCGTGTCAGATTGCGTATCAAGTGAAAAAAAAAAAA |
| | | TTTTAAATCTTTAACCCAATCAGGTAATAACTGTCGCC |
| | | TCTTTTATCTGCCGCACTGCATGAGGTGTCCCCTTAGT |
| | | GGGAAAGAGTACTGAGCCAACCCTGGAGGACAGCAA |
| | | GGGAAAAATACCTACAACTTGCTTCATAATGGTCGTA |
| | | AAAACAATCCTTGTCGGATATAAGTGTTGTAGACTGT |
| | | CCCTTATCCTCTGCGATGTTCTTCCTCTCAAAGTTTGC |
| | | GATTTCTCTCTATCAGAATTGCCATCAAGAGACTCAGG |
| | | ACTAATTTCGCAGTCCCACACGCACTCGTACATGATTG |
| | | GCTGAAATTTCCCTAAAGAATTTCTTTTTCACGAAAAT |
| | | TTTTTTTTTACACAAGATTTTCAGCAGATATAAAATGG |
| | | AGAGCAGGACCTCCGCTGTGACTCTTCTTTTTTTTCTTT |
| | | TATTCTCACTACATACATTTTAGTTATTCGCCAAC |
| 28 | PpTEF1 TT | ATTGCTTGAAGCTTTAATTTATTTTATTAACATAATAA |
| | | TAATACAAGCATGATATATTTGTATTTTGTTCGTTAAC |
| | | ATTGATGTTTTCTTCATTTACTGTTATTGTTTGTAACTT |
| | | TGATCGATTTATCTTTTCTACTTTACTGTAATATGGCTG |
| | | GCGGGTGAGCCTTGAACTCCCTGTATTACTTTACCTTG |
| | | CTATTACTTAATCTATTGACTAGCAGCGACCTCTTCAA |
| | | CCGAAGGGCAAGTACACAGCAAGTTCATGTCTCCGTA |
| | | AGTGTCATCAACCCTGGAAACAGTGGGCCATGTC |
| 29 | PpALG3 TT | ATTTACAATTAGTAATATTAAGGTGGTAAAAACATTC |
| | | GTAGAATTGAAATGAATTAATATAGTATGACAATGGT |
| | | TCATGTCTATAAATCTCCGGCTTCGGTACCTTCTCCCC |
| | | AATTGAATACATTGTCAAAATGAATGGTTGAACTATT |
| | | AGGTTCGCCAGTTTCGTTATTAAGAAAACTGTTAAAAT |
| | | CAAATTCCATATCATCGGTTCCAGTGGGAGGACCAGT |
| | | TCCATCGCCAAAATCCTGTAAGAATCCATTGTCAGAA |
| | | CCTGTAAAGTCAGTTTGAGATGAAATTTTTCCGGTCTT |
| | | TGTTGACTTGGAAGCTTCGTTAAGGTTAGGTGAAACA |
| | | GTTTGATCAACCAGCGGCTCCCGTTTTCGTCGCTTAGT |
| | | AG |
| 30 | PpTRP1 5′ | GCGGAAACGGCAGTAAACAATGGAGCTTCATTAGTGG |
| | region and ORF | GTGTTATTATGGTCCCTGGCCGGGAACGAACGGTGAA |
| | | ACAAGAGGTTGCGAGGGAAATTTCGCAGATGGTGCGG |
| | | GAAAAGAGAATTTCAAAGGGCTCAAAATACTTGGATT |
| | | CCAGACAACTGAGGAAAGAGTGGGACGACTGTCCTCT |
| | | GGAAGACTGGTTTGAGTACAACGTGAAAGAAATAAAC |
| | | AGCAGTGGTCCATTTTTAGTTGGAGTTTTTCGTAATCA |
| | | AAGTATAGATGAAATCCAGCAAGCTATCCACACTCAT |
| | | GGTTTGGATTTCGTCCAACTACATGGGTCTGAGGATTT |
| | | TGATTCGTATATACGCAATATCCCAGTTCCTGTGATTA |
| | | CCAGATACACAGATAATGCCGTCGATGGTCTTACCGG |
| | | AGAAGACCTCGCTATAAATAGGGCCCTGGTGCTACTG |
| | | GACAGCGAGCAAGGAGGTGAAGGAAAAACCATCGAT |
| | | TGGGCTCGTGCACAAAAATTTGGAGAACGTAGAGGAA |
| | | AATATTTACTAGCCGGAGGTTTGACACCTGATAATGTT |
| | | GCTCATGCTCGATCTCATACTGGCTGTATTGGTGTTGA |
| | | CGTCTCTGGTGGGGTAGAAACAAATGCCTCAAAAGAT |
| | | ATGGACAAGATCACACAATTTATCAGAAACGCTACAT |
| | | AA |
| 31 | PpTRP1 3′ | AAGTCAATTAAATACACGCTTGAAAGGACATTACATA |
| | region | GCTTTCGATTTAAGCAGAACCAGAAATGTAGAACCAC |
| | | TTGTCAATAGATTGGTCAATCTTAGCAGGAGCGGCTG |
| | | GGCTAGCAGTTGGAACAGCAGAGGTTGCTGAAGGTGA |
| | | GAAGGATGGAGTGGATTGCAAAGTGGTGTTGGTTAAG |
| | | TCAATCTCACCAGGGCTGGTTTTGCCAAAAATCAACTT |
| | | CTCCCAGGCTTCACGGCATTCTTGAATGACCTCTTCTG |
| | | CATACTTCTTGTTCTTGCATTCACCAGAGAAAGCAAAC |
| | | TGGTTCTCAGGTTTTCCATCAGGGATCTTGTAAATTCT |
| | | GAACCATTCGTTGGTAGCTCTCAACAAGCCCGGCATG |
| | | TGCTTTTCAACATCCTCGATGTCATTGAGCTTAGGAGC |
| | | CAATGGGTCGTTGATGTCGATGACGATGACCTTCCAG |
| | | TCAGTCTCTCCCTCATCCAACAAAGCCATAACACCGA |
| | | GGACCTTGACTTGCTTGACCTGTCCAGTGTAACCTACG |
| | | GCTTCACCAATTTCGCAAACGTCCAATGGATCATTGTC |
| | | ACCCTTGGCCTTGGTCTCTGGATGAGTGACGTTAGGGT |
| | | CTTCCCATGTCTGAGGGAAGGCACCGTAGTTGTGAAT |
| | | GTATCCGTGGTGAGGGAAACAGTTACGAACGAAACGA |
| | | AGTTTTCCCTTCTTTGTGTCCTGAAGAATTGGGTTCAG |
| | | TTTCTCCTCCTTGGAAATCTCCAACTTGGCGTTGGTCC |
| | | AACGGGGGACTTCAACAACCATGTTGAGAACCTTCTT |
| | | GGATTCGTCAGCATAAAGTGGGATGTCGTGGAAAGGA |
| | | GATACGACTT |
| 32 | ScARR3 ORF | ATGTCAGAAGATCAAAAAAGTGAAAATTCCGTACCTT |
| | | CTAAGGTTAATATGGTGAATCGCACCGATATACTGAC |
| | | TACGATCAAGTCATTGTCATGGCTTGACTTGATGTTGC |
| | | CATTTACTATAATTCTCTCCATAATCATTGCAGTAATA |
| | | ATTTCTGTCTATGTGCCTTCTTCCCGTCACACTTTTGAC |
| | | GCTGAAGGTCATCCCAATCTAATGGGAGTGTCCATTC |
| | | CTTTGACTGTTGGTATGATTGTAATGATGATTCCCCCG |
| | | ATCTGCAAAGTTTCCTGGGAGTCTATTCACAAGTACTT |
| | | CTACAGGAGCTATATAAGGAAGCAACTAGCCCTCTCG |
| | | TTATTTTTGAATTGGGTCATCGGTCCTTTGTTGATGAC |
| | | AGCATTGGCGTGGATGGCGCTATTCGATTATAAGGAA |
| | | TACCGTCAAGGCATTATTATGATCGGAGTAGCTAGAT |
| | | GCATTGCCATGGTGCTAATTTGGAATCAGATTGCTGG |
| | | AGGAGACAATGATCTCTGCGTCGTGCTTGTTATTACAA |
| | | ACTCGCTTTTACAGATGGTATTATATGCACCATTGCAG |
| | | ATATTTTACTGTTATGTTATTTCTCATGACCACCTGAA |
| | | TACTTCAAATAGGGTATTATTCGAAGAGGTTGCAAAG |
| | | TCTGTCGGAGTTTTTCTCGGCATACCACTGGGAATTGG |
| | | CATTATCATACGTTTGGGAAGTCTTACCATAGCTGGTA |
| | | AAAGTAATTATGAAAAATACATTTTGAGATTTATTTCT |
| | | CCATGGGCAATGATCGGATTTCATTACACTTTATTTGT |
| | | TATTTTTATTAGTAGAGGTTATCAATTTATCCACGAAA |
| | | TTGGTTCTGCAATATTGTGCTTTGTCCCATTGGTGCTTT |
| | | ACTTCTTTATTGCATGGTTTTTGACCTTCGCATTAATG |
| | | AGGTACTTATCAATATCTAGGAGTGATACACAAAGAG |
| | | AATGTAGCTGTGACCAAGAACTACTTTTAAAGAGGGT |
| | | CTGGGGAAGAAAGTCTTGTGAAGCTAGCTTTTCTATTA |
| | | CGATGACGCAATGTTTCACTATGGCTTCAAATAATTTT |
| | | GAACTATCCCTGGCAATTGCTATTTCCTTATATGGTAA |
| | | CAATAGCAAGCAAGCAATAGCTGCAACATTTGGGCCG |
| | | TTGCTAGAAGTTCCAATTTTATTGATTTTGGCAATAGT |
| | | CGCGAGAATCCTTAAACCATATTATATATGGAACAAT |
| | | AGAAATTAA |
| 33 | URA6 region | CAAATGCAAGAGGACATTAGAAATGTGTTTGGTAAGA |
| | | ACATGAAGCCGGAGGCATACAAACGATTCACAGATTT |
| | | GAAGGAGGAAAACAAACTGCATCCACCGGAAGTGCC |
| | | AGCAGCCGTGTATGCCAACCTTGCTCTCAAAGGCATT |
| | | CCTACGGATCTGAGTGGGAAATATCTGAGATTCACAG |
| | | ACCCACTATTGGAACAGTACCAAACCTAGTTTGGCCG |
| | | ATCCATGATTATGTAATGCATATAGTTTTTGTCGATGC |
| | | TCACCCGTTTCGAGTCTGTCTCGTATCGTCTTACGTAT |
| | | AAGTTCAAGCATGTTTACCAGGTCTGTTAGAAACTCCT |
| | | TTGTGAGGGCAGGACCTATTCGTCTCGGTCCCGTTGTT |
| | | TCTAAGAGACTGTACAGCCAAGCGCAGAATGGTGGCA |
| | | TTAACCATAAGAGGATTCTGATCGGACTTGGTCTATTG |
| | | GCTATTGGAACCACCCTTTACGGGACAACCAACCCTA |
| | | CCAAGACTCCTATTGCATTTGTGGAACCAGCCACGGA |
| | | AAGAGCGTTTAAGGACGGAGACGTCTCTGTGATTTTT |
| | | GTTCTCGGAGGTCCAGGAGCTGGAAAAGGTACCCAAT |
| | | GTGCCAAACTAGTGAGTAATTACGGATTTGTTCACCTG |
| | | TCAGCTGGAGACTTGTTACGTGCAGAACAGAAGAGGG |
| | | AGGGGTCTAAGTATGGAGAGATGATTTCCCAGTATAT |
| | | CAGAGATGGACTGATAGTACCTCAAGAGGTCACCATT |
| | | GCGCTCTTGGAGCAGGCCATGAAGGAAAACTTCGAGA |
| | | AAGGGAAGACACGGTTCTTGATTGATGGATTCCCTCG |
| | | TAAGATGGACCAGGCCAAAACTTTTGAGGAAAAAGTC |
| | | GCAAAGTCCAAGGTGACACTTTTCTTTGATTGTCCCGA |
| | | ATCAGTGCTCCTTGAGAGATTACTTAAAAGAGGACAG |
| | | ACAAGCGGAAGAGAGGATGATAATGCGGAGAGTATC |
| | | AAAAAAAGATTCAAAACATTCGTGGAAACTTCGATGC |
| | | CTGTGGTGGACTATTTCGGGAAGCAAGGACGCGTTTT |
| | | GAAGGTATCTTGTGACCACCCTGTGGATCAAGTGTATT |
| | | CACAGGTTGTGTCGGTGCTAAAAGAGAAGGGGATCTT |
| | | TGCCGATAACGAGACGGAGAATAAATAA |
| 34 | NatR ORF | ATGGGTACCACTCTTGACGACACGGCTTACCGGTACC |
| | | GCACCAGTGTCCCGGGGGACGCCGAGGCCATCGAGGC |
| | | ACTGGATGGGTCCTTCACCACCGACACCGTCTTCCGCG |
| | | TCACCGCCACCGGGGACGGCTTCACCCTGCGGGAGGT |
| | | GCCGGTGGACCCGCCCCTGACCAAGGTGTTCCCCGAC |
| | | GACGAATCGGACGACGAATCGGACGACGGGGAGGAC |
| | | GGCGACCCGGACTCCCGGACGTTCGTCGCGTACGGGG |
| | | ACGACGGCGACCTGGCGGGCTTCGTGGTCGTCTCGTA |
| | | CTCCGGCTGGAACCGCCGGCTGACCGTCGAGGACATC |
| | | GAGGTCGCCCCGGAGCACCGGGGGCACGGGGTCGGG |
| | | CGCGCGTTGATGGGGCTCGCGACGGAGTTCGCCCGCG |
| | | AGCGGGGCGCCGGGCACCTCTGGCTGGAGGTCACCAA |
| | | CGTCAACGCACCGGCGATCCACGCGTACCGGCGGATG |
| | | GGGTTCACCCTCTGCGGCCTGGACACCGCCCTGTACG |
| | | ACGGCACCGCCTCGGACGGCGAGCAGGCGCTCTACAT |
| | | GAGCATGCCCTGCCCCTAATCAGTACTG |
| 35 | Sequence of the | ATGGCCAAGTTGACCAGTGCCGTTCCGGTGCTCACCG |
| | Sh ble ORF | CGCGCGACGTCGCCGGAGCGGTCGAGTTCTGGACCGA |
| | (Zeocin | CCGGCTCGGGTTCTCCCGGGACTTCGTGGAGGACGAC |
| | resistance | TTCGCCGGTGTGGTCCGGGACGACGTGACCCTGTTCAT |
| | marker): | CAGCGCGGTCCAGGACCAGGTGGTGCCGGACAACACC |
| | | CTGGCCTGGGTGTGGGTGCGCGGCCTGGACGAGCTGT |
| | | ACGCCGAGTGGTCGGAGGTCGTGTCCACGAACTTCCG |
| | | GGACGCCTCCGGGCCGGCCATGACCGAGATCGGCGAG |
| | | CAGCCGTGGGGGCGGGAGTTCGCCCTGCGCGACCCGG |
| | | CCGGCAACTGCGTGCACTTCGTGGCCGAGGAGCAGGA |
| | | CTGA |
| 36 | PpAOX1 TT | TCAAGAGGATGTCAGAATGCCATTTGCCTGAGAGATG |
| | | CAGGCTTCATTTTGATACTTTTTTATTTGTAACCTATAT |
| | | AGTATAGGATTTTTTTTGTCATTTTGTTTCTTCTCGTAC |
| | | GAGCTTGCTCCTGATCAGCCTATCTCGCAGCTGATGAA |
| | | TATCTTGTGGTAGGGGTTTGGGAAAATCATTCGAGTTT |
| | | GATGTTTTTCTTGGTATTTCCCACTCCTCTTCAGAGTAC |
| | | AGAAGATTAAGTGAGACGTTCGTTTGTGCA |
| 37 | ScTEF1 | GATCCCCCACACACCATAGCTTCAAAATGTTTCTACTC |
| | promoter | CTTTTTTACTCTTCCAGATTTTCTCGGACTCCGCGCATC |
| | | GCCGTACCACTTCAAAACACCCAAGCACAGCATACTA |
| | | AATTTCCCCTCTTTCTTCCTCTAGGGTGTCGTTAATTAC |
| | | CCGTACTAAAGGTTTGGAAAAGAAAAAAGAGACCGC |
| | | CTCGTTTCTTTTTCTTCGTCGAAAAAGGCAATAAAAAT |
| | | TTTTATCACGTTTCTTTTTCTTGAAAATTTTTTTTTTTG |
| | | ATTTTTTTCTCTTTCGATGACCTCCCATTGATATTTAAG |
| | | TTAATAAACGGTCTTCAATTTCTCAAGTTTCAGTTTCA |
| | | TTTTTCTTGTTCTATTACAACTTTTTTTACTTCTTGCTC |
| | | ATTAGAAAGAAAGCATAGCAATCTAATCTAAGTTTTA |
| | | ATTACAAA |
| 38 | S. cerevisiae | AGGCCTCGCAACAACCTATAATTGAGTTAAGTGCCTTT |
| | invertase gene | CCAAGCTAAAAAGTTTGAGGTTATAGGGGCTTAGCAT |
| | (ScSUC2) ORF | CCACACGTCACAATCTCGGGTATCGAGTATAGTATGT |
| | underlined | AGAATTACGGCAGGAGGTTTCCCAATGAACAAAGGAC |
| | | AGGGGCACGGTGAGCTGTCGAAGGTATCCATTTTATC |
| | | ATGTTTCGTTTGTACAAGCACGACATACTAAGACATTT |
| | | ACCGTATGGGAGTTGTTGTCCTAGCGTAGTTCTCGCTC |
| | | CCCCAGCAAAGCTCAAAAAAGTACGTCATTTAGAATA |
| | | GTTTGTGAGCAAATTACCAGTCGGTATGCTACGTTAG |
| | | AAAGGCCCACAGTATTCTTCTACCAAAGGCGTGCCTTT |
| | | GTTGAACTCGATCCATTATGAGGGCTTCCATTATTCCC |
| | | CGCATTTTTATTACTCTGAACAGGAATAAAAAGAAAA |
| | | AACCCAGTTTAGGAAATTATCCGGGGGCGAAGAAATA |
| | | CGCGTAGCGTTAATCGACCCCACGTCCAGGGTTTTTCC |
| | | ATGGAGGTTTCTGGAAAAACTGACGAGGAATGTGATT |
| | | ATAAATCCCTTTATGTGATGTCTAAGACTTTTAAGGTA |
| | | CGCCCGATGTTTGCCTATTACCATCATAGAGACGTTTC |
| | | TTTTCGAGGAATGCTTAAACGACTTTGTTTGACAAAAA |
| | | TGTTGCCTAAGGGCTCTATAGTAAACCATTTGGAAGA |
| | | AAGATTTGACGACTTTTTTTTTTTGGATTTCGATCCTAT |
| | | AATCCTTCCTCCTGAAAAGAAACATATAAATAGATAT |
| | | GTATTATTCTTCAAAACATTCTCTTGTTCTTGTGCTTTT |
| | | TTTTTACCATATATCTTACTTTTTTTTTTCTCTCAGAGA |
| | | AACAAGCAAAACAAAAAGCTTTTCTTTTCACTAACGT |
| | | ATATGATGCTTTTGCAAGCTTTCCTTTTCCTTTTGGCTG |
| | | GTTTTGCAGCCAAAATATCTGCATCAATGACAAACGA |
| | | AACTAGCGATAGACCTTTGGTCCACTTCACACCCAAC |
| | | AAGGGCTGGATGAATGACCCAAATGGGTTGTGGTACG |
| | | ATGAAAAAGATGCCAAATGGCATCTGTACTTTCAATA |
| | | CAACCCAAATGACACCGTATGGGGTACGCCATTGTTT |
| | | TGGGGCCATGCTACTTCCGATGATTTGACTAATTGGGA |
| | | AGATCAACCCATTGCTATCGCTCCCAAGCGTAACGAT |
| | | TCAGGTGCTTTCTCTGGCTCCATGGTGGTTGATTACAA |
| | | CAACACGAGTGGGTTTTTCAATGATACTATTGATCCAA |
| | | GACAAAGATGCGTTGCGATTTGGACTTATAACACTCC |
| | | TGAAAGTGAAGAGCAATACATTAGCTATTCTCTTGAT |
| | | GGTGGTTACACTTTTACTGAATACCAAAAGAACCCTG |
| | | TTTTAGCTGCCAACTCCACTCAATTCAGAGATCCAAAG |
| | | GTGTTCTGGTATGAACCTTCTCAAAAATGGATTATGAC |
| | | GGCTGCCAAATCACAAGACTACAAAATTGAAATTTAC |
| | | TCCTCTGATGACTTGAAGTCCTGGAAGCTAGAATCTGC |
| | | ATTTGCCAATGAAGGTTTCTTAGGCTACCAATACGAAT |
| | | GTCCAGGTTTGATTGAAGTCCCAACTGAGCAAGATCC |
| | | TTCCAAATCTTATTGGGTCATGTTTATTTCTATCAACC |
| | | CAGGTGCACCTGCTGGCGGTTCCTTCAACCAATATTTT |
| | | GTTGGATCCTTCAATGGTACTCATTTTGAAGCGTTTGA |
| | | CAATCAATCTAGAGTGGTAGATTTTGGTAAGGACTAC |
| | | TATGCCTTGCAAACTTTCTTCAACACTGACCCAACCTA |
| | | CGGTTCAGCATTAGGTATTGCCTGGGCTTCAAACTGG |
| | | GAGTACAGTGCCTTTGTCCCAACTAACCCATGGAGAT |
| | | CATCCATGTCTTTGGTCCGCAAGTTTTCTTTGAACACT |
| | | GAATATCAAGCTAATCCAGAGACTGAATTGATCAATT |
| | | TGAAAGCCGAACCAATATTGAACATTAGTAATGCTGG |
| | | TCCCTGGTCTCGTTTTGCTACTAACACAACTCTAACTA |
| | | AGGCCAATTCTTACAATGTCGATTTGAGCAACTCGACT |
| | | GGTACCCTAGAGTTTGAGTTGGTTTACGCTGTTAACAC |
| | | CACACAAACCATATCCAAATCCGTCTTTGCCGACTTAT |
| | | CACTTTGGTTCAAGGGTTTAGAAGATCCTGAAGAATA |
| | | TTTGAGAATGGGTTTTGAAGTCAGTGCTTCTTCCTTCT |
| | | TTTTGGACCGTGGTAACTCTAAGGTCAAGTTTGTCAAG |
| | | GAGAACCCATATTTCACAAACAGAATGTCTGTCAACA |
| | | ACCAACCATTCAAGTCTGAGAACGACCTAAGTTACTA |
| | | TAAAGTGTACGGCCTACTGGATCAAAACATCTTGGAA |
| | | TTGTACTTCAACGATGGAGATGTGGTTTCTACAAATAC |
| | | CTACTTCATGACCACCGGTAACGCTCTAGGATCTGTGA |
| | | ACATGACCACTGGTGTCGATAATTTGTTCTACATTGAC |
| | | AAGTTCCAAGTAAGGGAAGTAAAATAGAGGTTATAA |
| | | AACTTATTGTCTTTTTTATTTTTTTCAAAAGCCATTCTA |
| | | AAGGGCTTTAGCTAACGAGTGACGAATGTAAAACTTT |
| | | ATGATTTCAAAGAATACCTCCAAACCATTGAAAATGT |
| | | ATTTTTATTTTTATTTTCTCCCGACCCCAGTTACCTGGA |
| | | ATTTGTTCTTTATGTACTTTATATAAGTATAATTCTCTT |
| | | AAAAATTTTTACTACTTTGCAATAGACATCATTTTTTC |
| | | ACGTAATAAACCCACAATCGTAATGTAGTTGCCTTAC |
| | | ACTACTAGGATGGACCTTTTTGCCTTTATCTGTTTTGTT |
| | | ACTGACACAATGAAACCGGGTAAAGTATTAGTTATGT |
| | | GAAAATTTAAAAGCATTAAGTAGAAGTATACCATATT |
| | | GTAAAAAAAAAAAGCGTTGTCTTCTACGTAAAAGTGT |
| | | TCTCAAAAAGAAGTAGTGAGGGAAATGGATACCAAGC |
| | | TATCTGTAACAGGAGCTAAAAAATCTCAGGGAAAAGC |
| | | TTCTGGTTTGGGAAACGGTCGAC |
| 39 | Sequence of the | ATCGGCCTTTGTTGATGCAAGTTTTACGTGGATCATGG |
| | 5′-Region used | ACTAAGGAGTTTTATTTGGACCAAGTTCATCGTCCTAG |
| | for knock out of | ACATTACGGAAAGGGTTCTGCTCCTCTTTTTGGAAACT |
| | PpURA5: | TTTTGGAACCTCTGAGTATGACAGCTTGGTGGATTGTA |
| | | CCCATGGTATGGCTTCCTGTGAATTTCTATTTTTTCTAC |
| | | ATTGGATTCACCAATCAAAACAAATTAGTCGCCATGG |
| | | CTTTTTGGCTTTTGGGTCTATTTGTTTGGACCTTCTTGG |
| | | AATATGCTTTGCATAGATTTTTGTTCCACTTGGACTAC |
| | | TATCTTCCAGAGAATCAAATTGCATTTACCATTCATTT |
| | | CTTATTGCATGGGATACACCACTATTTACCAATGGATA |
| | | AATACAGATTGGTGATGCCACCTACACTTTTCATTGTA |
| | | CTTTGCTACCCAATCAAGACGCTCGTCTTTTCTGTTCT |
| | | ACCATATTACATGGCTTGTTCTGGATTTGCAGGTGGAT |
| | | TCCTGGGCTATATCATGTATGATGTCACTCATTACGTT |
| | | CTGCATCACTCCAAGCTGCCTCGTTATTTCCAAGAGTT |
| | | GAAGAAATATCATTTGGAACATCACTACAAGAATTAC |
| | | GAGTTAGGCTTTGGTGTCACTTCCAAATTCTGGGACAA |
| | | AGTCTTTGGGACTTATCTGGGTCCAGACGATGTGTATC |
| | | AAAAGACAAATTAGAGTATTTATAAAGTTATGTAAGC |
| | | AAATAGGGGCTAATAGGGAAAGAAAAATTTTGGTTCT |
| | | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTCGAGGGGTACCAAAAAA |
| | | GGTTTTTGGATGCTGCAGTGGCTTCGC |
| 40 | Sequence of the | GGTCTTTTCAACAAAGCTCCATTAGTGAGTCAGCTGGC |
| | 3′-Region used | TGAATCTTATGCACAGGCCATCATTAACAGCAACCTG |
| | for knock out of | GAGATAGACGTTGTATTTGGACCAGCTTATAAAGGTA |
| | PpURA5: | TTCCTTTGGCTGCTATTACCGTGTTGAAGTTGTACGAG |
| | | CTCGGCGGCAAAAAATACGAAAATGTCGGATATGCGT |
| | | TCAATAGAAAAGAAAAGAAAGACCACGGAGAAGGTG |
| | | GAAGCATCGTTGGAGAAAGTCTAAAGAATAAAAGAGT |
| | | ACTGATTATCGATGATGTGATGACTGCAGGTACTGCT |
| | | ATCAACGAAGCATTTGCTATAATTGGAGCTGAAGGTG |
| | | GGAGAGTTGAAGGTAGTATTATTGCCCTAGATAGAAT |
| | | GGAGACTACAGGAGATGACTCAAATACCAGTGCTACC |
| | | CAGGCTGTTAGTCAGAGATATGGTACCCCTGTCTTGA |
| | | GTATAGTGACATTGGACCATATTGTGGCCCATTTGGGC |
| | | GAAACTTTCACAGCAGACGAGAAATCTCAAATGGAAA |
| | | CGTATAGAAAAAAGTATTTGCCCAAATAAGTATGAAT |
| | | CTGCTTCGAATGAATGAATTAATCCAATTATCTTCTCA |
| | | CCATTATTTTCTTCTGTTTCGGAGCTTTGGGCACGGCG |
| | | GCGGGTGGTGCGGGCTCAGGTTCCCTTTCATAAACAG |
| | | ATTTAGTACTTGGATGCTTAATAGTGAATGGCGAATGC |
| | | AAAGGAACAATTTCGTTCATCTTTAACCCTTTCACTCG |
| | | GGGTACACGTTCTGGAATGTACCCGCCCTGTTGCAACT |
| | | CAGGTGGACCGGGCAATTCTTGAACTTTCTGTAACGTT |
| | | GTTGGATGTTCAACCAGAAATTGTCCTACCAACTGTAT |
| | | TAGTTTCCTTTTGGTCTTATATTGTTCATCGAGATACTT |
| | | CCCACTCTCCTTGATAGCCACTCTCACTCTTCCTGGAT |
| | | TACCAAAATCTTGAGGATGAGTCTTTTCAGGCTCCAG |
| | | GATGCAAGGTATATCCAAGTACCTGCAAGCATCTAAT |
| | | ATTGTCTTTGCCAGGGGGTTCTCCACACCATACTCCTT |
| | | TTGGCGCATGC |
| 41 | Sequence of the | TCTAGAGGGACTTATCTGGGTCCAGACGATGTGTATC |
| | PpURA5 | AAAAGACAAATTAGAGTATTTATAAAGTTATGTAAGC |
| | auxotrophic | AAATAGGGGCTAATAGGGAAAGAAAAATTTTGGTTCT |
| | marker: | TTATCAGAGCTGGCTCGCGCGCAGTGTTTTTCGTGCTC |
| | | CTTTGTAATAGTCATTTTTGACTACTGTTCAGATTGAA |
| | | ATCACATTGAAGATGTCACTGGAGGGGTACCAAAAAA |
| | | GGTTTTTGGATGCTGCAGTGGCTTCGCAGGCCTTGAAG |
| | | TTTGGAACTTTCACCTTGAAAAGTGGAAGACAGTCTC |
| | | CATACTTCTTTAACATGGGTCTTTTCAACAAAGCTCCA |
| | | TTAGTGAGTCAGCTGGCTGAATCTTATGCTCAGGCCAT |
| | | CATTAACAGCAACCTGGAGATAGACGTTGTATTTGGA |
| | | CCAGCTTATAAAGGTATTCCTTTGGCTGCTATTACCGT |
| | | GTTGAAGTTGTACGAGCTGGGCGGCAAAAAATACGAA |
| | | AATGTCGGATATGCGTTCAATAGAAAAGAAAAGAAAG |
| | | ACCACGGAGAAGGTGGAAGCATCGTTGGAGAAAGTCT |
| | | AAAGAATAAAAGAGTACTGATTATCGATGATGTGATG |
| | | ACTGCAGGTACTGCTATCAACGAAGCATTTGCTATAA |
| | | TTGGAGCTGAAGGTGGGAGAGTTGAAGGTTGTATTAT |
| | | TGCCCTAGATAGAATGGAGACTACAGGAGATGACTCA |
| | | AATACCAGTGCTACCCAGGCTGTTAGTCAGAGATATG |
| | | GTACCCCTGTCTTGAGTATAGTGACATTGGACCATATT |
| | | GTGGCCCATTTGGGCGAAACTTTCACAGCAGACGAGA |
| | | AATCTCAAATGGAAACGTATAGAAAAAAGTATTTGCC |
| | | CAAATAAGTATGAATCTGCTTCGAATGAATGAATTAA |
| | | TCCAATTATCTTCTCACCATTATTTTCTTCTGTTTCGGA |
| | | GCTTTGGGCACGGCGGCGGATCC |
| 42 | Sequence of the | CCTGCACTGGATGGTGGCGCTGGATGGTAAGCCGCTG |
| | part of the Ec | GCAAGCGGTGAAGTGCCTCTGGATGTCGCTCCACAAG |
| | lacZ gene that | GTAAACAGTTGATTGAACTGCCTGAACTACCGCAGCC |
| | was used to | GGAGAGCGCCGGGCAACTCTGGCTCACAGTACGCGTA |
| | construct the | GTGCAACCGAACGCGACCGCATGGTCAGAAGCCGGGC |
| | PpURA5 blaster | ACATCAGCGCCTGGCAGCAGTGGCGTCTGGCGGAAAA |
| | (recyclable | CCTCAGTGTGACGCTCCCCGCCGCGTCCCACGCCATCC |
| | auxotrophic | CGCATCTGACCACCAGCGAAATGGATTTTTGCATCGA |
| | marker) | GCTGGGTAATAAGCGTTGGCAATTTAACCGCCAGTCA |
| | | GGCTTTCTTTCACAGATGTGGATTGGCGATAAAAAAC |
| | | AACTGCTGACGCCGCTGCGCGATCAGTTCACCCGTGC |
| | | ACCGCTGGATAACGACATTGGCGTAAGTGAAGCGACC |
| | | CGCATTGACCCTAACGCCTGGGTCGAACGCTGGAAGG |
| | | CGGCGGGCCATTACCAGGCCGAAGCAGCGTTGTTGCA |
| | | GTGCACGGCAGATACACTTGCTGATGCGGTGCTGATT |
| | | ACGACCGCTCACGCGTGGCAGCATCAGGGGAAAACCT |
| | | TATTTATCAGCCGGAAAACCTACCGGATTGATGGTAG |
| | | TGGTCAAATGGCGATTACCGTTGATGTTGAAGTGGCG |
| | | AGCGATACACCGCATCCGGCGCGGATTGGCCTGAACT |
| | | GCCAG |
| 43 | Sequence of the | AAAACCTTTTTTCCTATTCAAACACAAGGCATTGCTTC |
| | 5′-Region used | AACACGTGTGCGTATCCTTAACACAGATACTCCATACT |
| | for knock out of | TCTAATAATGTGATAGACGAATACAAAGATGTTCACT |
| | PpOCH1: | CTGTGTTGTGTCTACAAGCATTTCTTATTCTGATTGGG |
| | | GATATTCTAGTTACAGCACTAAACAACTGGCGATACA |
| | | AACTTAAATTAAATAATCCGAATCTAGAAAATGAACT |
| | | TTTGGATGGTCCGCCTGTTGGTTGGATAAATCAATACC |
| | | GATTAAATGGATTCTATTCCAATGAGAGAGTAATCCA |
| | | AGACACTCTGATGTCAATAATCATTTGCTTGCAACAAC |
| | | AAACCCGTCATCTAATCAAAGGGTTTGATGAGGCTTA |
| | | CCTTCAATTGCAGATAAACTCATTGCTGTCCACTGCTG |
| | | TATTATGTGAGAATATGGGTGATGAATCTGGTCTTCTC |
| | | CACTCAGCTAACATGGCTGTTTGGGCAAAGGTGGTAC |
| | | AATTATACGGAGATCAGGCAATAGTGAAATTGTTGAA |
| | | TATGGCTACTGGACGATGCTTCAAGGATGTACGTCTA |
| | | GTAGGAGCCGTGGGAAGATTGCTGGCAGAACCAGTTG |
| | | GCACGTCGCAACAATCCCCAAGAAATGAAATAAGTGA |
| | | AAACGTAACGTCAAAGACAGCAATGGAGTCAATATTG |
| | | ATAACACCACTGGCAGAGCGGTTCGTACGTCGTTTTG |
| | | GAGCCGATATGAGGCTCAGCGTGCTAACAGCACGATT |
| | | GACAAGAAGACTCTCGAGTGACAGTAGGTTGAGTAAA |
| | | GTATTCGCTTAGATTCCCAACCTTCGTTTTATTCTTTCG |
| | | TAGACAAAGAAGCTGCATGCGAACATAGGGACAACTT |
| | | TTATAAATCCAATTGTCAAACCAACGTAAAACCCTCT |
| | | GGCACCATTTTCAACATATATTTGTGAAGCAGTACGC |
| | | AATATCGATAAATACTCACCGTTGTTTGTAACAGCCCC |
| | | AACTTGCATACGCCTTCTAATGACCTCAAATGGATAA |
| | | GCCGCAGCTTGTGCTAACATACCAGCAGCACCGCCCG |
| | | CGGTCAGCTGCGCCCACACATATAAAGGCAATCTACG |
| | | ATCATGGGAGGAATTAGTTTTGACCGTCAGGTCTTCA |
| | | AGAGTTTTGAACTCTTCTTCTTGAACTGTGTAACCTTT |
| | | TAAATGACGGGATCTAAATACGTCATGGATGAGATCA |
| | | TGTGTGTAAAAACTGACTCCAGCATATGGAATCATTC |
| | | CAAAGATTGTAGGAGCGAACCCACGATAAAAGTTTCC |
| | | CAACCTTGCCAAAGTGTCTAATGCTGTGACTTGAAATC |
| | | TGGGTTCCTCGTTGAAGACCCTGCGTACTATGCCCAAA |
| | | AACTTTCCTCCACGAGCCCTATTAACTTCTCTATGAGT |
| | | TTCAAATGCCAAACGGACACGGATTAGGTCCAATGGG |
| | | TAAGTGAAAAACACAGAGCAAACCCCAGCTAATGAG |
| | | CCGGCCAGTAACCGTCTTGGAGCTGTTTCATAAGAGT |
| | | CATTAGGGATCAATAACGTTCTAATCTGTTCATAACAT |
| | | ACAAATTTTATGGCTGCATAGGGAAAAATTCTCAACA |
| | | GGGTAGCCGAATGACCCTGATATAGACCTGCGACACC |
| | | ATCATACCCATAGATCTGCCTGACAGCCTTAAAGAGC |
| | | CCGCTAAAAGACCCGGAAAACCGAGAGAACTCTGGAT |
| | | TAGCAGTCTGAAAAAGAATCTTCACTCTGTCTAGTGG |
| | | AGCAATTAATGTCTTAGCGGCACTTCCTGCTACTCCGC |
| | | CAGCTACTCCTGAATAGATCACATACTGCAAAGACTG |
| | | CTTGTCGATGACCTTGGGGTTATTTAGCTTCAAGGGCA |
| | | ATTTTTGGGACATTTTGGACACAGGAGACTCAGAAAC |
| | | AGACACAGAGCGTTCTGAGTCCTGGTGCTCCTGACGT |
| | | AGGCCTAGAACAGGAATTATTGGCTTTATTTGTTTGTC |
| | | CATTTCATAGGCTTGGGGTAATAGATAGATGACAGAG |
| | | AAATAGAGAAGACCTAATATTTTTTGTTCATGGCAAAT |
| | | CGCGGGTTCGCGGTCGGGTCACACACGGAGAAGTAAT |
| | | GAGAAGAGCTGGTAATCTGGGGTAAAAGGGTTCAAAA |
| | | GAAGGTCGCCTGGTAGGGATGCAATACAAGGTTGTCT |
| | | TGGAGTTTACATTGACCAGATGATTTGGCTTTTTCTCT |
| | | GTTCAATTCACATTTTTCAGCGAGAATCGGATTGACGG |
| | | AGAAATGGCGGGGTGTGGGGTGGATAGATGGCAGAA |
| | | ATGCTCGCAATCACCGCGAAAGAAAGACTTTATGGAA |
| | | TAGAACTACTGGGTGGTGTAAGGATTACATAGCTAGT |
| | | CCAATGGAGTCCGTTGGAAAGGTAAGAAGAAGCTAAA |
| | | ACCGGCTAAGTAACTAGGGAAGAATGATCAGACTTTG |
| | | ATTTGATGAGGTCTGAAAATACTCTGCTGCTTTTTCAG |
| | | TTGCTTTTTCCCTGCAACCTATCATTTTCCTTTTCATAA |
| | | GCCTGCCTTTTCTGTTTTCACTTATATGAGTTCCGCCG |
| | | AGACTTCCCCAAATTCTCTCCTGGAACATTCTCTATCG |
| | | CTCTCCTTCCAAGTTGCGCCCCCTGGCACTGCCTAGTA |
| | | ATATTACCACGCGACTTATATTCAGTTCCACAATTTCC |
| | | AGTGTTCGTAGCAAATATCATCAGCCATGGCGAAGGC |
| | | AGATGGCAGTTTGCTCTACTATAATCCTCACAATCCAC |
| | | CCAGAAGGTATTACTTCTACATGGCTATATTCGCCGTT |
| | | TCTGTCATTTGCGTTTTGTACGGACCCTCACAACAATT |
| | | ATCATCTCCAAAAATAGACTATGATCCATTGACGCTCC |
| | | GATCACTTGATTTGAAGACTTTGGAAGCTCCTTCACAG |
| | | TTGAGTCCAGGCACCGTAGAAGATAATCTTCG |
| 44 | Sequence of the | AAAGCTAGAGTAAAATAGATATAGCGAGATTAGAGA |
| | 3′-Region used | ATGAATACCTTCTTCTAAGCGATCGTCCGTCATCATAG |
| | for knock out of | AATATCATGGACTGTATAGTTTTTTTTTTGTACATATA |
| | PpOCH1: | ATGATTAAACGGTCATCCAACATCTCGTTGACAGATCT |
| | | CTCAGTACGCGAAATCCCTGACTATCAAAGCAAGAAC |
| | | CGATGAAGAAAAAAACAACAGTAACCCAAACACCAC |
| | | AACAAACACTTTATCTTCTCCCCCCCAACACCAATCAT |
| | | CAAAGAGATGTCGGAACCAAACACCAAGAAGCAAAA |
| | | ACTAACCCCATATAAAAACATCCTGGTAGATAATGCT |
| | | GGTAACCCGCTCTCCTTCCATATTCTGGGCTACTTCAC |
| | | GAAGTCTGACCGGTCTCAGTTGATCAACATGATCCTC |
| | | GAAATGGGTGGCAAGATCGTTCCAGACCTGCCTCCTC |
| | | TGGTAGATGGAGTGTTGTTTTTGACAGGGGATTACAA |
| | | GTCTATTGATGAAGATACCCTAAAGCAACTGGGGGAC |
| | | GTTCCAATATACAGAGACTCCTTCATCTACCAGTGTTT |
| | | TGTGCACAAGACATCTCTTCCCATTGACACTTTCCGAA |
| | | TTGACAAGAACGTCGACTTGGCTCAAGATTTGATCAA |
| | | TAGGGCCCTTCAAGAGTCTGTGGATCATGTCACTTCTG |
| | | CCAGCACAGCTGCAGCTGCTGCTGTTGTTGTCGCTACC |
| | | AACGGCCTGTCTTCTAAACCAGACGCTCGTACTAGCA |
| | | AAATACAGTTCACTCCCGAAGAAGATCGTTTTATTCTT |
| | | GACTTTGTTAGGAGAAATCCTAAACGAAGAAACACAC |
| | | ATCAACTGTACACTGAGCTCGCTCAGCACATGAAAAA |
| | | CCATACGAATCATTCTATCCGCCACAGATTTCGTCGTA |
| | | ATCTTTCCGCTCAACTTGATTGGGTTTATGATATCGAT |
| | | CCATTGACCAACCAACCTCGAAAAGATGAAAACGGGA |
| | | ACTACATCAAGGTACAAGGCCTTCCA |
| 45 | K. lactis UDP- | AAACGTAACGCCTGGCACTCTATTTTCTCAAACTTCTG |
| | GlcNAc | GGACGGAAGAGCTAAATATTGTGTTGCTTGAACAAAC |
| | transporter gene | CCAAAAAAACAAAAAAATGAACAAACTAAAACTACA |
| | (KIMNN2-2) | CCTAAATAAACCGTGTGTAAAACGTAGTACCATATTA |
| | ORF underlined | CTAGAAAAGATCACAAGTGTATCACACATGTGCATCT |
| | | CATATTACATCTTTTATCCAATCCATTCTCTCTATCCCG |
| | | TCTGTTCCTGTCAGATTCTTTTTCCATAAAAAGAAGAA |
| | | GACCCCGAATCTCACCGGTACAATGCAAAACTGCTGA |
| | | AAAAAAAAGAAAGTTCACTGGATACGGGAACAGTGC |
| | | CAGTAGGCTTCACCACATGGACAAAACAATTGACGAT |
| | | AAAATAAGCAGGTGAGCTTCTTTTTCAAGTCACGATC |
| | | CCTTTATGTCTCAGAAACAATATATACAAGCTAAACC |
| | | CTTTTGAACCAGTTCTCTCTTCATAGTTATGTTCACAT |
| | | AAATTGCGGGAACAAGACTCCGCTGGCTGTCAGGTAC |
| | | ACGTTGTAACGTTTTCGTCCGCCCAATTATTAGCACAA |
| | | CATTGGCAAAAAGAAAAACTGCTCGTTTTCTCTACAG |
| | | GTAAATTACAATTTTTTTCAGTAATTTTCGCTGAAAAA |
| | | TTTAAAGGGCAGGAAAAAAAGACGATCTCGACTTTGC |
| | | ATAGATGCAAGAACTGTGGTCAAAACTTGAAATAGTA |
| | | ATTTTGCTGTGCGTGAACTAATAAATATATATATATAT |
| | | ATATATATATATTTGTGTATTTTGTATATGTAATTGTGC |
| | | ACGTCTTGGCTATTGGATATAAGATTTTCGCGGGTTGA |
| | | TGACATAGAGCGTGTACTACTGTAATAGTTGTATATTC |
| | | AAAAGCTGCTGCGTGGAGAAAGACTAAAATAGATAA |
| | | AAAGCACACATTTTGACTTCGGTACCGTCAACTTAGTG |
| | | GGACAGTCTTTTATATTTGGTGTAAGCTCATTTCTGGT |
| | | ACTATTCGAAACAGAACAGTGTTTTCTGTATTACCGTC |
| | | CAATCGTTTGTCATGAGTTTTGTATTGATTTTGTCGTT |
| | | AGTGTTCGGAGGATGTTGTTCCAATGTGATTAGTTTCG |
| | | AGCACATGGTGCAAGGCAGCAATATAAATTTGGGAAA |
| | | TATTGTTACATTCACTCAATTCGTGTCTGTGACGCTAA |
| | | TTCAGTTGCCCAATGCTTTGGACTTCTCTCACTTTCCGT |
| | | TTAGGTTGCGACCTAGACACATTCCTCTTAAGATCCAT |
| | | ATGTTAGCTGTGTTTTTGTTCTTTACCAGTTCAGTCGCC |
| | | AATAACAGTGTGTTTAAATTTGACATTTCCGTTCCGAT |
| | | TCATATTATCATTAGATTTTCAGGTACCACTTTGACGA |
| | | TGATAATAGGTTGGGCTGTTTGTAATAAGAGGTACTCC |
| | | AAACTTCAGGTGCAATCTGCCATCATTATGACGCTTGG |
| | | TGCGATTGTCGCATCATTATACCGTGACAAAGAATTTT |
| | | CAATGGACAGTTTAAAGTTGAATACGGATTCAGTGGG |
| | | TATGACCCAAAAATCTATGTTTGGTATCTTTGTTGTGC |
| | | TAGTGGCCACTGCCTTGATGTCATTGTTGTCGTTGCTC |
| | | AACGAATGGACGTATAACAAGTACGGGAAACATTGGA |
| | | AAGAAACTTTGTTCTATTCGCATTTCTTGGCTCTACCG |
| | | TTGTTTATGTTGGGGTACACAAGGCTCAGAGACGAAT |
| | | TCAGAGACCTCTTAATTTCCTCAGACTCAATGGATATT |
| | | CCTATTGTTAAATTACCAATTGCTACGAAACTTTTCAT |
| | | AATAGCAAATAACGTGACCCAGTTCATTTGTATC |
| | | AAAGGTGTTAACATGCTAGCTAGTAACACGGATGCTT |
| | | TGACACTTTCTGTCGTGCTTCTAGTGCGTAAATTTGTT |
| | | AGTCTTTTACTCAGTGTCTACATCTACAAGAACGTCCT |
| | | ATCCGTGACTGCATACCTAGGGACCATCACCGTGTTCC |
| | | TGGGAGCTGGTTTGTATTCATATGGTTCGGTCAAAACT |
| | | GCACTGCCTCGCTGAAACAATCCACGTCTGTATGATA |
| | | CTCGTTTCAGAATTTTTTTGATTTTCTGCCGGATATGGT |
| | | TTCTCATCTTTACAATCGCATTCTTAATTATACCAGAA |
| | | CGTAATTCAATGATCCCAGTGACTCGTAACTCTTATAT |
| | | GTCAATTTAAGC |
| 46 | Sequence of the | GGCCGAGCGGGCCTAGATTTTCACTACAAATTTCAAA |
| | 5′-Region used | ACTACGCGGATTTATTGTCTCAGAGAGCAATTTGGCAT |
| | for knock out of | TTCTGAGCGTAGCAGGAGGCTTCATAAGATTGTATAG |
| | PpBMT2: | GACCGTACCAACAAATTGCCGAGGCACAACACGGTAT |
| | | GCTGTGCACTTATGTGGCTACTTCCCTACAACGGAATG |
| | | AAACCTTCCTCTTTCCGCTTAAACGAGAAAGTGTGTCG |
| | | CAATTGAATGCAGGTGCCTGTGCGCCTTGGTGTATTGT |
| | | TTTTGAGGGCCCAATTTATCAGGCGCCTTTTTTCTTGG |
| | | TTGTTTTCCCTTAGCCTCAAGCAAGGTTGGTCTATTTC |
| | | ATCTCCGCTTCTATACCGTGCCTGATACTGTTGGATGA |
| | | GAACACGACTCAACTTCCTGCTGCTCTGTATTGCCAGT |
| | | GTTTTGTCTGTGATTTGGATCGGAGTCCTCCTTACTTG |
| | | GAATGATAATAATCTTGGCGGAATCTCCCTAAACGGA |
| | | GGCAAGGATTCTGCCTATGATGATCTGCTATCATTGGG |
| | | AAGCTTCAACGACATGGAGGTCGACTCCTATGTCACC |
| | | AACATCTACGACAATGCTCCAGTGCTAGGATGTACGG |
| | | ATTTGTCTTATCATGGATTGTTGAAAGTCACCCCAAAG |
| | | CATGACTTAGCTTGCGATTTGGAGTTCATAAGAGCTCA |
| | | GATTTTGGACATTGACGTTTACTCCGCCATAAAAGACT |
| | | TAGAAGATAAAGCCTTGACTGTAAAACAAAAGGTTGA |
| | | AAAACACTGGTTTACGTTTTATGGTAGTTCAGTCTTTC |
| | | TGCCCGAACACGATGTGCATTACCTGGTTAGACGAGT |
| | | CATCTTTTCGGCTGAAGGAAAGGCGAACTCTCCAGTA |
| | | ACATC |
| 47 | Sequence of the | CCATATGATGGGTGTTTGCTCACTCGTATGGATCAAAA |
| | 3′-Region used | TTCCATGGTTTCTTCTGTACAACTTGTACACTTATTTGG |
| | for knock out of | ACTTTTCTAACGGTTTTTCTGGTGATTTGAGAAGTCCT |
| | PpBMT2: | TATTTTGGTGTTCGCAGCTTATCCGTGATTGAACCATC |
| | | AGAAATACTGCAGCTCGTTATCTAGTTTCAGAATGTGT |
| | | TGTAGAATACAATCAATTCTGAGTCTAGTTTGGGTGGG |
| | | TCTTGGCGACGGGACCGTTATATGCATCTATGCAGTGT |
| | | TAAGGTACATAGAATGAAAATGTAGGGGTTAATCGAA |
| | | AGCATCGTTAATTTCAGTAGAACGTAGTTCTATTCCCT |
| | | ACCCAAATAATTTGCCAAGAATGCTTCGTATCCACAT |
| | | ACGCAGTGGACGTAGCAAATTTCACTTTGGACTGTGA |
| | | CCTCAAGTCGTTATCTTCTACTTGGACATTGATGGTCA |
| | | TTACGTAATCCACAAAGAATTGGATAGCCTCTCGTTTT |
| | | ATCTAGTGCACAGCCTAATAGCACTTAAGTAAGAGCA |
| | | ATGGACAAATTTGCATAGACATTGAGCTAGATACGTA |
| | | ACTCAGATCTTGTTCACTCATGGTGTACTCGAAGTACT |
| | | GCTGGAACCGTTACCTCTTATCATTTCGCTACTGGCTC |
| | | GTGAAACTACTGGATGAAAAAAAAAAAAGAGCTGAA |
| | | AGCGAGATCATCCCATTTTGTCATCATACAAATTCACG |
| | | CTTGCAGTTTTGCTTCGTTAACAAGACAAGATGTCTTT |
| | | ATCAAAGACCCGTTTTTTCTTCTTGAAGAATACTTCCC |
| | | TGTTGAGCACATGCAAACCATATTTATCTCAGATTTCA |
| | | CTCAACTTGGGTGCTTCCAAGAGAAGTAAAATTCTTCC |
| | | CACTGCATCAACTTCCAAGAAACCCGTAGACCAGTTT |
| | | CTCTTCAGCCAAAAGAAGTTGCTCGCCGATCACCGCG |
| | | GTAACAGAGGAGTCAGAAGGTTTCACACCCTTCCATC |
| | | CCGATTTCAAAGTCAAAGTGCTGCGTTGAACCAAGGT |
| | | TTTCAGGTTGCCAAAGCCCAGTCTGCAAAAACTAGTT |
| | | CCAAATGGCCTATTAATTCCCATAAAAGTGTTGGCTAC |
| | | GTATGTATCGGTACCTCCATTCTGGTATTTGCTATTGT |
| | | TGTCGTTGGTGGGTTGACTAGACTGACCGAATCCGGT |
| | | CTTTCCATAACGGAGTGGAAACCTATCACTGGTTCGGT |
| | | TCCCCCACTGACTGAGGAAGACTGGAAGTTGGAATTT |
| | | GAAAAATACAAACAAAGCCCTGAGTTTCAGGAACTAA |
| | | ATTCTCACATAACATTGGAAGAGTTCAAGTTTATATTT |
| | | TCCATGGAATGGGGACATAGATTGTTGGGAAGGGTCA |
| | | TCGGCCTGTCGTTTGTTCTTCCCACGTTTTACTTCATTG |
| | | CCCGTCGAAAGTGTTCCAAAGATGTTGCATTGAAACT |
| | | GCTTGCAATATGCTCTATGATAGGATTCCAAGGTTTCA |
| | | TCGGCTGGTGGATGGTGTATTCCGGATTGGACAAACA |
| | | GCAATTGGCTGAACGTAACTCCAAACCAACTGTGTCT |
| | | CCATATCGCTTAACTACCCATCTTGGAACTGCATTTGT |
| | | TATTTACTGTTACATGATTTACACAGGGCTTCAAGTTT |
| | | TGAAGAACTATAAGATCATGAAACAGCCTGAAGCGTA |
| | | TGTTCAAATTTTCAAGCAAATTGCGTCTCCAAAATTGA |
| | | AAACTTTCAAGAGACTCTCTTCAGTTCTATTAGGCCTG |
| | | GTG |
| 48 | DNA encodes | ATGTCTGCCAACCTAAAATATCTTTCCTTGGGAATTTT |
| | MmSLC35A3 | GGTGTTTCAGACTACCAGTCTGGTTCTAACGATGCGGT |
| | UDP-GlcNAc | ATTCTAGGACTTTAAAAGAGGAGGGGCCTCGTTATCT |
| | transporter | GTCTTCTACAGCAGTGGTTGTGGCTGAATTTTTGAAGA |
| | | TAATGGCCTGCATCTTTTTAGTCTACAAAGACAGTAAG |
| | | TGTAGTGTGAGAGCACTGAATAGAGTACTGCATGATG |
| | | AAATTCTTAATAAGCCCATGGAAACCCTGAAGCTCGC |
| | | TATCCCGTCAGGGATATATACTCTTCAGAACAACTTAC |
| | | TCTATGTGGCACTGTCAAACCTAGATGCAGCCACTTAC |
| | | CAGGTTACATATCAGTTGAAAATACTTACAACAGCAT |
| | | TATTTTCTGTGTCTATGCTTGGTAAAAAATTAGGTGTG |
| | | TACCAGTGGCTCTCCCTAGTAATTCTGATGGCAGGAGT |
| | | TGCTTTTGTACAGTGGCCTTCAGATTCTCAAGAGCTGA |
| | | ACTCTAAGGACCTTTCAACAGGCTCACAGTTTGTAGG |
| | | CCTCATGGCAGTTCTCACAGCCTGTTTTTCAAGTGGCT |
| | | TTGCTGGAGTTTATTTTGAGAAAATCTTAAAAGAAAC |
| | | AAAACAGTCAGTATGGATAAGGAACATTCAACTTGGT |
| | | TTCTTTGGAAGTATATTTGGATTAATGGGTGTATACGT |
| | | TTATGATGGAGAATTGGTCTCAAAGAATGGATTTTTTC |
| | | AGGGATATAATCAACTGACGTGGATAGTTGTTGCTCT |
| | | GCAGGCACTTGGAGGCCTTGTAATAGCTGCTGTCATC |
| | | AAATATGCAGATAACATTTTAAAAGGATTTGCGACCT |
| | | CCTTATCCATAATATTGTCAACAATAATATCTTATTTT |
| | | TGGTTGCAAGATTTTGTGCCAACCAGTGTCTTTTTCCT |
| | | TGGAGCCATCCTTGTAATAGCAGCTACTTTCTTGTATG |
| | | GTTACGATCCCAAACCTGCAGGAAATCCCACTAAAGC |
| | | ATAG |
| 49 | Sequence of the | GATCTGGCCATTGTGAAACTTGACACTAAAGACAAAA |
| | 5′-Region used | CTCTTAGAGTTTCCAATCACTTAGGAGACGATGTTTCC |
| | for knock out of | TACAACGAGTACGATCCCTCATTGATCATGAGCAATTT |
| | PpMNN4L1: | GTATGTGAAAAAAGTCATCGACCTTGACACCTTGGAT |
| | | AAAAGGGCTGGAGGAGGTGGAACCACCTGTGCAGGC |
| | | GGTCTGAAAGTGTTCAAGTACGGATCTACTACCAAAT |
| | | ATACATCTGGTAACCTGAACGGCGTCAGGTTAGTATA |
| | | CTGGAACGAAGGAAAGTTGCAAAGCTCCAAATTTGTG |
| | | GTTCGATCCTCTAATTACTCTCAAAAGCTTGGAGGAA |
| | | ACAGCAACGCCGAATCAATTGACAACAATGGTGTGGG |
| | | TTTTGCCTCAGCTGGAGACTCAGGCGCATGGATTCTTT |
| | | CCAAGCTACAAGATGTTAGGGAGTACCAGTCATTCAC |
| | | TGAAAAGCTAGGTGAAGCTACGATGAGCATTTTCGAT |
| | | TTCCACGGTCTTAAACAGGAGACTTCTACTACAGGGC |
| | | TTGGGGTAGTTGGTATGATTCATTCTTACGACGGTGAG |
| | | TTCAAACAGTTTGGTTTGTTCACTCCAATGACATCTAT |
| | | TCTACAAAGACTTCAACGAGTGACCAATGTAGAATGG |
| | | TGTGTAGCGGGTTGCGAAGATGGGGATGTGGACACTG |
| | | AAGGAGAACACGAATTGAGTGATTTGGAACAACTGCA |
| | | TATGCATAGTGATTCCGACTAGTCAGGCAAGAGAGAG |
| | | CCCTCAAATTTACCTCTCTGCCCCTCCTCACTCCTTTTG |
| | | GTACGCATAATTGCAGTATAAAGAACTTGCTGCCAGC |
| | | CAGTAATCTTATTTCATACGCAGTTCTATATAGCACAT |
| | | AATCTTGCTTGTATGTATGAAATTTACCGCGTTTTAGT |
| | | TGAAATTGTTTATGTTGTGTGCCTTGCATGAAATCTCT |
| | | CGTTAGCCCTATCCTTACATTTAACTGGTCTCAAAACC |
| | | TCTACCAATTCCATTGCTGTACAACAATATGAGGCGG |
| | | CATTACTGTAGGGTTGGAAAAAAATTGTCATTCCAGC |
| | | TAGAGATCACACGACTTCATCACGCTTATTGCTCCTCA |
| | | TTGCTAAATCATTTACTCTTGACTTCGACCCAGAAAAG |
| | | TTCGCC |
| 50 | Sequence of the | GCATGTCAAACTTGAACACAACGACTAGATAGTTGTT |
| | 3′-Region used | TTTTCTATATAAAACGAAACGTTATCATCTTTAATAAT |
| | for knock out of | CATTGAGGTTTACCCTTATAGTTCCGTATTTTCGTTTCC |
| | PpMNN4L1: | AAACTTAGTAATCTTTTGGAAATATCATCAAAGCTGGT |
| | | GCCAATCTTCTTGTTTGAAGTTTCAAACTGCTCCACCA |
| | | AGCTACTTAGAGACTGTTCTAGGTCTGAAGCAACTTC |
| | | GAACACAGAGACAGCTGCCGCCGATTGTTCTTTTTTGT |
| | | GTTTTTCTTCTGGAAGAGGGGCATCATCTTGTATGTCC |
| | | AATGCCCGTATCCTTTCTGAGTTGTCCGACACATTGTC |
| | | CTTCGAAGAGTTTCCTGACATTGGGCTTCTTCTATCCG |
| | | TGTATTAATTTTGGGTTAAGTTCCTCGTTTGCATAGCA |
| | | GTGGATACCTCGATTTTTTTGGCTCCTATTTACCTGAC |
| | | ATAATATTCTACTATAATCCAACTTGGACGCGTCATCT |
| | | ATGATAACTAGGCTCTCCTTTGTTCAAAGGGGACGTCT |
| | | TCATAATCCACTGGCACGAAGTAAGTCTGCAACGAGG |
| | | CGGCTTTTGCAACAGAACGATAGTGTCGTTTCGTACTT |
| | | GGACTATGCTAAACAAAAGGATCTGTCAAACATTTCA |
| | | ACCGTGTTTCAAGGCACTCTTTACGAATTATCGACCAA |
| | | GACCTTCCTAGACGAACATTTCAACATATCCAGGCTA |
| | | CTGCTTCAAGGTGGTGCAAATGATAAAGGTATAGATA |
| | | TTAGATGTGTTTGGGACCTAAAACAGTTCTTGCCTGAA |
| | | GATTCCCTTGAGCAACAGGCTTCAATAGCCAAGTTAG |
| | | AGAAGCAGTACCAAATCGGTAACAAAAGGGGGAAGC |
| | | ATATAAAACCTTTACTATTGCGACAAAATCCATCCTTG |
| | | AAAGTAAAGCTGTTTGTTCAATGTAAAGCATACGAAA |
| | | CGAAGGAGGTAGATCCTAAGATGGTTAGAGAACTTAA |
| | | CGGGACATACTCCAGCTGCATCCCATATTACGATCGCT |
| | | GGAAGACTTTTTTCATGTACGTATCGCCCACCAACCTT |
| | | TCAAAGCAAGCTAGGTATGATTTTGACAGTTCTCACA |
| | | ATCCATTGGTTTTCATGCAACTTGAAAAAACCCAACTC |
| | | AAACTTCATGGGGATCCATACAATGTAAATCATTACG |
| | | AGAGGGCGAGGTTGAAAAGTTTCCATTGCAATCACGT |
| | | CGCATCATGGCTACTGAAAGGCCTTAAC |
| 51 | Sequence of the | TCATTCTATATGTTCAAGAAAAGGGTAGTGAAAGGAA |
| | 5′-Region used | AGAAAAGGCATATAGGCGAGGGAGAGTTAGCTAGCA |
| | for knock out of | TACAAGATAATGAAGGATCAATAGCGGTAGTTAAAGT |
| | PpPNO1 and | GCACAAGAAAAGAGCACCTGTTGAGGCTGATGATAAA |
| | PpMNN4: | GCTCCAATTACATTGCCACAGAGAAACACAGTAACAG |
| | | AAATAGGAGGGGATGCACCACGAGAAGAGCATTCAG |
| | | TGAACAACTTTGCCAAATTCATAACCCCAAGCGCTAA |
| | | TAAGCCAATGTCAAAGTCGGCTACTAACATTAATAGT |
| | | ACAACAACTATCGATTTTCAACCAGATGTTTGCAAGG |
| | | ACTACAAACAGACAGGTTACTGCGGATATGGTGACAC |
| | | TTGTAAGTTTTTGCACCTGAGGGATGATTTCAAACAGG |
| | | GATGGAAATTAGATAGGGAGTGGGAAAATGTCCAAA |
| | | AGAAGAAGCATAATACTCTCAAAGGGGTTAAGGAGAT |
| | | CCAAATGTTTAATGAAGATGAGCTCAAAGATATCCCG |
| | | TTTAAATGCATTATATGCAAAGGAGATTACAAATCAC |
| | | CCGTGAAAACTTCTTGCAATCATTATTTTTGCGAACAA |
| | | TGTTTCCTGCAACGGTCAAGAAGAAAACCAAATTGTA |
| | | TTATATGTGGCAGAGACACTTTAGGAGTTGCTTTACCA |
| | | GCAAAGAAGTTGTCCCAATTTCTGGCTAAGATACATA |
| | | ATAATGAAAGTAATAAAGTTTAGTAATTGCATTGCGTT |
| | | GACTATTGATTGCATTGATGTCGTGTGATACTTTCACC |
| | | GAAAAAAAACACGAAGCGCAATAGGAGCGGTTGCAT |
| | | ATTAGTCCCCAAAGCTATTTAATTGTGCCTGAAACTGT |
| | | TTTTTAAGCTCATCAAGCATAATTGTATGCATTGCGAC |
| | | GTAACCAACGTTTAGGCGCAGTTTAATCATAGCCCAC |
| | | TGCTAAGCC |
| 52 | Sequence of the | CGGAGGAATGCAAATAATAATCTCCTTAATTACCCAC |
| | 3′-Region used | TGATAAGCTCAAGAGACGCGGTTTGAAAACGATATAA |
| | for knock out of | TGAATCATTTGGATTTTATAATAAACCCTGACAGTTTT |
| | PpPNO1 and | TCCACTGTATTGTTTTAACACTCATTGGAAGCTGTATT |
| | PpMNN4: | GATTCTAAGAAGCTAGAAATCAATACGGCCATACAAA |
| | | AGATGACATTGAATAAGCACCGGCTTTTTTGATTAGC |
| | | ATATACCTTAAAGCATGCATTCATGGCTACATAGTTGT |
| | | TAAAGGGCTTCTTCCATTATCAGTATAATGAATTACAT |
| | | AATCATGCACTTATATTTGCCCATCTCTGTTCTCTCACT |
| | | CTTGCCTGGGTATATTCTATGAAATTGCGTATAGCGTG |
| | | TCTCCAGTTGAACCCCAAGCTTGGCGAGTTTGAAGAG |
| | | AATGCTAACCTTGCGTATTCCTTGCTTCAGGAAACATT |
| | | CAAGGAGAAACAGGTCAAGAAGCCAAACATTTTGATC |
| | | CTTCCCGAGTTAGCATTGACTGGCTACAATTTTCAAAG |
| | | CCAGCAGCGGATAGAGCCTTTTTTGGAGGAAACAACC |
| | | AAGGGAGCTAGTACCCAATGGGCTCAAAAAGTATCCA |
| | | AGACGTGGGATTGCTTTACTTTAATAGGATACCCAGA |
| | | AAAAAGTTTAGAGAGCCCTCCCCGTATTTACAACAGT |
| | | GCGGTACTTGTATCGCCTCAGGGAAAAGTAATGAACA |
| | | ACTACAGAAAGTCCTTCTTGTATGAAGCTGATGAACA |
| | | TTGGGGATGTTCGGAATCTTCTGATGGGTTTCAAACAG |
| | | TAGATTTATTAATTGAAGGAAAGACTGTAAAGACATC |
| | | ATTTGGAATTTGCATGGATTTGAATCCTTATAAATTTG |
| | | AAGCTCCATTCACAGACTTCGAGTTCAGTGGCCATTGC |
| | | TTGAAAACCGGTACAAGACTCATTTTGTGCCCAATGG |
| | | CCTGGTTGTCCCCTCTATCGCCTTCCATTAAAAAGGAT |
| | | CTTAGTGATATAGAGAAAAGCAGACTTCAAAAGTTCT |
| | | ACCTTGAAAAAATAGATACCCCGGAATTTGACGTTAA |
| | | TTACGAATTGAAAAAAGATGAAGTATTGCCCACCCGT |
| | | ATGAATGAAACGTTGGAAACAATTGACTTTGAGCCTT |
| | | CAAAACCGGACTACTCTAATATAAATTATTGGATACT |
| | | AAGGTTTTTTCCCTTTCTGACTCATGTCTATAAACGAG |
| | | ATGTGCTCAAAGAGAATGCAGTTGCAGTCTTATGCAA |
| | | CCGAGTTGGCATTGAGAGTGATGTCTTGTACGGAGGA |
| | | TCAACCACGATTCTAAACTTCAATGGTAAGTTAGCATC |
| | | GACACAAGAGGAGCTGGAGTTGTACGGGCAGACTAAT |
| | | AGTCTCAACCCCAGTGTGGAAGTATTGGGGGCCCTTG |
| | | GCATGGGTCAACAGGGAATTCTAGTACGAGACATTGA |
| | | ATTAACATAATATACAATATACAATAAACACAAATAA |
| | | AGAATACAAGCCTGACAAAAATTCACAAATTATTGCC |
| | | TAGACTTGTCGTTATCAGCAGCGACCTTTTTCCAATGC |
| | | TCAATTTCACGATATGCCTTTTCTAGCTCTGCTTTAAG |
| | | CTTCTCATTGGAATTGGCTAACTCGTTGACTGCTTGGT |
| | | CAGTGATGAGTTTCTCCAAGGTCCATTTCTCGATGTTG |
| | | TTGTTTTCGTTTTCCTTTAATCTCTTGATATAATCAACA |
| | | GCCTTCTTTAATATCTGAGCCTTGTTCGAGTCCCCTGT |
| | | TGGCAACAGAGCGGCCAGTTCCTTTATTCCGTGGTTTA |
| | | TATTTTCTCTTCTACGCCTTTCTACTTCTTTGTGATTCT |
| | | CTTTACGCATCTTATGCCATTCTTCAGAACCAGTGGCT |
| | | GGCTTAACCGAATAGCCAGAGCCTGAAGAAGCCGCAC |
| | | TAGAAGAAGCAGTGGCATTGTTGACTATGG |
| 53 | DNA encodes | TCAGTCAGTGCTCTTGATGGTGACCCAGCAAGTTTGAC |
| | human GnTI | CAGAGAAGTGATTAGATTGGCCCAAGACGCAGAGGTG |
| | catalytic domain | GAGTTGGAGAGACAACGTGGACTGCTGCAGCAAATCG |
| | (NA) | GAGATGCATTGTCTAGTCAAAGAGGTAGGGTGCCTAC |
| | Codon- | CGCAGCTCCTCCAGCACAGCCTAGAGTGCATGTGACC |
| | optimized | CCTGCACCAGCTGTGATTCCTATCTTGGTCATCGCCTG |
| | | TGACAGATCTACTGTTAGAAGATGTCTGGACAAGCTG |
| | | TTGCATTACAGACCATCTGCTGAGTTGTTCCCTATCAT |
| | | CGTTAGTCAAGACTGTGGTCACGAGGAGACTGCCCAA |
| | | GCCATCGCCTCCTACGGATCTGCTGTCACTCACATCAG |
| | | ACAGCCTGACCTGTCATCTATTGCTGTGCCACCAGACC |
| | | ACAGAAAGTTCCAAGGTTACTACAAGATCGCTAGACA |
| | | CTACAGATGGGCATTGGGTCAAGTCTTCAGACAGTTT |
| | | AGATTCCCTGCTGCTGTGGTGGTGGAGGATGACTTGG |
| | | AGGTGGCTCCTGACTTCTTTGAGTACTTTAGAGCAACC |
| | | TATCCATTGCTGAAGGCAGACCCATCCCTGTGGTGTGT |
| | | CTCTGCCTGGAATGACAACGGTAAGGAGCAAATGGTG |
| | | GACGCTTCTAGGCCTGAGCTGTTGTACAGAACCGACT |
| | | TCTTTCCTGGTCTGGGATGGTTGCTGTTGGCTGAGTTG |
| | | TGGGCTGAGTTGGAGCCTAAGTGGCCAAAGGCATTCT |
| | | GGGACGACTGGATGAGAAGACCTGAGCAAAGACAGG |
| | | GTAGAGCCTGTATCAGACCTGAGATCTCAAGAACCAT |
| | | GACCTTTGGTAGAAAGGGAGTGTCTCACGGTCAATTC |
| | | TTTGACCAACACTTGAAGTTTATCAAGCTGAACCAGC |
| | | AATTTGTGCACTTCACCCAACTGGACCTGTCTTACTTG |
| | | CAGAGAGAGGCCTATGACAGAGATTTCCTAGCTAGAG |
| | | TCTACGGAGCTCCTCAACTGCAAGTGGAGAAAGTGAG |
| | | GACCAATGACAGAAAGGAGTTGGGAGAGGTGAGAGT |
| | | GCAGTACACTGGTAGGGACTCCTTTAAGGCTTTCGCTA |
| | | AGGCTCTGGGTGTCATGGATGACCTTAAGTCTGGAGT |
| | | TCCTAGAGCTGGTTACAGAGGTATTGTCACCTTTCAAT |
| | | TCAGAGGTAGAAGAGTCCACTTGGCTCCTCCACCTAC |
| | | TTGGGAGGGTTATGATCCTTCTTGGAATTAG |
| 54 | DNA encodes | ATGCCCAGAAAAATATTTAACTACTTCATTTTGACTGT |
| | Pp SEC12 (10) | ATTCATGGCAATTCTTGCTATTGTTTTACAATGGTCTA |
| | The last 9 | TAGAGAATGGACATGGGCGCGCC |
| | nucleotides are | |
| | the linker | |
| | containing the | |
| | AscI restriction | |
| | site used for | |
| | fusion to | |
| | proteins of | |
| | interest. | |
| 55 | Sequence of the | GAAGTAAAGTTGGCGAAACTTTGGGAACCTTTGGTTA |
| | PpSEC4 | AAACTTTGTAATTTTTGTCGCTACCCATTAGGCAGAAT |
| | promoter: | CTGCATCTTGGGAGGGGGATGTGGTGGCGTTCTGAGA |
| | | TGTACGCGAAGAATGAAGAGCCAGTGGTAACAACAG |
| | | GCCTAGAGAGATACGGGCATAATGGGTATAACCTACA |
| | | AGTTAAGAATGTAGCAGCCCTGGAAACCAGATTGAAA |
| | | CGAAAAACGAAATCATTTAAACTGTAGGATGTTTTGG |
| | | CTCATTGTCTGGAAGGCTGGCTGTTTATTGCCCTGTTC |
| | | TTTGCATGGGAATAAGCTATTATATCCCTCACATAATC |
| | | CCAGAAAATAGATTGAAGCAACGCGAAATCCTTACGT |
| | | ATCGAAGTAGCCTTCTTACACATTCACGTTGTACGGAT |
| | | AAGAAAACTACTCAAACGAACAATC |
| 56 | Sequence of the | AATAGATATAGCGAGATTAGAGAATGAATACCTTCTT |
| | PpOCH1 | CTAAGCGATCGTCCGTCATCATAGAATATCATGGACT |
| | terminator: | GTATAGTTTTTTTTTTGTACATATAATGATTAAACGGT |
| | | CATCCAACATCTCGTTGACAGATCTCTCAGTACGCGA |
| | | AATCCCTGACTATCAAAGCAAGAACCGATGAAGAAAA |
| | | AAACAACAGTAACCCAAACACCACAACAAACACTTTA |
| | | TCTTCTCCCCCCCAACACCAATCATCAAAGAGATGTCG |
| | | GAACACAAACACCAAGAAGCAAAAACTAACCCCATA |
| | | TAAAAACATCCTGGTAGATAATGCTGGTAACCCGCTC |
| | | TCCTTCCATATTCTGGGCTACTTCACGAAGTCTGACCG |
| | | GTCTCAGTTGATCAACATGATCCTCGAAATGG |
| 57 | DNA encodes | GAGCCCGCTGACGCCACCATCCGTGAGAAGAGGGCAA |
| | Mm ManI | AGATCAAAGAGATGATGACCCATGCTTGGAATAATTA |
| | catalytic domain | TAAACGCTATGCGTGGGGCTTGAACGAACTGAAACCT |
| | (FB) | ATATCAAAAGAAGGCCATTCAAGCAGTTTGTTTGGCA |
| | | ACATCAAAGGAGCTACAATAGTAGATGCCCTGGATAC |
| | | CCTTTTCATTATGGGCATGAAGACTGAATTTCAAGAA |
| | | GCTAAATCGTGGATTAAAAAATATTTAGATTTTAATGT |
| | | GAATGCTGAAGTTTCTGTTTTTGAAGTCAACATACGCT |
| | | TCGTCGGTGGACTGCTGTCAGCCTACTATTTGTCCGGA |
| | | GAGGAGATATTTCGAAAGAAAGCAGTGGAACTTGGGG |
| | | TAAAATTGCTACCTGCATTTCATACTCCCTCTGGAATA |
| | | CCTTGGGCATTGCTGAATATGAAAAGTGGGATCGGGC |
| | | GGAACTGGCCCTGGGCCTCTGGAGGCAGCAGTATCCT |
| | | GGCCGAATTTGGAACTCTGCATTTAGAGTTTATGCACT |
| | | TGTCCCACTTATCAGGAGACCCAGTCTTTGCCGAAAA |
| | | GGTTATGAAAATTCGAACAGTGTTGAACAAACTGGAC |
| | | AAACCAGAAGGCCTTTATCCTAACTATCTGAACCCCA |
| | | GTAGTGGACAGTGGGGTCAACATCATGTGTCGGTTGG |
| | | AGGACTTGGAGACAGCTTTTATGAATATTTGCTTAAGG |
| | | CGTGGTTAATGTCTGACAAGACAGATCTCGAAGCCAA |
| | | GAAGATGTATTTTGATGCTGTTCAGGCCATCGAGACTC |
| | | ACTTGATCCGCAAGTCAAGTGGGGGACTAACGTACAT |
| | | CGCAGAGTGGAAGGGGGGCCTCCTGGAACACAAGAT |
| | | GGGCCACCTGACGTGCTTTGCAGGAGGCATGTTTGCA |
| | | CTTGGGGCAGATGGAGCTCCGGAAGCCCGGGCCCAAC |
| | | ACTACCTTGAACTCGGAGCTGAAATTGCCCGCACTTGT |
| | | CATGAATCTTATAATCGTACATATGTGAAGTTGGGAC |
| | | CGGAAGCGTTTCGATTTGATGGCGGTGTGGAAGCTAT |
| | | TGCCACGAGGCAAAATGAAAAGTATTACATCTTACGG |
| | | CCCGAGGTCATCGAGACATACATGTACATGTGGCGAC |
| | | TGACTCACGACCCCAAGTACAGGACCTGGGCCTGGGA |
| | | AGCCGTGGAGGCTCTAGAAAGTCACTGCAGAGTGAAC |
| | | GGAGGCTACTCAGGCTTACGGGATGTTTACATTGCCC |
| | | GTGAGAGTTATGACGATGTCCAGCAAAGTTTCTTCCTG |
| | | GCAGAGACACTGAAGTATTTGTACTTGATATTTTCCGA |
| | | TGATGACCTTCTTCCACTAGAACACTGGATCTTCAACA |
| | | CCGAGGCTCATCCTTTCCCTATACTCCGTGAACAGAAG |
| | | AAGGAAATTGATGGCAAAGAGAAATGA |
| 58 | DNA encodes | ATGAACACTATCCACATAATAAAATTACCGCTTAACT |
| | ScSEC12 (8) | ACGCCAACTACACCTCAATGAAACAAAAAATCTCTAA |
| | The last 9 | ATTTTTCACCAACTTCATCCTTATTGTGCTGCTTTCTTA |
| | nucleotides are | CATTTTACAGTTCTCCTATAAGCACAATTTGCATTCCA |
| | the linker | TGCTTTTCAATTACGCGAAGGACAATTTTCTAACGAAA |
| | containing the | AGAGACACCATCTCTTCGCCCTACGTAGTTGATGAAG |
| | AscI restriction | ACTTACATCAAACAACTTTGTTTGGCAACCACGGTAC |
| | site used for | AAAAACATCTGTACCTAGCGTAGATTCCATAAAAGTG |
| | fusion to | CATGGCGTGGGGCGCGCC |
| | proteins of | |
| | interest | |
| 59 | Sequence of the | GAGTCGGCCAAGAGATGATAACTGTTACTAAGCTTCT |
| | 5′-region that | CCGTAATTAGTGGTATTTTGTAACTTTTACCAATAATC |
| | was used to | GTTTATGAATACGGATATTTTTCGACCTTATCCAGTGC |
| | knock into the | CAAATCACGTAACTTAATCATGGTTTAAATACTCCACT |
| | PpADE1 locus: | TGAACGATTCATTATTCAGAAAAAAGTCAGGTTGGCA |
| | | GAAACACTTGGGCGCTTTGAAGAGTATAAGAGTATTA |
| | | AGCATTAAACATCTGAACTTTCACCGCCCCAATATACT |
| | | ACTCTAGGAAACTCGAAAAATTCCTTTCCATGTGTCAT |
| | | CGCTTCCAACACACTTTGCTGTATCCTTCCAAGTATGT |
| | | CCATTGTGAACACTGATCTGGACGGAATCCTACCTTTA |
| | | ATCGCCAAAGGAAAGGTTAGAGACATTTATGCAGTCG |
| | | ATGAGAACAACTTGCTGTTCGTCGCAACTGACCGTAT |
| | | CTCCGCTTACGATGTGATTATGACAAACGGTATTCCTG |
| | | ATAAGGGAAAGATTTTGACTCAGCTCTCAGTTTTCTGG |
| | | TTTGATTTTTTGGCACCCTACATAAAGAATCATTTGGT |
| | | TGCTTCTAATGACAAGGAAGTCTTTGCTTTACTACCAT |
| | | CAAAACTGTCTGAAGAAAAaTACAAATCTCAATTAGA |
| | | GGGACGATCCTTGATAGTAAAAAAGCACAGACTGATA |
| | | CCTTTGGAAGCCATTGTCAGAGGTTACATCACTGGAA |
| | | GTGCATGGAAAGAGTACAAGAACTCAAAAACTGTCCA |
| | | TGGAGTCAAGGTTGAAAACGAGAACCTTCAAGAGAGC |
| | | GACGCCTTTCCAACTCCGATTTTCACACCTTCAACGAA |
| | | AGCTGAACAGGGTGAACACGATGAAAACATCTCTATT |
| | | GAACAAGCTGCTGAGATTGTAGGTAAAGACATTTGTG |
| | | AGAAGGTCGCTGTCAAGGCGGTCGAGTTGTATTCTGC |
| | | TGCAAAAAACCTCGCCCTTTTGAAGGGGATCATTATT |
| | | GCTGATACGAAATTCGAATTTGGACTGGACGAAAACA |
| | | ATGAATTGGTACTAGTAGATGAAGTTTTAACTCCAGAT |
| | | TCTTCTAGATTTTGGAATCAAAAGACTTACCAAGTGG |
| | | GTAAATCGCAAGAGAGTTACGATAAGCAGTTTCTCAG |
| | | AGATTGGTTGACGGCCAACGGATTGAATGGCAAAGAG |
| | | GGCGTAGCCATGGATGCAGAAATTGCTATCAAGAGTA |
| | | AAGAAAAGTATATTGAAGCTTATGAAGCAATTACTGG |
| | | CAAGAAATGGGCTTGA |
| 60 | Sequence of the | ATGATTAGTACCCTCCTCGCCTTTTTCAGACATCTGAA |
| | 3′-region that | ATTTCCCTTATTCTTCCAATTCCATATAAAATCCTATTT |
| | was used to | AGGTAATTAGTAAACAATGATCATAAAGTGAAATCAT |
| | knock into the | TCAAGTAACCATTCCGTTTATCGTTGATTTAAAATCAA |
| | PpADE1 locus: | TAACGAATGAATGTCGGTCTGAGTAGTCAATTTGTTGC |
| | | CTTGGAGCTCATTGGCAGGGGGTCTTTTGGCTCAGTAT |
| | | GGAAGGTTGAAAGGAAAACAGATGGAAAGTGGTTCG |
| | | TCAGAAAAGAGGTATCCTACATGAAGATGAATGCCAA |
| | | AGAGATATCTCAAGTGATAGCTGAGTTCAGAATTCTT |
| | | AGTGAGTTAAGCCATCCCAACATTGTGAAGTACCTTC |
| | | ATCACGAACATATTTCTGAGAATAAAACTGTCAATTT |
| | | ATACATGGAATACTGTGATGGTGGAGATCTCTCCAAG |
| | | CTGATTCGAACACATAGAAGGAACAAAGAGTACATTT |
| | | CAGAAGAAAAAATATGGAGTATTTTTACGCAGGTTTT |
| | | ATTAGCATTGTATCGTTGTCATTATGGAACTGATTTCA |
| | | CGGCTTCAAAGGAGTTTGAATCGCTCAATAAAGGTAA |
| | | TAGACGAACCCAGAATCCTTCGTGGGTAGACTCGACA |
| | | AGAGTTATTATTCACAGGGATATAAAACCCGACAACA |
| | | TCTTTCTGATGAACAATTCAAACCTTGTCAAACTGGGA |
| | | GATTTTGGATTAGCAAAAATTCTGGACCAAGAAAACG |
| | | ATTTTGCCAAAACATACGTCGGTACGCCGTATTACATG |
| | | TCTCCTGAAGTGCTGTTGGACCAACCCTACTCACCATT |
| | | ATGTGATATATGGTCTCTTGGGTGCGTCATGTATGAGC |
| | | TATGTGCATTGAGGCCTCCTT |
| 61 | DNA encodes | ATGACAGCTCAGTTACAAAGTGAAAGTACTTCTAAAA |
| | ScGAL10 | TTGTTTTGGTTACAGGTGGTGCTGGATACATTGGTTCA |
| | | CACACTGTGGTAGAGCTAATTGAGAATGGATATGACT |
| | | GTGTTGTTGCTGATAACCTGTCGAATTCAACTTATGAT |
| | | TCTGTAGCCAGGTTAGAGGTCTTGACCAAGCATCACA |
| | | TTCCCTTCTATGAGGTTGATTTGTGTGACCGAAAAGGT |
| | | CTGGAAAAGGTTTTCAAAGAATATAAAATTGATTCGG |
| | | TAATTCACTTTGCTGGTTTAAAGGCTGTAGGTGAATCT |
| | | ACACAAATCCCGCTGAGATACTATCACAATAACATTT |
| | | TGGGAACTGTCGTTTTATTAGAGTTAATGCAACAATAC |
| | | AACGTTTCCAAATTTGTTTTTTCATCTTCTGCTACTGTC |
| | | TATGGTGATGCTACGAGATTCCCAAATATGATTCCTAT |
| | | CCCAGAAGAATGTCCCTTAGGGCCTACTAATCCGTAT |
| | | GGTCATACGAAATACGCCATTGAGAATATCTTGAATG |
| | | ATCTTTACAATAGCGACAAAAAAAGTTGGAAGTTTGC |
| | | TATCTTGCGTTATTTTAACCCAATTGGCGCACATCCCT |
| | | CTGGATTAATCGGAGAAGATCCGCTAGGTATACCAAA |
| | | CAATTTGTTGCCATATATGGCTCAAGTAGCTGTTGGTA |
| | | GGCGCGAGAAGCTTTACATCTTCGGAGACGATTATGA |
| | | TTCCAGAGATGGTACCCCGATCAGGGATTATATCCAC |
| | | GTAGTTGATCTAGCAAAAGGTCATATTGCAGCCCTGC |
| | | AATACCTAGAGGCCTACAATGAAAATGAAGGTTTGTG |
| | | TCGTGAGTGGAACTTGGGTTCCGGTAAAGGTTCTACA |
| | | GTTTTTGAAGTTTATCATGCATTCTGCAAAGCTTCTGG |
| | | TATTGATCTTCCATACAAAGTTACGGGCAGAAGAGCA |
| | | GGTGATGTTTTGAACTTGACGGCTAAACCAGATAGGG |
| | | CCAAACGCGAACTGAAATGGCAGACCGAGTTGCAGGT |
| | | TGAAGACTCCTGCAAGGATTTATGGAAATGGACTACT |
| | | GAGAATCCTTTTGGTTACCAGTTAAGGGGTGTCGAGG |
| | | CCAGATTTTCCGCTGAAGATATGCGTTATGACGCAAG |
| | | ATTTGTGACTATTGGTGCCGGCACCAGATTTCAAGCCA |
| | | CGTTTGCCAATTTGGGCGCCAGCATTGTTGACCTGAAA |
| | | GTGAACGGACAATCAGTTGTTCTTGGCTATGAAAATG |
| | | AGGAAGGGTATTTGAATCCTGATAGTGCTTATATAGG |
| | | CGCCACGATCGGCAGGTATGCTAATCGTATTTCGAAG |
| | | GGTAAGTTTAGTTTATGCAACAAAGACTATCAGTTAA |
| | | CCGTTAATAACGGCGTTAATGCGAATCATAGTAGTAT |
| | | CGGTTCTTTCCACAGAAAAAGATTTTTGGGACCCATCA |
| | | TTCAAAATCCTTCAAAGGATGTTTTTACCGCCGAGTAC |
| | | ATGCTGATAGATAATGAGAAGGACACCGAATTTCCAG |
| | | GTGATCTATTGGTAACCATACAGTATACTGTGAACGTT |
| | | GCCCAAAAAAGTTTGGAAATGGTATATAAAGGTAAAT |
| | | TGACTGCTGGTGAAGCGACGCCAATAAATTTAACAAA |
| | | TCATAGTTATTTCAATCTGAACAAGCCATATGGAGAC |
| | | ACTATTGAGGGTACGGAGATTATGGTGCGTTCAAAAA |
| | | AATCTGTTGATGTCGACAAAAACATGATTCCTACGGG |
| | | TAATATCGTCGATAGAGAAATTGCTACCTTTAACTCTA |
| | | CAAAGCCAACGGTCTTAGGCCCCAAAAATCCCCAGTT |
| | | TGATTGTTGTTTTGTGGTGGATGAAAATGCTAAGCCAA |
| | | GTCAAATCAATACTCTAAACAATGAATTGACGCTTATT |
| | | GTCAAGGCTTTTCATCCCGATTCCAATATTACATTAGA |
| | | AGTTTTAAGTACAGAGCCAACTTATCAATTTTATACCG |
| | | GTGATTTCTTGTCTGCTGGTTACGAAGCAAGACAAGG |
| | | TTTTGCAATTGAGCCTGGTAGATACATTGATGCTATCA |
| | | ATCAAGAGAACTGGAAAGATTGTGTAACCTTGAAAAA |
| | | CGGTGAAACTTACGGGTCCAAGATTGTCTACAGATTTT |
| | | CCTGA |
| 62 | Sequence of the | TAAGCTTCACGATTTGTGTTCCAGTTTATCCCCCCTTT |
| | PpPMA1 | ATATACCGTTAACCCTTTCCCTGTTGAGCTGACTGTTG |
| | terminator: | TTGTATTACCGCAATTTTTCCAAGTTTGCCATGCTTTTC |
| | | GTGTTATTTGACCGATGTCTTTTTTCCCAAATCAAACT |
| | | ATATTTGTTACCATTTAAACCAAGTTATCTTTTGTATT |
| | | AAGAGTCTAAGTTTGTTCCCAGGCTTCATGTGAGAGT |
| | | GATAACCATCCAGACTATGATTCTTGTTTTTTATTGGG |
| | | TTTGTTTGTGTGATACATCTGAGTTGTGATTCGTAAAG |
| | | TATGTCAGTCTATCTAGATTTTTAATAGTTAATTGGTA |
| | | ATCAATGACTTGTTTGTTTTAACTTTTAAATTGTGGGT |
| | | CGTATCCACGCGTTTAGTATAGCTGTTCATGGCTGTTA |
| | | GAGGAGGGCGATGTTTATATACAGAGGACAAGAATGA |
| | | GGAGGCGGCGTGTATTTTTAAAATGGAGACGCGACTC |
| | | CTGTACACCTTATCGGTTGG |
| 63 | hGalT codon | GGTAGAGATTTGTCTAGATTGCCACAGTTGGTTGGTGT |
| | optimized (XB) | TTCCACTCCATTGCAAGGAGGTTCTAACTCTGCTGCTG |
| | | CTATTGGTCAATCTTCCGGTGAGTTGAGAACTGGTGG |
| | | AGCTAGACCACCTCCACCATTGGGAGCTTCCTCTCAAC |
| | | CAAGACCAGGTGGTGATTCTTCTCCAGTTGTTGACTCT |
| | | GGTCCAGGTCCAGCTTCTAACTTGACTTCCGTTCCAGT |
| | | TCCACACACTACTGCTTTGTCCTTGCCAGCTTGTCCAG |
| | | AAGAATCCCCATTGTTGGTTGGTCCAATGTTGATCGAG |
| | | TTCAACATGCCAGTTGACTTGGAGTTGGTTGCTAAGCA |
| | | GAACCCAAACGTTAAGATGGGTGGTAGATACGCTCCA |
| | | AGAGACTGTGTTTCCCCACACAAAGTTGCTATCATCAT |
| | | CCCATTCAGAAACAGACAGGAGCACTTGAAGTACTGG |
| | | TTGTACTACTTGCACCCAGTTTTGCAAAGACAGCAGTT |
| | | GGACTACGGTATCTACGTTATCAACCAGGCTGGTGAC |
| | | ACTATTTTCAACAGAGCTAAGTTGTTGAATGTTGGTTT |
| | | CCAGGAGGCTTTGAAGGATTACGACTACACTTGTTTC |
| | | GTTTTCTCCGACGTTGACTTGATTCCAATGAACGACCA |
| | | CAACGCTTACAGATGTTTCTCCCAGCCAAGACACATTT |
| | | CTGTTGCTATGGACAAGTTCGGTTTCTCCTTGCCATAC |
| | | GTTCAATACTTCGGTGGTGTTTCCGCTTTGTCCAAGCA |
| | | GCAGTTCTTGACTATCAACGGTTTCCCAAACAATTACT |
| | | GGGGATGGGGTGGTGAAGATGACGACATCTTTAACAG |
| | | ATTGGTTTTCAGAGGAATGTCCATCTCTAGACCAAAC |
| | | GCTGTTGTTGGTAGATGTAGAATGATCAGACACTCCA |
| | | GAGACAAGAAGAACGAGCCAAACCCACAAAGATTCG |
| | | ACAGAATCGCTCACACTAAGGAAACTATGTTGTCCGA |
| | | CGGATTGAACTCCTTGACTTACCAGGTTTTGGACGTTC |
| | | AGAGATACCCATTGTACACTCAGATCACTGTTGACAT |
| | | CGGTACTCCATCCTAG |
| 64 | DNA encodes | ATGGCCCTCTTTCTCAGTAAGAGACTGTTGAGATTTAC |
| | ScMnt1 (Kre2) | CGTCATTGCAGGTGCGGTTATTGTTCTCCTCCTAACAT |
| | (33) | TGAATTCCAACAGTAGAACTCAGCAATATATTCCGAG |
| | | TTCCATCTCCGCTGCATTTGATTTTACCTCAGGATCTA |
| | | TATCCCCTGAACAACAAGTCATCGGGCGCGCC |
| 65 | DNA encodes | ATGAATAGCATACACATGAACGCCAATACGCTGAAGT |
| | DmUGT | ACATCAGCCTGCTGACGCTGACCCTGCAGAATGCCAT |
| | | CCTGGGCCTCAGCATGCGCTACGCCCGCACCCGGCCA |
| | | GGCGACATCTTCCTCAGCTCCACGGCCGTACTCATGGC |
| | | AGAGTTCGCCAAACTGATCACGTGCCTGTTCCTGGTCT |
| | | TCAACGAGGAGGGCAAGGATGCCCAGAAGTTTGTACG |
| | | CTCGCTGCACAAGACCATCATTGCGAATCCCATGGAC |
| | | ACGCTGAAGGTGTGCGTCCCCTCGCTGGTCTATATCGT |
| | | TCAAAACAATCTGCTGTACGTCTCTGCCTCCCATTTGG |
| | | ATGCGGCCACCTACCAGGTGACGTACCAGCTGAAGAT |
| | | TCTCACCACGGCCATGTTCGCGGTTGTCATTCTGCGCC |
| | | GCAAGCTGCTGAACACGCAGTGGGGTGCGCTGCTGCT |
| | | CCTGGTGATGGGCATCGTCCTGGTGCAGTTGGCCCAA |
| | | ACGGAGGGTCCGACGAGTGGCTCAGCCGGTGGTGCCG |
| | | CAGCTGCAGCCACGGCCGCCTCCTCTGGCGGTGCTCC |
| | | CGAGCAGAACAGGATGCTCGGACTGTGGGCCGCACTG |
| | | GGCGCCTGCTTCCTCTCCGGATTCGCGGGCATCTACTT |
| | | TGAGAAGATCCTCAAGGGTGCCGAGATCTCCGTGTGG |
| | | ATGCGGAATGTGCAGTTGAGTCTGCTCAGCATTCCCTT |
| | | CGGCCTGCTCACCTGTTTCGTTAACGACGGCAGTAGG |
| | | ATCTTCGACCAGGGATTCTTCAAGGGCTACGATCTGTT |
| | | TGTCTGGTACCTGGTCCTGCTGCAGGCCGGCGGTGGA |
| | | TTGATCGTTGCCGTGGTGGTCAAGTACGCGGATAACA |
| | | TTCTCAAGGGCTTCGCCACCTCGCTGGCCATCATCATC |
| | | TCGTGCGTGGCCTCCATATACATCTTCGACTTCAATCT |
| | | CACGCTGCAGTTCAGCTTCGGAGCTGGCCTGGTCATC |
| | | GCCTCCATATTTCTCTACGGCTACGATCCGGCCAGGTC |
| | | GGCGCCGAAGCCAACTATGCATGGTCCTGGCGGCGAT |
| | | GAGGAGAAGCTGCTGCCGCGCGTCTAG |
| 66 | Sequence of the | TGGACACAGGAGACTCAGAAACAGACACAGAGCGTT |
| | PpOCH1 | CTGAGTCCTGGTGCTCCTGACGTAGGCCTAGAACAGG |
| | promoter: | AATTATTGGCTTTATTTGTTTGTCCATTTCATAGGCTTG |
| | | GGGTAATAGATAGATGACAGAGAAATAGAGAAGACC |
| | | TAATATTTTTTGTTCATGGCAAATCGCGGGTTCGCGGT |
| | | CGGGTCACACACGGAGAAGTAATGAGAAGAGCTGGT |
| | | AATCTGGGGTAAAAGGGTTCAAAAGAAGGTCGCCTGG |
| | | TAGGGATGCAATACAAGGTTGTCTTGGAGTTTACATTG |
| | | ACCAGATGATTTGGCTTTTTCTCTGTTCAATTCACATTT |
| | | TTCAGCGAGAATCGGATTGACGGAGAAATGGCGGGGT |
| | | GTGGGGTGGATAGATGGCAGAAATGCTCGCAATCACC |
| | | GCGAAAGAAAGACTTTATGGAATAGAACTACTGGGTG |
| | | GTGTAAGGATTACATAGCTAGTCCAATGGAGTCCGTT |
| | | GGAAAGGTAAGAAGAAGCTAAAACCGGCTAAGTAAC |
| | | TAGGGAAGAATGATCAGACTTTGATTTGATGAGGTCT |
| | | GAAAATACTCTGCTGCTTTTTCAGTTGCTTTTTCCCTGC |
| | | AACCTATCATTTTCCTTTTCATAAGCCTGCCTTTTCTGT |
| | | TTTCACTTATATGAGTTCCGCCGAGACTTCCCCAAATT |
| | | CTCTCCTGGAACATTCTCTATCGCTCTCCTTCCAAGTT |
| | | GCGCCCCCTGGCACTGCCTAGTAATATTACCACGCGA |
| | | CTTATATTCAGTTCCACAATTTCCAGTGTTCGTAGCAA |
| | | ATATCATCAGCC |
| 67 | Sequence of the | AATATATACCTCATTTGTTCAATTTGGTGTAAAGAGTG |
| | PpALG12 | TGGCGGATAGACTTCTTGTAAATCAGGAAAGCTACAA |
| | terminator: | TTCCAATTGCTGCAAAAAATACCAATGCCCATAAACC |
| | | AGTATGAGCGGTGCCTTCGACGGATTGCTTACTTTCCG |
| | | ACCCTTTGTCGTTTGATTCTTCTGCCTTTGGTGAGTCA |
| | | GTTTGTTTCGACTTTATATCTGACTCATCAACTTCCTTT |
| | | ACGGTTGCGTTTTTAATCATAATTTTAGCCGTTGGCTT |
| | | ATTATCCCTTGAGTTGGTAGGAGTTTTGATGATGCTG |
| 68 | Sequence of the | TAACTGGCCCTTTGACGTTTCTGACAATAGTTCTAGAG |
| | 5′-Region used | GAGTCGTCCAAAAACTCAACTCTGACTTGGGTGACAC |
| | for knock out of | CACCACGGGATCCGGTTCTTCCGAGGACCTTGATGAC |
| | PpHIS1: | CTTGGCTAATGTAACTGGAGTTTTAGTATCCATTTTAA |
| | | GATGTGTGTTTCTGTAGGTTCTGGGTTGGAAAAAAATT |
| | | TTAGACACCAGAAGAGAGGAGTGAACTGGTTTGCGTG |
| | | GGTTTAGACTGTGTAAGGCACTACTCTGTCGAAGTTTT |
| | | AGATAGGGGTTACCCGCTCCGATGCATGGGAAGCGAT |
| | | TAGCCCGGCTGTTGCCCGTTTGGTTTTTGAAGGGTAAT |
| | | TTTCAATATCTCTGTTTGAGTCATCAATTTCATATTCA |
| | | AAGATTCAAAAACAAAATCTGGTCCAAGGAGCGCATT |
| | | TAGGATTATGGAGTTGGCGAATCACTTGAACGATAGA |
| | | CTATTATTTGC |
| 69 | Sequence of the | GTGACATTCTTGTCTTTGAGATCAGTAATTGTAGAGCA |
| | 3′-Region used | TAGATAGAATAATATTCAAGACCAACGGCTTCTCTTC |
| | for knock out of | GGAAGCTCCAAGTAGCTTATAGTGATGAGTACCGGCA |
| | PpHIS1: | TATATTTATAGGCTTAAAATTTCGAGGGTTCACTATAT |
| | | TCGTTTAGTGGGAAGAGTTCCTTTCACTCTTGTTATCT |
| | | ATATTGTCAGCGTGGACTGTTTATAACTGTACCAACTT |
| | | AGTTTCTTTCAACTCCAGGTTAAGAGACATAAATGTCC |
| | | TTTGATGCTGACAATAATCAGTGGAATTCAAGGAAGG |
| | | ACAATCCCGACCTCAATCTGTTCATTAATGAAGAGTTC |
| | | GAATCGTCCTTAAATCAAGCGCTAGACTCAATTGTCA |
| | | ATGAGAACCCTTTCTTTGACCAAGAAACTATAAATAG |
| | | ATCGAATGACAAAGTTGGAAATGAGTCCATTAGCTTA |
| | | CATGATATTGAGCAGGCAGACCAAAATAAACCGTCCT |
| | | TTGAGAGCGATATTGATGGTTCGGCGCCGTTGATAAG |
| | | AGACGACAAATTGCCAAAGAAACAAAGCTGGGGGCT |
| | | GAGCAATTTTTTTTCAAGAAGAAATAGCATATGTTTAC |
| | | CACTACATGAAAATGATTCAAGTGTTGTTAAGACCGA |
| | | AAGATCTATTGCAGTGGGAACACCCCATCTTCAATAC |
| | | TGCTTCAATGGAATCTCCAATGCCAAGTACAATGCATT |
| | | TACCTTTTTCCCAGTCATCCTATACGAGCAATTCAAAT |
| | | TTTTTTTCAATTTATACTTTACTTTAGTGGCTCTCTCTC |
| | | AAGCGATACCGCAACTTCGCATTGGATATCTTTCTTCG |
| | | TATGTCGTCCCACTTTTGTTTGTACTCATAGTGACCAT |
| | | GTCAAAAGAGGCGATGGATGATATTCAACGCCGAAGA |
| | | AGGGATAGAGAACAGAACAATGAACCATATGAGGTTC |
| | | TGTCCAGCCCATCACCAGTTTTGTCCAAAAACTTAAAA |
| | | TGTGGTCACTTGGTTCGATTGCATAAGGGAATGAGAG |
| | | TGCCCGCAGATATGGTTCTTGTCCAGTCAAGCGAATCC |
| | | ACCGGAGAGTCATTTATCAAGACAGATCAGCTGGATG |
| | | GTGAGACTGATTGGAAGCTTCGGATTGTTTCTCCAGTT |
| | | ACACAATCGTTACCAATGACTGAACTTCAAAATGTCG |
| | | CCATCACTGCAAGCGCACCCTCAAAATCAATTCACTC |
| | | CTTTCTTGGAAGATTGACCTACAATGGGCAATCATATG |
| | | GTCTTACGATAGACAACACAATGTGGTGTAATACTGT |
| | | ATTAGCTTCTGGTTCAGCAATTGGTTGTATAATTTACA |
| | | CAGGTAAAGATACTCGACAATCGATGAACACAACTCA |
| | | GCCCAAACTGAAAACGGGCTTGTTAGAACTGGAAATC |
| | | AATAGTTTGTCCAAGATCTTATGTGTTTGTGTGTTTGC |
| | | ATTATCTGTCATCTTAGTGCTATTCCAAGGAATAGCTG |
| | | ATGATTGGTACGTCGATATCATGCGGTTTCTCATTCTA |
| | | TTCTCCACTATTATCCCAGTGTCTCTGAGAGTTAACCT |
| | | TGATCTTGGAAAGTCAGTCCATGCTCATCAAATAGAA |
| | | ACTGATAGCTCAATACCTGAAACCGTTGTTAGAACTA |
| | | GTACAATACCGGAAGACCTGGGAAGAATTGAATACCT |
| | | ATTAAGTGACAAAACTGGAACTCTTACTCAAAATGAT |
| | | ATGGAAATGAAAAAACTACACCTAGGAACAGTCTCTT |
| | | ATGCTGGTGATACCATGGATATTATTTCTGATCATGTT |
| | | AAAGGTCTTAATAACGCTAAAACATCGAGGAAAGATC |
| | | TTGGTATGAGAATAAGAGATTTGGTTACAACTCTGGC |
| | | CATCTG |
| 70 | DNA encodes | AGAGACGATCCAATTAGACCTCCATTGAAGGTTGCTA |
| | Drosophila | GATCCCCAAGACCAGGTCAATGTCAAGATGTTGTTCA |
| | melanogaster | GGACGTCCCAAACGTTGATGTCCAGATGTTGGAGTTG |
| | ManII codon- | TACGATAGAATGTCCTTCAAGGACATTGATGGTGGTG |
| | optimized (KD) | TTTGGAAGCAGGGTTGGAACATTAAGTACGATCCATT |
| | | GAAGTACAACGCTCATCACAAGTTGAAGGTCTTCGTT |
| | | GTCCCACACTCCCACAACGATCCTGGTTGGATTCAGA |
| | | CCTTCGAGGAATACTACCAGCACGACACCAAGCACAT |
| | | CTTGTCCAACGCTTTGAGACATTTGCACGACAACCCA |
| | | GAGATGAAGTTCATCTGGGCTGAAATCTCCTACTTCGC |
| | | TAGATTCTACCACGATTTGGGTGAGAACAAGAAGTTG |
| | | CAGATGAAGTCCATCGTCAAGAACGGTCAGTTGGAAT |
| | | TCGTCACTGGTGGATGGGTCATGCCAGACGAGGCTAA |
| | | CTCCCACTGGAGAAACGTTTTGTTGCAGTTGACCGAA |
| | | GGTCAAACTTGGTTGAAGCAATTCATGAACGTCACTC |
| | | CAACTGCTTCCTGGGCTATCGATCCATTCGGACACTCT |
| | | CCAACTATGCCATACATTTTGCAGAAGTCTGGTTTCAA |
| | | GAATATGTTGATCCAGAGAACCCACTACTCCGTTAAG |
| | | AAGGAGTTGGCTCAACAGAGACAGTTGGAGTTCTTGT |
| | | GGAGACAGATCTGGGACAACAAAGGTGACACTGCTTT |
| | | GTTCACCCACATGATGCCATTCTACTCTTACGACATTC |
| | | CTCATACCTGTGGTCCAGATCCAAAGGTTTGTTGTCAG |
| | | TTCGATTTCAAAAGAATGGGTTCCTTCGGTTTGTCTTG |
| | | TCCATGGAAGGTTCCACCTAGAACTATCTCTGATCAA |
| | | AATGTTGCTGCTAGATCCGATTTGTTGGTTGATCAGTG |
| | | GAAGAAGAAGGCTGAGTTGTACAGAACCAACGTCTTG |
| | | TTGATTCCATTGGGTGACGACTTCAGATTCAAGCAGA |
| | | ACACCGAGTGGGATGTTCAGAGAGTCAACTACGAAAG |
| | | ATTGTTCGAACACATCAACTCTCAGGCTCACTTCAATG |
| | | TCCAGGCTCAGTTCGGTACTTTGCAGGAATACTTCGAT |
| | | GCTGTTCACCAGGCTGAAAGAGCTGGACAAGCTGAGT |
| | | TCCCAACCTTGTCTGGTGACTTCTTCACTTACGCTGAT |
| | | AGATCTGATAACTACTGGTCTGGTTACTACACTTCCAG |
| | | ACCATACCATAAGAGAATGGACAGAGTCTTGATGCAC |
| | | TACGTTAGAGCTGCTGAAATGTTGTCCGCTTGGCACTC |
| | | CTGGGACGGTATGGCTAGAATCGAGGAAAGATTGGAG |
| | | CAGGCTAGAAGAGAGTTGTCCTTGTTCCAGCACCACG |
| | | ACGGTATTACTGGTACTGCTAAAACTCACGTTGTCGTC |
| | | GACTACGAGCAAAGAATGCAGGAAGCTTTGAAAGCTT |
| | | GTCAAATGGTCATGCAACAGTCTGTCTACAGATTGTTG |
| | | ACTAAGCCATCCATCTACTCTCCAGACTTCTCCTTCTC |
| | | CTACTTCACTTTGGACGACTCCAGATGGCCAGGTTCTG |
| | | GTGTTGAGGACTCTAGAACTACCATCATCTTGGGTGA |
| | | GGATATCTTGCCATCCAAGCATGTTGTCATGCACAAC |
| | | ACCTTGCCACACTGGAGAGAGCAGTTGGTTGACTTCT |
| | | ACGTCTCCTCTCCATTCGTTTCTGTTACCGACTTGGCT |
| | | AACAATCCAGTTGAGGCTCAGGTTTCTCCAGTTTGGTC |
| | | TTGGCACCACGACACTTTGACTAAGACTATCCACCCA |
| | | CAAGGTTCCACCACCAAGTACAGAATCATCTTCAAGG |
| | | CTAGAGTTCCACCAATGGGTTTGGCTACCTACGTTTTG |
| | | ACCATCTCCGATTCCAAGCCAGAGCACACCTCCTACG |
| | | CTTCCAATTTGTTGCTTAGAAAGAACCCAACTTCCTTG |
| | | CCATTGGGTCAATACCCAGAGGATGTCAAGTTCGGTG |
| | | ATCCAAGAGAGATCTCCTTGAGAGTTGGTAACGGTCC |
| | | AACCTTGGCTTTCTCTGAGCAGGGTTTGTTGAAGTCCA |
| | | TTCAGTTGACTCAGGATTCTCCACATGTTCCAGTTCAC |
| | | TTCAAGTTCTTGAAGTACGGTGTTAGATCTCATGGTGA |
| | | TAGATCTGGTGCTTACTTGTTCTTGCCAAATGGTCCAG |
| | | CTTCTCCAGTCGAGTTGGGTCAGCCAGTTGTCTTGGTC |
| | | ACTAAGGGTAAATTGGAGTCTTCCGTTTCTGTTGGTTT |
| | | GCCATCTGTCGTTCACCAGACCATCATGAGAGGTGGT |
| | | GCTCCAGAGATTAGAAATTTGGTCGATATTGGTTCTTT |
| | | GGACAACACTGAGATCGTCATGAGATTGGAGACTCAT |
| | | ATCGACTCTGGTGATATCTTCTACACTGATTTGAATGG |
| | | ATTGCAATTCATCAAGAGGAGAAGATTGGACAAGTTG |
| | | CCATTGCAGGCTAACTACTACCCAATTCCATCTGGTAT |
| | | GTTCATTGAGGATGCTAATACCAGATTGACTTTGTTGA |
| | | CCGGTCAACCATTGGGTGGATCTTCTTTGGCTTCTGGT |
| | | GAGTTGGAGATTATGCAAGATAGAAGATTGGCTTCTG |
| | | ATGATGAAAGAGGTTTGGGTCAGGGTGTTTTGGACAA |
| | | CAAGCCAGTTTTGCATATTTACAGATTGGTCTTGGAGA |
| | | AGGTTAACAACTGTGTCAGACCATCTAAGTTGCATCC |
| | | AGCTGGTTACTTGACTTCTGCTGCTCACAAAGCTTCTC |
| | | AGTCTTTGTTGGATCCATTGGACAAGTTCATCTTCGCT |
| | | GAAAATGAGTGGATCGGTGCTCAGGGTCAATTCGGTG |
| | | GTGATCATCCATCTGCTAGAGAGGATTTGGATGTCTCT |
| | | GTCATGAGAAGATTGACCAAGTCTTCTGCTAAAACCC |
| | | AGAGAGTTGGTTACGTTTTGCACAGAACCAATTTGAT |
| | | GCAATGTGGTACTCCAGAGGAGCATACTCAGAAGTTG |
| | | GATGTCTGTCACTTGTTGCCAAATGTTGCTAGATGTGA |
| | | GAGAACTACCTTGACTTTCTTGCAGAATTTGGAGCACT |
| | | TGGATGGTATGGTTGCTCCAGAAGTTTGTCCAATGGA |
| | | AACCGCTGCTTACGTCTCTTCTCACTCTTCTTGA |
| 71 | DNA encodes | ATGCTGCTTACCAAAAGGTTTTCAAAGCTGTTCAAGCT |
| | Mnn2 leader | GACGTTCATAGTTTTGATATTGTGCGGGCTGTTCGTCA |
| | (53) | TTACAAACAAATACATGGATGAGAACACGTCG |
| 72 | Sequence of the | CAAGTTGCGTCCGGTATACGTAACGTCTCACGATGAT |
| | PpHIS1 | CAAAGATAATACTTAATCTTCATGGTCTACTGAATAAC |
| | auxotrophic | TCATTTAAACAATTGACTAATTGTACATTATATTGAAC |
| | marker: | TTATGCATCCTATTAACGTAATCTTCTGGCTTCTCTCTC |
| | | AGACTCCATCAGACACAGAATATCGTTCTCTCTAACTG |
| | | GTCCTTTGACGTTTCTGACAATAGTTCTAGAGGAGTCG |
| | | TCCAAAAACTCAACTCTGACTTGGGTGACACCACCAC |
| | | GGGATCCGGTTCTTCCGAGGACCTTGATGACCTTGGCT |
| | | AATGTAACTGGAGTTTTAGTATCCATTTTAAGATGTGT |
| | | GTTTCTGTAGGTTCTGGGTTGGAAAAAAATTTTAGACA |
| | | CCAGAAGAGAGGAGTGAACTGGTTTGCGTGGGTTTAG |
| | | ACTGTGTAAGGCACTACTCTGTCGAAGTTTTAGATAG |
| | | GGGTTACCCGCTCCGATGCATGGGAAGCGATTAGCCC |
| | | GGCTGTTGCCCGTTTGGTTTTTGAAGGGTAATTTTCAA |
| | | TATCTCTGTTTGAGTCATCAATTTCATATTCAAAGATT |
| | | CAAAAACAAAATCTGGTCCAAGGAGCGCATTTAGGAT |
| | | TATGGAGTTGGCGAATCACTTGAACGATAGACTATTA |
| | | TTTGCTGTTCCTAAAGAGGGCAGATTGTATGAGAAAT |
| | | GCGTTGAATTACTTAGGGGATCAGATATTCAGTTTCGA |
| | | AGATCCAGTAGATTGGATATAGCTTTGTGCACTAACCT |
| | | GCCCCTGGCATTGGTTTTCCTTCCAGCTGCTGACATTC |
| | | CCACGTTTGTAGGAGAGGGTAAATGTGATTTGGGTAT |
| | | AACTGGTATTGACCAGGTTCAGGAAAGTGACGTAGAT |
| | | GTCATACCTTTATTAGACTTGAATTTCGGTAAGTGCAA |
| | | GTTGCAGATTCAAGTTCCCGAGAATGGTGACTTGAAA |
| | | GAACCTAAACAGCTAATTGGTAAAGAAATTGTTTCCT |
| | | CCTTTACTAGCTTAACCACCAGGTACTTTGAACAACTG |
| | | GAAGGAGTTAAGCCTGGTGAGCCACTAAAGACAAAA |
| | | ATCAAATATGTTGGAGGGTCTGTTGAGGCCTCTTGTGC |
| | | CCTAGGAGTTGCCGATGCTATTGTGGATCTTGTTGAGA |
| | | GTGGAGAAACCATGAAAGCGGCAGGGCTGATCGATAT |
| | | TGAAACTGTTCTTTCTACTTCCGCTTACCTGATCTCTTC |
| | | GAAGCATCCTCAACACCCAGAACTGATGGATACTATC |
| | | AAGGAGAGAATTGAAGGTGTACTGACTGCTCAGAAGT |
| | | ATGTCTTGTGTAATTACAACGCACCTAGAGGTAACCTT |
| | | CCTCAGCTGCTAAAACTGACTCCAGGCAAGAGAGCTG |
| | | CTACCGTTTCTCCATTAGATGAAGAAGATTGGGTGGG |
| | | AGTGTCCTCGATGGTAGAGAAGAAAGATGTTGGAAGA |
| | | ATCATGGACGAATTAAAGAAACAAGGTGCCAGTGACA |
| | | TTCTTGTCTTTGAGATCAGTAATTGTAGAGCATAGATA |
| | | GAATAATATTCAAGACCAACGGCTTCTCTTCGGAAGC |
| | | TCCAAGTAGCTTATAGTGATGAGTACCGGCATATATTT |
| | | ATAGGCTTAAAATTTCGAGGGTTCACTATATTCGTTTA |
| | | GTGGGAAGAGTTCCTTTCACTCTTGTTATCTATATTGT |
| | | CAGCGTGGACTGTTTATAACTGTACCAACTTAGTTTCT |
| | | TTCAACTCCAGGTTAAGAGACATAAATGTCCTTTGATGC |
| 73 | DNA encodes | TCCTTGGTTTACCAATTGAACTTCGACCAGATGTTGAG |
| | Rat GnT II | AAACGTTGACAAGGACGGTACTTGGTCTCCTGGTGAG |
| | (TC) | TTGGTTTTGGTTGTTCAGGTTCACAACAGACCAGAGTA |
| | Codon- | CTTGAGATTGTTGATCGACTCCTTGAGAAAGGCTCAA |
| | optimized | GGTATCAGAGAGGTTTTGGTTATCTTCTCCCACGATTT |
| | | CTGGTCTGCTGAGATCAACTCCTTGATCTCCTCCGTTG |
| | | ACTTCTGTCCAGTTTTGCAGGTTTTCTTCCCATTCTCCA |
| | | TCCAATTGTACCCATCTGAGTTCCCAGGTTCTGATCCA |
| | | AGAGACTGTCCAAGAGACTTGAAGAAGAACGCTGCTT |
| | | TGAAGTTGGGTTGTATCAACGCTGAATACCCAGATTCT |
| | | TTCGGTCACTACAGAGAGGCTAAGTTCTCCCAAACTA |
| | | AGCATCATTGGTGGTGGAAGTTGCACTTTGTTTGGGAG |
| | | AGAGTTAAGGTTTTGCAGGACTACACTGGATTGATCTT |
| | | GTTCTTGGAGGAGGATCATTACTTGGCTCCAGACTTCT |
| | | ACCACGTTTTCAAGAAGATGTGGAAGTTGAAGCAACA |
| | | AGAGTGTCCAGGTTGTGACGTTTTGTCCTTGGGAACTT |
| | | ACACTACTATCAGATCCTTCTACGGTATCGCTGACAAG |
| | | GTTGACGTTAAGACTTGGAAGTCCACTGAACACAACA |
| | | TGGGATTGGCTTTGACTAGAGATGCTTACCAGAAGTT |
| | | GATCGAGTGTACTGACACTTTCTGTACTTACGACGACT |
| | | ACAACTGGGACTGGACTTTGCAGTACTTGACTTTGGCT |
| | | TGTTTGCCAAAAGTTTGGAAGGTTTTGGTTCCACAGGC |
| | | TCCAAGAATTTTCCACGCTGGTGACTGTGGAATGCAC |
| | | CACAAGAAAACTTGTAGACCATCCACTCAGTCCGCTC |
| | | AAATTGAGTCCTTGTTGAACAACAACAAGCAGTACTT |
| | | GTTCCCAGAGACTTTGGTTATCGGAGAGAAGTTTCCA |
| | | ATGGCTGCTATTTCCCCACCAAGAAAGAATGGTGGAT |
| | | GGGGTGATATTAGAGACCACGAGTTGTGTAAATCCTA |
| | | CAGAAGATTGCAGTAG |
| 74 | DNA encodes | ATGCTGCTTACCAAAAGGTTTTCAAAGCTGTTCAAGCT |
| | Mnn2 leader | GACGTTCATAGTTTTGATATTGTGCGGGCTGTTCGTCA |
| | (54) | TTACAAACAAATACATGGATGAGAACACGTCGGTCAA |
| | The last 9 | GGAGTACAAGGAGTACTTAGACAGATATGTCCAGAGT |
| | nucleotides are | TACTCCAATAAGTATTCATCTTCCTCAGACGCCGCCAG |
| | the linker | CGCTGACGATTCAACCCCATTGAGGGACAATGATGAG |
| | containing the | GCAGGCAATGAAAAGTTGAAAAGCTTCTACAACAACG |
| | AscI restriction | TTTTCAACTTTCTAATGGTTGATTCGCCCGGGCGCGCC |
| | site) | |
| 75 | Sequence of the | GATCTGGCCTTCCCTGAATTTTTACGTCCAGCTATACG |
| | 5′-Region used | ATCCGTTGTGACTGTATTTCCTGAAATGAAGTTTCAAC |
| | for knock out of | CTAAAGTTTTGGTTGTACTTGCTCCACCTACCACGGAA |
| | PpARG1: | ACTAATATCGAAACCAATGAAAAAGTAGAACTGGAAT |
| | | CGTCAATCGAAATTCGCAACCAAGTGGAACCCAAAGA |
| | | CTTGAATCTTTCTAAAGTCTATTCTAGTGACACTAATG |
| | | GCAACAGAAGATTTGAGCTGACTTTTCAAATGAATCT |
| | | CAATAATGCAATATCAACATCAGACAATCAATGGGCT |
| | | TTGTCTAGTGACACAGGATCAATTATAGTAGTGTCTTC |
| | | TGCAGGAAGAATAACTTCCCCGATCCTAGAAGTCGGG |
| | | GCATCCGTCTGTGTCTTAAGATCGTACAACGAACACCT |
| | | TTTGGCAATAACTTGTGAAGGAACATGCTTTTCATGGA |
| | | ATTTAAAGAAGCAAGAATGTGTTCTAAACAGCATTTC |
| | | ATTAGCACCTATAGTCAATTCACACATGCTAGTTAAG |
| | | AAAGTTGGAGATGCAAGGAACTATTCTATTGTATCTG |
| | | CCGAAGGAGACAACAATCCGTTACCCCAGATTCTAGA |
| | | CTGCGAACTTTCCAAAAATGGCGCTCCAATTGTGGCTC |
| | | TTAGCACGAAAGACATCTACTCTTATTCAAAGAAAAT |
| | | GAAATGCTGGATCCATTTGATTGATTCGAAATACTTTG |
| | | AATTGTTGGGTGCTGACAATGCACTGTTTGAGTGTGTG |
| | | GAAGCGCTAGAAGGTCCAATTGGAATGCTAATTCATA |
| | | GATTGGTAGATGAGTTCTTCCATGAAAACACTGCCGG |
| | | TAAAAAACTCAAACTTTACAACAAGCGAGTACTGGAG |
| | | GACCTTTCAAATTCACTTGAAGAACTAGGTGAAAATG |
| | | CGTCTCAATTAAGAGAGAAACTTGACAAACTCTATGG |
| | | TGATGAGGTTGAGGCTTCTTGACCTCTTCTCTCTATCT |
| | | GCGTTTCTTTTTTTTTTTTTTTTTTTTTTTTTTTCAGTTG |
| | | AGCCAGACCGCGCTAAACGCATACCAATTGCCAAATC |
| | | AGGCAATTGTGAGACAGTGGTAAAAAAGATGCCTGCA |
| | | AAGTTAGATTCACACAGTAAGAGAGATCCTACTCATA |
| | | AATGAGGCGCTTATTTAGTAGCTAGTGATAGCCACTG |
| | | CGGTTCTGCTTTATGCTATTTGTTGTATGCCTTACTATC |
| | | TTTGTTTGGCTCCTTTTTCTTGACGTTTTCCGTTGGAGG |
| | | GACTCCCTATTCTGAGTCATGAGCCGCACAGATTATCG |
| | | CCCAAAATTGACAAAATCTTCTGGCGAAAAAAGTATA |
| | | AAAGGAGAAAAAAGCTCACCCTTTTCCAGCGTAGAAA |
| | | GTATATATCAGTCATTGAAGAC |
| 76 | Sequence of the | GGGACTTTAACTCAAGTAAAAGGATAGTTGTACAATT |
| | 3′-Region used | ATATATACGAAGAATAAATCATTACAAAAAGTATTCG |
| | for knock out of | TTTCTTTGATTCTTAACAGGATTCATTTTCTGGGTGTCA |
| | PpARG1: | TCAGGTACAGCGCTGAATATCTTGAAGTTAACATCGA |
| | | GCTCATCATCGACGTTCATCACACTAGCCACGTTTCCG |
| | | CAACGGTAGCAATAATTAGGAGCGGACCACACAGTGA |
| | | CGACATCTTTCTCTTTGAAATGGTATCTGAAGCCTTCC |
| | | ATGACCAATTGATGGGCTCTAGCGATGAGTTGCAAGT |
| | | TATTAATGTGGTTGAACTCACGTGCTACTCGAGCACCG |
| | | AATAACCAGCCAGCTCCACGAGGAGAAACAGCCCAA |
| | | CTGTCGACTTCATCTGGGTCAGACCAAACCAAGTCAC |
| | | AAAATCCTCCTTCATGAGGGACCTCTTGCGCTCGGCTG |
| | | AGAACTCTGATTTGATCTAACATGCGAATATCGGGAG |
| | | AGAGACCACCATGGATACATAATATTTTACCATCAAT |
| | | GATGGCACTAAGGGTTAAAAAGTCGAACACCTGGCAA |
| | | CAGTACTTCCAGACAGTGGTGGAACCATATTTATTGA |
| | | GACATTCCTCATAAAATCCATAAACCTGAGTGATCTGT |
| | | CTGGATTCATGATTTCCCCTTACCAATGTGATATGTTG |
| | | AGGAAACTTAATTTTTAAAATCATGAGTAACGTGAAC |
| | | GTCTCCAACGAGAAATAGCCTCTATCCACATAGTCTCC |
| | | TAGGAAGATATAGTTCTGTTTTATTCCATTAGAGGAGG |
| | | ATCCGGGAAACCCACCACTAATCTTGAAAAGTTCCAG |
| | | TAGATCGTGAAATTGGCCGTGAATATCTCCGCATACT |
| | | GTCACTGGACTCTGCACTGGCTGTATATTGGATTCCTC |
| | | CATCAGCAAATCCTTCACCCGTTCGCAAAGATGCTTCA |
| | | TATCATTTTCACTTAAAGCCTTGCAGCTTTTGACTTCTT |
| | | CAAACCACTGATCTGGTCCTCTTTCTGGCATGATTAAG |
| | | GTCTATAATATTTCTGAGCTGAGATGTAAAAAAAAAT |
| | | AATAAAAATGGGGAGTGAAAAAGTGTGTAGCTTTTAG |
| | | GAGTTTGGGATTGATACCCCAAAATGATCTTTATGAG |
| | | AATTAAAAGGTAGATACGCTTTTAATAAGAACACCTA |
| | | TCTATAGTACTTTGTGGTCTTGAGTAATTGAGATGTTC |
| | | AGCTTCTGAGGTTTGCCGTTATTCTGGGATAGTAGTGC |
| | | GCGACCAAACAACCCGCCAGGCAAAGTGTGTTGTGCT |
| | | CGAAGACGATTGCCAGAAGAGTAAGTCCGTCCTGCCT |
| | | CAGATGTTACACACTTTCTTCCCTAGACAGTCGATGCA |
| | | TCATCGGATTTAAACCTGAAACTTTGATGCCATGATAC |
| | | GCCTAGTCACGTCGACTGAGATTTTAGATAAGCCCCG |
| | | ATCCCTTTAGTACATTCCTGTTATCCATGGATGGAATG |
| | | GCCTGATA |
| 77 | Sequence of the | AAGCTTGTTCACCGTTGGGACTTTTCCGTGGACAATGT |
| | 5′-Region used | TGACTACTCCAGGAGGGATTCCAGCTTTCTCTACTAGC |
| | for knock out of | TCAGCAATAATCAATGCAGCCCCAGGCGCCCGTTCTG |
| | BMT4 | ATGGCTTGATGACCGTTGTATTGCCTGTCACTATAGCC |
| | | AGGGGTAGGGTCCATAAAGGAATCATAGCAGGGAAA |
| | | TTAAAAGGGCATATTGATGCAATCACTCCCAATGGCT |
| | | CTCTTGCCATTGAAGTCTCCATATCAGCACTAACTTCC |
| | | AAGAAGGACCCCTTCAAGTCTGACGTGATAGAGCACG |
| | | CTTGCTCTGCCACCTGTAGTCCTCTCAAAACGTCACCT |
| | | TGTGCATCAGCAAAGACTTTACCTTGCTCCAATACTAT |
| | | GACGGAGGCAATTCTGTCAAAATTCTCTCTCAGCAATT |
| | | CAACCAACTTGAAAGCAAATTGCTGTCTCTTGATGAT |
| | | GGAGACTTTTTTCCAAGATTGAAATGCAATGTGGGAC |
| | | GACTCAATTGCTTCTTCCAGCTCCTCTTCGGTTGATTG |
| | | AGGAACTTTTGAAACCACAAAATTGGTCGTTGGGTCA |
| | | TGTACATCAAACCATTCTGTAGATTTAGATTCGACGAA |
| | | AGCGTTGTTGATGAAGGAAAAGGTTGGATACGGTTTG |
| | | TCGGTCTCTTTGGTATGGCCGGTGGGGTATGCAATTGC |
| | | AGTAGAAGATAATTGGACAGCCATTGTTGAAGGTAGA |
| | | GAAAAGGTCAGGGAACTTGGGGGTTATTTATACCATT |
| | | TTACCCCACAAATAACAACTGAAAAGTACCCATTCCA |
| | | TAGTGAGAGGTAACCGACGGAAAAAGACGGGCCCAT |
| | | GTTCTGGGACCAATAGAACTGTGTAATCCATTGGGAC |
| | | TAATCAACAGACGATTGGCAATATAATGAAATAGTTC |
| | | GTTGAAAAGCCACGTCAGCTGTCTTTTCATTAACTTTG |
| | | GTCGGACACAACATTTTCTACTGTTGTATCTGTCCTAC |
| | | TTTGCTTATCATCTGCCACAGGGCAAGTGGATTTCCTT |
| | | CTCGCGCGGCTGGGTGAAAACGGTTAACGTGAA |
| 78 | Sequence of the | GCCTTGGGGGACTTCAAGTCTTTGCTAGAAACTAGAT |
| | 3′-Region used | GAGGTCAGGCCCTCTTATGGTTGTGTCCCAATTGGGCA |
| | for knock out of | ATTTCACTCACCTAAAAAGCATGACAATTATTTAGCG |
| | BMT4 | AAATAGGTAGTATATTTTCCCTCATCTCCCAAGCAGTT |
| | | TCGTTTTTGCATCCATATCTCTCAAATGAGCAGCTACG |
| | | ACTCATTAGAACCAGAGTCAAGTAGGGGTGAGCTCAG |
| | | TCATCAGCCTTCGTTTCTAAAACGATTGAGTTCTTTTG |
| | | TTGCTACAGGAAGCGCCCTAGGGAACTTTCGCACTTT |
| | | GGAAATAGATTTTGATGACCAAGAGCGGGAGTTGATA |
| | | TTAGAGAGGCTGTCCAAAGTACATGGGATCAGGCCGG |
| | | CCAAATTGATTGGTGTGACTAAACCATTGTGTACTTGG |
| | | ACACTCTATTACAAAAGCGAAGATGATTTGAAGTATT |
| | | ACAAGTCCCGAAGTGTTAGAGGATTCTATCGAGCCCA |
| | | GAATGAAATCATCAACCGTTATCAGCAGATTGATAAA |
| | | CTCTTGGAAAGCGGTATCCCATTTTCATTATTGAAGAA |
| | | CTACGATAATGAAGATGTGAGAGACGGCGACCCTCTG |
| | | AACGTAGACGAAGAAACAAATCTACTTTTGGGGTACA |
| | | ATAGAGAAAGTGAATCAAGGGAGGTATTTGTGGCCAT |
| | | AATACTCAACTCTATCATTAATG |
| 79 | Sequence of the | CATATGGTGAGAGCCGTTCTGCACAACTAGATGTTTTC |
| | 5′-Region used | GAGCTTCGCATTGTTTCCTGCAGCTCGACTATTGAATT |
| | for knock out of | AAGATTTCCGGATATCTCCAATCTCACAAAAACTTATG |
| | BMT1 | TTGACCACGTGCTTTCCTGAGGCGAGGTGTTTTATATG |
| | | CAAGCTGCCAAAAATGGAAAACGAATGGCCATTTTTC |
| | | GCCCAGGCAAATTATTCGATTACTGCTGTCATAAAGA |
| | | CAGTGTTGCAAGGCTCACATTTTTTTTTAGGATCCGAG |
| | | ATAAAGTGAATACAGGACAGCTTATCTCTATATCTTGT |
| | | ACCATTCGTGAATCTTAAGAGTTCGGTTAGGGGGACT |
| | | CTAGTTGAGGGTTGGCACTCACGTATGGCTGGGCGCA |
| | | GAAATAAAATTCAGGCGCAGCAGCACTTATCGATG |
| 80 | Sequence of the | GAATTCACAGTTATAAATAAAAACAAAAACTCAAAAA |
| | 3′-Region used | GTTTGGGCTCCACAAAATAACTTAATTTAAATTTTTGT |
| | for knock out of | CTAATAAATGAATGTAATTCCAAGATTATGTGATGCA |
| | BMT1 | AGCACAGTATGCTTCAGCCCTATGCAGCTACTAATGTC |
| | | AATCTCGCCTGCGAGCGGGCCTAGATTTTCACTACAA |
| | | ATTTCAAAACTACGCGGATTTATTGTCTCAGAGAGCA |
| | | ATTTGGCATTTCTGAGCGTAGCAGGAGGCTTCATAAG |
| | | ATTGTATAGGACCGTACCAACAAATTGCCGAGGCACA |
| | | ACACGGTATGCTGTGCACTTATGTGGCTACTTCCCTAC |
| | | AACGGAATGAAACCTTCCTCTTTCCGCTTAAACGAGA |
| | | AAGTGTGTCGCAATTGAATGCAGGTGCCTGTGCGCCT |
| | | TGGTGTATTGTTTTTGAGGGCCCAATTTATCAGGCGCC |
| | | TTTTTTCTTGGTTGTTTTCCCTTAGCCTCAAGCAAGGTT |
| | | GGTCTATTTCATCTCCGCTTCTATACCGTGCCTGATAC |
| | | TGTTGGATGAGAACACGACTCAACTTCCTGCTGCTCTG |
| | | TATTGCCAGTGTTTTGTCTGTGATTTGGATCGGAGTCC |
| | | TCCTTACTTGGAATGATAATAATCTTGGCGGAATCTCC |
| | | CTAAACGGAGGCAAGGATTCTGCCTATGATGATCTGC |
| | | TATCATTGGGAAGCTT |
| 81 | Sequence of the | GATATCTCCCTGGGGACAATATGTGTTGCAACTGTTCG |
| | 5′-Region used | TTGTTGGTGCCCCAGTCCCCCAACCGGTACTAATCGGT |
| | for knock out of | CTATGTTCCCGTAACTCATATTCGGTTAGAACTAGAAC |
| | BMT3 | AATAAGTGCATCATTGTTCAACATTGTGGTTCAATTGT |
| | | CGAACATTGCTGGTGCTTATATCTACAGGGAAGACGA |
| | | TAAGCCTTTGTACAAGAGAGGTAACAGACAGTTAATT |
| | | GGTATTTCTTTGGGAGTCGTTGCCCTCTACGTTGTCTC |
| | | CAAGACATACTACATTCTGAGAAACAGATGGAAGACT |
| | | CAAAAATGGGAGAAGCTTAGTGAAGAAGAGAAAGTT |
| | | GCCTACTTGGACAGAGCTGAGAAGGAGAACCTGGGTT |
| | | CTAAGAGGCTGGACTTTTTGTTCGAGAGTTAAACTGC |
| | | ATAATTTTTTCTAAGTAAATTTCATAGTTATGAAATTT |
| | | CTGCAGCTTAGTGTTTACTGCATCGTTTACTGCATCAC |
| | | CCTGTAAATAATGTGAGCTTTTTTCCTTCCATTGCTTG |
| | | GTATCTTCCTTGCTGCTGTTT |
| 82 | Sequence of the | ACAAAACAGTCATGTACAGAACTAACGCCTTTAAGAT |
| | 3′-Region used | GCAGACCACTGAAAAGAATTGGGTCCCATTTTTCTTG |
| | for knock out of | AAAGACGACCAGGAATCTGTCCATTTTGTTTACTCGTT |
| | BMT3 | CAATCCTCTGAGAGTACTCAACTGCAGTCTTGATAAC |
| | | GGTGCATGTGATGTTCTATTTGAGTTACCACATGATTT |
| | | TGGCATGTCTTCCGAGCTACGTGGTGCCACTCCTATGC |
| | | TCAATCTTCCTCAGGCAATCCCGATGGCAGACGACAA |
| | | AGAAATTTGGGTTTCATTCCCAAGAACGAGAATATCA |
| | | GATTGCGGGTGTTCTGAAACAATGTACAGGCCAATGT |
| | | TAATGCTTTTTGTTAGAGAAGGAACAAACTTTTTTGCT |
| | | GAGC |
| 83 | DNA encodes Tr | CGCGCCGGATCTCCCAACCCTACGAGGGCGGCAGCAG |
| | ManI catalytic | TCAAGGCCGCATTCCAGACGTCGTGGAACGCTTACCA |
| | domain | CCATTTTGCCTTTCCCCATGACGACCTCCACCCGGTCA |
| | | GCAACAGCTTTGATGATGAGAGAAACGGCTGGGGCTC |
| | | GTCGGCAATCGATGGCTTGGACACGGCTATCCTCATG |
| | | GGGGATGCCGACATTGTGAACACGATCCTTCAGTATG |
| | | TACCGCAGATCAACTTCACCACGACTGCGGTTGCCAA |
| | | CCAAGGCATCTCCGTGTTCGAGACCAACATTCGGTAC |
| | | CTCGGTGGCCTGCTTTCTGCCTATGACCTGTTGCGAGG |
| | | TCCTTTCAGCTCCTTGGCGACAAACCAGACCCTGGTAA |
| | | ACAGCCTTCTGAGGCAGGCTCAAACACTGGCCAACGG |
| | | CCTCAAGGTTGCGTTCACCACTCCCAGCGGTGTCCCGG |
| | | ACCCTACCGTCTTCTTCAACCCTACTGTCCGGAGAAGT |
| | | GGTGCATCTAGCAACAACGTCGCTGAAATTGGAAGCC |
| | | TGGTGCTCGAGTGGACACGGTTGAGCGACCTGACGGG |
| | | AAACCCGCAGTATGCCCAGCTTGCGCAGAAGGGCGAG |
| | | TCGTATCTCCTGAATCCAAAGGGAAGCCCGGAGGCAT |
| | | GGCCTGGCCTGATTGGAACGTTTGTCAGCACGAGCAA |
| | | CGGTACCTTTCAGGATAGCAGCGGCAGCTGGTCCGGC |
| | | CTCATGGACAGCTTCTACGAGTACCTGATCAAGATGT |
| | | ACCTGTACGACCCGGTTGCGTTTGCACACTACAAGGA |
| | | TCGCTGGGTCCTTGCTGCCGACTCGACCATTGCGCATC |
| | | TCGCCTCTCACCCGTCGACGCGCAAGGACTTGACCTTT |
| | | TTGTCTTCGTACAACGGACAGTCTACGTCGCCAAACTC |
| | | AGGACATTTGGCCAGTTTTGCCGGTGGCAACTTCATCT |
| | | TGGGAGGCATTCTCCTGAACGAGCAAAAGTACATTGA |
| | | CTTTGGAATCAAGCTTGCCAGCTCGTACTTTGCCACGT |
| | | ACAACCAGACGGCTTCTGGAATCGGCCCCGAAGGCTT |
| | | CGCGTGGGTGGACAGCGTGACGGGCGCCGGCGGCTCG |
| | | CCGCCCTCGTCCCAGTCCGGGTTCTACTCGTCGGCAGG |
| | | ATTCTGGGTGACGGCACCGTATTACATCCTGCGGCCG |
| | | GAGACGCTGGAGAGCTTGTACTACGCATACCGCGTCA |
| | | CGGGCGACTCCAAGTGGCAGGACCTGGCGTGGGAAGC |
| | | GTTCAGTGCCATTGAGGACGCATGCCGCGCCGGCAGC |
| | | GCGTACTCGTCCATCAACGACGTGACGCAGGCCAACG |
| | | GCGGGGGTGCCTCTGACGATATGGAGAGCTTCTGGTT |
| | | TGCCGAGGCGCTCAAGTATGCGTACCTGATCTTTGCG |
| | | GAGGAGTCGGATGTGCAGGTGCAGGCCAACGGCGGG |
| | | AACAAATTTGTCTTTAACACGGAGGCGCACCCCTTTA |
| | | GCATCCGTTCATCATCACGACGGGGCGGCCACCTTGC |
| | | TTAA |
| 84 | 5′ARG1 and | TACCAATTGCCAAATCAGGCAATTGTGAGACAGTGGT |
| | ORF | AAAAAAGATGCCTGCAAAGTTAGATTCACACAGTAAG |
| | | AGAGATCCTACTCATAAATGAGGCGCTTATTTAGTAG |
| | | CTAGTGATAGCCACTGCGGTTCTGCTTTATGCTATTTG |
| | | TTGTATGCCTTACTATCTTTGTTTGGCTCCTTTTTCTTG |
| | | ACGTTTTCCGTTGGAGGGACTCCCTATTCTGAGTCATG |
| | | AGCCGCACAGATTATCGCCCAAAATTGACAAAATCTT |
| | | CTGGCGAAAAAAGTATAAAAGGAGAAAAAAGCTCAC |
| | | CCTTTTCCAGCGTAGAAAGTATATATCAGTCATTGAAG |
| | | ACTATTATTTAAATAACACAATGTCTAAAGGAAAAGT |
| | | TTGTTTGGCCTACTCCGGTGGTTTGGATACCTCCATCA |
| | | TCCTAGCTTGGTTGTTGGAGCAGGGATACGAAGTCGT |
| | | TGCCTTTTTAGCCAACATTGGTCAAGAGGAAGACTTTG |
| | | AGGCTGCTAGAGAGAAAGCTCTGAAGATCGGTGCTAC |
| | | CAAGTTTATCGTCAGTGACGTTAGGAAGGAATTTGTTG |
| | | AGGAAGTTTTGTTCCCAGCAGTCCAAGTTAACGCTATC |
| | | TACGAGAACGTCTACTTACTGGGTACCTCTTTGGCCAG |
| | | ACCAGTCATTGCCAAGGCCCAAATAGAGGTTGCTGAA |
| | | CAAGAAGGTTGTTTTGCTGTTGCCCACGGTTGTACCGG |
| | | AAAGGGTAACGATCAGGTTAGATTTGAGCTTTCCTTTT |
| | | ATGCTCTGAAGCCTGACGTTGTCTGTATCGCCCCATGG |
| | | AGAGACCCAGAATTCTTCGAAAGATTCGCTGGTAGAA |
| | | ATGACTTGCTGAATTACGCTGCTGAGAAGGATATTCC |
| | | AGTTGCTCAGACTAAAGCCAAGCCATGGTCTACTGAT |
| | | GAGAACATGGCTCACATCTCCTTCGAGGCTGGTATTCT |
| | | AGAAGATCCAAACACTACTCCTCCAAAGGACATGTGG |
| | | AAGCTCACTGTTGACCCAGAAGATGCACCAGACAAGC |
| | | CAGAGTTCTTTGACGTCCACTTTGAGAAGGGTAAGCC |
| | | AGTTAAATTAGTTCTCGAGAACAAAACTGAGGTCACC |
| | | GATCCGGTTGAGATCTTTTTGACTGCTAACGCCATTGC |
| | | TAGAAGAAACGGTGTTGGTAGAATTGACATTGTCGAG |
| | | AACAGATTCATCGGAATCAAGTCCAGAGGTTGTTATG |
| | | AAACTCCAGGTTTGACTCTACTGAGAACCACTCACAT |
| | | CGACTTGGAAGGTCTTACCGTTGACCGTGAAGTTAGA |
| | | TCGATCAGAGACACTTTTGTTACCCCAACCTACTCTAA |
| | | GTTGTTATACAACGGGTTGTACTTTACCCCAGAAGGTG |
| | | AGTACGTCAGAACTATGATTCAGCCTTCTCAAAACAC |
| | | CGTCAACGGTGTTGTTAGAGCCAAGGCCTACAAAGGT |
| | | AATGTGTATAACCTAGGAAGATACTCTGAAACCGAGA |
| | | AATTGTACGATGCTACCGAATCTTCCATGGATGAGTTG |
| | | ACCGGATTCCACCCTCAAGAAGCTGGAGGATTTATCA |
| | | CAACACAAGCCATCAGAATCAAGAAGTACGGAGAAA |
| | | GTGTCAGAGAGAAGGGAAAGTTTTTGGGACTTTAACT |
| | | CAAGTAAAAGGATAGTTGTACAATTATATATACGAAG |
| | | AATAAATCATTACAAAAAGTATTCGTTTCTTTGATTCT |
| | | TAACAGGATTCATTTTCTGGGTGTCATCAGGTACAGCG |
| | | CTGAATATCTTGAAGTTAACATCGAGCTCATCATCGAC |
| | | GTTCATCACACTAGCCACGTTTCCGCAACGGTAG |
| 85 | PpCITI TT | CCGGCCATTTAAATATGTGACGACTGGGTGATCCGGG |
| | | TTAGTGAGTTGTTCTCCCATCTGTATATTTTTCATTTAC |
| | | GATGAATACGAAATGAGTATTAAGAAATCAGGCGTAG |
| | | CAATATGGGCAGTGTTCAGTCCTGTCATAGATGGCAA |
| | | GCACTGGCACATCCTTAATAGGTTAGAGAAAATCATT |
| | | GAATCATTTGGGTGGTGAAAAAAAATTGATGTAAACA |
| | | AGCCACCCACGCTGGGAGTCGAACCCAGAATCTTTTG |
| | | ATTAGAAGTCAAACGCGTTAACCATTACGCTACGCAG |
| | | GCATGTTTCACGTCCATTTTTGATTGCTTTCTATCATAA |
| | | TCTAAAGATGTGAACTCAATTAGTTGCAATTTGACCA |
| | | ATTCTTCCATTACAAGTCGTGCTTCCTCCGTTGATGCA |
| | | AC |
| 86 | Ashbya gossypii | GATCTGTTTAGCTTGCCTCGTCCCCGCCGGGTCACCCG |
| | TEF1 promoter | GCCAGCGACATGGAGGCCCAGAATACCCTCCTTGACA |
| | | GTCTTGACGTGCGCAGCTCAGGGGCATGATGTGACTG |
| | | TCGCCCGTACATTTAGCCCATACATCCCCATGTATAAT |
| | | CATTTGCATCCATACATTTTGATGGCCGCACGGCGCGA |
| | | AGCAAAAATTACGGCTCCTCGCTGCAGACCTGCGAGC |
| | | AGGGAAACGCTCCCCTCACAGACGCGTTGAATTGTCC |
| | | CCACGCCGCGCCCCTGTAGAGAAATATAAAAGGTTAG |
| | | GATTTGCCACTGAGGTTCTTCTTTCATATACTTCCTTTT |
| | | AAAATCTTGCTAGGATACAGTTCTCACATCACATCCG |
| | | AACATAAACAACC |
| 87 | Ashbya gossypii | TAATCAGTACTGACAATAAAAAGATTCTTGTTTTCAAG |
| | TEF1 | AACTTGTCATTTGTATAGTTTTTTTATATTGTAGTTGTT |
| | termination | CTATTTTAATCAAATGTTAGCGTGATTTATATTTTTTTT |
| | sequence | CGCCTCGACATCATCTGCCCAGATGCGAAGTTAAGTG |
| | | CGCAGAAAGTAATATCATGCGTCAATCGTATGTGAAT |
| | | GCTGGTCGCTATACTGCTGTCGATTCGATACTAACGCC |
| | | GCCATCCAGTGTCGAAAAC |
| 88 | Alpha amylase | MVAWWSLFLY GLQVAAPALA |
| | signal sequence | |
| | (from | |
| | Aspergillus | |
| | niger α-amylase) | |
| 89 | Sequence of the | AAATGCGTACCTCTTCTACGAGATTCAAGCGAATGAG |
| | PpPMA1 | AATAATGTAATATGCAAGATCAGAAAGAATGAAAGG |
| | promoter: | AGTTGAAAAAAAAAACCGTTGCGTTTTGACCTTGAAT |
| | | GGGGTGGAGGTTTCCATTCAAAGTAAAGCCTGTGTCT |
| | | TGGTATTTTCGGCGGCACAAGAAATCGTAATTTTCATC |
| | | TTCTAAACGATGAAGATCGCAGCCCAACCTGTATGTA |
| | | GTTAACCGGTCGGAATTATAAGAAAGATTTTCGATCA |
| | | ACAAACCCTAGCAAATAGAAAGCAGGGTTACAACTTT |
| | | AAACCGAAGTCACAAACGATAAACCACTCAGCTCCCA |
| | | CCCAAATTCATTCCCACTAGCAGAAAGGAATTATTTA |
| | | ATCCCTCAGGAAACCTCGATGATTCTCCCGTTCTTCCA |
| | | TGGGCGGGTATCGCAAAATGAGGAATTTTTCAAATTT |
| | | CTCTATTGTCAAGACTGTTTATTATCTAAGAAATAGCC |
| | | CAATCCGAAGCTCAGTTTTGAAAAAATCACTTCCGCG |
| | | TTTCTTTTTTACAGCCCGATGAATATCCAAATTTGGAA |
| | | TATGGATTACTCTATCGGGACTGCAGATAATATGACA |
| | | ACAACGCAGATTACATTTTAGGTAAGGCATAAACACC |
| | | AGCCAGAAATGAAACGCCCACTAGCCATGGTCGAATA |
| | | GTCCAATGAATTCAGATAGCTATGGTCTAAAAGCTGA |
| | | TGTTTTTTATTGGGTAATGGCGAAGAGTCCAGTACGAC |
| | | TTCCAGCAGAGCTGAGATGGCCATTTTTGGGGGTATT |
| | | AGTAACTTTTTGAGCTCTTTTCACTTCGATGAAGTGTC |
| | | CCATTCGGGATATAATCGGATCGCGTCGTTTTCTCGAA |
| | | AATACAGCTTAGCGTCGTCCGCTTGTTGTAAAAGCAG |
| | | CACCACATTCCTAATCTCTTATATAAACAAAACAACCC |
| | | AAATTATCAGTGCTGTTTTCCCACCAGATATAAGTTTC |
| | | TTTTCTCTTCCGCTTTTTGATTTTTTATCTCTTTCCTTTA |
| | | AAAACTTCTTTACCTTAAAGGGCGGCC |
| 90 | Sequence of the | GAAGGGCCATCGAATTGTCATCGTCTCCTCAGGTGCC |
| | 5′-region that | ATCGCTGTGGGCATGAAGAGAGTCAACATGAAGCGGA |
| | was used to | AACCAAAAAAGTTACAGCAAGTGCAGGCATTGGCTGC |
| | knock into the | TATAGGACAAGGCCGTTTGATAGGACTTTGGGACGAC |
| | PpPRO1 locus: | CTTTTCCGTCAGTTGAATCAGCCTATTGCGCAGATTTT |
| | | ACTGACTAGAACGGATTTGGTCGATTACACCCAGTTT |
| | | AAGAACGCTGAAAATACATTGGAACAGCTTATTAAAA |
| | | TGGGTATTATTCCTATTGTCAATGAGAATGACACCCTA |
| | | TCCATTCAAGAAATCAAATTTGGTGACAATGACACCT |
| | | TATCCGCCATAACAGCTGGTATGTGTCATGCAGACTA |
| | | CCTGTTTTTGGTGACTGATGTGGACTGTCTTTACACGG |
| | | ATAACCCTCGTACGAATCCGGACGCTGAGCCAATCGT |
| | | GTTAGTTAGAAATATGAGGAATCTAAACGTCAATACC |
| | | GAAAGTGGAGGTTCCGCCGTAGGAACAGGAGGAATG |
| | | ACAACTAAATTGATCGCAGCTGATTTGGGTGTATCTGC |
| | | AGGTGTTACAACGATTATTTGCAAAAGTGAACATCCC |
| | | GAGCAGATTTTGGACATTGTAGAGTACAGTATCCGTG |
| | | CTGATAGAGTCGAAAATGAGGCTAAATATCTGGTCAT |
| | | CAACGAAGAGGAAACTGTGGAACAATTTCAAGAGATC |
| | | AATCGGTCAGAACTGAGGGAGTTGAACAAGCTGGACA |
| | | TTCCTTTGCATACACGTTTCGTTGGCCACAGTTTTAAT |
| | | GCTGTTAATAACAAAGAGTTTTGGTTACTCCATGGACT |
| | | AAAGGCCAACGGAGCCATTATCATTGATCCAGGTTGT |
| | | TATAAGGCTATCACTAGAAAAAACAAAGCTGGTATTC |
| | | TTCCAGCTGGAATTATTTCCGTAGAGGGTAATTTCCAT |
| | | GAATACGAGTGTGTTGATGTTAAGGTAGGACTAAGAG |
| | | ATCCAGATGACCCACATTCACTAGACCCCAATGAAGA |
| | | ACTTTACGTCGTTGGCCGTGCCCGTTGTAATTACCCCA |
| | | GCAATCAAATCAACAAAATTAAGGGTCTACAAAGCTC |
| | | GCAGATCGAGCAGGTTCTAGGTTACGCTGACGGTGAG |
| | | TATGTTGTTCACAGGGACAACTTGGCTTTCCCAGTATT |
| | | TGCCGATCCAGAACTGTTGGATGTTGTTGAGAGTACC |
| | | CTGTCTGAACAGGAGAGAGAATCCAAACCAAATAAAT |
| | | AG |
| 91 | Sequence of the | AATTTCACATATGCTGCTTGATTATGTAATTATACCTT |
| | 3′-region that | GCGTTCGATGGCATCGATTTCCTCTTCTGTCAATCGCG |
| | was used to | CATCGCATTAAAAGTATACTTTTTTTTTTTTCCTATAGT |
| | knock into the | ACTATTCGCCTTATTATAAACTTTGCTAGTATGAGTTC |
| | PpPRO1 locus: | TACCCCCAAGAAAGAGCCTGATTTGACTCCTAAGAAG |
| | | AGTCAGCCTCCAAAGAATAGTCTCGGTGGGGGTAAAG |
| | | GCTTTAGTGAGGAGGGTTTCTCCCAAGGGGACTTCAG |
| | | CGCTAAGCATATACTAAATCGTCGCCCTAACACCGAA |
| | | GGCTCTTCTGTGGCTTCGAACGTCATCAGTTCGTCATC |
| | | ATTGCAAAGGTTACCATCCTCTGGATCTGGAAGCGTT |
| | | GCTGTGGGAAGTGTGTTGGGATCTTCGCCATTAACTCT |
| | | TTCTGGAGGGTTCCACGGGCTTGATCCAACCAAGAAT |
| | | AAAATAGACGTTCCAAAGTCGAAACAGTCAAGGAGA |
| | | CAAAGTGTTCTTTCTGACATGATTTCCACTTCTCATGC |
| | | AGCTAGAAATGATCACTCAGAGCAGCAGTTACAAACT |
| | | GGACAACAATCAGAACAAAAAGAAGAAGATGGTAGT |
| | | CGATCTTCTTTTTCTGTTTCTTCCCCCGCAAGAGATATC |
| | | CGGCACCCAGATGTACTGAAAACTGTCGAGAAACATC |
| | | TTGCCAATGACAGCGAGATCGACTCATCTTTACAACTT |
| | | CAAGGTGGAGATGTCACTAGAGGCATTTATCAATGGG |
| | | TAACTGGAGAAAGTAGTCAAAAAGATAACCCGCCTTT |
| | | GAAACGAGCAAATAGTTTTAATGATTTTTCTTCTGTGC |
| | | ATGGTGACGAGGTAGGCAAGGCAGATGCTGACCACG |
| | | ATCGTGAAAGCGTATTCGACGAGGATGATATCTCCAT |
| | | TGATGATATCAAAGTTCCGGGAGGGATGCGTCGAAGT |
| | | TTTTTATTACAAAAGCATAGAGACCAACAACTTTCTGG |
| | | ACTGAATAAAACGGCTCACCAACCAAAACAACTTACT |
| | | AAACCTAATTTCTTCACGAACAACTTTATAGAGTTTTT |
| | | GGCATTGTATGGGCATTTTGCAGGTGAAGATTTGGAG |
| | | GAAGACGAAGATGAAGATTTAGACAGTGGTTCCGAAT |
| | | CAGTCGCAGTCAGTGATAGTGAGGGAGAATTCAGTGA |
| | | GGCTGACAACAATTTGTTGTATGATGAAGAGTCTCTCC |
| | | TATTAGCACCTAGTACCTCCAACTATGCGAGATCAAG |
| | | AATAGGAAGTATTCGTACTCCTACTTATGGATCTTTCA |
| | | GTTCAAATGTTGGTTCTTCGTCTATTCATCAGCAGTTA |
| | | ATGAAAAGTCAAATCCCGAAGCTGAAGAAACGTGGA |
| | | CAGCACAAGCATAAAACACAATCAAAAATACGCTCGA |
| | | AGAAGCAAACTACCACCGTAAAAGCAGTGTTGCTGCT |
| | | ATTAAA |
| 92 | Sequence of the | GGTTTCTCAATTACTATATACTACTAACCATTTACCTG |
| | PpTRP2 gene | TAGCGTATTTCTTTTCCCTCTTCGCGAAAGCTCAAGGG |
| | integration | CATCTTCTTGACTCATGAAAAATATCTGGATTTCTTCT |
| | locus: | GACAGATCATCACCCTTGAGCCCAACTCTCTAGCCTAT |
| | | GAGTGTAAGTGATAGTCATCTTGCAACAGATTATTTTG |
| | | GAACGCAACTAACAAAGCAGATACACCCTTCAGCAGA |
| | | ATCCTTTCTGGATATTGTGAAGAATGATCGCCAAAGTC |
| | | ACAGTCCTGAGACAGTTCCTAATCTTTACCCCATTTAC |
| | | AAGTTCATCCAATCAGACTTCTTAACGCCTCATCTGGC |
| | | TTATATCAAGCTTACCAACAGTTCAGAAACTCCCAGTC |
| | | CAAGTTTCTTGCTTGAAAGTGCGAAGAATGGTGACAC |
| | | CGTTGACAGGTACACCTTTATGGGACATTCCCCCAGA |
| | | AAAATAATCAAGACTGGGCCTTTAGAGGGTGCTGAAG |
| | | TTGACCCCTTGGTGCTTCTGGAAAAAGAACTGAAGGG |
| | | CACCAGACAAGCGCAACTTCCTGGTATTCCTCGTCTAA |
| | | GTGGTGGTGCCATAGGATACATCTCGTACGATTGTATT |
| | | AAGTACTTTGAACCAAAAACTGAAAGAAAACTGAAAG |
| | | ATGTTTTGCAACTTCCGGAAGCAGCTTTGATGTTGTTC |
| | | GACACGATCGTGGCTTTTGACAATGTTTATCAAAGATT |
| | | CCAGGTAATTGGAAACGTTTCTCTATCCGTTGATGACT |
| | | CGGACGAAGCTATTCTTGAGAAATATTATAAGACAAG |
| | | AGAAGAAGTGGAAAAGATCAGTAAAGTGGTATTTGAC |
| | | AATAAAACTGTTCCCTACTATGAACAGAAAGATATTA |
| | | TTCAAGGCCAAACGTTCACCTCTAATATTGGTCAGGA |
| | | AGGGTATGAAAACCATGTTCGCAAGCTGAAAGAACAT |
| | | ATTCTGAAAGGAGACATCTTCCAAGCTGTTCCCTCTCA |
| | | AAGGGTAGCCAGGCCGACCTCATTGCACCCTTTCAAC |
| | | ATCTATCGTCATTTGAGAACTGTCAATCCTTCTCCATA |
| | | CATGTTCTATATTGACTATCTAGACTTCCAAGTTGTTG |
| | | GTGCTTCACCTGAATTACTAGTTAAATCCGACAACAA |
| | | CAACAAAATCATCACACATCCTATTGCTGGAACTCTTC |
| | | CCAGAGGTAAAACTATCGAAGAGGACGACAATTATGC |
| | | TAAGCAATTGAAGTCGTCTTTGAAAGACAGGGCCGAG |
| | | CACGTCATGCTGGTAGATTTGGCCAGAAATGATATTA |
| | | ACCGTGTGTGTGAGCCCACCAGTACCACGGTTGATCG |
| | | TTTATTGACTGTGGAGAGATTTTCTCATGTGATGCATC |
| | | TTGTGTCAGAAGTCAGTGGAACATTGAGACCAAACAA |
| | | GACTCGCTTCGATGCTTTCAGATCCATTTTCCCAGCAG |
| | | GAACCGTCTCCGGTGCTCCGAAGGTAAGAGCAATGCA |
| | | ACTCATAGGAGAATTGGAAGGAGAAAAGAGAGGTGT |
| | | TTATGCGGGGGCCGTAGGACACTGGTCGTACGATGGA |
| | | AAATCGATGGACACATGTATTGCCTTAAGAACAATGG |
| | | TCGTCAAGGACGGTGTCGCTTACCTTCAAGCCGGAGG |
| | | TGGAATTGTCTACGATTCTGACCCCTATGACGAGTACA |
| | | TCGAAACCATGAACAAAATGAGATCCAACAATAACAC |
| | | CATCTTGGAGGCTGAGAAAATCTGGACCGATAGGTTG |
| | | GCCAGAGACGAGAATCAAAGTGAATCCGAAGAAAAC |
| | | GATCAATGAACGGAGGACGTAAGTAGGAATTTATG |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A composition comprising Her2 antibody molecules with N-glycans, wherein less than 20 mole % of the N-glycans comprise a Man5 core structure, and the N-glycan G0+G1+G2 content of the Her2 antibody molecules is more than 75 mole %.

2. The composition of claim 1, wherein 15 mole % or less of the N-glycans comprise a Man5 core structure.

3. The composition of claim 1, wherein 10 mole % or less of the N-glycans comprise a Man5 core structure.

4. The composition of claim 1, wherein 6-9 mole % of the N-glycans comprise a Man5 core structure.

5. The composition of claim 1, wherein 5-12 mole % of the N-glycans comprise a Man5 core structure.

6. The composition of claim 1, wherein the N-glycan G0+G1+G2 content of the Her2 antibody molecules is 80 mole % or more.

7. The composition of claim 1, wherein 50-65 mole % of the N-glycan is G0, 5-25 mole % of the N-glycan is G1 and 1-10 mole % of the N-glycan is G2.

8. The composition of claim 1, wherein 50-61 mole % of the N-glycan is G0, 15-25 mole % of the N-glycan is G1 and 2-5 mole % of the N-glycan is G2.

9. The composition of claim 1, wherein 59-60 mole % of the N-glycan is G0, 21-23 mole % of the N-glycan is G1 and 2-3 mole % of the N-glycan is G2.

10. The composition of claim 1, wherein the N-glycans of the Her2 antibody molecules lack fucose.

11. The composition of claim 1, wherein the Her2 antibody molecules comprise hybrid N-glycans of 10 mole % or less.

12. The composition of claim 1, wherein the N-glycosylation site occupancy is 75-89 mole %.

13. The composition of claim 1, wherein the Her2 antibody molecules in the composition comprise O-mannose, wherein the occupancy of the O-mannose is 1-3 mol/antibody mol.

14. The composition of claim 13, wherein the occupancy of the O-mannose is 1 mol/antibody mol.

15. The composition of claim 13, wherein more than 99% of the O-mannose contains a single mannose at the O-glycosylation site.

16. The composition of claim 1, wherein the Her2 antibody has a light chain amino acid sequence according to SEQ ID NO: 18 and a heavy chain amino acid sequence according to SEQ ID NO: 16 or SEQ ID NO: 20.

17. The composition of claim 1, wherein 5-12 mole % of the N-glycans comprise a Man5 core structure, the N-glycan G0+G1+G2 content of the Her2 antibody molecules is 77-86 mole %, the hybrid N-glycans is 9-11 mole %, the N-glycosylation site occupancy is 82-88 mole %, the N-glycans lack fucose and the Her2 antibody has a light chain amino acid sequence according to SEQ ID NO: 18 and a heavy chain amino acid sequence according to SEQ ID NO: 16 or 20.

18. The composition of claim 1, wherein 1-15 mole % of the N-glycans comprise a Man5 core structure, the N-glycan G0+G1+G2 content of the Her2 antibody molecules is 75-90 mole %, the hybrid N-glycans is 1-12 mole %, the N-glycosylation site occupancy is 80-90 mole %, and the Her2 antibody has a light chain amino acid sequence according to SEQ ID NO: 18 and a heavy chain amino acid sequence according to SEQ ID NO: 16 or 20.
